(19) United States
(12) Patent Application Publication
(10) Pub. No.: US 2023/0338564 A1
(43) Pub. Date: Oct. 26, 2023

(54) NEODEGRADER CONJUGATES

(71) Applicant: Orum Therapeutics, Inc., Yuseong-gu, Daejeon (FR)

(72) Inventors: Nathan FISHKIN, Cambridge, MA (US); Peter U. PARK, Cambridge, MA (US)

(73) Assignee: Orum Therapeutics, Inc., Yuseong-gu, Daejeon (FR)

(21) Appl. No.: 17/995,006

(22) PCT Filed: Mar. 31, 2021
(86) PCT No.: PCT/IB2021/052713

§ 371 Date: Sep. 29, 2022

Related U.S. Application Data
(60) Provisional application No. 63/067,967, filed on Aug. 20, 2020.
(60) Provisional application No. 63/003,179, filed on Mar. 31, 2020.

(51) Int. Cl. A61K 47/68; C07D 401/04; A61P 35/00

(52) U.S. Cl. CPC A61K 47/6849; A61K 47/6851; A61P 35/00; C07D 401/04

(57) ABSTRACT

The present disclosure provides neoDegraders and neoDegraders conjugated to binding moieties. Also provided are compositions comprising the conjugates. The compounds and compositions are useful for treating a disease or condition, e.g., cancer, in a subject in need thereof.

### FIELD

The present disclosure provides neoDegrader conjugates, wherein the neoDegrader is conjugated to a binding moiety. Also provided are compositions comprising the conjugates. The conjugates and compositions are useful for treating cancer in a subject in need thereof.

### BACKGROUND

Protein degradation has been validated as a therapeutic strategy by the effectiveness of immunomodulatory imide drugs. These compounds have the ability to bind to cereblon (CRBN) and promote recruitment and ubiquitination of substrate proteins mediated by CRL4^{CRBN} E3 ubiquitin ligase. It is thought that immunomodulatory imides act as “molecular glues,” filling the binding interface as a hydrophobic patch that reprograms protein interactions between the ligase and neosubstrates.

Despite the excitement for these compounds as novel treatments for cancer, thus far they have been limited to use in hematologic malignancies such as multiple myeloma and myelodysplastic syndrome (MDS). Expanding the library of compounds that can function by degrading other oncoproteins, many of which have been considered ‘undruggable,’ is an active area of drug development. Thus there is a continuing need for new compounds that can target these alternative oncoproteins and treat a wide array of cancers.

### SUMMARY

In certain aspects, the present disclosure provides a conjugate of formula (I):

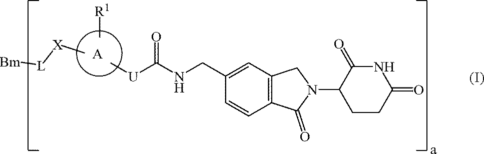

or a pharmaceutically acceptable salt thereof, wherein:
a is an integer from 1 to 10;
A is phenyl or a C₄-C₁₀cycloalkyl ring;
U is selected from NH and CF₂;
R¹ is independently selected from hydrogen and halo;
X is selected from —NR²—, ═C(CH₃)—, —Q—(CH₂)ₙ—, and —Q(CH₂)ₘQ′(CH₂)ₙ—; wherein
   Q and Q′ are each independently O, S, or N(R²)ᵥ;
   v is 1 or 2;
   each R² is independently hydrogen or C₁-C₆alkyl;
   n is an integer from 1 to 6;
   m is an integer from 2 to 6;
wherein the left side of each group is attached to L and the right side is attached to A;
   provided that when X is NH or —Q—(CH₂)ₙ—, R¹ is halo;
   L is a cleavable linker or non-cleavable linker; and
   Bm is a binding moiety that is capable of specifically binding to a protein.

In some aspects, the binding moiety is an antibody, antibody fragment, or an antigen-binding fragment.

In some aspects, the present disclosure provides a conjugate of formula (I), or a pharmaceutically acceptable salt thereof, wherein a is an integer from 2 to 8.

In certain aspects, the present disclosure provdes a conjugate of formula (I), or a pharmaceutically acceptable salt thereof, wherein L is a non-cleavable linker. In some aspects, L is selected from the group consisting of

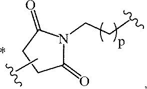

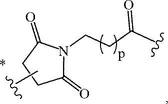

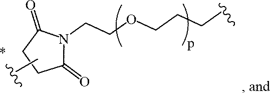

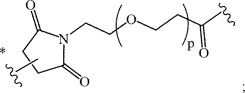

wherein:
p is an integer from 1 to 10;

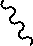

is the point of attachment to X; and

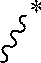

is the point of attachment to the binding moiety.

In some aspects, L is

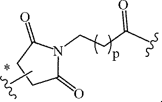

In some aspects, p is 5.

In certain aspects, the present disclosure provides a conjugate of formula (I), or a pharmaceutically acceptable salt thereof, wherein L is a cleavable linker. In some aspects, the cleavable linker is cleavable by a protease. In some aspects, L is selected from the group consisting of

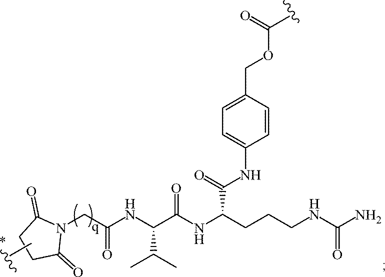

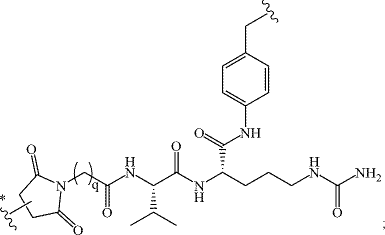

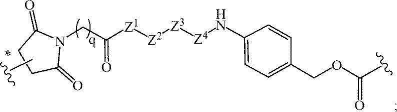

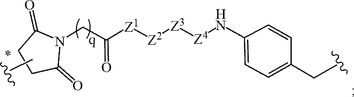

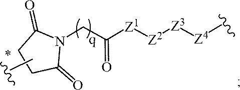

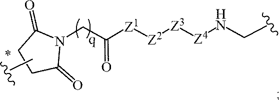

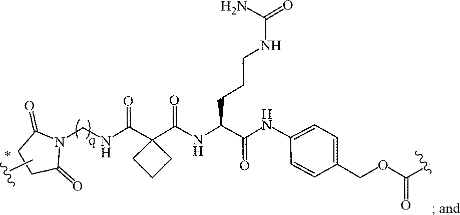

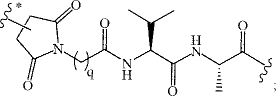

wherein:
q is an integer from 2 to 10;
Z¹, Z², Z³, and Z⁴ are each independently absent or a naturally-occurring amino acid residue in the L- or D-configuration, provided that at least two of Z¹, Z², Z³, and Z⁴ are amino acid residues;

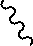

is the point of attachment to X; and

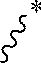

is the point of attachment to the binding moiety.

In some aspects, Z¹, Z², Z³, and Z⁴ are independently absent or selected from the group consisting of L-valine, D-valine, L-citrulline, D-citrulline, L-alanine, D-alanine, L-glutamine, D-glutaimine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, and Z⁴ are amino acid residues.

In some aspects, Z¹ is absent or glycine; Z² is absent or selected from the group consisting of L-glutamine, D-glutamine, L-glutamic acid, D-glutamic acid, L-aspartic acid, D-aspartic acid, L-alanine, D-alanine, and glycine; Z³ is selected from the group consisting of L-valine, D-valine, L-alanine, D-alanine, L-phenylalanine, D-phenylalanine, and glycine; and Z⁴ is selected from the group consisting of L-alanine, D-alanine, L-citrulline, D-citrulline, L-asparagine, D-asparagine, L-lysine, D-lysine, L-phenylalamine, D-phenylalanine, and glycine.

In certain aspects, L is

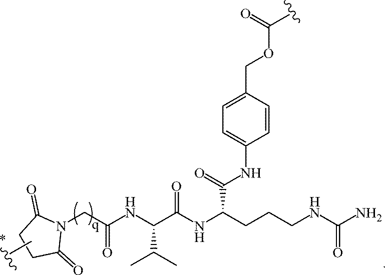

In some aspects, q is 5.

In certain aspects, the present disclosure provides a conjugate of formula (I), or a pharmaceutically acceptable salt thereof, wherein L is a bioreducible linker. In some aspects, L is selected from the group consisting of

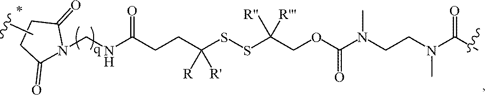

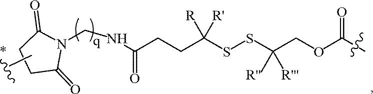

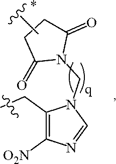

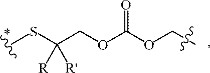

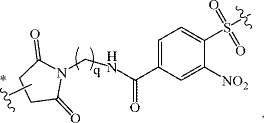

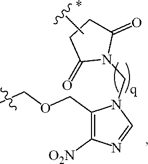

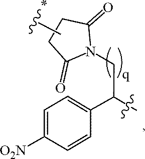

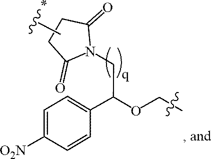

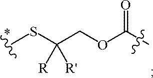

wherein:
q is an integer from 2 to 10;
R, R′, R″, and R‴ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring;

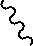

is the point of attachment to X; and

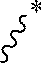

is the point of attachment to the binding moiety.

In certain aspects, the present disclosure provides a conjugate of formula (I), or a pharmaceutically acceptable salt thereof, wherein L is an acid cleavable linker. In some aspects, L is selected from the group consisting of

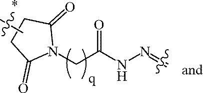

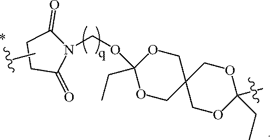

wherein:
q is an integer from 2 to 10;

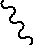

is the point of attachment to X; and

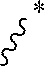

is the point of attachment to the binding moiety.

In certain aspects, the present disclosure provides a conjugate of formula (I), or a pharmaceutically acceptable salt thereof, wherein L is a click-to-release linker. In some aspects, L is selected from

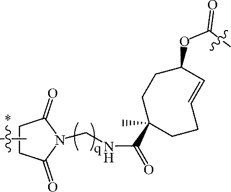

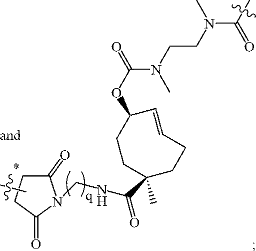

wherein:
q is an integer from 2 to 10;

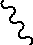

is the point of attachment to X; and

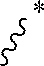

is the point of attachment to the binding moiety.

In certain aspects, the present disclosure provides a conjugate of formula (I), or a pharmaceutically acceptable salt thereof, wherein L is a pyrophosphatase cleavable linker. In some aspects, L is

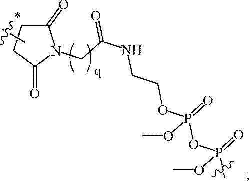

wherein:
q is an integer from 2 to 10;

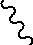

is the point of attachment to X; and

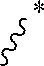

is the point of attachment to the binding moiety.

In certain aspects, the present disclosure provides a conjugate of formula (I), or a pharmaceutically acceptable salt thereof, wherein L is a beta-glucoronidase cleavable linker. In some embodiments, L is selected from

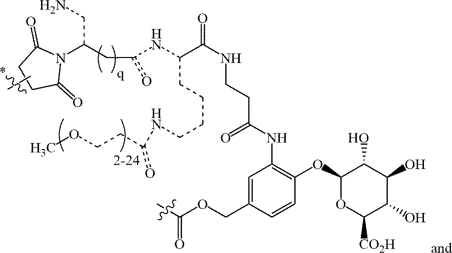

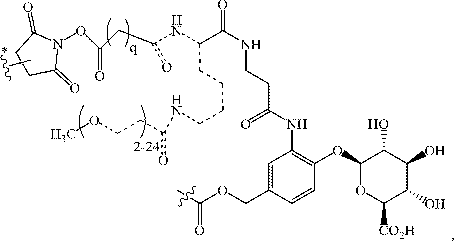

wherein:
q is an integer from 2 to 10;

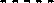

is absent or a bond;

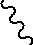

is the point of attachment to X; and

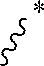

is the point of attachment to the binding moiety.

In certain aspects, the present disclosure provides a conjugate of formula (I), or a pharmaceutically acceptable salt thereof, wherein Bm is an antibody or antigen binding portion thereof. In some aspects, the protein that the binding moiety binds to is a surface antigen.

In some aspects, the surface antigen comprises 5T4, ACE, ADRB3, AKAP-4, ALK, Androgen receptor, AOC3, APP, Axin1, AXL, B7H3, B7-H4, BCL2, BCMA, bcr-abl, BORIS, BST2, C242, C4.4a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudin 3, Claudin 18.2, CLDN6, CLEC12A, CLL-1, cll3, c-MET, Crypto 1 growth factor, CS1, CTLA-4, CXCR2, CXORF61, Cyclin B1, CYP1B1, Cadherin-3, Cadherin-6, DLL3, E7, EDNRB, EFNA4, EGFR, EGFRvIII, ELF2M, EMR2, ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GloboH, GM3, GPC1, GPC2, GPC3, gplOO, GPNMB, GPR20, GPRC5D, GUCY2C, HAVCR1, HER2, HER3, HGF, HMI.24, HMWMAA, HPV E6, hTERT, human telomerase reverse transcriptase, ICAM, ICOS-L, IFN- α, IFN-γ, IGF-I receptor, IGLL1, IL-2 receptor, IL-4 receptor, IL-13Ra2, IL-1 1Ra, IL-1, IL-12, IL-23, IL-13, IL-22, IL-4, IL-5, IL-6, interferon receptor, integrins (including α₄, αᵥβ₃, αᵥβ₅, αᵥβ₆, α₁β₄, α₄β₁, α₄β₇, α₅β₁, α₆β₄, α_{11b}β₃ intergins), Integrin alphaV, intestinal carboxyl esterase, KIT, LAGE-la, LAIR1, LAMP-1, LCK, Legumain, LewisY, LFA-1(CD11a), L-selectin(CD62L), LILRA2, LIV-1, LMP2, LRRC15, LY6E, LY6K, LY75, MAD-CT-1, MAD-CT-2, MAGE A1, MelanA/MART1, Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2, OY-TES1, p53, p53 mutant, PANX3, PAP, PAX3, PAX5, p-CAD, PCTA- ⅟Galectin 8, PD-L1, PD-L2, PDGFR, PDGFR-beta, phosphatidylserine, PIK3CA, PLAC1, Polysialic acid, Prostase, prostatic carcinoma cell, prostein, Pseudomonas aeruginosa, rabies, survivin and telomerase, PRSS21, PSCA, PSMA, PTK7, RAGE-1, RANKL, Ras mutant, respiratory syncytial virus, Rhesus factor, RhoC, RON, ROR1, ROR2, RU1, RU2, sarcoma translocation breakpoints, SART3, SLAMF7, SLC44A4, sLe, SLITRK6, sperm protein 17, sphingosine-1-phosphate, SSEA-4, SSX2, STEAP1, TAG72, TARP, TCRβ, TEM1/CD248, TEM7R, tenascin C, TF, TGF-1, TGF- β2, TNF-α, TGS5, Tie 2, TIM-1, Tn Ag, TRAC, TRAIL-R1, TRAIL-R2, TROP-2, TRP-2, TRPV1, TSHR, tumor antigen CTAA16.88, tyrosinase, UPK2, VEGF, VEGFR1, VEGFR2, vimentin, WT1, XAGE1, or combinations thereof.

In certain aspects, the surface antigen comprises HER2, CD20, CD38, CD33, BCMA, CD138, EGFR, FGFR4, GD2, PDGFR, TEM1/CD248, TROP-2, or combinations thereof.

In some aspects, Bm is an antibody wherein the antibody is selected from the group consisting of rituximab, trastuzumab, gemtuzumab, pertuzumab, obinutuzumab, ofatumumab, olaratumab, ontuximab, isatuximab, Sacituzumab, U3-1784, daratumumab, STI-6129, lintuzumab, huMy9-6, balantamab, indatuximab, cetuximab, dinutuximab, anti-CD38 A2 antibody, HuAT13/5 antibody, alemtuzumab, ibritumomab, tositumomab, bevacizumab, panitumumab, tremelimumab, ticilimumab, catumaxomab, oregovomab, and veltuzumab.In some aspects, the antibody is rituximab, trastuzumab, pertuzumab, OR000213 (huMy9-6 IgG4 S228P), lintuzumab, or gemtuzumab.

In certain aspects, the present disclosure provides a conjugate of formula (I), or a pharmaceutically acceptable salt thereof, wherein:
A is phenyl;
U is NH;
R¹ is halo; and
X is —N(R²)ᵥ(CH₂)ₘO(CH₂)ₙ—; wherein:
   v is 1;
   m and n are 2; and
   R² is methyl.

In certain aspects, the present disclosure provides a conjugate of formula (I), or a pharmaceutically acceptable salt thereof, wherein:
A is phenyl;
U is NH;
R¹ is halo; and
X is —N(R²)ᵥ(CH₂)ₘO(CH₂)ₙ—; wherein:
   v is 2;
   m and n are 2; and
   each R² is methyl.

In certain aspects, the present disclosure provides a conjugate of formula (I), or a pharmaceutically acceptable salt thereof, wherein:
A is phenyl;
U is NH;
R¹ is halo; and
X is —O(CH₂)ₙ—; wherein:
   n is 2.

In certain aspects, the present disclosure provides a conjugate of formula (I), or a pharmaceutically acceptable salt thereof, wherein:
A is phenyl;
U is NH;
R¹ is halo; and
X is —S(CH₂)ₙ—; wherein:
   n is 2.

In certain aspects, the present disclosure provides a conjugate of formula (I), or a pharmaceutically acceptable salt thereof, wherein:
A is phenyl;
U is NH;
R¹ is hydrogen; and
X is —NR²—; wherein:
   R² is methyl.

In certain aspects, the present disclosure provides a conjugate of formula (I), or a pharmaceutically acceptable salt thereof, wherein:
A is phenyl;
U is NH;
R¹ is halo; and
X is —NR²—; wherein:
   R² is hydrogen.

In certain aspects, the present disclosure provides a conjugate of formula (I), or a pharmaceutically acceptable salt thereof, wherein:
A is phenyl;
U is NH;
R¹ is hydrogen; and
X is —C(CH₃)═.

In certain aspects, the present disclosure provides a conjugate of formula (I), or a pharmaceutically acceptable salt thereof, wherein:
A is a C₄-C₁₀cycloalkyl ring;
U is NH;
R¹ is hydrogen; and
X is —N(R²)(CH₂)ₘO(CH₂)ₙ—; wherein:
   n is 1;
   m is 2; and
   R² is methyl.

In certain aspects, the present disclosure provides a compound of formula (II):

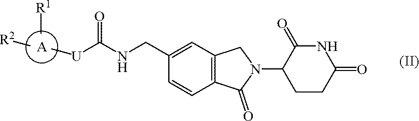

or a pharmaceutically acceptable salt thereof, wherein:
A is phenyl or a C₄-C₁₀cycloalkyl ring;
R¹ is independently selected from hydrogen and halo;
U is selected from NH and CF₂; and
R² is selected from —C(O)R³, —N(R⁴)₂, —(CH₂)ₙOH, —(CH₂)ₙSH, —(CH₂)ₙN(R⁴)₂, —(CH₂)ₙQ′(CH₂)ₘOH, —(CH₂)ₙQ′(CH₂)ₘSH, and —(CH₂)ₙQ′(CH₂)ₘN(R⁴)₂; wherein
   R³ is hydrogen or C₁-C₆alkyl;
   each R⁴ is independently hydrogen or C₁-C₆alkyl;
   Q′ is O, S, or NR⁴;
   n is 1-6; and
   m is 2-5;
provided that when R² is NH₂, —(CH₂)ₙNH₂, or —(CH₂)ₙOH then R¹ is halo.

In certain aspects, the present disclosure provides a compound of formula (III):

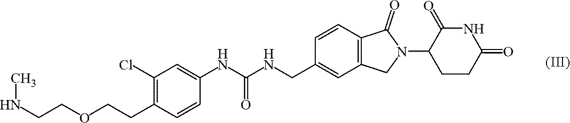 [SMILES: CNCCOCCc1ccc(NC(=O)NCc2ccc3c(c2)CN(C2CCC(=O)NC2=O)C3=O)cc1Cl]

or a pharmaceutically acceptable salt thereof.

In certain aspects, the present disclosure provides a compound of formula (IV):

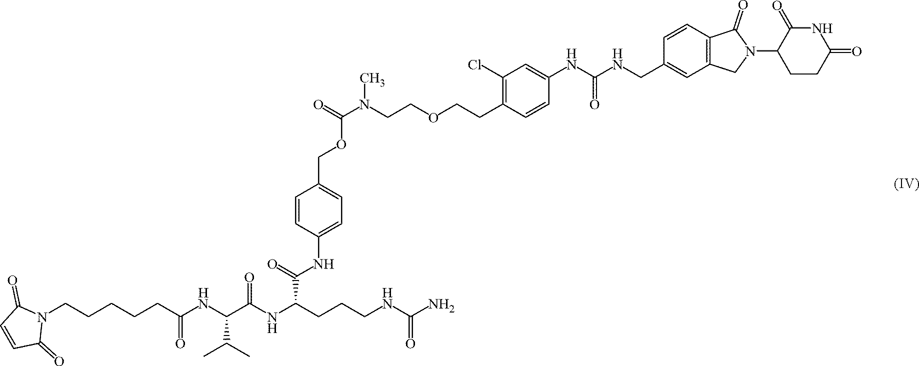 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CCOCCc2ccc(NC(=O)NCc3ccc4c(c3)CN(C3CCC(=O)NC3=O)C4=O)cc2Cl)cc1]

or a pharmaceutically acceptable salt thereof.

In some asepcts, the present disclosure provides a conjugate of formula (V):

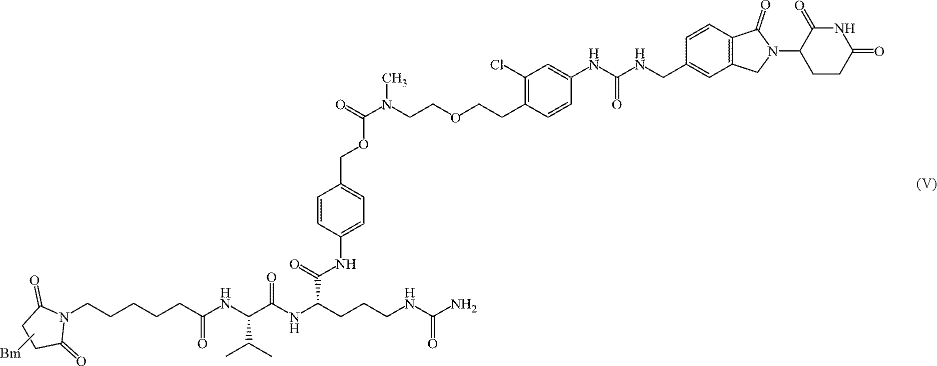

or a pharmaceutically acceptable salt thereof, wherein Bm is a binding moiety that specifically binds to a protein. In some aspects, Bm is an antibody or antigen binding portion thereof. In some aspects, the protein that the binding moiety specifically binds to is a surface antigen.

In some aspects, the surface antigen comprises 5T4, ACE, ADRB3, AKAP-4, ALK, Androgen receptor, AOC3, APP, Axin1, AXL, B7H3, B7-H4, BCL2, BCMA, bcr-abl, BORIS, BST2, C242, C4.4a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudin 3, Claudin 18.2, CLDN6, CLEC12A, CLL-1, cll3, c-MET, Crypto 1 growth factor, CS1, CTLA-4, CXCR2, CXORF61, Cyclin B1, CYP1B1, Cadherin-3, Cadherin-6, DLL3, E7, EDNRB, EFNA4, EGFR, EGFRvIII, ELF2M, EMR2, ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GloboH, GM3, GPC1, GPC2, GPC3, gplOO, GPNMB, GPR20, GPRC5D, GUCY2C, HAVCR1, HER2, HER3, HGF, HMI.24, HMWMAA, HPV E6, hTERT, human telomerase reverse transcriptase, ICAM, ICOS-L, IFN- α, IFN-γ, IGF-I receptor, IGLL1, IL-2 receptor, IL-4 receptor, IL-13Ra2, IL-1 1Ra, IL-1, IL-12, IL-23, IL-13, IL-22, IL-4, IL-5, IL-6, interferon receptor, integrins (including α₄, αᵥβ₃, αᵥβ₅, αᵥβ₆, α₁β₄, α₄β₁, α₄β₇, α₅β₁, α₆β₄, α_{IIb}β₃ intergins), Integrin alphaV, intestinal carboxyl esterase, KIT, LAGE-la, LAIR1, LAMP-1, LCK, Legumain, LewisY, LFA-1(CD11a), L-selectin(CD62L), LILRA2, LIV-1, LMP2, LRRC15, LY6E, LY6K, LY75, MAD-CT-1, MAD-CT-2, MAGE A1, MelanA/MART1, Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2, OY-TES1, p53, p53 mutant, PANX3, PAP, PAX3, PAX5, p-CAD, PCTA- ⅟Galectin 8, PD-L1, PD-L2, PDGFR, PDGFR-beta, phosphatidylserine, PIK3CA, PLAC1, Polysialic acid, Prostase, prostatic carcinoma cell, prostein, Pseudomonas aeruginosa, rabies, survivin and telomerase, PRSS21, PSCA, PSMA, PTK7, RAGE-1, RANKL, Ras mutant, respiratory syncytial virus, Rhesus factor, RhoC, RON, ROR1, ROR2, RU1, RU2, sarcoma translocation breakpoints, SART3, SLAMF7, SLC44A4, sLe, SLITRK6, sperm protein 17, sphingosine-1-phosphate, SSEA-4, SSX2, STEAP1, TAG72, TARP, TCRβ, TEM1/CD248, TEM7R, tenascin C, TF, TGF-1, TGF- β2, TNF-α, TGS5, Tie 2, TIM-1, Tn Ag, TRAC, TRAIL-R1, TRAIL-R2, TROP-2, TRP-2, TRPV1, TSHR, tumor antigen CTAA16.88, tyrosinase, UPK2, VEGF, VEGFR1, VEGFR2, vimentin, WT1, XAGE1, or combinations thereof.

In some aspects, the surface antigen comprises HER2, CD20, CD38, CD33, BCMA, CD138, EGFR, FGFR4, GD2, PDGFR, TEM1/CD248, TROP-2, or combinations thereof.

In some aspects, Bm is an antibody wherein the antibody comprises rituximab, trastuzumab, gemtuzumab, pertuzumab, obinutuzumab, ofatumumab, olaratumab, ontuximab, isatuximab, Sacituzumab, U3-1784, daratumumab, STI-6129, lintuzumab, huMy9-6, balantamab, indatuximab, cetuximab, dinutuximab, anti-CD38 A2 antibody, HuAT13/5 antibody, alemtuzumab, ibritumomab, tositumomab, bevacizumab, panitumumab, tremelimumab, ticilimumab, catumaxomab, oregovomab, or veltuzumab. In some aspects, the antibody is rituximab, trastuzumab, pertuzumab, OR000213, lintuzumab, or gemtuzumab.

In certain aspects, the present disclosure provides a pharmaceutical composition comprising a conjugate or compound of any one of the preceding aspects, or a pharmaceutically acceptable salt thereof, and one or more pharmaceutically acceptable carriers.

In certain asepcts, the present disclosure provides a method of treating cancer in a subject in need thereof, the method comprising administering to the subject a pharmaceutically acceptable amount of a conjugate, compound, or composition of any of the proceeding aspects, or a pharmaceutically acceptable salt thereof. In some aspects, the cancer is breast cancer, gastric cancer, lymphoma, acute myeloid leukemia, multiple myeloma, head and neck cancer, squamous cell carcinoma, and/or hepatocellular carcinoma.

In some aspects, the method further comprises administering to the subject a pharmaceutically acceptable amount of an additional agent prior to, after, or simultaneously with the conjugate or compound of any one the preceding aspects, or a pharmaceutically acceptable salt thereof. In some aspects, the additional agent is a cytotoxic agent or an immune response modifier. In some aspects, the immune response modifier is a checkpoint inhibitor. In some aspects, the checkpoint inhibitor comprises a PD-1 inhibitor, a PD-L1 inhibitor, a CTLA-4 inhibitor, a TIM3 inhibitor, and/or a LAG-3 inhibitor.

In certain aspects, the present disclosure provides a method of preparing a conjugate of formula (I), or a pharmaceutically acceptable salt thereof, the process comprising reacting a binding moiety with a compound of formula (I-1):

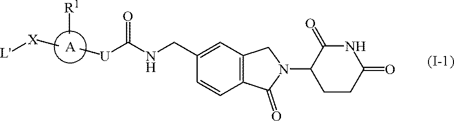

or a pharmaceutically acceptable salt thereof, wherein:
a is an integer from 1 to 10;
A is phenyl or a C₄-C₁₀cycloalkyl ring;
R¹ is independently selected from hydrogen and halo;
U is selected from NH and CF₂; and
X is selected from —N(R²)ᵥ—, ═C(CH₃)—, —Q—(CH₂)ₙ—, and —Q(CH₂)ₘQ′(CH₂)ₙ—; wherein
   v is 1 or 2;
   Q and Q′ are each independently O, S, or NR²;
   each R² is independently hydrogen or C₁-C₆alkyl;
   n is an integer from 1 to 6; and
   m is an integer from 2 to 6;
   wherein the left side of each group is attached to L′ and the right side is attached to A;
provided that when X is NH or —Q—(CH₂)ₙ—, R¹ is halo; and
L′ is a cleavable or non-cleavable linker precursor that conjugates to the binding moiety.

In some aspects, the metod further comprises reducing the binding moiety prior to reacting with the compound of formula (I-1).

In some aspects, a is an integer from 2 to 8.

In some aspects, L′ is a non-cleavable linker precursor. In some aspects, L′ is selected from the group consisting of

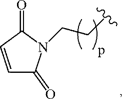

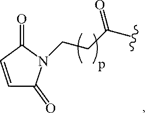

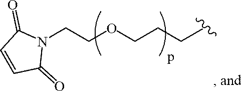

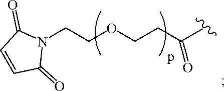

wherein:
p is an integer from 1 to 10; and

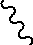

is the point of attachment to X.

In some asepcts, L′ is

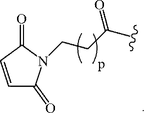

In some aspects, p is 5.

In certain aspects, L′ is a cleavable linker precursor. In some aspects, the cleavable linker precursor is cleavable by a protease. In some aspects, L′ is selected from the group consisting of

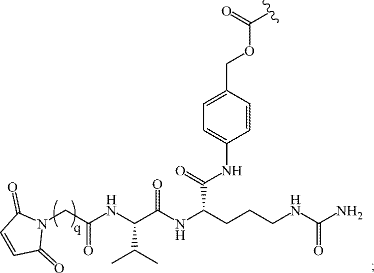

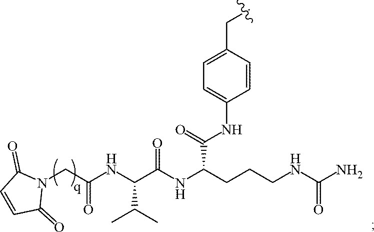

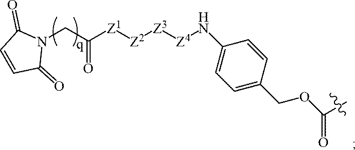

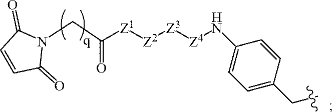

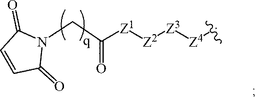

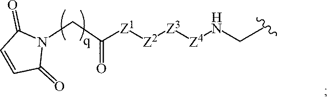

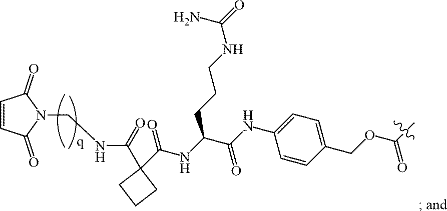

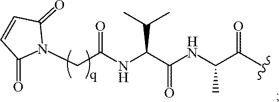

wherein:
q is an integer from 2 to 10;
Z¹, Z², Z³, and Z⁴ are each independently absent or a naturally-occurring amino acid residue in the L- or D-configuration, provided that at least two of Z¹, Z², Z³, and Z⁴ are amino acid residues; and

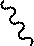

is the point of attachment to X.

In some aspects, Z¹, Z², Z³, and Z⁴ are independently absent selected from the group consisting of L-valine, D-valine, L-citrulline, D-citrulline, L-alanine, D-alanine, L-glutamine, D-glutaimine, L-glutamic acid, D-glutamic acid, L-aspartic acid, D-aspartic acid, L-asparagine, D-asparagine, L-phenylalanine, D-phenylalanine, L-lysine, D-lysine, and glycine, provided that at least two of Z¹, Z², Z³, and Z⁴ are amino acid residues.

In certain aspects, Z¹ is absent or glycine; Z² is absent or selected from the group consisting of L-glutamine, D-glutamine, L-glutamic acid, D-glutamic acid, L-aspartic acid, D-aspartic acid, L-alanine, D-alanine, and glycine; Z³ is selected from the group consisting of L-valine, D-valine, L-alanine, D-alanine, L-phenylalanine, D-phenylalanine, and glycine; and Z⁴ is selected from the group consisting of L-alanine, D-alanine, L-citrulline, D-citrulline, L-asparagine, D-asparagine, L-lysine, D-lysine, L-phenylalamine, D-phenylalanine, and glycine.

In some aspects, L′ is

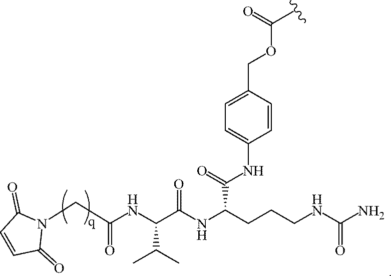

In some aspects, q is 5.

In certain aspects, L′ is a bioreducible linker precursor. In some aspects, L′ is selected from the group consisting of

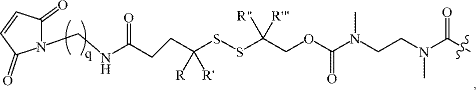

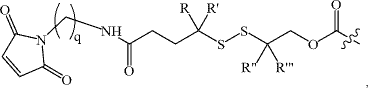

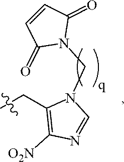

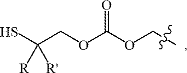

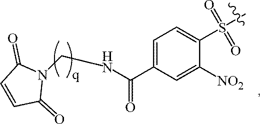

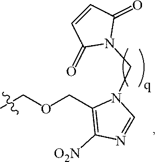

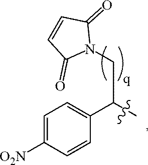

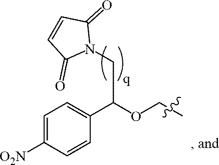

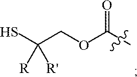

wherein:
q is an integer from 2 to 10;
R, R′, R″, and R‴ are each independently selected from hydrogen, C₁-C₆alkoxyC₁-C₆alkyl, (C₁-C₆)₂NC₁-C₆alkyl, and C₁-C₆alkyl, or two geminal R groups, together with the carbon atom to which they are attached, can form a cyclobutyl or cyclopropyl ring; and

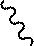

is the point of attachment to X.

In certain aspects, L′ is an acid cleavable linker precursor. In some asepcts, L′ is selected from the group consisting of

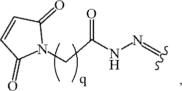

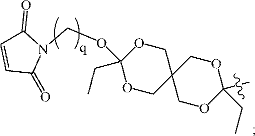

wherein:
q is an integer from 2 to 10; and

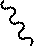

is the point of attachment to X.

In certain aspects, L′ is a click-to-release linker precursor. In some aspects, L′ is selected from

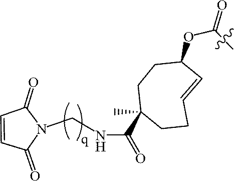

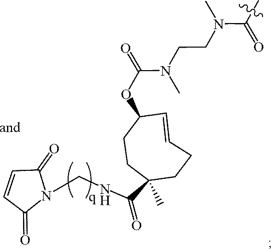

wherein:
q is an integer from 2 to 10; and

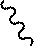

is the point of attachment to X.

In certain aspects, L′ is a pyrophosphatase cleavable linker precursor. In some aspects, L′ is

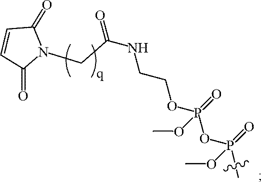

wherein:
q is an integer from 2 to 10;

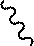

is the point of attachment to X.

In some asepcts, L′ is a beta-glucoronidase cleavable linker precursor. In certain aspects, L′ is selected from

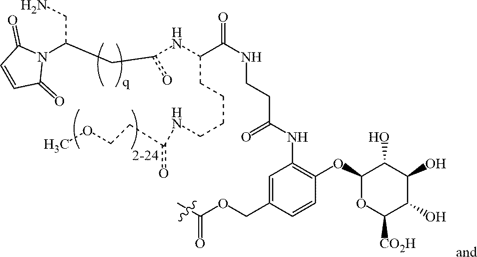

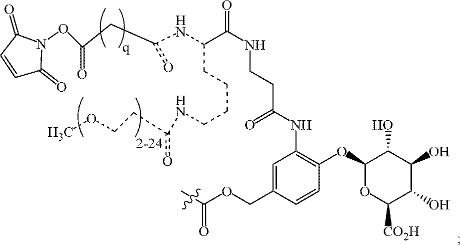

wherein:
q is an integer from 2 to 10;

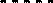

is absent or a bond; and

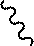

is the point of attachment to X.

In some aspects, the compound of formula (I-1) is reacted with a binding moiety, which comprises an antibody or an antigen binding portion thereof. In some aspects, the antibody or antigen binding portion thereof binds to a surface antigen.

In certain aspects, the surface antigen comprises 5T4, ACE, ADRB3, AKAP-4, ALK, Androgen receptor, AOC3, APP, Axin1, AXL, B7H3, B7-H4, BCL2, BCMA, bcr-abl, BORIS, BST2, C242, C4.4a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudin 3, Claudin 18.2, CLDN6, CLEC12A, CLL-1, cll3, c-MET, Crypto 1 growth factor, CS1, CTLA-4, CXCR2, CXORF61, Cyclin B1, CYP1B1, Cadherin-3, Cadherin-6, DLL3, E7, EDNRB, EFNA4, EGFR, EGFRvIII, ELF2M, EMR2, ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GloboH, GM3, GPC1, GPC2, GPC3, gplOO, GPNMB, GPR20, GPRC5D, GUCY2C, HAVCR1, HER2, HER3, HGF, HMI.24, HMWMAA, HPV E6, hTERT, human telomerase reverse transcriptase, ICAM, ICOS-L, IFN- α, IFN-γ, IGF-I receptor, IGLL1, IL-2 receptor, IL-4 receptor, IL-13Ra2, IL-1 1Ra, IL-1, IL-12, IL-23, IL-13, IL-22, IL-4, IL-5, IL-6, interferon receptor, integrins (including α₄, αᵥβ₃, αᵥβ₅, αᵥβ₆, α₁β₄, α₄β₁, α₄β₇, α₅β₁, α₆β₄, α_{IIb}β₃ intergins), Integrin alphaV, intestinal carboxyl esterase, KIT, LAGE-la, LAIR1, LAMP-1, LCK, Legumain, LewisY, LFA-1(CD11a), L-selectin(CD62L), LILRA2, LIV-1, LMP2, LRRC15, LY6E, LY6K, LY75, MAD-CT-1, MAD-CT-2, MAGE A1, MelanA/MART1, Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2, OY-TES1, p53, p53 mutant, PANX3, PAP, PAX3, PAX5, p-CAD, PCTA- ⅟Galectin 8, PD-L1, PD-L2, PDGFR, PDGFR-beta, phosphatidylserine, PIK3CA, PLAC1, Polysialic acid, Prostase, prostatic carcinoma cell, prostein, Pseudomonas aeruginosa, rabies, survivin and telomerase, PRSS21, PSCA, PSMA, PTK7, RAGE-1, RANKL, Ras mutant, respiratory syncytial virus, Rhesus factor, RhoC, RON, ROR1, ROR2, RU1, RU2, sarcoma translocation breakpoints, SART3, SLAMF7, SLC44A4, sLe, SLITRK6, sperm protein 17, sphingosine-1-phosphate, SSEA-4, SSX2, STEAP1, TAG72, TARP, TCRβ, TEM1/CD248, TEM7R, tenascin C, TF, TGF-1, TGF- β2, TNF-α, TGS5, Tie 2, TIM-1, Tn Ag, TRAC, TRAIL-R1, TRAIL-R2, TROP-2, TRP-2, TRPV1, TSHR, tumor antigen CTAA16.88, tyrosinase, UPK2, VEGF, VEGFR1, VEGFR2, vimentin, WT1, XAGE1, or combinations thereof.

In some asepcts, the surface antigen comprises HER2, CD20, CD38, CD33, BCMA, CD138, EGFR, FGFR4, GD2, PDGFR, TEM1/CD248, TROP-2 or combinations thereof.

In some aspects, Bm is an antibody wherein the antibody comprises rituximab, trastuzumab, gemtuzumab, pertuzumab, obinutuzumab, ofatumumab, olaratumab, ontuximab, isatuximab, Sacituzumab, U3-1784, daratumumab, STI-6129, lintuzumab, huMy9-6, balantamab, indatuximab, cetuximab, dinutuximab, anti-CD38 A2 antibody, HuAT13/5 antibody, alemtuzumab, ibritumomab, tositumomab, bevacizumab, panitumumab, tremelimumab, ticilimumab, catumaxomab, oregovomab, or veltuzumab. In certain aspects, the antibody is rituximab, trastuzumab, pertuzumab, OR000213, lintuzumab, or gemtuzumab .

In certain aspects the present disclosure provides a method for preparing a conjugate of formula (I) from a compound of formula (I-1), wherein:
A is phenyl;
U is NH;
R¹ is halo; and
X is —N(R²)ᵥ(CH₂)ₘO(CH₂)ₙ—; wherein:
   v is 1;
   m and n are 2; and
   R² is methyl.

In some aspects:
A is phenyl;
U is NH;
R¹ is halo; and
X is —N(R²)ᵥ(CH₂)ₘO(CH₂)ₙ—; wherein:
   v is 2;
   m and n are 2; and
   each R² is methyl.

In certain aspects:
A is phenyl;
U is NH;
R¹ is halo; and
X is —O(CH₂)ₙ—; wherein:
   n is 2.

In certain aspects:
A is phenyl;
U is NH;
R¹ is halo; and
X is —S(CH₂)ₙ—; wherein:
   n is 2.

In some aspects:
A is phenyl;
U is NH;
R¹ is hydrogen; and
X is —NR²—; wherein:
   R² is methyl.

In some aspects:
A is phenyl;
U is NH;
R¹ is halo; and
X is —NR²—; wherein:
   R² is hydrogen.

In certain aspects:
A is phenyl;
U is NH;
R¹ is hydrogen; and
X is —C(CH₃)═.

In some aspects:
A is a C₄-C₁₀cycloalkyl ring;
U is NH;
R¹ is hydrogen; and
X is —N(R²)(CH₂)ₘO(CH₂)ₙ—; wherein:
   n is 1;
   m is 2; and
   R² is methyl.

In some aspects, the compound of formula (I-1) is:

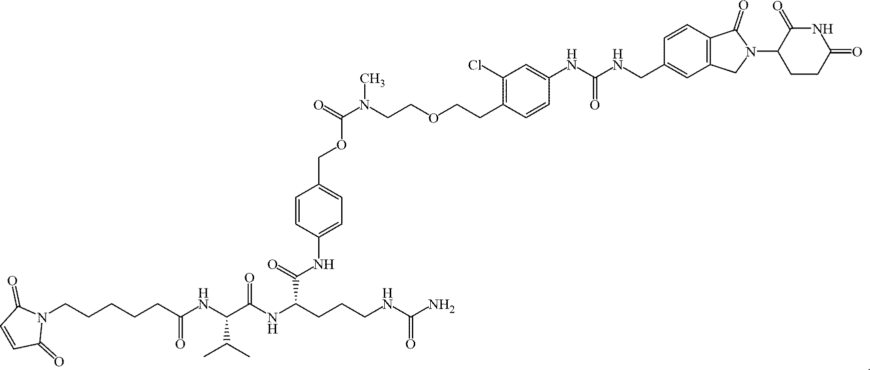

### BRIEF DESCRIPTION OF THE FIGURES

FIG. 1 depicts in vitro activity of representative neoDegrader conjugates against the BT-474 cell line. The X axis shows log antibody concentration (M). The Y axis shows % viability of the BT-474 cells when treated with trastuzumab - L-P1 (e.g., trastuzumab-Compound (Ia)) (triangle, solid line), trastuzumab alone (triangle, dotted line), Kadcyla (diamond), neoDegrader P1 alone (cross), and rituximab-L-P1 (e.g., rituximab - Compound (Ia)) (circle). L is a linker.

FIG. 2 depicts in vitro activity of representative neoDegrader conjugates against the BT-474 cell line. The X axis shows log antibody concentration (M). The Y axis shows % viability of the BT-474 cells when treated with pertuzumab -L-P1 (e.g., pertuzumab-Compound (Ia)) (triangle, solid line), pertuzumab alone (triangle, dotted line), Kadcyla (diamond), neoDegrader P1 alone (cross), and rituximab-L-P1 (e.g., rituximab- Compound (Ia)) (circle).

FIG. 3 depicts in vitro activity of representative neoDegrader conjugates against the BT-474 cancer cell line. The X axis shows log antibody concentration (M). The Y axis shows % viability of the BT-474 cells when treated with trastuzumab -L-P4 (e.g., trastuzumab-Compound (Ic)) (triangle, solid line), trastuzumab (triangle, dotted line), Kadcyla (diamond), neoDegrader P4 alone (cross), and rituximab-L-P4 (e.g., rituximab-Compound (Ic)) (circle).

FIG. 4 depicts in vitro activity of representative neoDegrader conjugates against the BT-474 cell line. The X axis shows log antibody concentration (M). The Y axis shows % viability of the BT-474 cells when treated with pertuzumab - L-P4 (e.g., pertuzumab-Compound (Ic)) (triangle, solid line), pertuzumab (triangle, dotted line), Kadcyla (diamond), neoDegrader P4 alone (cross), and rituximab-L-P4 (e.g., rituximab-Compound (Ic)) (circle).

FIG. 5 depicts in vitro activity of representative neoDegrader conjugates with varied drug:antibody ratios (DAR) against the BT-474 cell line. The X axis shows log antibody concentration (M). The Y axis shows % viability of the BT-474 cells when treated with trastuzumab - L-P1 (e.g., trastuzumab-Compound (Ia)) DAR 1.6 (upward triangle, solid line), trastuzumab - L-P3 (e.g., trastuzumab-Compound (Ib)) DAR 1.5 (downward triangle, solid line), trastuzumab - L-P4 (e.g., trastuzumab-Compound (Ic)) DAR 1.6 (circle, solid line), trastuzumab L-P1 (e.g, trastuzumab-Compound (Id)) DAR 1.6 (square, solid line), trastuzumabL-P1 (e.g, trastuzumab-Compound (Ia) DAR 8 (triangle, dotted line), trastuzumab - L-P4 (e.g., trastuzumab-Compound (Ic)) DAR 8 (darkened circle, dotted line), ENHERTU^{®} (diamond, dotted line), and Trastuzumab (circle, dotted line).

FIG. 6 depicts in vitro activity of representative neoDegrader against the BT-474 cell line. The X axis shows log antibody concentration (M). The Y axis shows % viability of the BT-474 cells when treated with pertuzumab -L-P1 (e.g., pertuzumab-Compound (Ia)) DAR 8 (upward triangle, solid line), pertuzumab - L-P4 (e.g., pertuzumab-Compound (Ic)) DAR 8 (downward triangle, solid line), and ENHERTU^{®} (diamond, dotted line).

FIG. 7 depicts in vitro activity of representative neoDegrader conjugates against the SK-BR-3 cell line. The X axis shows log antibody concentration (M). The Y axis shows % viability of the SK-BR-3 cells when treated with trastuzumab - L-P1 (e.g., trastuzumab-Compound (Ia) (triangle, solid line), trastuzumab (triangle, dotted line), Kadcyla (diamond), neoDegrader P1 alone (circle), and rituximab-L-P1 (e.g., rituximab-Compound (Ia)) (cross).

FIG. 8 depicts in vitro activity of representative neoDegrader conjugates against the SK-BR-3 cell line. The X axis shows log antibody concentration (M). The Y axis shows % viability of the SK-BR-3 cells when treated with pertuzumab-L-P1 (e.g., pertuzumab-Compound (Ia)) (triangle, solid line), pertuzumab a (triangle, dotted line), Kadcyla (diamond), neoDegrader P1 alone (circle), and rituximab-L-P1 (e.g., rituximab-Compound (Ia)) (cross).

FIG. 9 depicts in vitro activity of representative neoDegrader conjugates against the HL-60 cell line. The X axis shows log antibody concentration (M). The Y axis shows % viability of HL-60 cells when treated with OR000213-L-P1 (e.g., OR000213-Compound (Ia)) (triangle), MYLOTARG^{®} (diamond), and trastuzumab-L-P1 (e.g., trastuzumab-Compound (Ia)) (circle).

FIG. 10 depicts in vitro activity of representative neoDegrader conjugates against the HL-60 cell line. The X axis shows log antibody concentration (M). The Y axis shows % viability of HL-60 cells when treated with huMy9-6(IgG1)-L-P1 (e.g., huMy9-6(IgG1)-Compound (Ia)) DAR 8 (upward darkened triangle, solid line), huMy9-6(IgG1)-L-P1) (e.g., huMy9-6(IgG1)-Compound (Id)) DAR 8 (downward darkened triangle, solid line), lintuzumab IgG1-L-P1 (e.g., lintuzumab IgG1-Compound (Ia)) DAR 8 (upward open triangle, dotted line), lintuzumab IgG1-L-P1 (e.g., lintuzumab IgG1-Compound (Id)) DAR 8 (downward open triangle, dotted line), OR000213-L-P1 (e.g., OR000213-Compound (Ia)) DAR 8 (square), and riuximab-L-P4 (e.g., rituximab-Compound (Ic)) (circle, dotted line).

FIG. 11 depicts in vitro activity of conjugates of Compound (Ia) with varied drug:antibody ratios (DAR) against the HL60 cell line. The X axis shows log antibody concentration (M). The Y axis shows % viability of the HL-60 cells whent treated with huMy9-6 IgG1-L-P1 (e.g., huMy9-6 IgG1-Compound (Ia)) DAR 1.9 (upward triangle, solid line), huMy9-6 IgG1-L-P1 (e.g., huMy9-6 IgG1 - Compound (Ia)) DAR 3.9 (downward triangle, solid line), huMy9-6 IgG1-L-P1 (e.g., huMy9-6 IgG1 - Compound (Ia)) DAR 5.5 (diamond, solid line), huMy9-6 IgG1-L-P1 (e.g., huMy9-6 IgG1-Compound (Ia)) DAR 8 (square, solid line), and rituximab-L-P4 (e.g., rituximab - Compound (Ic)) (circle, dotted line).

FIG. 12 depicts in vitro activity of conjugates of Compound (Ia) with varied drug:antibody ratios (DAR) against the HL60 cell line. The X axis shows log antibody concentration (M). The Y axis shows % viability of the HL-60 cells whent treated with OR000213-L-P1 (e.g., OR000213-Compound (Ia)) DAR 1.2 (upward triangle, solid line), OR000213-L-P1 (e.g., OR000213 - Compound (Ia)) DAR 1.8 (downward triangle, solid line), OR000213- L-P1 (e.g., OR000213- Compound (Ia)) DAR 2.3 (diamond, solid line), OR000213-L-P1 (e.g., OR000213-Compound (Ia)) DAR 8 (square, solid line), and Rituximab-L-P4 (e.g., rituximab -Compound (Ic)) (triangle, dotted line).

FIG. 13 depicts in vitro activity of representative neoDegrader conjugates against the Ramos cell line. The X axis shows log antibody concentration (M), and the Y axis shows % viability of Ramos cells when treated with rituximab-L-P4 (e.g., rituximab-Compound (Ic)) (upward triangle, solid line), rituximab-L-P1 (e.g., rituximab-Compound (Ia)) (downward triangle, solid line), rituximab (triangle, dotted line), neoDegrader P1 alone (cross, dotted line), neoDegrader P4 alone (star, dotted line), and trastuzumab-L-P1 (e.g., trastuzumab - Compound (Ia)) (circle, dotted line).

FIG. 14 depicts in vitro activity of representative neoDegrader conjugates against the Daudi cell line. The X axis shows log antibody concentration (M), and the Y axis shows % viability of Daudi cells when treated with rituximab-L-P1 (e.g., rituximab-Compound (Ia)) (upward triangle, solid line), rituximab (triangle, dotted line), and trastuzumab-L-P1 (e.g., trastuzumab - Compound (Ia)) (circle, dotted line).

FIG. 15 depicts in vitro activity of representative neoDegrader conjugates against the Ramos cell line. The X axis shows log antibody concentration (M), and the Y axis shows % viability of Ramos cells when treated with rituximab-L-P4 (e.g., rituximab-Compound (Ic)) (upward triangle, solid line), rituximab-L-P1 (e.g., rituximab-Compound (Ia)) (downward triangle, solid line), and neoDegrader P1 alone.

FIG. 16 depicts in vitro of representative neoDegrader conjugates against the NCI-N87 cancer cell line. The X axis shows log antibody concentration (M). The Y axis shows % viability of NCI-N87 cells when treated with trastuzumab-L-P1 (e.g., trastuzumab - Compound (Ia)) (triangle, solid line), trastuzumab (triangle, dotted line), Kadcyla (diamond), neoDegrader P4 alone (cross), and rituximab-L-P1 (e.g., rituximab-Compound (Ia)) (circle).

FIG. 17 depicts in vitro activity of representative neoDegrader conjugates against the NCI-N87 cancer cell line. The X axis shows log antibody concentration (M). The Y axis shows % viability of NCI-N87 cells when treated with pertuzumab-L-P1 (e.g., pertuzumab - Compound (Ia)) (triangle, solid line), pertuzumab (triangle, dotted line), Kadcyla (diamond), neoDegrader P1 alone (cross), and rituximab-L-P1 (e.g., rituximab-Compound (Ia)) (circle).

FIG. 18 depicts in vitro activity of representative neoDegrader conjugates after 3-day incubation with human serum against the BT-474 cell line. The X axis shows log antibody concentration (M). The Y axis shows % viability of BT-474 cells when treated with trastuzumab-L-P1 (e.g., trastuzumab-Compound (Ia)) (human serum) (upward triangle, solid line), pertuzumab-L-P1 (e.g., pertuzumab-Compound (Ia)) (human serum) (downward triangle, solid line), OR000213-L-P1 (e.g., OR000213-Compound (Ia)) (human serum) (circle, solid line), human serum only (star), trastuzumab-L-P1 (e.g., trastuzumab - Compound (Ia)) (upward triangle, dotted line), pertuzumab-L-P1 (e.g., pertuzumab-Compound (Ia)) (downward triangle, dotted line), and OR000213-L-P1 (e.g., OR000213-Compound (Ia)) (circle, dotted line).

FIG. 19 depicts in vitro activity of representative neoDegrader conjugates after 3-day incubation with mouse serum against the BT-474 cell line. The X axis shows log antibody concentration (M). The Y axis shows % viability of BT-474 cells when treated with trastuzumab-L-P1 (e.g., trastuzumab-Compound (Ia)) (mouse serum) (upward triangle, solid line), pertuzumab-L-P1 (e.g., pertuzumab-Compound (Ia)) (mouse serum) (downward triangle, solid line), OR000213-L-P1 (e.g., OR000213-Compound (Ia)) (mouse serum) (circle, solid line), mouse serum only (star), trastuzumab-L-P1 (e.g., trastuzumab - Compound (Ia)) (upward triangle, dotted line), pertuzumab-L-P1 (e.g., pertuzumab-Compound (Ia)) (downward triangle, dotted line), and OR000213-L-P1 (e.g., OR000213-Compound (Ia)) (circle, dotted line).

FIG. 20 depicts in vivo activity of representative neoDegrader conjugates against BT-474 (Her2+) tumors in mice. The X axis shows the day after dosing. The Y axis shows the tumor volume (mm³) after dosing with vehicle (darkened circle), 5 mg/kg trastuzumab-L-P1 (e.g., trastuzumab - Compound (Ia)) (square), 5 mg/kg rituximab - L-P1 (e.g., rituximab-Compound (Ia)) (triangle), and 5 mg/kg pertuzumab-L-P1 (e.g., pertuzumab-Compound (Ia)) (open circle).

FIG. 21 depicts in vivo activity of representative neoDegrader conjugates against Daudi (CD20+) tumors. The X axis shows the day after dosing. The Y axis shows the tumor volume (mm³) after dosing with vehicle (darkened circle), 5 mg/kg trastuzumab-L-P1 (e.g., trastuzumab - Compound (Ia)) (square), 1 mg/kg rituximab -L-P1 (e.g., rituximab-Compound (Ia)) (triangle), and 5 mg/kg rituximab-L-P1 (e.g., rituximab-Compound (Ia)) (open circle).

FIG. 22 depicts in vivo activity of representative neoDegrader conjugates against HL-60 (CD33+) tumors. The X axis shows the day after dosing. The Y axis shows the tumor volume (mm³) after dosing with vehicle (darkened circle), 5 mg/kg trastuzumab-L-P1 (e.g., trastuzumab - Compound (Ia)) (square), 1 mg/kg OR000213-L-P1 (e.g., OR000213-Compound (Ia)) (triangle), and 5 mg/kg OR000213-L-P1 (e.g., OR000213-Compound (Ia)) (open circle).

FIG. 23 depicts in vitro activity of a neoDegrader conjugate against the HCC2157 cell line. The X axis shows log antibody concentration (M), and the Y axis shows % viability of HCC2157 cells when treated with sacituzumab-L-P1 (e.g., sacituzumab-Compound (Ia)) (line 1), sacituzumab alone (line 2), and neoDegrader P1 alone (line 3).

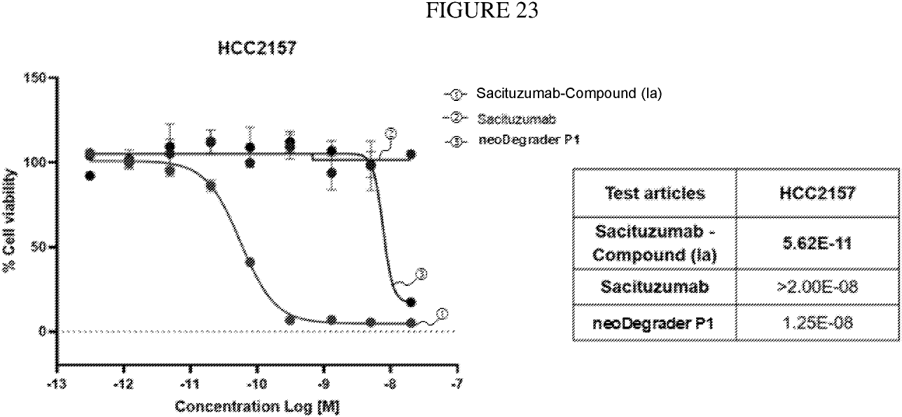

FIG. 24 depicts in vitro activity of a neoDegrader conjugate against the LP1 cell line. The X axis shows log antibody concentration (M), and the Y axis shows % viability of LP1 cells when treated with HuAT 13/5-L-P1 (e.g., HuAT 13/5-Compound (Ia)) (line 1), HuAT 13/5 alone (line 2), and neoDegrader P1 alone (line 3).

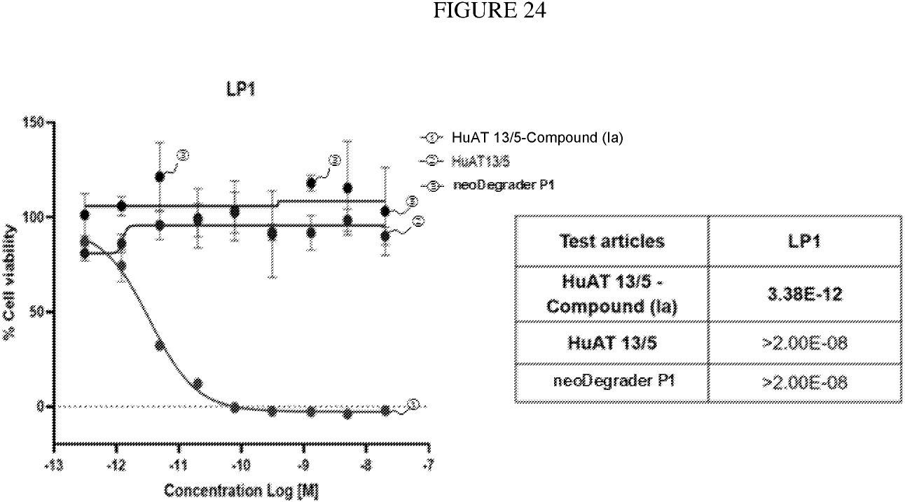

FIG. 25 depicts in vivo activity of of representative neoDegrader conjugates against NCI-H929 (CD38+) tumors. The X axis shows the day after dosing. The Y axis shows the tumor volume (mm3) after dosing with vehicle (circle), 5 mg/kg HuAT13/5-L-P1 (e.g., HuAT13/5 - Compound (Ia)) (square).

### DETAILED DESCRIPTION

The present disclosure is directed to a conjugate of formula (I):

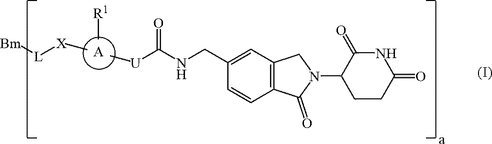

or a pharmaceutically acceptable salt thereof, wherein:
a is an integer from 1 to 10;
A is phenyl or a C₄-C₁₀cycloalkyl ring;
R¹ is independently selected from hydrogen and halo;
U is selected from NH and CF₂;
X is selected from —N(R²)ᵥ—, ═C(CH₃)—, —Q—(CH₂)ₙ—, and —Q(CH₂)ₘQ′(CH₂)ₙ—; wherein
   Q and Q′ are each independently O, S, or N(R²)ᵥ;
   v is 1 or 2;
   each R² is independently hydrogen or C₁-C₆alkyl;
   n is an integer from 1 to 6;
   m is an integer from 2 to 6;
wherein the left side of each group is attached to L and the right side is attached to A;
provided that when X is NH or —Q—(CH₂)ₙ—, R¹ is halo;
L is a cleavable linker or non-cleavable linker; and
Bm is a binding moiety that is capable of specifically binding to protein. In some aspects, the binding moiety is an antibody, antibody fragment, or an antigen-binding fragment.

The present disclosure also provides the compound above that is fused to the binding moiety, the composition comprising the compound or the conjugate, or the method of using or making the compound or the conjugate.

### I Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “DAR,” as used herein, refers to the drug antibody ratio of the conjugate, which is the average number of neoDegrader-linker comples linked to each antibody. In certain aspects, the DAR of the conjugates described herein is from 1 to 10. In some aspects, the DAR of the conjugates described herein is from 1 to 8. In some aspects, the DAR of the conjugates described herein is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.

The term “antibody,” as used herein, also refers to a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

The term “single domain antibody,” also known as a nanobody, is an antibody fragment consisting of a single monomeric variable antibody domain with a molecular weight of from about 12 kDa to about 15 kDa. Single body antibodies can be based on heavy chain variable domains or light chains. Examples of single domain antibodies include, but are not limited to, V_{H}H fragments and V_{NAR} fragments.

“Antibody fragments” comprise a portion of an intact antibody, generally the antigen binding or variable region thereof. Examples of antibody fragments include Fab, Fab′, F(ab′).sub.2, and Fv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

An “intact antibody” is one which comprises an antigen-binding variable region as well as a light chain constant domain (CL) and heavy chain constant domains, CH1, CH2 and CH3. The constant domains may be native sequence constant domains (e.g., human native sequence constant domains) or amino acid sequence variant thereof.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method, or may be made by recombinant DNA methods. The “monoclonal antibodies” may also be isolated from phage antibody libraries.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies of interest herein include “primatized” antibodies comprising variable domain antigen-binding sequences derived from a non-human primate (e.g., Old World Monkey, Ape etc.) and human constant region sequences.

Various methods have been employed to produce monoclonal antibodies (MAbs). Hybridoma technology, which refers to a cloned cell line that produces a single type of antibody, uses the cells of various species, including mice (murine), hamsters, rats, and humans. Another method to prepare MAbs uses genetic engineering including recombinant DNA techniques. Monoclonal antibodies made from these techniques include, among others, chimeric antibodies and humanized antibodies. A chimeric antibody combines DNA encoding regions from more than one type of species. For example, a chimeric antibody may derive the variable region from a mouse and the constant region from a human. A humanized antibody comes predominantly from a human, even though it contains nonhuman portions. Like a chimeric antibody, a humanized antibody may contain a completely human constant region. But unlike a chimeric antibody, the variable region may be partially derived from a human. The nonhuman, synthetic portions of a humanized antibody often come from CDRs in murine antibodies. In any event, these regions are crucial to allow the antibody to recognize and bind to a specific antigen. While useful for diagnostics and short-term therapies, murine antibodies cannot be administered to people long-term without increasing the risk of a deleterious immunogenic response. This response, called Human Anti-Mouse Antibody (HAMA), occurs when a human immune system recognizes the murine antibody as foreign and attacks it. A HAMA response can cause toxic shock or even death.

Chimeric and humanized antibodies reduce the likelihood of a HAMA response by minimizing the nonhuman portions of administered antibodies. Furthermore, chimeric and humanized antibodies can have the additional benefit of activating secondary human immune responses, such as antibody dependent cellular cytotoxicity.

The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g., B cell receptor; BCR), etc.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called .alpha., .delta., .epsilon., .gamma., and .mu., respectively. The subunit structures and three-dimensional configurations of different classes of immunoglobulins are well known.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The terms “administration,” “administering,” and grammatical variants thereof refer to introducing a composition, such as an EV (e.g., exosome) of the present disclosure, into a subject via a pharmaceutically acceptable route. The introduction of a composition, such as an EV (e.g., exosome) of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function. In some aspects of the present disclosure, the biologically active molecule is an antibody or a molecule comprising an antigen binding fragment thereof.

The terms “antibody-drug conjugate” and “ADC” are used interchangeably and refer to an antibody linked, e.g., covalently, to a therapeutic agent (sometimes referred to herein as agent, drug, or active pharmaceutical ingredient) or agents. In some aspects of the present disclosure, the biologically active molecule is an antibody-drug conjugate.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least about 70% identical, at least about 80% identical, at least about 90% identical, or at least about 95% identical to one another. In some aspects, two or more sequences are said to be “conserved” if they are at least about 30% identical, at least about 40% identical, at least about 50% identical, at least about 60% identical, at least about 70% identical, at least about 80% identical, at least about 90% identical, or at least about 95% identical to one another. Conservation of sequence can apply to the entire length of an polynucleotide or polypeptide or can apply to a portion, region or feature thereof.

As used herein, the terms “linking” and “conjugating” are used interchangeably an each refer to the covalent or non-covalent attachment of two or more moieties comprising a neodegrader and a binding moiety. In some aspects the linking or conjugating can comprise a linker.

The term “amino acid sequence variant” refers to polypeptides having amino acid sequences that differ to some extent from a native sequence polypeptide. Ordinarily, amino acid sequence variants will possess at least about 70% sequence identity with at least one receptor binding domain of a native antibody or with at least one ligand binding domain of a native receptor, and typically, they will be at least about 80%, more typically, at least about 90% homologous by sequence with such receptor or ligand binding domains. The amino acid sequence variants possess substitutions, deletions, and/or insertions at certain positions within the amino acid sequence of the native amino acid sequence. Amino acids are designated by the conventional names, one-letter and three-letter codes.

“Sequence identity” is defined as the percentage of residues in the amino acid sequence variant that are identical after aligning the sequences and introducing gaps, if necessary, to achieve the maximum percent sequence identity. Methods and computer programs for the alignment are well known in the art. One such computer program is “Align 2,” authored by Genentech, Inc., which was filed with user documentation in the United States Copyright Office, Washington, D.C. 20559, on Dec. 10, 1991.

The terms “Fc receptor” or “FcR” are used to describe a receptor that binds to the Fc region of an antibody. An exemplary FcR is a native sequence human FcR. Moreover, a FcR may be one which binds an IgG antibody (a gamma receptor) and includes receptors of the Fc.gamma.RI, Fc.gamma.RII, and Fc.gamma. RIII subclasses, including allelic variants and alternatively spliced forms of these receptors. Fc.gamma.RII receptors include Fc.gamma.RIIA (an “activating receptor”) and Fc.gamma.RIIB (an “inhibiting receptor”), which have similar amino acid sequences that differ primarily in the cytoplasmic domains thereof. Activating receptor Fc.gamma.RIIA contains an immunoreceptor tyrosine-based activation motif (ITAM) in its cytoplasmic domain. Inhibiting receptor Fc.gamma.RIIB contains an immunoreceptor tyrosine-based inhibition motif (ITIM) in its cytoplasmic domain. Other FcRs, including those to be identified in the future, are encompassed by the term “FcR” herein. The term also includes the neonatal receptor, FcRn, which is responsible for the transfer of maternal IgGs to the fetus.

“Complement dependent cytotoxicity” or “CDC” refers to the ability of a molecule to lyse a target in the presence of complement. The complement activation pathway is initiated by the binding of the first component of the complement system (C1q) to a molecule (e.g., an antibody) complexed with a cognate antigen. To assess complement activation, a CDC assay may be performed.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a .beta.-sheet configuration, connected by three hypervariable regions, which form loops connecting, and in some cases forming part of, the .beta.-sheet structure. The hypervariable regions in each chain are held together in close proximity by the FRs and, with the hypervariable regions from the other chain, contribute to the formation of the antigen-binding site of antibodies. The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (H3) in the heavy chain variable domain; Kabat et al supra) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain). “Framework Region” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (.kappa.) and lambda (.lamda.), based on the amino acid sequences of their constant domains.

“Single-chain Fv” or “scFv” antibody fragments comprise the VH and VL domains of antibody, wherein these domains are present in a single polypeptide chain. The Fv polypeptide may further comprise a polypeptide linker between the VH and VL domains which enables the scFv to form the desired structure for antigen binding.

The term “diabodies” refers to small antibody fragments with two antigen-binding sites, which fragments comprise a variable heavy domain (VH) connected to a variable light domain (VL) in the same polypeptide chain (VH-VL). By using a linker that is too short to allow pairing between the two domains on the same chain, the domains are forced to pair with the complementary domains of another chain and create two antigen-binding sites.

“Humanized” forms of non-human (e.g., rodent) antibodies are chimeric antibodies that contain minimal sequence derived from non-human immunoglobulin. Humanization is a method to transfer the murine antigen binding information to a non-immunogenic human antibody acceptor, and has resulted in many therapeutically useful drugs. The method of humanization generally begins by transferring all six murine complementarity determining regions (CDRs) onto a human antibody framework. These CDR-grafted antibodies generally do not retain their original affinity for antigen binding, and in fact, affinity is often severely impaired. Besides the CDRs, select non-human antibody framework residues must also be incorporated to maintain proper CDR conformation. The transfer of key mouse framework residues to the human acceptor in order to support the structural conformation of the grafted CDRs has been shown to restore antigen binding and affinity. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding nonhuman residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In certain aspects, the antibody will be purified (1) to greater than 95% by weight of antibody as determined by the Lowry method, or more than 99% by weight, (2) to a degree sufficient to obtain at least 15 residues of N-terminal or internal amino acid sequence by use of a gas phase protein sequencer, or (3) to homogeneity by SDS-PAGE under reducing or nonreducing conditions using Coomassie blue or silver stain. Isolated antibody includes the antibody in situ within recombinant cells since at least one component of the antibody’s natural environment will not be present. Ordinarily, however, isolated antibody will be prepared by at least one purification step.

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate’s body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell. As used herein, the term “T cell” and “T lymphocytes” are interchangeable and refer to any lymphocytes produced or processed by the thymus gland. In some aspects, a T cell is a CD4+ T cell. In some aspects, a T cell is a CD8+ T cell. In some aspects, a T cell is a NKT cell.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein.

The term “therapeutically effective amount” or “therapeutically effective dosage” refers to an amount of an agent (e.g., neoDegrader or neoDegrader conjugate disclosed herein) that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the composition can, for example, (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer.

In some aspects, a “therapeutically effective amount” is the amount of the neoDegrader or neoDegrader conjugate clinically proven to affect a significant decrease in cancer or slowing of progression (regression) of cancer, such as an advanced solid tumor. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the term “standard of care” refers to a treatment that is accepted by medical experts as a proper treatment for a certain type of disease and that is widely used by healthcare professionals. The term can be used interchangeable with any of the following terms: “best practice,” “standard medical care,” and “standard therapy.”

By way of example, an “anti-cancer agent” promotes cancer regression in a subject or prevents further tumor growth. In certain aspects, a therapeutically effective amount of the drug promotes cancer regression to the point of eliminating the cancer.

The terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

As used herein, the term “immune checkpoint inhibitor” refers to molecules that totally or partially reduce, inhibit, interfere with or modulate one or more checkpoint proteins. Checkpoint proteins regulate T-cell activation or function. Numerous checkpoint proteins are known, such as CTLA-4 and its ligands CD80 and CD86; and PD-1 with its ligands PD-L1 and PD-L2. Pardoll, D.M., Nat Rev Cancer 12(4):252-64 (2012). These proteins are responsible for costimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies.

The terms “treat” or “treatment” refer to both therapeutic treatment and prophylactic or preventative measures, wherein the object is to prevent or slow down (lessen) an undesired physiological change or disorder, such as the development or spread of cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

### II. NeoDegraders

The present disclosure provides neoDegraders of formula (II):

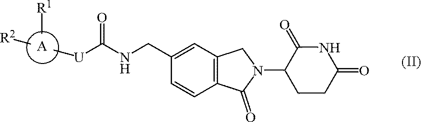

or pharmaceutically acceptable salts thereof, wherein:
A is phenyl or a C₄-C₁₀cycloalkyl ring;
U is selected from NH and CF₂;
R¹ is independently selected from hydrogen and halo;
R² is selected from —C(O)R³, —N(R⁴)₂, —(CH₂)ₙOH, —(CH₂)ₙSH, —(CH₂)ₙN(R⁴)₂, —(CH₂)ₙQ′(CH₂)ₘOH, —(CH₂)ₙQ′(CH₂)ₘSH, and —(CH₂)ₙQ′(CH₂)ₘN(R⁴)₂; wherein
   R³ is hydrogen or C₁-C₆alkyl;
   each R⁴ is independently hydrogen or C₁-C₆alkyl;
   Q′ is O, S, or NR⁴;
   n is 1-6; and
   m is 2-5;
provided that when R² is NH₂, —(CH₂)ₙNH₂, or —(CH₂)ₙOH then R¹ is halo.

In certain aspects, the present disclosure provides compounds of formula (II), or pharmaceutically acceptable salts thereof, wherein:
A is a phenyl ring or a C₄-C₁₀cycloalkyl ring;
U is NH;
R¹ is selected from hydrogen and halo;
R² is selected from —(CH₂)ₙQ′(CH₂)ₘN(R⁴)₂, —(CH₂)ₙOH, —(CH₂)ₙSH, —N(R⁴)₂, and -C(O)R³; wherein:
   m is 2;
   n is 2;
   Q′ is —O—;
   R³ is methyl; and
   each R⁴ is independently selected from hydrogen and methyl; provided that when R² is NH₂ or-(CH₂)ₙOH, then R¹ is halo.

As used herein, the term “C₁-C₆alkoxy,” as used herein, refers to a C₁-C₆alkyl group attached to the parent molecular moiety through an oxygen atom.

As used herein, the term “C₁-C₆alkoxyC₁-C₆alkyl” refers to a C₁-C₆alkoxy group attached to the parent molecular moiety through a C₁-C₆alkyl group.

As used herein, the term “C₁-C₆alkyl” refers to a group derived from a straight or branched chain saturated hydrocarbon containing from one to six carbon atoms.

As used herein, the term “C₄-C₁₀cycloalkyl” refers to a a saturated monocyclic, hydrocarbon ring system having four to ten carbon atoms and zero heteroatoms. Representative examples of cycloalkyl groups include, but are not limited to, cyclobutyl, cyclopentyl, and cyclohexyl. The cycloalkyl groups containing between seven and ten atoms may be monocyclic or fused, spirocyclic, or bridged bicyclic structures.

As used herein, the term “halo” refers to F, Cl, Br, or I.

In some aspects, the neoDegrader of formula (II) is a compound selected from the group consisting of:

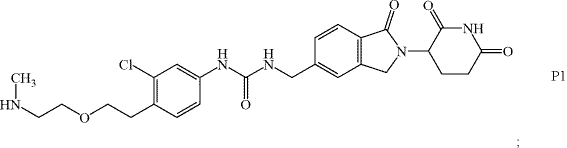

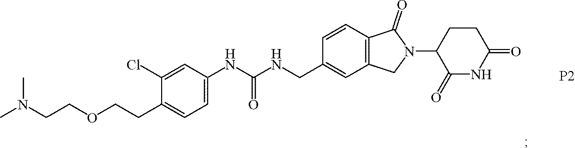

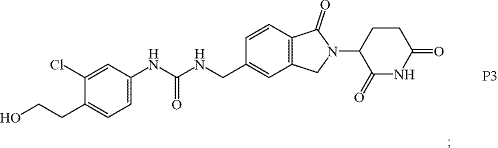

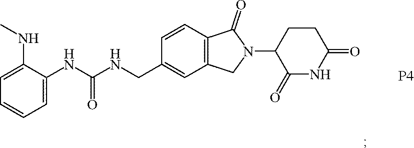

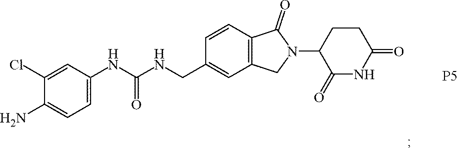

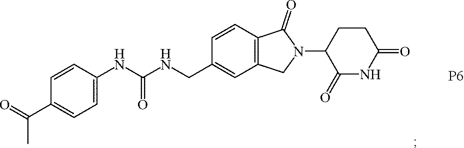

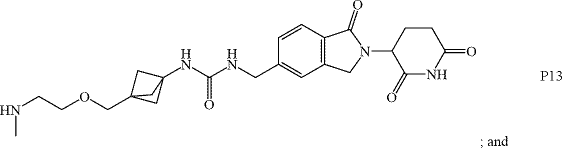

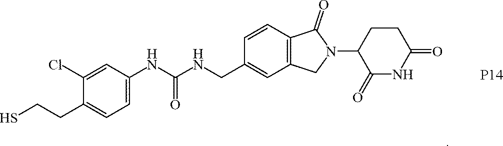

In some aspects, the neoDegrader of formula (II) is

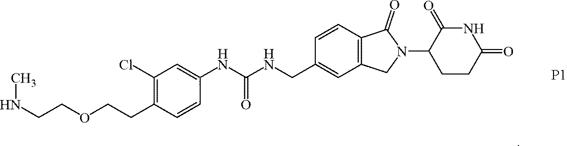

In some aspects, the neoDegrader of formula (II) is

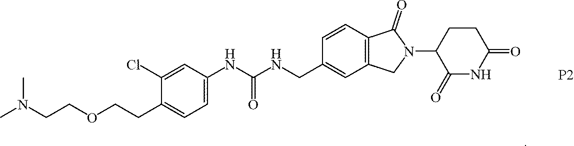

In some aspects, the neoDegrader of formula (II) is

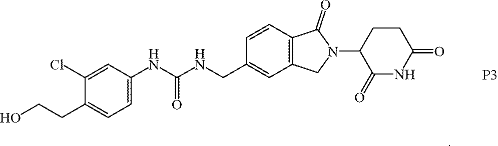

In some aspects, the neoDegrader of formula (II) is

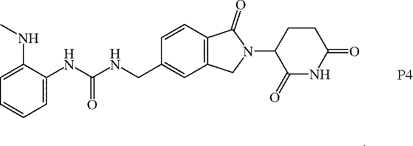

In some aspects, the neoDegrader of formula (II) is

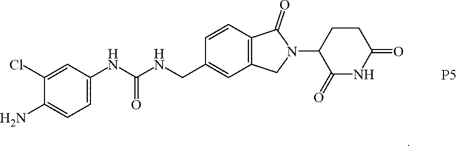

In some aspects, the neoDegrader of formula (II) is

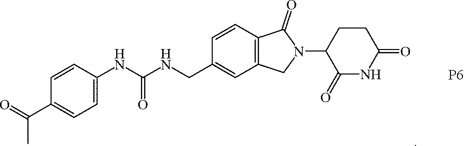

In some aspects, the neoDegrader of formula (II) is

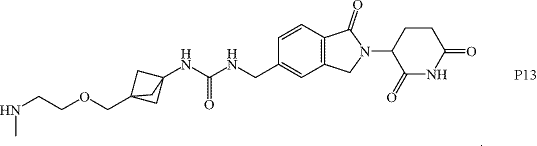

In some aspects, the neoDegrader of formula (II) is

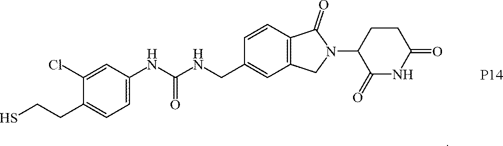

In some aspects, the present disclosure provides neoDegraders of formula (II), or pharmaceutically acceptable salts thereof, wherein A is phenyl; U is NH; R¹ is halo; and R² is —(CH₂)ₙQ′(CH₂)ₘN(R⁴)₂, wherein m and n are 2, Q′ is O, one R⁴ is hydrogen and the other is methyl.

In some aspects, the present disclosure provides neoDegraders of formula (II), wherein A is phenyl; U is NH; R¹ is halo; and R² is —(CH₂)ₙQ′(CH₂)ₘN(R⁴)₂, wherein m and n are 2, Q′ is O, and each R⁴ is methyl.

In some aspects, the present disclosure provides neoDegraders of formula (II), wherein A is phenyl; U is NH— R¹ is halo; and R² is — (CH₂)ₙOH, wherein n is 2.

In some aspects, the present disclosure provides neoDegraders of formula (II), wherein A is phenyl; U is NH— R¹ is halo; and R² is — (CH₂)ₙSH, wherein n is 2.

In some aspects, the present disclosure provides neoDegraders of formula (II), wherein A is phenyl; U is NH; R¹ is hydrogen; and R² is —N(R⁴)₂, wherein one R⁴ is hydrogen and the other is methyl.

In some aspects, the present disclosure provides neoDegraders of formula (II), wherein A is phenyl; U is NH; R¹ is halo; and R² is —N(R⁴)₂, wherein each R⁴ is hydrogen. In some aspects, the present disclosure provides neoDegraders of formula (II), wherein A is phenyl; R¹ is hydrogen; and R² —C(O)R³, wherein R³ is methyl.

In some aspects, the present disclosure provides neoDegraders of formula (II), wherein A is a C₄-C₁₀cycloalkyl ring; U is NH; R¹ is hydrogen; and R² is —(CH₂)ₙQ′(CH₂)ₘN(R⁴)₂, wherein m and n are 2, Q′ is O, one R⁴ is hydrogen and the other is methyl.

### III. NeoDegrader Conjugates

The present disclosure provides conjugates of one or more neoDegraders disclosed herein and a binding moiety. These conjugates can degrade proteins by binding to cereblon (CRBN), promoting recruitment and ubiquitination of substrate proteins mediated by CRL4^{CRBN} E3 ubiquitin ligase. These agents act as “molecular glues,” filling the binding interface as a hydrophobic patch that reprograms protein interactions between the ligase and neosubstrates.

In some aspects, the present disclosure provides a compound of formula (I),

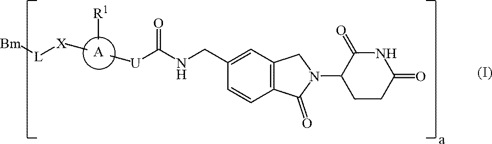

or a pharmaceutically acceptable salt thereof, wherein:
a is an integer from 1 to 10;
A is phenyl or a C₄-C₁₀cycloalkyl ring;
R¹ is selected from hydrogen and halo;
U is selected from NH and CF₂;
X is selected from —NR²—, ═C(CH₃)—, —Q—(CH₂)ₙ—, and —Q(CH₂)ₘQ′(CH₂)ₙ—; wherein:
   Q and Q′ are each independently O, S, or NR²;
   R² is hydrogen or C₁-C₆alkyl;
   n is an integer from 1 to 6;
   m is an integer from 2 to 6;
wherein the left side of each group is attached to L and the right side is attached to A;
provided that when X is NH or —Q—(CH₂)ₙ—, R¹ is halo;
L is a cleavable linker or non-cleavable linker; and
Bm is a binding moiety.

In some aspects, U is NH.

In some asspects, the neoDegrader conjugate described herein has in vitro anti-proliferative activity against a tumor cell line. In some aspects, the neoDegrader conjugate comprising a neoDegrader and a binding moiety has in vitro anti-proliferative activity at least about 50%, at least about 60%, at least about 70%, at least about 80%, at least about 90%, at least about 95%, or at least about 100% higher than the neoDegrader alone or the binding moiety alone. In some aspects, the neoDegrader conjugate comprising a neoDegrader and a binding moiety has in vitro anti-proliferative activity at least about 2 fold, at least about 3 fold, at least about 4 fold, at least about 5 fold, at least about 6 fold, at least about 7 fold, at least about 8 fold, at least about 9 fold, at least about 10 fold higher than the neoDegrader alone or the binding moiety alone.

In some aspects, the neoDegrader conjugates described herein have in vitro anti-proliferative activity against a BT-474 breast cancer cell line, e.g., higher anti-proliferative activity against a BT-474 breast cancer cell line, compared to the neoDegrader alone or the binding moiety alone. In some aspects, the neoDegrader conjugates described herein have in vitro anti-proliferative activity against an SK-BR-3 breast cancer cell line, e.g., higher anti-proliferative activity against an SK-BR-3 breast cancer cell line, compared to the neoDegrader alone or the binding moiety alone. In some aspects, the neoDegrader conjugates described herein have in vitro anti-proliferative activity against an NCI-N87 gastric cancer cell line, e.g., higher anti-proliferative activity against a NCI-N87 gastric cancer cell line, compared to the neoDegrader alone or the binding moiety alone. In some aspects, the neoDegrader conjugates described herein have in vitro anti-proliferative activity against a Daudi lymphoma cell line, e.g., higher anti-proliferative activity against a Daudi lymphoma cell line, compared to the neoDegrader alone or the binding moiety alone. In some aspects the neoDegrader conjugates described herein have in vitro anti-proliferative activity against the HL-60 acute myeloid leukemia cell line, e.g., higher anti-proliferative activity against a HL-60 acute myeloid leukemia cell line, compared to the neoDegrader alone or the binding moiety alone. In some aspects, the neoDegrader conjugates described herein have in vitro anti-proliferative activity against a Ramos non-Hodgkins lymphoma cell line,, e.g., higher anti-proliferative activity against a Ramos non-Hodgkins lymphoma cell line, compared to the neoDegrader alone or the binding moiety alone. In some aspects the neoDegrader conjugates described herein is capable of maintaining their anti-proliferative activity in the presence of human serum. The neoDegrader conjugates described herein can be used in the treatment of cancers.

### III.A. Linker

The neoDegrader of the present disclosure can be linked to the binding moiety via a linker. As used herein, the term “linker” refers to any chemical moiety capable of connecting the binding moiety (Bm) to group X within the compounds of formula (I).

In certain aspects, the linker can contain a heterobifunctional group. In the present disclosure, the term “heterobifunctional group” refers to a chemical moiety that connects the linker of which it is a part to the binding moiety. Heterobifunctional groups are characterized as having different reactive groups at either end of the chemical moiety. Attachment to “Bm,” can be accomplished through chemical or enzymatic conjugation, or a combination of both. Chemical conjugation involves the controlled reaction of accessible amino acid residues on the surface of the binding moiety with a reaction handle on the heterobifunctional group. Examples of chemical conjugation include, but are not limited to, lysine amide coupling, cysteine coupling, and coupling via a non-natural amino acid incorporated by genetic engineering, wherein non-natural amino acid residues with a desired reaction handle are installed onto “Bm.” In enzymatic conjugation, an enzyme mediates the coupling of the linker with an accessible amino residue on the binding moiety. Examples of enzymatic conjugation include, but are not limited to, transpeptidation using sortase, transpeptidation using microbial transglutaminase, and N-glycan engineering. Chemical conjugation and enzymatic conjugation may also be used sequentially. For example, enzymatic conjugation can also be used for installing unique reaction handles on “Bm” to be utilized in subsequent chemical conjugation.

In some aspects, the heterobifunctional group is selected from:

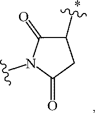

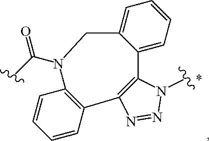

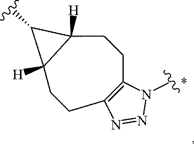

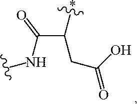

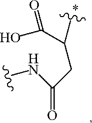

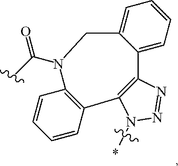

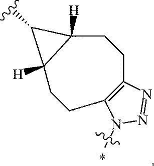

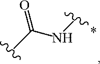

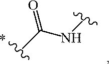

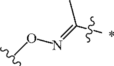

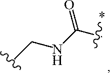

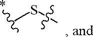

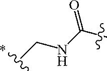

wherein

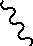

is the point of attachment to the remaining portion of the linker; and

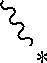

the point of attachment to Bm.

In certain aspects, linker “L” is non-cleavable. As used here, the term “non-cleavable linker” is any chemical moiety that is capable of linking the binding moiety to the neoDegrader in a stable, covalent manner and does not fall under the categories defined herein as “cleavable linkers”. Thus, non-cleavable linkers are substantially resistant to acid-induced cleavage, light-induced cleavage, bioreductive cleavge, peptidase-induced cleavage, esterase-induced cleavage, and disulfide bond cleavage. “Substantially resistant to cleavage” means that the chemical bond in the linker or adjoining the linker in at least 80%, preferably at least 85%, more preferably at least 90%, even more preferably at least 95%, and most preferably at least 99% of the antibody neoDegrader conjugate population remains non-cleavable by an acid, a photolabile-cleaving agent, a bioreductive agent, a peptidase, an esterase, or a chemical or a physiological compound that cleaves the chemical bond (for example, a disulfide bond) in a cleavable linker, for within a few hours to several days of treatment with any of the agents described above. In certain aspects the linker is not susceptible to acid-induced cleavage, photo-induced cleavage, bioreductive cleavage, enzymatic cleavage, or the like, at conditions under which the neoDegrader and/or binding moiety can remain active. ADC catabolites generated from non-cleavable linkers contain a residual amino acid from the antibody. These catabolites can exert unique and unexpected properties in the target cells to which they are delivered.

A person of ordinary skill in the art would readily distinguish non-cleavable from cleavable linkers.

Examples of non-cleavable linkers include, but are not limited to, SMCC (succinimidyl 4-(N-maleimidomethyl)cyclohexane-1-carboxylate) linkers, succinimide thioether linkers, and linkers such as:

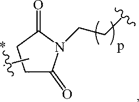

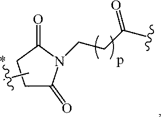

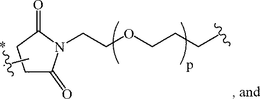

and

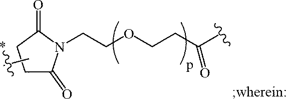

wherein:
p is an integer from 1 to 10;

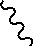

is the point of attachment to X; and

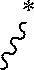

is the point of attachment to the binding moiety.

In some aspects, the linker is:

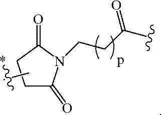

In some aspects, p is 5.

In certain aspects the linker can be cleavable. In some aspects, the linker can be susceptible to acid-induced cleavage, photo-induced cleavage, bioreductive cleavage, enzymatic cleavage, or the like, at conditions under which the neoDegrader and/or binding moiety can remain active.

In some aspects, the cleavable linker can be cleaved enzymatically. In some aspects, the cleavable linker can be cleaved by a protease, peptidase, esterase, beta-gluroronidase, glycosidase, phosphodiesterase, phosphatase, pyrophosphatase, or lipase.

In some aspects, the cleavable linker can be cleaved by a protease. Examples of proteases include, but are not limited to, cathepsin B, VAGP tetrapeptide, and the like.

In certain aspects, the cleavable linker contains a peptide. In some aspects, the peptide is the site of cleavage of the linker, thereby facilitating release of the drug upon exposure to intracellular proteases, such as lysosomal enzymes. Peptides can be designed and optimized for enzymatic cleavage by a particular enzyme, for example, a tumor-associated protease, cathepsin B, C and D, or a plasmin protease. Examples of peptides having two amino acids include, but are not limited to, alanine-alanine (ala-ala), valine-alanine (val-ala), valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Examples of peptides having three amino acids include, but are not limited to, glycine-valine-citrulline (gly-val-cit), aspartic acid-valine-citrulline (asp-val-cit), alanine-alanine-asparagine (ala-ala-asn), alanine-phenylalanine-lysine (ala-phe-lys), glycine-glycine-phenylalanine (gly-gly-phe), and glycine-glycine-glycine (gly-gly-gly). Examples of peptides having four amino acids include, but are not limited to, glycine-glycine-valine-citrulline (gly-gly-val-cit) and glycine-glycine-phenylalanine-glycine (gly-gly-phe-gly). The amino acid combinations above can also be present in the reverse order (i.e., cit-val).

The peptides of the present disclosure can comprise L- or D- isomers of amino acid residues. The term “naturally-occurring amino acid” refers to Ala, Asp, Asx, Cit, Cys, Glu, Phe, Glx, Gly, His, Ile, Lys, Leu, Met, Asn, Pro, Gln, Arg, Ser, Thr, Val, Trp, and Tyr. “D-” designates an amino acid having the “D” (dextrorotary) configuration, as opposed to the configuration in the naturally occurring (“L-”) amino acids. The amino acids described herein can be purchased commercially (Sigma Chemical Co., Advanced Chemtech) or synthesized using methods known in the art.

In certain aspects, the linker (“L”) is a protease cleavable linker selected from

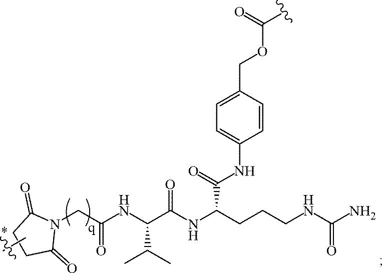

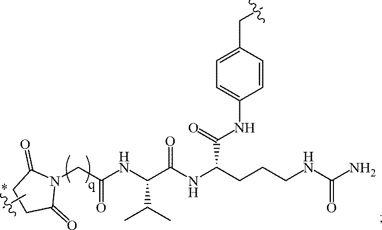

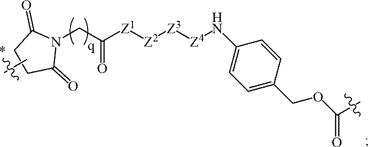

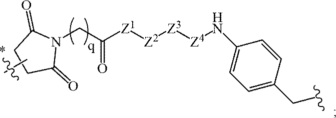

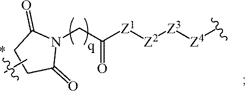

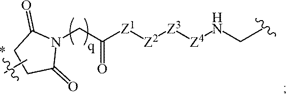

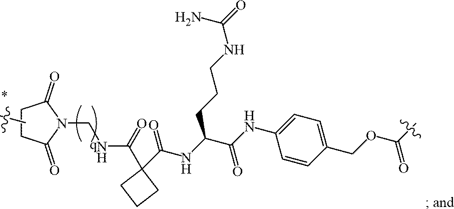

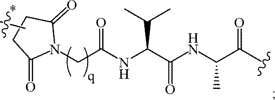

wherein:
q is an integer from 2 to 10;
Z¹, Z², Z³, and Z⁴ are each independently absent or a naturally-occurring amino acid residue in the L- or D-configuration, provided that at least two of Z¹, Z², Z³, and Z⁴ are amino acid residues;

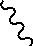

is the point of attachment to X; and

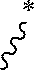

is the point of attachment to the binding moiety.

In certain aspects, Z¹, Z², Z³, and Z⁴ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, and Z⁴ are amino acid residues.

In some aspects, Z¹ is absent or glycine; Z² is absent or selected from L-glutamine, D-glutamine, L-glutamic acid, D-glutamic acid, L-aspartic acid, D-aspartic acid, L-alanine, D-alanine, and glycine; Z³ is selected from L-valine, D-valine, L-alanine, D-alanine, L-phenylalanine, D-phenylalanine, and glycine; and Z⁴ is selected from L-alanine, D-alanine, L-citrulline, D-citrulline, L-asparagine, D-asparagine, L-lysine, D-lysine, L-phenylalamine, D-phenylalanine, and glycine.

In some aspects, L is

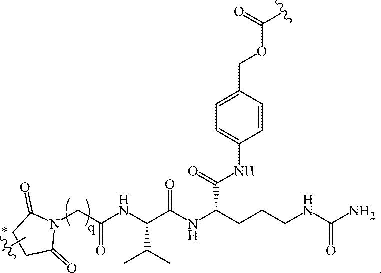

In some aspects, q is 5.

In certain aspects, L is a pyrophosphatase cleavable linker.

In some aspects, L is a pyrophosphatase cleavable linker which is:

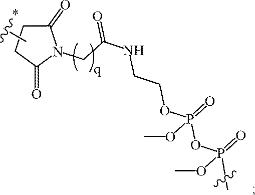

wherein:
q is an integer from 2 to 10;

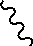

is the point of attachment to X; and

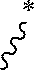

is the point of attachment to the binding moiety.

In certain aspects, L is a beta-glucoronidase cleavable linker.

In some aspects, L is a beta-glucoronidase cleavable linker selected from:

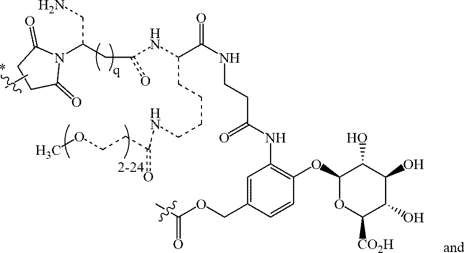

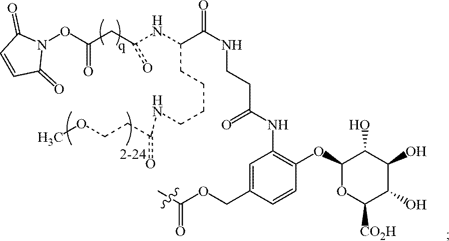

wherein:
q is an integer from 2 to 10;

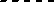

is absent or a bond;

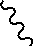

is the point of attachment to X; and

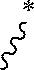

is the point of attachment to the binding moiety.

In some aspects, the linker is bioreducible. Bioreducible linkers take advantage of the difference in reduction potential in the intracellular compartment versus plasma. Reduced glutathione presented in tumor cells’ cytoplasma is up to 1000-fold higher than that present in normal cells’ cytoplasma, and the tumor cells also contain enzymes which can contribute to reduction in cellular compartments. The linkers keep conjugates intact during systemic circulation, and are selectively cleaved by the high intracellular concentration of glutathione, releasing the active drugs at the tumor sites from the non-toxic prodrugs.

In some aspects, L is a bioreducible linker selected from:

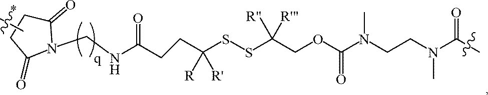

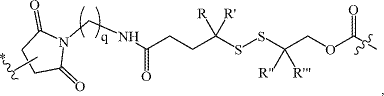

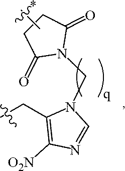

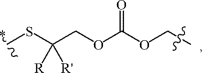

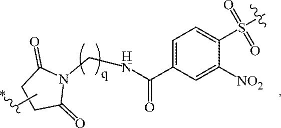

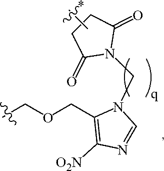

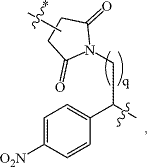

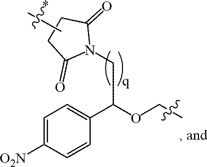

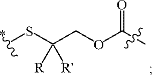

wherein:
q is an integer from 2 to 10;
R, R′, R″, and R‴ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring;

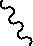

is the point of attachment to X; and

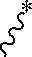

is the point of attachment to the binding moiety.

In certain aspects, the linker is acid cleavable. Acid-cleavable linkers are specifically designed to remain stable at the neutral pH of blood circulation, but undergo hydrolysis and release the cytotoxic drug in the acidic environment of the cellular compartments.

In some aspects, L is an acid cleavable linker selected from

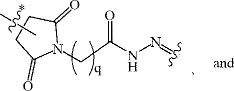

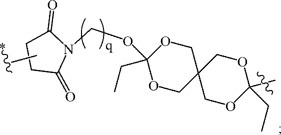

wherein:
q is an integer from 2 to 10;

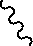

is the point of attachment to X; and

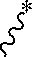

is the point of attachment to the binding moiety.

In certain aspects, L is wherein L is a click-to-release linker, where release of the neoDegrader is chemically triggered by a tetrazine or related compound.

In some aspects, L is a click-to-release linker selected from

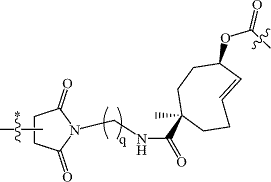

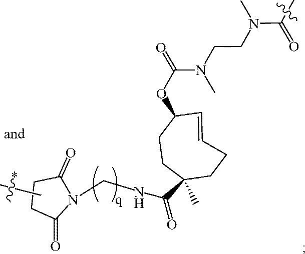

wherein:
q is an integer from 2 to 10;

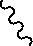

is the point of attachment to X; and

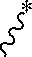

is the point of attachment to the binding moiety.

### III.B. Binding Moiety

The present disclosure provides neoDegraders conjugated to binding moieties. The term “binding moiety,” as used herein, refers to any molecule that recongnizes and binds to a cell surface marker or receptor. In certain aspects, the binding moiety binds to a protein, not limited to a polypeptide moiety. The binding moiety, in addition to targeting the neoDegrader to a specific cell, tissue, or location, may also have certain therapeutic effect such as antiproliferative (cytostatic and/or cytotoxic) activity against a target cell or pathway. In certain aspects the binding moiety can comprise or can be engineered to comprise at least one chemically reactive group such as a carboxylic acid, amine, thiol, or chemically reactive amino acid moiety or side chain. In some aspects, the binding moiety can comprise a targeting moiety which binds or complexes with a cell surface molecule, such as a cell surface receptor or antigen, for a given target cell population. Following specific binding or complexing with the receptor, the cell is permissive for uptake of the targeting moiety or the neoDegrader conjugate, which is then internalized into the cell.

In some aspects, group “Bm” can be a moiety that can specifically bind to a cell surface molecule. In some aspects, group “Bm” can be a peptide or a protein that binds to a cell surface receptor or antigen.

In certain aspects, group “Bm” can be an antibody, antibody fragment, or an antigen-binding fragment. An antibody is a protein generated by the immune system that is capable of recognizing and binding to a specific antigen. A target antigen generally has numerous binding sites, also called epitopes, recognized by CDRs on multiple antibodies. Each antibody that specifically binds to a different epitope has a different structure. Thus, one antigen may have more than one corresponding antibody. The term “antibody” herein is used in the broadest sense and specifically covers monoclonal antibodies, single domain antibodies, polyclonal antibodies, multispecific antibodies (e.g., bispecific antibodies), and antibody fragments, so long as they exhibit the desired biological activity. Antibodies may be murine, human, humanized, chimeric, or derived from other species.

Monoclonal antibodies that can be conjugated to the neoDegrader are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture. These include, but are not limited to, the hybridoma technique, the human B cell hybridoma technique, and the EBV-hybridoma technique. Such antibodies may be of any immunoglobulin class including IgG, IgM, IgE, IgA, and IgD and any subclass thereof. The hybridoma producing the mAbs of use in this disclosure may be cultivated in vitro or in vivo.

Useful monoclonal antibodies include, but are not limited to, human monoclonal antibodies, humanized monoclonal antibodies, antibody fragments, or chimeric human-mouse (or other species) monoclonal antibodies. Human monoclonal antibodies may be made by any of numerous techniques known in the art.

The antibody can also be a bispecific antibody. Methods for making bispecific antibodies are known in the art. Traditional production of full-length bispecific antibodies is based on the coexpression of two immunoglobulin heavy chain-light chain pairs, where the two chains have different specificities. Because of the random assortment of immunoglobulin heavy and light chains, these hybridomas (quadromas) produce a potential mixture of 10 different antibody molecules, of which only one has the correct bispecific structure. Purification of the correct molecule, which is usually performed using affinity chromatography steps, is rather cumbersome, and the product yields are low.

According to a different approach, antibody variable domains with the desired binding specificities (antibody-antigen combining sites) are fused to immunoglobulin constant domain sequences. The fusion may be with an immunoglobulin heavy chain constant domain, comprising at least part of the hinge, C.sub.H2, and C.sub.H3 regions. The first heavy-chain constant region (C.sub.H1) may contain the site necessary for light chain binding, present in at least one of the fusions. Nucleic acids with sequences encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in aspects when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

Bispecific antibodies may have a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. Using such techniques, bispecific antibodies can be prepared for conjugation to the neoDegraders in the treatment or prevention of disease as defined herein.

Hybrid or bifunctional antibodies can be derived either biologically, i.e., by cell fusion techniques, or chemically, especially with cross-linking agents or disulfide-bridge forming reagents, and may comprise whole antibodies or fragments thereof.

The antibody can be a functionally active fragment, derivative or analog of an antibody that immunospecifically binds to cancer cell antigens, viral antigens, or microbial antigens or other antibodies bound to tumor cells or matrix. In this regard, “functionally active” means that the fragment, derivative or analog is able to elicit anti-anti-idiotype antibodies that recognize the same antigen that the antibody from which the fragment, derivative or analog is derived recognized. Specifically, in an exemplary aspect the antigenicity of the idiotype of the immunoglobulin molecule can be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

Other useful antibodies include fragments of antibodies such as, but not limited to, F(ab′)2 fragments, which contain the variable region, the light chain constant region and the CH1 domain of the heavy chain can be produced by pepsin digestion of the antibody molecule, and Fab fragments, which can be generated by reducing the disulfide bridges of the F(ab′)2 fragments. Other useful antibodies are heavy chain and light chain dimers of antibodies, or any minimal fragment thereof such as Fvs or single chain antibodies (SCAs), or any other molecule with the same specificity as the antibody.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as those having a variable region derived from a murine monoclonal and human immunoglobulin constant regions. Humanized antibodies are antibody molecules from non-human species having one or more complementarity determining regions (CDRs) from the non-human species and a framework region from a human immunoglobulin molecule. Such chimeric and humanized monoclonal antibodies can be produced by recombinant DNA techniques known in the art.

Completely human antibodies can be produced using transgenic mice that are incapable of expressing endogenous immunoglobulin heavy and light chains genes, but which can express human heavy and light chain genes. The transgenic mice are immunized in the normal fashion with a selected antigen, e.g., all or a portion of a polypeptide of the disclosure. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). Other human antibodies can be obtained commercially from, for example, Abgenix, Inc. (Freemont, Calif.) and Genpharm (San Jose, Calif.).

Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope. Human antibodies can also be produced using various techniques known in the art, including phage display libraries.

The antibody can be a fusion protein of an antibody, or a functionally active fragment thereof, for example in which the antibody is fused via a covalent bond (e.g., a peptide bond), at either the N-terminus or the C-terminus to an amino acid sequence of another protein (or portion thereof, such as at least 10, 20 or 50 amino acid portion of the protein) that is not the antibody. The antibody or fragment thereof may be covalently linked to the other protein at the N-terminus of the constant domain.

Antibodies include analogs and derivatives that are either modified, i.e., by the covalent attachment of any type of molecule as long as such covalent attachment permits the antibody to retain its antigen binding immunospecificity. For example, but not by way of limitation, the derivatives and analogs of the antibodies include those that have been further modified, e.g., by glycosylation, acetylation, pegylation, phosphorylation, amidation, derivatization by known protecting/blocking groups, proteolytic cleavage, linkage to a cellular antibody unit or other protein, etc. Any of numerous chemical modifications can be carried out by known techniques, including, but not limited to specific chemical cleavage, acetylation, formylation, metabolic synthesis in the presence of tunicamycin, etc. Additionally, the analog or derivative can contain one or more unnatural amino acids.

The antibodies in neoDegrader conjugates can include antibodies having modifications (e.g., substitutions, deletions or additions) in amino acid residues that interact with Fc receptors. In particular, antibodies include antibodies having modifications in amino acid residues identified as involved in the interaction between the anti-Fc domain and the FcRn receptor. Antibodies immunospecific for a cancer cell antigen can be obtained commercially, for example, from Genentech (San Francisco, Calif.) or produced by any method known to one of skill in the art such as, e.g., chemical synthesis or recombinant expression techniques. The nucleotide sequence encoding antibodies immunospecific for a cancer cell antigen can be obtained, e.g., from the GenBank database or a database like it, the literature publications, or by routine cloning and sequencing.

In certain aspects, the antibody of the neoDegrader conjugates can be a monoclonal antibody, e.g. a murine monoclonal antibody, a chimeric antibody, or a humanized antibody. In some aspects, the antibody can be an antibody fragment, e.g. a Fab fragment.

Known antibodies for the treatment or prevention of cancer can be conjugated to the neoDegraders described herein. Antibodies immunospecific for a cancer cell antigen can be obtained commercially or produced by any method known to one of skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing. Examples of antibodies available for the treatment of cancer include, but are not limited to, humanized anti-HER2 monoclonal antibody for the treatment of patients with metastatic breast cancer; RITUXAN.RTM. (rituximab; Genentech) which is a chimeric anti-CD20 monoclonal antibody for the treatment of patients with non-Hodgkin’s lymphoma; OvaRex (oregovomab; AltaRex Corporation, MA) which is a murine antibody for the treatment of ovarian cancer; Panorex (edrecolomab, Glaxo Wellcome, NC) which is a murine IgG.sub.2a antibody for the treatment of colorectal cancer; Cetuximab Erbitux (cetuximab, Imclone Systems Inc., NY) which is an anti-EGFR IgG chimeric antibody for the treatment of epidermal growth factor positive cancers, such as head and neck cancer; Vitaxin (etaracizumab, MedImmune, Inc., MD) which is a humanized antibody for the treatment of sarcoma; Campath I/H (alemtuzumab, Leukosite, MA) which is a humanized IgG.sub.1 antibody for the treatment of chronic lymphocytic leukemia (CLL); Smart MI95 (Protein Design Labs, Inc., CA) which is a humanized anti-CD33 IgG antibody for the treatment of acute myeloid leukemia (AML); LymphoCide (epratuzumab, Immunomedics, Inc., NJ) which is a humanized anti-CD22 IgG antibody for the treatment of non-Hodgkin’s lymphoma; Smart ID10 (Protein Design Labs, Inc., CA) which is a humanized anti-HLA-DR antibody for the treatment of non-Hodgkin’s lymphoma; Oncolym (Techniclone, Inc., CA) which is a radiolabeled murine anti-HLA-Dr10 antibody for the treatment of non-Hodgkin’s lymphoma; Allomune (BioTransplant, CA) which is a humanized anti-CD2 mAb for the treatment of Hodgkin’s Disease or non-Hodgkin’s lymphoma; Avastin (bevacizumab, Genentech, Inc., CA) which is an anti-VEGF humanized antibody for the treatment of lung and colorectal cancers; Epratuzamab (Immunomedics, Inc., NJ and Amgen, CA) which is an anti-CD22 antibody for the treatment of non-Hodgkin’s lymphoma; and CEAcide (Immunomedics, NJ) which is a humanized anti-CEA antibody for the treatment of colorectal cancer.

Other antibodies useful for the neoDegrader conjugates include, but are not limited to, trastuzumab, gemtuzumab, pertuzumab, obinutuzumab, ofatumumab, daratumumab, STI-6129, lintuzumab, huMy9-6, balantamab, indatuximab, dinutuximab, anti-CD38 A2 antibody, HuAT13/5 H3s antibody, ibritumomab, tositumomab, panitumumab, tremelimumab, ticilimumab, catumaxomab, and veltuzumab. In certain aspects, the antibody is selected from the group consisting of rituximab, trastuzumab, pertuzumab, OR000213, lintuzumab, and gemtuzumab.

Other antibodies useful for the neoDegrader conjugates include, but are not limited to, antibodies against the following antigens: CA125 (ovarian), CA15-3 (carcinomas), CA19-9 (carcinomas), L6 (carcinomas), Lewis Y (carcinomas), Lewis X (carcinomas), alpha fetoprotein (carcinomas), CA 242 (colorectal), placental alkaline phosphatase (carcinomas), prostate specific antigen (prostate), prostatic acid phosphatase (prostate), epidermal growth factor (carcinomas), MAGE-1 (carcinomas), MAGE-2 (carcinomas), MAGE-3 (carcinomas), MAGE-4 (carcinomas), anti-transferrin receptor (carcinomas), p97 (melanoma), MUC1-KLH (breast cancer), CEA (colorectal), gp100 (melanoma), MART1 (melanoma), PSA (prostate), IL-2 receptor (T-cell leukemia and lymphomas), CD20 (non-Hodgkin’s lymphoma), CD52 (leukemia), CD33 (leukemia), CD22 (lymphoma), human chorionic gonadotropin (carcinoma), CD38 (multiple myeloma), CD40 (lymphoma), mucin (carcinomas), P21 (carcinomas), MPG (melanoma), and Neu oncogene product (carcinomas). Some specific, useful antibodies include, but are not limited to, BR96 mAb (Trail, P. A., et al Science (1993) 261, 212-215), BR64 (Trail, P A, et al Cancer Research (1997) 57, 100-105), mAbs against the CD40 antigen, such as S2C6 mAb (Francisco, J. A., et al Cancer Res. (2000) 60:3225-3231), mAbs against the CD70 antigen, such as 1F6 mAb, and mAbs against the CD30 antigen, such as AC10. Many other internalizing antibodies that bind to tumor associated antigens can be used and have been reviewed.

Other antigens that the present conjugates can bind to include, but are not limited to, 5T4, ACE, ADRB3, AKAP-4, ALK, Androgen receptor, AOC3, APP, Axin1, AXL, B7H3, B7-H4, BCL2, BCMA, bcr-abl, BORIS, BST2, C242, C4.4a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudin 3, Claudin 18.2, CLDN6, CLEC12A, CLL-1, cll3, c-MET, Cripto protein, CS1, CTLA-4, CXCR2, CXORF61, Cyclin Bl, CYP1B1, Cadherin-3, Cadherin-6, DLL3, E7, EDNRB, EFNA4, EGFR, EGFRvIII, ELF2M, EMR2, ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GloboH, GM3, GPC1, GPC2, GPC3, gplOO, GPNMB, GPR20, GPRC5D, GUCY2C, HAVCR1, HER2, HER3, HGF, HMI.24, HMWMAA, HPV E6, hTERT, human telomerase reverse transcriptase, ICAM, ICOS-L, IFN- α, IFN-γ, IGF-I receptor, IGLL1, IL-2 receptor, IL-4 receptor, IL-13Ra2, II,-11Ra, IL-1, IL-12, IL-23, IL-13, IL-22, IL-4, IL-5, IL-6, interferon receptor, integrins (including α4, αvβ3, αvβ5, αvβ6, α1β4, α4β1, α4β7, α5β1, α6β4, αIIbβ3 intergins), Integrin alphaV, intestinal carboxyl esterase, KIT, LAGE-la, LAIR1, LAMP-1, LCK, Legumain, LewisY, LFA-1(CD11a), L-selectin(CD62L), LILRA2, LIV-1, LMP2, LRRC15, LY6E, LY6K, LY75, MAD-CT-1, MAD-CT-2, MAGE Al, MelanA/MARTl, Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2, OY-TES1, p53, p53 mutant, PANX3, PAP, PAX3, PAX5, p-CAD, PCTA- ⅟Galectin 8, PD-L1, PD-L2, PDGFR, PDGFR-beta, phosphatidylserine, PIK3CA, PLAC1, Polysialic acid, Prostase, prostatic carcinoma cell, prostein, Pseudomonas aeruginosa, rabies, survivin and telomerase, PRSS21, PSCA, PSMA, PTK7, RAGE-1, RANKL, Ras mutant, respiratory syncytial virus, Rhesus factor, RhoC, RON, ROR1, ROR2, RU1, RU2, sarcoma translocation breakpoints, SART3, SLAMF7, SLC44A4, sLe, SLITRK6, sperm protein 17, sphingosine-1-phosphate, SSEA-4, SSX2, STEAP1, TAG72, TARP, TCRβ, TEM1/CD248, TEM7R, tenascin C, TF, TGF-1, TGF- β2, TNF-α, TGS5, Tie 2, TIM-1, Tn Ag, TRAC, TRAIL-R1, TRAIL-R2, TROP-2, TRP-2, TRPV1, TSHR, tumor antigen CTAA16.88, tyrosinase, UPK2, VEGF, VEGFR1, VEGFR2, vimentin, WTl, and/or XAGE1.

Antibodies that bind to antigens associated with antigen presenting cells such as CD40, OX40L, Endoglin, DEC-205, 4-1BBL, CD36, CD36, CD204, MARCO, DC-SIGN, CLEC9A, CLEC5A, Dectin 2, CLEC10A, CD206, CD64, CD32A, CD1A, HVEM, CD32B, PD-L1, BDCA-2, XCR-1, and CCR2 can also be conjugated to the neoDegraders.

Antibodies of a neoDegrader conjugate can bind to both a receptor or a receptor complex expressed on an activated lymphocyte. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein. Non-limiting examples of suitable immunoglobulin superfamily members are CD2, CD3, CD4, CD8, CD 19, CD22, CD28, CD79, CD90, CD 152/CTLA-4, PD-1, and ICOS. Non-limiting examples of suitable TNF receptor superfamily members are CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, and APO-3. Non-limiting examples of suitable integrins are CD11a, CD11b, CD11c, CD18, CD29, CD41, CD49a, CD49b, CD49c, CD49d, CD49e, CD49f, CD 103, and CD 104. Non-limiting examples of suitable lectins are C-type, S-type, and I-type lectin.

In some aspects, the antibodies that can are useful for the present disclosure include, but are not limited to, 3F8, 8H9, abagovomab, abciximab (REOPRO^{®}), abituzumab, abrezekimab, abrilumab, actoxumab, adalimumab (HUMIRA^{®}), adecatumumab, aducanumab, afasevikumab, afelimomab, afutuzumab, alacizumab, ALD518, alemtuzumab (CAMPATH^{®}), alirocumab (PRALUENT^{®}), altumomab, amatuximab, anatumomab, andecaliximab, anetumab, anifrolumab, anrukinzumab, apolizumab, aprutumab, arcitumomab (CEA-SCAN^{®}), ascrinvacumab, aselizumab, atidortoxumab, atlizumab (tocilizumab, ACTEMRA^{®}, ROACTEMRA^{®}), atezolizumab (TECENTRIQ^{®}), atinumab, atorolimumab, avelumab (Bavencio), azintuxizumab, balantamab, bapineuzumab, basiliximab (SIMULECT^{®}), bavituximab, BCD-100, bectumomab (LYMPHOSCAN^{®}), begelomab, belantamab, belimumab (BENLYSTA^{®}), bemarituzumab, benralizumab (FASENRA^{®}), bermekimab, bersanlimab, bertilimumab, besilesomab (SCINITIMUN^{®}), bevacizumab (AVASTIN^{®}), bezlotoxumab (ZINPLAVA^{®}), biciromab (FIBRISCINT^{®}), bimagrumab, bimekizumab, birtamimab, bivatuzumab, bleselumab, blinatumomab, blontuvetmab, blosozumab, bococizumab, brazikumab, brentuximab, briakinumab, brodalumab (SILIQ™), brolucizumab (BEOVU^{®} brontictuzumab, burosumab (CRYSVITA^{®}), cabiralizumab, caplacizumab (CABLIVI^{®}), camidanlumab, camrelizumab, canakinumab (ILARIS^{®}), cantuzumab, capromab, carlumab, carotuximab, catumaxomab (REMOVAB^{®}), cBR96, CC49, cedelizumab, cemiplimab (LIBTAYO^{®}), cergutuzumab, certrelimab, certolizumab, cetuximab (ERBITUX^{®}), cibisatamab, cirmtuzumab, citatuzumab, cixutumumab, clazakizumab, clenoliximab, clivatuzumab, codrituzumab, cofetuzumab, coltuximab, conatumumab, concizumab, cosfroviximab, CR6261, crenezumab, crizanlizumab (ADAKVEO^{®}), crotedumab, cusatuzumab, dacetuzumab, daclizumab (ZINBRYTA^{®}), dalotuzumab, dapirolizumab, daratumumab (DARZALEX^{®}), dectrekumab, demcizumab, denintuzumab, denosumab (PROLIA^{®}), depatuxizumab, derlotuximab, detumomab, dezamizumab, dinutuximab (UNITUXIN^{®}), diridavumab, domagrozumab, dostarlimab, dorlimomab, dorlixizumab, drozitumab, DS-8201, duligotuzumab, dupilumab (DUPIXENT^{®}), durvalumab (IMFINZI^{®}), dusigitumab, ecromeximab, eculizumab (SOLIRIS^{®}), edobacomab, edrecolomab (PANOREX^{®}), efalizumab (RAPTIVA^{®}), efungumab (MYCOGRAB^{®}), eldelumab, elezanumab, elgemtumab, elotuzumab (EMPLICITI^{®}), elsilimomab, emactuzumab emapalumab (GAMIFANT^{®}), emibetuzumab, emicizumab (HEMLIBRA^{®}), enapotamab, enavatuzumab, enfortumab (PADCEV^{®}), enlimomab, enoblituzumab, enokizumab, enoticumab, ensituximab, epitumomab, eptinezumab (VYEPTI^{®}epratuzumab, erenumab (AIMOVIG^{®}), erlizumab, ertumaxomab (REXOMUN^{®}), etaracizumab (ABEGRIN^{®}), etigilimab, etrolizumab, evinacumab, evolocumab (REPATHA^{®}), exbivirumab, fanolesomab (NEUTROSPEC^{®}), faralimomab, faricimab, farletuzumab, fasinumab, FBTA05, felvizumab, fezakinumab, fibatuzumab, ficlatuzumab, figitumumab, firivumab, flanvotumab, fletikumab, flotetuzumab, fontolizumab (HUZAF^{®}), foralumab, foravirumab, fremanezumab (AJOVY^{®}), fresolimumab, frovocimab, frunevetmab, fulranumab, futuximab, galcanezumab (EMGALITY^{®}), galiximab, gancotamab, ganitumab, gantenerumab, gavilimomab, gedivumab, gemtuzumab, gevokizumab, gilvetmab, gimsilumab, girentuximab, glembatumumab, golimumab (SIMPONI^{®}), gomiliximab, guselkumab (TREMFYA^{®}), huMy9-6, OR000213, ianalumab, ibalizumab (TROGARZO^{®}), IBI308, ibritumomab, icrucumab, idarucizumab (PRAXBIND^{®}), ifabotuzumab, igovomab (INDIMACIS-125), iladatuzumab, IMAB362, imalumab, imaprelimab, imciromab (MYOSCINT^{®}), imgatuzumab, inclacumab, indatuximab, indusatumab, inebilizumab, infliximab (REMICADE^{®}), intetumumab, inolimomab, inotuzumab, iomab-B, ipilimumab, iratumumab, isatuximab (SARCLISA^{®}), iscalimab, istiratumab, itolizumab, ixekizumab (TALTZ^{®}), keliximab, labetuzumab (CEA-CIDE™), lacnotuzumab, ladiratuzumab, lampalizumab, lanadelumab (TAKHZYRO^{®}), landogrozumab, laprituximab, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab, ligelizumab, lilotomab, lintuzumab, lirilumab, lodelcizumab, lokivetmab, loncastuximab, lorvotuzumab, losatuxizumab, lucatumumab, lulizumab, lumiliximab, lumretuzumab, lupartumab, lutikizumab, mapatumumab, margetuximab, marstacimab, maslimomab, matuzumab, mavrilimumab, mepolizumab (NUCALA^{®}), metelimumab, milatuzumab, minretumomab, mirikizumab, mirvetuximab, mitumomab, modotuximab, molalizumab, mogamulizumab (POTELIGEO^{®}), morolimumab, mosunetuzumab, motavizumab (NUMAX^{®}), moxetumomab (LUMOXITI^{®}), muromonab-CD3 (ORTHOCLONE OKT3^{®}), nacolomab, namilumab, naptumomab, naratuximab, narnatumab, natalizumab (TYSABRI^{®}), navicixizumab, navivumab, naxitamab, nebacumab, necitumumab (PORTRAZZA^{®}), nemolizumab, NEOD001, nerelimomab, nesvacumab, netakimab, nimotuzumab (THERACIM^{®}), nirsevimab, nivolumab, nofetumomab, obiltoxaximab (ANTHIM^{®}), obinutuzumab, ocaratuzumab, ocrelizumab (OCREVUS^{®}), odulimomab, ofatumumab (ARZERRA^{®}), olaratumab (LARTRUVO^{®}), oleclumab, olendalizumab, olokizumab, omalizumab (XOLAIR^{®}), omburtamab, OMS721, onartuzumab, ontecizumab, ontuxizumab, onvatilimab, opicinumab, oportuzumab, oregovomab (OVAREX), orticumab, otelixizumab, otilimab, otlertuzumab, oxelumab, ozanezumab, ozogamicin, ozoralizumab, pagibaximab, palivizumab (SYNAGIS^{®}), pamrevlumab, panitumumab (VECTIBIX^{®}), pankomab, panobacumab, parsatuzumab, pascolizumab, pasotuxizumab, pateclizumab, patritumab, PDR001, pembrolizumab, pemtumomab (THERAGYN^{®}), perakizumab, pertuzumab (OMNITARG^{®}), pexelizumab, pidilizumab, pinatuzumab, pintumomab, placulumab, polatuzumab (Polivy), prezalumab, plozalizumab, pogalizumab, ponezumab, porgaviximab, prasinezumab, prezalizumab, priliximab, pritoxaximab, pritumumab, PRO 140, quilizumab, racotumomab, radretumab, rafivirumab, ralpancizumab, ramucirumab, ranevetmab, ranibizumab (LUCENTIS^{®}), ravagalimab, ravulizumab (ULTOMIRIS^{®}), raxibacumab, refanezumab, regavirumab, REGN-EB3, renatlimab, remtolumab, reslizumab (CINQAIR^{®}), rilotumumab, rinucumab, risankizumab (SKYRIZI^{®}), rituximab (RITUXAN^{®}), rivabazumab, rmab, robatumumab, roledumab, romilkimab, romosozumab (EVENITY^{®}), rontalizumab, rosmantuzumab, rovalpituzumab, rovelizumab (LEUKARREST^{®}), rozanolixizumab, ruplizumab (ANTOVA), SA237, sacituzumab, samalizumab, samrotamab, sarilumab (KEVZARA^{®}), satralizumab, satumomab pendetide, secukinumab (COSENTYX^{®}), selicrelumab, seribantumab, setoxaximab, setrusumab, sevirumab, SGN-CD19A, SHP647, sibrotuzumab, sifalimumab, siltuximab, simtuzumab, siplizumab, sirtratumab, sirukumab, sofituzumab, solanezumab, solitomab, sonepcizumab, sontuzumab, spartalizumab, stamulumab, STI-6129, sulesomab (LEUKOSCAN^{®}), suptavumab, sutimlimab, suvizumab, suvratoxumab, tabalumab, tacatuzumab (AFP-CIDE^{®}), tadocizumab, talacotuzumab, talizumab, tamtuvetmab, tanezumab, taplitumomab paptox, tarextumab, tavolimab, tefibazumab (AUREXIS^{®}), telimomab, telisotuzumab, tesidolumab, tetraxetan, tetulomab, tenatumomab, teneliximab, teprotumumab (TEPEZZA^{®}), teplizumab, tezepelumab, TGN1412, tibulizumab,ticilimumab (TREMELIMUMAB^{®}), tigatuzumab, timigutuzumab, timolumab, tiragolumab, tiragotumab, tislelizumab, tisotumab, tiuxetan, tildrakizumab (ILUMYA^{®}), TNX-650, tocilizumab (atlizumab, ACTEMRA^{®}), tomuzotuximab, toralizumab, tosatoxumab, tositumomab (BEXXAR^{®}), tovetumab, tralokinumab, trastuzumab (HERCEPTIN^{®}), TRBS07, tregalizumab, tremelimumab, trevogrumab, tucotuzumab, tuvirumab, urtoxazumab, ustekinumab (STELERA^{®}), ublituximab, ulocuplumab, urelumab, utomilumab, vadastuximab, vanalimab, vandortuzumab, vantictumab, vanucizumab, vapaliximab, varisacumab, varlilumab, vatelizumab, vedolizumab, veltuzumab, vepalimomab, vesencumab, visilizumab (NUVION^{®}), vobarilizumab, volociximab (HUMASPECT^{®}), vonlerolizumab, vopratelimab, vorsetuzumab, votumumab, vunakizumab, xentuzumab, XMAB-5574, zalutumumab (HuMEX-EGFr), zanolimumab (HuMAX-CD4), zatuximab, zenocutuzumab, ziralimumab, zolbetuximab or zolimomab.

An antibody “which binds” a molecular target or an antigen of interest is one capable of binding that antigen with sufficient affinity such that the antibody is useful in targeting a cell expressing the antigen.

In the present disclosure, group “Bm” can be conjugated to more than one neoDegrader. In some aspects, “Bm” can be conjugated to from 1 to 10 neoDegraders. In some aspects, “Bm” can be conjugated to from 1 to 9 neoDegraders. In some aspects, “Bm” can be conjugated to from 1 to 8 neoDegraders. In some aspects, “Bm” can be conjugated to 1, 2, 3, 4, 5, 6, 7, 8, 9, or 10 neoDegraders. In some aspects, “Bm” can be conjugated to 7 or 8 neoDegraders. In some aspects, “Bm” is conjugated to 5 neoDegraders. In some aspects, “Bm” is conjugated to 6 neoDegraders. In some aspects, “Bm” is conjugated to 7 neoDegraders. In some aspects, “Bm” is conjugated to 8 neoDegraders. In some aspects, “Bm” is conjugated to 9 neoDegraders.

### IV. Compositions and Methods of Using

The conjugates and/or compounds described herein can be in the form of pharmaceutically or pharmaceutically acceptable salts. In some aspects, such salts are derived from inorganic or organic acids or bases.

Examples of suitable acid addition salts include acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Examples of suitable base addition salts include ammonium salts; alkali metal salts, such as sodium and potassium salts; alkaline earth metal salts, such as calcium and magnesium salts; salts with organic bases, such as dicyclohexylamine salts, N-methyl-D-glucamine; and salts with amino acids such as arginine, lysine, and the like.

For example, Berge lists the following FDA-approved commercially marketed salts: anions acetate, besylate (benzenesulfonate), benzoate, bicarbonate, bitartrate, bromide, calcium edetate (ethylenediaminetetraacetate), camsylate (camphorsulfonate), carbonate, chloride, citrate, dihydrochloride, edetate (ethylenediaminetetraacetate), edisylate (1,2-ethanedisulfonate), estolate (lauryl sulfate), esylate (ethanesulfonate), fumarate, gluceptate (glucoheptonate), gluconate, glutamate, glycollylarsanilate (glycollamidophenylarsonate), hexylresorcinate, hydrabamine (N,N′-di(dehydroabietyl)ethylenediamine), hydrobromide, hydrochloride, hydroxynaphthoate, iodide, isethionate (2-hydroxyethanesulfonate), lactate, lactobionate, malate, maleate, mandelate, mesylate (methanesulfonate), methylbromide, methylnitrate, methylsulfate, mucate, napsylate (2-naphthalenesulfonate), nitrate, pamoate (embonate), pantothenate, phosphate/diphosphate, polygalacturonate, salicylate, stearate, subacetate, succinate, sulfate, tannate, tartrate, teoclate (8-chlorotheophyllinate) and triethiodide; organic cations benzathine (N,N′-dibenzylethylenediamine), chloroprocaine, choline, diethanolamine, ethylenediamine, meglumine (N-methylglucamine) and procaine; and metallic cations aluminum, calcium, lithium, magnesium, potassium, sodium and zinc.

Berge additionally lists the following non-FDA-approved commercially marketed (outside the United States) salts: anions adipate, alginate, aminosalicylate, anhydromethylenecitrate, arecoline, aspartate, bisulfate, butylbromide, camphorate, digluconate, dihydrobromide, disuccinate, glycerophosphate, hemisulfate, hydrofluoride, hydroiodide, methylenebis(salicylate), napadisylate (1,5-naphthalenedisulfonate), oxalate, pectinate, persulfate, phenylethylbarbiturate, picrate, propionate, thiocyanate, tosylate and undecanoate; organic cations benethamine (N-benzylphenethylamine), clemizole (1-p-chlorobenzyl-2-pyrrolildine-1′-ylmethylbenzimidazole), diethylamine, piperazine and tromethamine (tris(hydroxymethyl)aminomethane); and metallic cations barium and bismuth.

Pharmaceutical compositions comprising the neoDegrader conjugates described herein may also comprise suitable carriers, excipients, and auxiliaries that may differ depending on the mode of administration.

In some aspects, the pharmaceutical compositions can be formulated as a suitable parenteral dosage form. Said formulations can be prepared by various methods known in the art. The pharmaceutical compositions can be administered directly into the bloodstream, into muscle, or directly into an organ. Suitable means for parenteral administration include intravenous, intraarterial, intraperitoneal, intrathecal, intraventricular, intraurethral, intrasternal, intracranial, intramuscular, and subcutaneous. Suitable devices for parenteral administration include needle injectors, needle-free injectors, and infusion techniques.

Parenteral compositions are typically aqueous solutions which may contain excipients such as salts, carbohydrates and buffering agents. However, the composition may also be formulated a sterile non-aqueous solution or as a dried form to be used in conjunction with a suitable vehicle such as sterile pyrogen-free water.

The preparation of parenteral compositions under sterile conditions, for example, by lyophilization, can be readily accomplished using standard techniques known well to those of skill in the art.

Compositions for parenteral administration can be formulated to be immediate and/or modified release. Modified release formulations include delayed-, sustained-, pulsed-, controlled-, targeted, and programmed release. Thus, the compositions can be formulated as a solid, semi-solid, or thixotropic liquid for administration as an implanted depot providing modified release of the active agent.

The parenteral formulations can be admixed with other suitable pharmaceutically acceptable excipients used in parenteral dosage forms such as, but not limited to, preservatives.

In another aspect, the pharmaceutical compositions can be formulated as suitable oral dosage forms such as tablets, capsules, powders, pellets, suspensions, solutions, emulsions, and the like. Other suitable carriers can be present such as disintegrants, diluents, chelating agents, binders, glidants, lubricants, fillers, bulking agents, anti-adherants, and the like.

Oral dosage formulations may also contain other suitable pharmaceutical excipients such as sweeteners, vehicle/wetting agents, coloring agents, flavoring agents, preservatives, viscosity enhancing/thickening agents, and the like.

The neoDegrader conjugates described herein can be used to treat various cancers. Certain conjugates of the present disclosure can be superior in terms of efficacy expression, pharmacokinetics (e.g., absorption, distribution, metabolism, excretion), solubility (e.g., water solubility), interaction with other medicaments (e.g., drug-metabolizing enzyme inhibitory action), safety (e.g., acute toxicity, chronic toxicity, genetic toxicity, reproductive toxicity, cardiotoxicity, carcinogenicity, central toxicity) and/or stability (e.g., chemical stability, stability to an enzyme), and can be useful as a medicament.

The neoDegrader conjugates of the present disclosure can be used as medicaments such as an agents for the prophylaxis or treatment of diseases, for example, cancers —e.g., colorectal cancers (e.g., colorectal cancer, rectal cancer, anus cancer, familial colorectal cancer, hereditary nonpolyposis colorectal cancer, gastrointestinal stromal tumor), lung cancers (e.g., non-small-cell lung cancer, small-cell lung cancer, malignant mesothelioma), mesothelioma, pancreatic cancers (e.g., pancreatic ductal carcinoma, pancreatic endocrine tumor), pharynx cancer, larynx cancer, esophageal cancer, stomach/gastric cancers (e.g., papillary adenocarcinoma, mucinous adenocarcinoma, adenosquamous carcinoma), duodenal cancer, small intestinal cancer, breast cancers (e.g., invasive ductal carcinoma, non-invasive ductal carcinoma, inflammatory breast cancer), ovarian cancers (e.g., ovarian epithelial cancer, extragonadal germ cell tumor, ovarian germ cell tumor, ovarian low-malignant potential tumor), testis tumor, prostate cancers (e.g., hormone-dependent prostate cancer, non-hormone dependent prostate cancer, castration-resistant prostate cancer), liver cancers (e.g., hepatocellular cancer, primary liver cancer, extrahepatic bile duct cancer), thyroid cancers (e.g., medullary thyroid carcinoma), renal cancers (e.g., renal cell cancers (e.g., clear cell renal cell cancer), transitional cell cancer of renal pelvis and ureter), uterine cancers (e.g., cervical cancer, uterine body cancer, uterus sarcoma), gestational choriocarcinoma, brain tumors (e.g., medulloblastoma, glioma, pineal astrocytic tumors, pilocytic astrocytoma, diffuse astrocytoma, anaplastic astrocytoma, pituitary adenoma), retinoblastoma, skin cancers (e.g., basalioma, malignant melanoma), sarcomas (e.g., rhabdomyosarcoma, leiomyosarcoma, soft tissue sarcoma, spindle cell sarcoma), malignant bone tumor, bladder cancer, hematological/blood cancers (e.g., multiple myeloma, leukemias (e.g., acute myelogenous leukemia), malignant lymphoma, Hodgkin’s disease, chronic myeloproliferative disease), cancer of unknown primary; a cancer growth inhibitor; a cancer metastasis inhibitor; an apoptosis promoter; an agent for the treatment of precancerous lesions (e.g., myelodysplastic syndromes); and the like.

In certain aspects, neoDegrader conjugates of the present disclosure can be used as a medicament for breast cancer, gastric cancer, ovarian cancer, uterine cancer, lung cancer, pancreatic cancer, liver cancer, lymphoma, or hematological cancers.

Furthermore, neoDegrader conjugates of the present disclosure or can be used concurrently with a non-drug therapy. To be precise, the conjugates can be combined with a non-drug therapy such as (1) surgery, (2) hypertensive chemotherapy using angiotensin II etc., (3) gene therapy, (4) thermotherapy, (5) cryotherapy, (6) laser cauterization and (7) radiotherapy.

For example, by using a neoDegrader conjugate of the present disclosure before or after the above-mentioned surgery and the like, effects such as prevention of emergence of resistance, prolongation of Disease-Free Survival, suppression of cancer metastasis or recurrence, prolongation of life and the like may be afforded.

In addition, it is possible to combine a treatment with neoDegrader conjugates of the present disclosure with a supportive therapy: (i) administration of antibiotic (e.g., β-lactam type such as pansporin and the like, macrolide type such as clarithromycin and the like) for the complication with various infectious diseases, (ii) administration of high-calorie transfusion, amino acid preparation or general vitamin preparation for the improvement of malnutrition, (iii) administration of morphine for pain mitigation, (iv) administration of a pharmaceutical agent for ameliorating side effects such as nausea, vomiting, anorexia, diarrhea, leucopenia, thrombocytopenia, decreased hemoglobin concentration, hair loss, hepatopathy, renopathy, DIC, fever and the like and (v) administration of a pharmaceutical agent for suppressing multiple drug resistance of cancer and the like.

In some aspects, the neoDegrader or neoDegrader conjugate of the disclosure can be used in combination with a standard of care therapy, e.g., one or more therapeutic agents (e.g., anti-cancer agents and/or immunomodulating agents). Accordingly, in certain aspects, a method of treating a tumor disclosed herein comprises administering the neoDegrader or neoDegrader conjugate of the disclosure in combination with one or more additional therapeutic agents. In some aspects, the neoDegrader or neoDegrader conjugate of the disclosure can be used in combination with one or more anti-cancer agents, such that multiple elements of the immune pathway can be targeted. In some aspects, an anti-cancer agent comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof. A comprehensive and non-limiting list of combination treatment is disclosed in detail in the Combination Treatments section of this application.

In some aspects, the neoDegrader or neoDegrader conjugate of the disclosure is administered to the subject prior to or after the administration of the additional therapeutic agent. In other aspects, the neoDegrader or neoDegrader conjugate of the disclosure is administered to the subject concurrently with the additional therapeutic agent. In certain aspects, the neoDegrader or neoDegrader conjugate of the disclosure and the additional therapeutic agent can be administered concurrently as a single composition in a pharmaceutically acceptable carrier. In other aspects, the neoDegrader or neoDegrader conjugate of the disclosure and the additional therapeutic agent are administered concurrently as separate compositions.

In some aspects, a subject that can be treated with the neoDegrader or neoDegrader conjugate of the present disclosure is a nonhuman animal such as a rat or a mouse. In some aspects, the subject that can be treated is a human.

### V. Methods of Preparing NeoDegraders and Compositions

The present disclosure provides a method of preparing the neoDegrader conjugates, the process comprising reacting a binding moiety with a compound of formula (I-1):

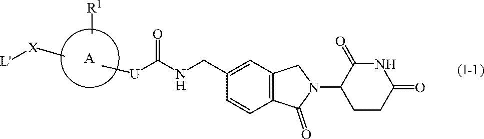

or a pharmaceutically acceptable salt thereof, wherein:
A is phenyl or a C₄-C₁₀cycloalkyl ring;
R¹ is independently selected from hydrogen and halo;
U is selected from NH and CF₂;
X is selected from —NR²—, ═C(CH₃)—, —Q—(CH₂)ₙ—, and —Q(CH₂)ₘQ′(CH₂)ₙ—; wherein
   Q and Q′ are each independently O, S, or NR²;
   R² is hydrogen or C₁-C₆alkyl;
   n is an integer from 1 to 6;
   m is an integer from 2 to 6; and
wherein the left side of each group is attached to L′ and the right side is attached to A;
provided that when X is NH or —Q—(CH₂)ₙ—, R¹ is halo;
L′ is a cleavable or non-cleavable linker precursor that conjugates to the binding moiety.

As described herein, the linker precursor contain a heterobifunctional group that connects to the binding moiety.

In some aspects, L′ is a non-cleavable linker precursor. In some aspects, L′ is selected from the group consisting of

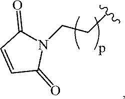

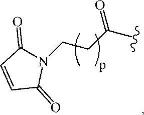

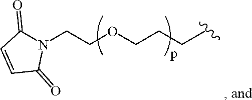

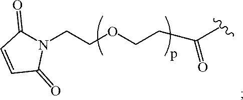

wherein:
p is an integer from 1 to 10; and

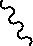

is the point of attachment to X.

In some aspects, L′ is

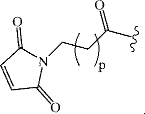

In some aspects, p is 5.

In certain aspects, L′ is a cleavable linker precursor.

In some aspects, the linker precursor is cleavable by a protease. In some aspects, the linker precursor is selected from the group consisting of

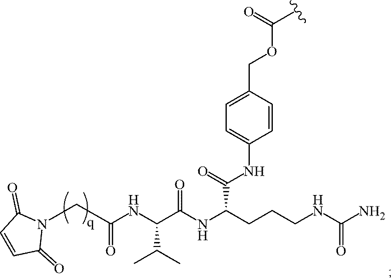

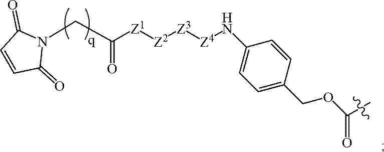

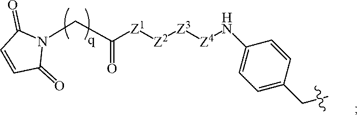

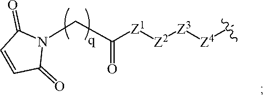

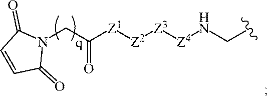

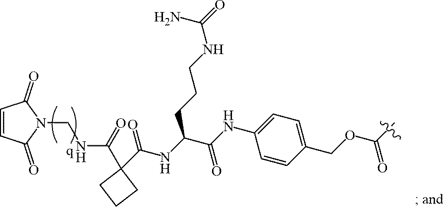

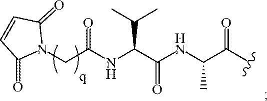

wherein:
q is an integer from 2 to 10;
Z¹, Z², Z³, and Z⁴ are each independently absent or a naturally-occurring amino acid residue in the L- or D-configuration, provided that at least two of Z¹, Z², Z³, and Z⁴ are amino acid residues; and

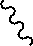

is the point of attachment to X.

In some aspects, Z¹, Z², Z³, and Z⁴ are independently absent selected from the group consisting of L-valine, D-valine, L-citrulline, D-citrulline, L-alanine, D-alanine, L-glutamine, D-glutaimine, L-glutamic acid, D-glutamic acid, L-aspartic acid, D-aspartic acid, L-asparagine, D-asparagine, L-phenylalanine, D-phenylalanine, L-lysine, D-lysine, and glycine, provided that at least two of Z¹, Z², Z³, and Z⁴ are amino acid residues.

In some aspects, Z¹ is absent or glycine; Z² is absent or selected from the group consisting of L-glutamine, D-glutamine, L-glutamic acid, D-glutamic acid, L-aspartic acid, D-aspartic acid, L-alanine, D-alanine, and glycine; Z³ is selected from the group consisting of L-valine, D-valine, L-alanine, D-alanine, L-phenylalanine, D-phenylalanine, and glycine; and Z⁴ is selected from the group consisting of L-alanine, D-alanine, L-citrulline, D-citrulline, L-asparagine, D-asparagine, L-lysine, D-lysine, L-phenylalamine, D-phenylalanine, and glycine.

In some aspects, L′ is

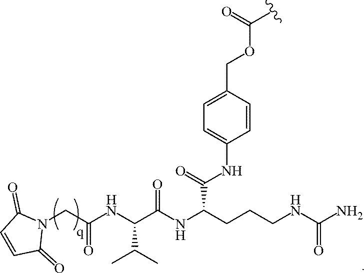

In some aspects, q is 5.

In some aspects, L′ is a bioreducible linker precursor. In some aspects, the bioreducible linker precursor is selected from the group consisting of

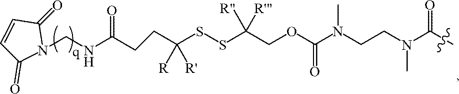

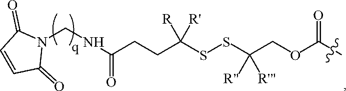

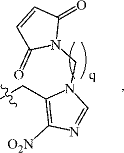

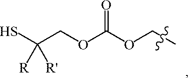

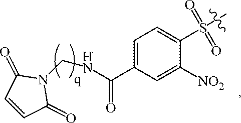

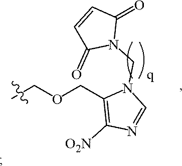

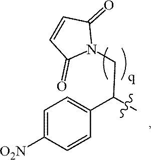

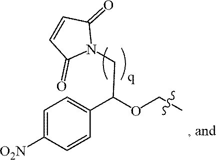

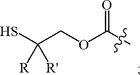

wherein:
q is an integer from 2 to 10;
R, R′, R″, and R‴ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring; and

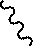

is the point of attachment to X.

In certain aspects, L′ is an acid cleavable linker precursor. In some aspects, L′ is selected from the group consisting of

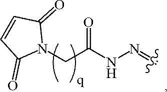

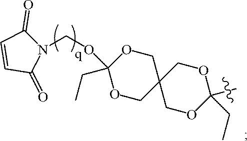

wherein:
q is an integer from 2 to 10; and

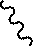

is the point of attachment to X.

In certain aspects, L′ is a click-to-release linker precursor. In some aspects, L′ is selected from

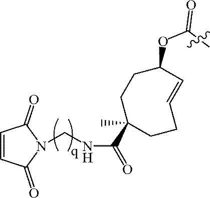

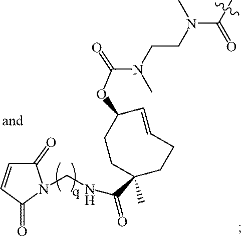

wherein:
q is an integer from 2 to 10; and

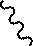

is the point of attachment to X.

In certain aspects, L′ is a pyrophosphatase cleavable linker precursor. In some aspects, L′ is

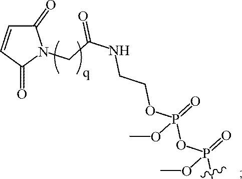

wherein:
q is an integer from 2 to 10;

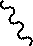

is the point of attachment to X.

In certain aspects, L′ is a beta-glucoronidase cleavable linker precursor. In some aspects, L′ is selected from

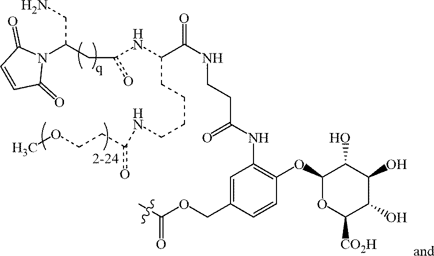

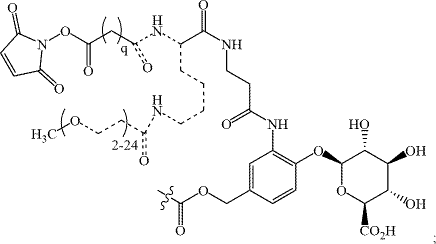

wherein:
q is an integer from 2 to 10;

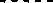

is absent or a bond; and

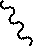

is the point of attachment to X.

In some aspects, the compound of formula (I-1) is selected from

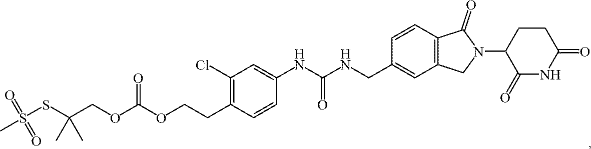

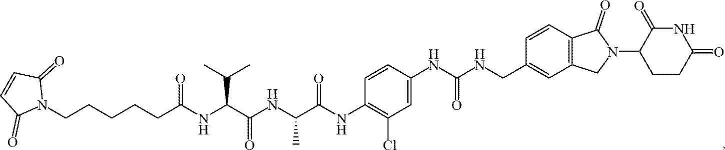

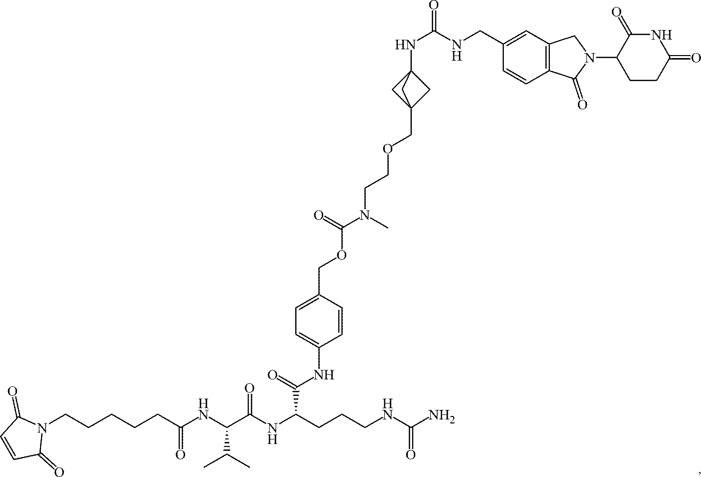

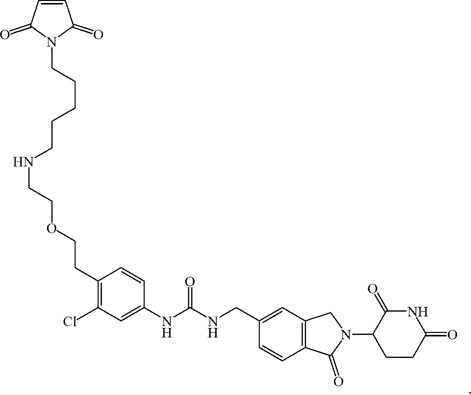

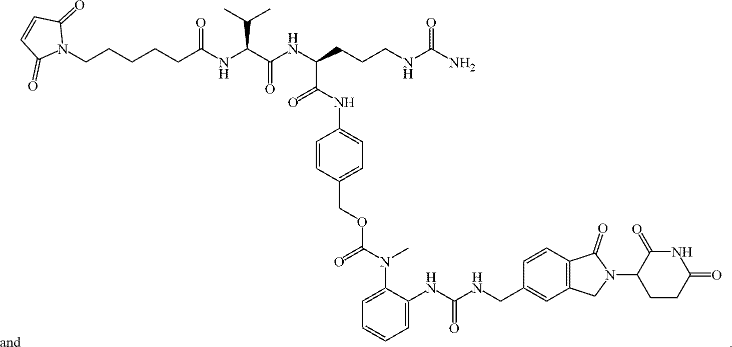

In some aspects, the binding moiety is pre-treated before it is reacted with the compound of formula (I-1). In certain aspects, the compound of formula (I-1) is reacted with a binding moiety, which comprises an antibody or an antigen binding portion thereof. In aspects where the binding moiety is an antibody, the antibody can be pretreated to reduce interchain disulfides prior to reaction with the compound of formula (I-1).

### Examples

### General Synthetic Methods and Intermediates

The compounds of the present disclosure can be prepared by one of ordinary skill in the art in light of the present disclosure and knowledge in the art, and/or by reference to the schemes shown below and the synthetic examples. Exemplary synthetic routes are set forth in Schemes below and in Examples. It should be understood that the variables, (for example “R” groups) appearing in the following schemes and examples are to be read independently from those appearing elsewhere in the application. One of ordinary skill in the art would readily understand how the schemes and examples shown below illustrate the preparation of the compounds described herein.

Abbreviations used in the schemes generally follow conventions used in the art. Chemical abbreviations used in the specification and examples are defined as follows: “THF” for tetrahydrofuran; “DMF” for N,N-dimethylformamide; “Me” for methyl; “Bu” for butyl; “FA” for formic acid; “PE” for petrolium ether; “MeOH” for methanol; “EtOH” for ethanol; “DCM” for dichlormethane; “BOC” or “Boc⁗TFA” for trifluoroacetic acid; “DMSO” for dimethylsulfoxide; “EtOAc” for ethyl acetate; “OAc” for acetate; “dppf” for 1,1′-bis(diphenylphosphino)ferrocene; “dba” for dibenzylideneacetone; “CDI” for 1,1′-carbonyldiimidazole; “TBAF” for tetrabutylammonium fluoride; “TBSCl” for tert-butyldimethylsilyl chloride; “Et₂O” for diethyl ether; “ACN” for acetonitrile; “h” for hours; “min” for mimutes; “rt” for room temperature or retention time (context will dictate); “aq.” for aqueous, “sat.” for saturated; “min” for minutes; “HOBt” for 1-hydroxybenzotriazole hydrate; “HATU” for 1-[bis(dimethylamino)methylene]-1H-1,2,3-triazolo[4,5-b]pyridinium 3-oxid hexafluorophosphate or N-[(dimethylamino)-1H-1,2,3-triazolo-[4,5-b]pyridin-1-ylmethylene]-N-methylmethanaminium hexafluorophosphate N-oxide; “DIEA” and “iPrNEt₂” for diisopropylethylamine; “Et₃N” and “TEA” for triethyl amine.

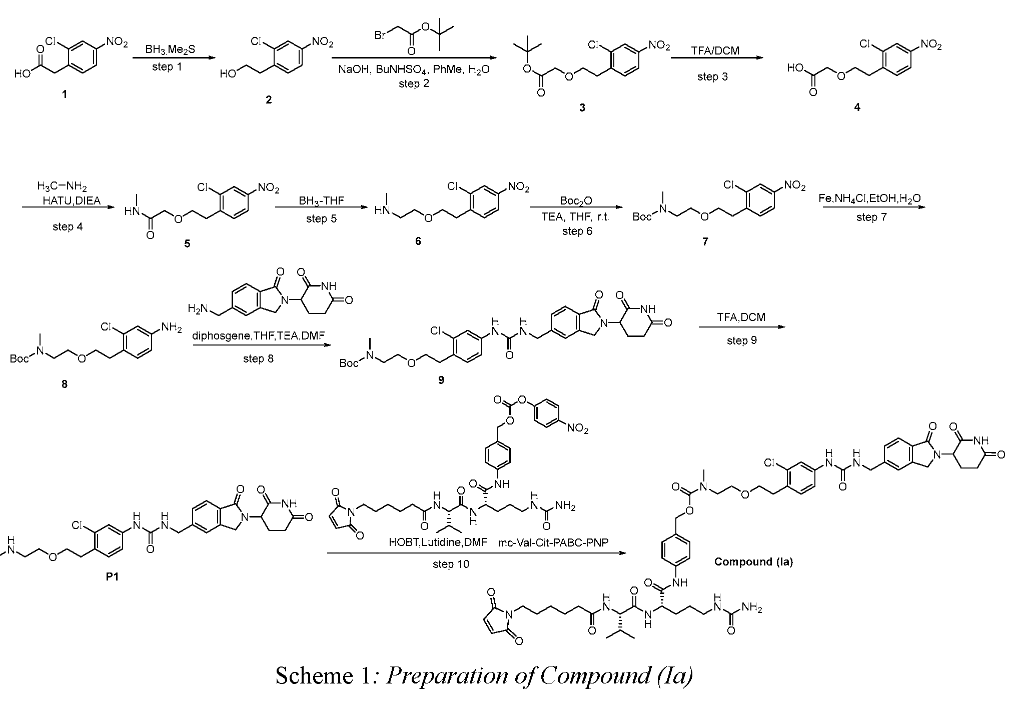 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CCOCCc2ccc(NC(=O)NCc3ccc4c(c3)CN(C3CCC(=O)NC3=O)C4=O)cc2Cl)cc1]

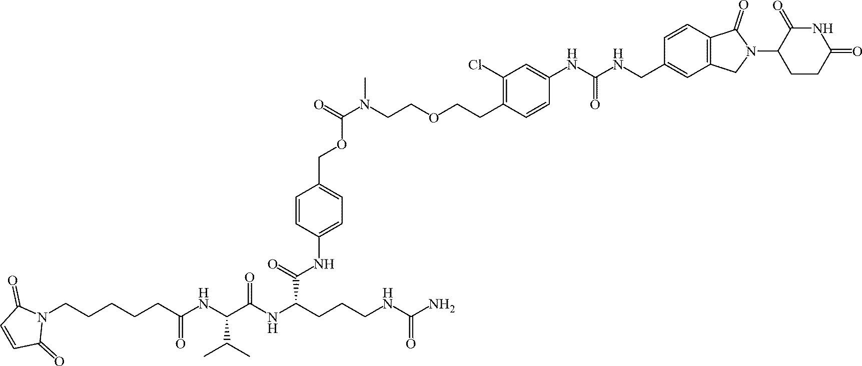 [SMILES: O=[N+]([O-])c1ccc(CCO)c(Cl)c1]

### Example 1: Synthesis of Compound (Ia)

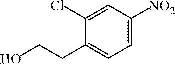 [SMILES: CC(C)(C)OC(=O)COCCc1ccc([N+](=O)[O-])cc1Cl]

### Step 1: Synthesis of Compound 2

To a stirred solution of 2-chloro-4-nitrophenyl)acetic acid (Compound 1, 5.00 g, 23.19 mmol, 1.00 equiv) in THF (75.00 mL) was added BH₃—Me₂S (10 M in THF) (5.80 mL, 58.0 mmol, 2.50 equiv) dropwise at 0° C. under nitrogen atmosphere. The resulting mixture was stirred for 2 h at 70° C. under nitrogen atmosphere. The mixture was cooled down to room temperature. The resulting mixture was concentrated under reduced pressure. The residue was purified by silica gel column chromatography (PE:EtOAc = 1:1) to afford 2-(2-chloro-4-nitrophenyl)ethanol (3 g, 64%) as a yellow solid. ¹H NMR (400 MHz, CDCl₃) δ 8.26 (d, J = 4.0 Hz, 1H), 8.10-8.05 (m, 1H), 7.50 (d, J = 8.0 Hz, 1H), 3.99-3.91 (m, 2H), 3.16-3.09 (m, 2H).

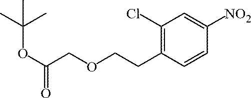 [SMILES: O=C(O)COCCc1ccc([N+](=O)[O-])cc1Cl]

### Step 2: Synthesis of Compound 3

To a stirred solution of 2-(2-chloro-4-nitrophenyl)ethanol (Compound 2, 5.00 g, 24.800 mmol, 1.00 equiv) and tert-butyl 2-bromoacetate (29.0 mL, 148.28 mmol, 8.00 equiv) in toluene (150.00 mL) was added Bu₄NHSO₄ (6.74 g, 19.84 mmol, 0.80 equiv). To the above mixture was added NaOH (5 M in H₂O) (500.00 mL) dropwise over 40 min at 0° C. The resulting mixture was stirred for additional 2 h at 25° C. The resulting mixture was extracted with EtOAc (3 × 500 mL). The combined organic layers were washed with brine (400 mL) and dried over anhydrous Na₂SO₄ . After filtration, the filtrate was concentrated under reduced pressure. The residue was purified by silica gel column chromatography (PE:EtOAc = 4:1) to afford tert-butyl 2-[2-(2-chloro-4-nitrophenyl)ethoxy]acetate (8 g, 65%) as a yellow oil. ¹H NMR (400 MHz, CDCl₃) δ 8.23 (d, J = 4.0 Hz, 1H), 8.10-8.04 (m, 1H), 7.60 (d, J = 8.0 Hz, 1H), 4.09 (s, 2H), 3.83-3.80 (m, 2H), 3.17-3.14(m, 2H), 1.45(s, 9H).

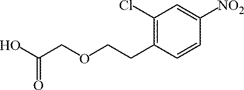 [SMILES: CNC(=O)COCCc1ccc([N+](=O)[O-])cc1Cl]

### Step 3: Synthesis of Compound 4

To a stirred solution of tert-butyl 2-[2-(2-chloro-4-nitrophenyl)ethoxy]acetate (Compound 3, 8.00 g, 16.14 mmol, 1.00 equiv, 63.7%) in DCM (80.00 mL) was added TFA (16.00 mL) dropwise at room temperature. The resulting mixture was stirred for 1 h at room temperature. The resulting mixture was concentrated under vacuum. The resulting mixture was diluted with water (500 mL). The mixture was extracted with EtOAc (3 × 500 mL). The combined organic layers were washed with brine (200 mL), dried over anhydrous Na2SO4. After filtration, the filtrate was concentrated under reduced pressure. This resulted in [2-(2-chloro-4-nitrophenyl)ethoxy]acetic acid (6.5 g, crude) as yellow oil. LCMS (ESI): 517 (2M-H)-

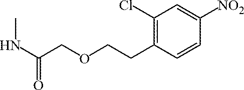 [SMILES: CNCCOCCc1ccc([N+](=O)[O-])cc1Cl]

### Step 4: Synthesis of Compound 5

To a stirred solution of [2-(2-chloro-4-nitrophenyl)ethoxy]acetic acid (Compound 4, 6.30 g, 21.84 mmol, 1.00 equiv, 90%) and HATU (12.46 g, 32.76 mmol, 1.50 equiv) in DMF (65.00 mL) was added CH₃NH₂.HCl (1.77 g, 26.21 mmol, 1.20 equiv) and DIEA (15.20 g, 117.8 mmol, 4.00 equiv) dropwise at room temperature. The resulting mixture was stirred for 2 h at room temperature. The resulting mixture was diluted with water. The resulting mixture was extracted with EtOAc (2 × 100 mL). The combined organic layers were washed with brine (50 mL), dried over anhydrous Na₂SO₄. After filtration, the filtrate was concentrated under reduced pressure. The residue was purified by silica gel column chromatography (DCM:MeOH = 10:1) to afford 2-[2-(2-chloro-4-nitrophenyl)ethoxy]-N-methylacetamide(10 g, purity:50%, yield:84%) as yellow oil. LCMS (ESI): 273.28 (M+H)⁺

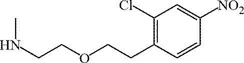 [SMILES: CN(CCOCCc1ccc([N+](=O)[O-])cc1Cl)C(=O)OC(C)(C)C]

### Step 5: Synthesis of Compound 6

To a stirred solution of 2-[2-(2-chloro-4-nitrophenyl)ethoxy]-N-methylacetamide (Compound 5, 3.3 g, 12.10 mmol, 1.00 equiv) in THF (35.00 mL) was added BH₃-THF (1 M in THF) (12.10 mL, 12.10 mmol, 1.00 equiv) dropwise at room temperature under nitrogen atmosphere. The resulting mixture was stirred for 2 h at 70° C. under nitrogen atmosphere. The reaction was quenched with MeOH. The residue was acidified to pH 6 with 1 N HCl. The resulting mixture was extracted with EtOAc (20 mL). The aqueous phase was basified to pH 8 with saturated NaHCO₃ (sat., aq.). The resulting mixture was extracted with EtOAc (3 × 100 mL), washed with brine (50 mL) and dried over anhydrous Na₂SO₄. After filtration, the filtrate was concentrated under reduced pressure. This resulted in [2-[2-(2-chloro-4-nitrophenyl)ethoxy]ethyl](methyl)amine (2.5 g, 80%) as yellow oil. LCMS (ESI): 259.26 (M+H)⁺

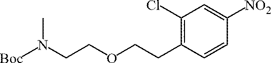 [SMILES: CN(CCOCCc1ccc(N)cc1Cl)C(=O)OC(C)(C)C]

### Step 6. Synthesis of Compound 7

To a stirred solution of [2-[2-(2-chloro-4-nitrophenyl)ethoxy]ethyl](methyl)amine (Compound 6, 2.50 g, 9.69 mmol, 1.00 equiv) and Boc₂O (2.53 g, 11.6 mmol, 1.20 equiv) in THF (40 mL) was added TEA (1.17 g, 11.6 mmol, 1.20 equiv) dropwise at 25° C. The mixture was stirred at 25° C. for 2 h. The resulting mixture was concentrated under vacuum. The residue was purified by silica gel column chromatography (DCM:MeOH = 5:1) to afford tert-butyl N-[2-[2-(2-chloro-4-nitrophenyl)ethoxy]ethyl]-N-methylcarbamate(1.70 g, 50%) as yellow oil. LCMS (ESI): 359.36 (M+H)⁺

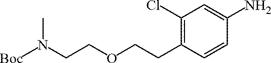 [SMILES: CN(CCOCCc1ccc(NC(=O)NCc2ccc3c(c2)CN(C2CCC(=O)NC2=O)C3=O)cc1Cl)C(=O)OC(C)(C)C]

### Step 7: Synthesis of Compound 8

To a stirred solution of tert-butyl N-[2-[2-(2-chloro-4-nitrophenyl)ethoxy]ethyl]-N-methylcarbamate (Compound 7, 1.70 g, 4.74 mmol, 1.00 equiv) and NH₄Cl (750 mg, 14.2 mmol, 3.00 equiv) in EtOH (85 mL) and H₂O (17 mL) was added Fe (1.3 g, 23.7 mmol, 5.00 equiv) at 25° C. The mixture was stirred at 80° C. for 2 h. The mixture was cooled down to room temperature. The resulting mixture was filtered, and the filter cake was washed with EtOH (3 × 50 mL). The filtrate was concentrated under reduced pressure. The residue was purified by silica gel column chromatography (PE : EtOAc = 4:1) to afford tert-butyl N-[2-[2-(4-amino-2-chlorophenyl)ethoxy]ethyl]-N-methylcarbamate (900 mg, 58%) as yellow oil. LCMS (ESI): 329.33 (M+H)⁺

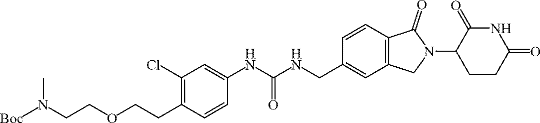 [SMILES: CNCCOCCc1ccc(NC(=O)NCc2ccc3c(c2)CN(C2CCC(=O)NC2=O)C3=O)cc1Cl]

### Step 8: Synthesis of Compound 9

To a stirred solution of tert-butyl N-[2-[2-(4-amino-2-chlorophenyl)ethoxy]ethyl]-N-methylcarbamate (Compound 8, 500 mg, 1.52 mmol, 1.00 equiv) in THF (10 mL) was added diphosgene (601 mg, 3.04 mmol, 2.00 equiv) dropwise at 25° C. The mixture was stirred at 25° C. for 1 h. The resulting mixture was concentrated under vacuum and re-dissolved in DMF (5 mL). To a stirred mixture of 3-[5-(aminomethyl)-1-oxo-3H-isoindol-2-yl]piperidine-2,6-dione (INT1, prepared as described below, 499 mg, 1.82 mmol, 1.20 equiv) and TEA (1.56 g, 15.45 mmol, 10.00 equiv) in DMF (20 mL) was added the solution mentioned above dropwise at 25° C. The mixture was stirred at 25° C. for 1 h. The resulting mixture was diluted with 40 mL of ice water. The resulting mixture was extracted with EtOAc (3 × 40 mL). The combined organic layers were washed with brine (5×40 mL) and dried over anhydrous Na₂SO₄. After filtration, the filtrate was concentrated under reduced pressure. The residue was purified by silica gel column chromatography (DCM: MeOH = 10:1) to afford tert-butyl (2-(2-chloro-4-(3-((2-(2,6-dioxopiperidin-3-yl)-1-oxoisoindolin-5-yl)methyl)ureido)phenethoxy)ethyl)(methyl)carbamate (670 mg, 70%) as a white solid. LCMS: (ESI): 628.63 (M+H)⁺

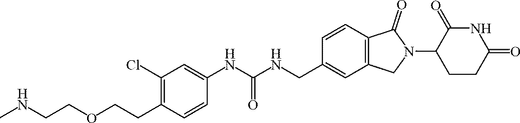 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CCOCCc2ccc(NC(=O)NCc3ccc4c(c3)CN(C3CCC(=O)NC3=O)C4=O)cc2Cl)cc1]

### Step 9: Synthesis of neoDegrader P1

To a stirred solution of tert-butyl N-[2-(2-[2-chloro-4-[([[2-(2,6-dioxopiperidin-3-yl)-1-oxo-3H-isoindol-5-yl]methyl]carbamoyl)amino]phenyl]ethoxy)ethyl]-N-methylcarbamate (Compound 9, 670 mg, 1.07 mmol, 1 eq) in DCM (10 mL) was added TFA (2.5 mL) dropwise at 0° C. The mixture was stirred at 25° C. for 1 h. The resulting mixture was concentrated under vacuum. The crude product was purified by Prep-HPLC with the following conditions: Column, SunFire C18 OBD Prep Column, 100 µm, 19×250 mm; mobile phase, water (0.05% TFA) and ACN (5% Phase B up to 60% in 30 min); Detector, UV 220 nm. The collected fraction was lyophilized to give 1-(3-chloro-4-[2-[2-(methylamino)ethoxy]ethyl]phenyl)-3-[[2-(2,6-dioxopiperidin-3-yl)-1-oxo-3H-isoindol-5-yl]methyl]urea (500 mg, 89%) as a white solid. LCMS (ESI): 528.53 (M+H)⁺. ¹H NMR (400 MHz, Methanol-d₄) δ 7.77 (d, J = 8.0 Hz, 1H), 7.57-7.53 (m, 2H), 7.49 (d, J = 8.0 Hz, 1H), 7.21 (d, J = 4.0 Hz, 2H), 5.19-5.1 (m, 1H), 4.55-4.41 (m, 4H), 3.75-3.67 (m, 4H), 3.21-3.15 (m,2H), 3.03-3.96 (m, 2H), 2.96-2.84 (m, 1H), 2.83-2.73 (m, 2H), 2.69 (s, 3H), 2.55-2.42 (m, 1H), 2.21-2.12 (m, 1H).

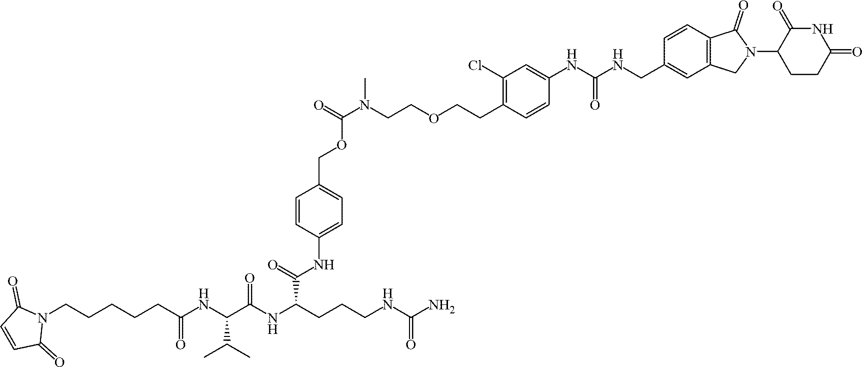 [SMILES: CC(C)(COC(=O)OCCc1ccc(NC(=O)NCc2ccc3c(c2)CN(C2CCC(=O)NC2=O)C3=O)cc1Cl)SS(C)(=O)=O]

### Step 10: Synthesis of Compound (Ia)

To a stirred mixture of 1-(3-chloro-4-[2-[2-(methylamino)ethoxy]ethyl]phenyl)-3-[[2-(2,6-dioxopiperidin-3-yl)-1-oxo-3H-isoindol-5-yl]methyl]urea (neoDegrader P1, 200 mg, 0.38 mmol, 1.00 equiv) and lutidine (81 mg, 0.76 mmol, 2.00 equiv) in DMF (10 mL) were added HOBT (26 mg, 0.19 mmol, 0.50 equiv) and [4-[(2S)-5-(carbamoylamino)-2-[(2S)-2-[6-(2,5-dioxopyrrol-1-yl)hexanamido]-3-methylbutanamido]pentanamido]phenyl]methyl 4-nitrophenyl carbonate (279 mg, 0.38 mmol, 1.00 equiv) in portions at room temperature. The reaction mixture was stirred for 12 hours at 40° C. under nitrogen atmosphere. After the reaction was cooled down to room temperature, the reaction was quenched with water (30 mL). The resulting mixture was extracted with DCM (3 × 30 mL). The combined organic layers were washed with water (2 × 30 mL), brine (30 mL), dried over Na₂SO₄. After filtration, the filtrate was concentrated to dryness under vacuum. The residue was purified by reverse phase column (C18, mobile phase A: 0.1% FA in water, B: ACN). The collected fraction was concentrated to dryness under vacuum. The crude product (60 mg) was purified by Prep-HPLC with the following conditions (Column: Xselect CSH OBD Column 30×150mm 5um, n; Mobile Phase A:Water(0.1%FA), Mobile Phase B:ACN; Flow rate:60 mL/min; Gradient:33 B to 50 B in 7 min; 220 nm; RT1:5.27 min). The collected fraction was lyophilized to afford [4-[(2S)-5-(carbamoylamino)-2-[(2S)-2-[6-(2,5-dioxopyrrol-1-yl)hexanamido]-3-methylbutanamido]pentanamido]phenyl]methyl N-[2-(2-[2-chloro-4-[([[2-(2,6-dioxopiperidin-3-yl)-1-oxo-3H-isoindol-5-yl]methyl]carbamoyl)amino]phenyl]ethoxy)ethyl]-N-methylcarbamate (23.8 mg, 5%) as a white solid. LCMS (ESI): 1126.11 (M+H)⁺. ¹H NMR (400 MHz, DMSO-d₆) δ 10.99(s, 1H), 10.00(s, 1H), 8.88(s, 1H), 8.12-8.08(m, 1H),7.85-7.81(m, 2H), 7.70-7.67(m, 2H), 7.60-7.58(m, 1H), 7.51(s, 1H), 7.47-7.44(m, 1H), 7.28-7.25(m, 2H), 7.18-7.12(m, 2H), 7.00(s, 2H), 6.90(br s, 1H), 5.97-5.95(m, 1H), 5.42(s, 2H), 5.12-5.05(m, 1H), 4.98(s, 2H), 4.42-4.32(m, 4H), 4.18-4.15(m, 1H), 3.56-3.40(m, 4H), 3.37-3.36(m, 3H),3.05-2.90(m, 3H), 2.89-2.85(m, 5H), 2.72-2.55(m, 2H), 2.40-2.33(m, 2H), 2.25-2.15(m, 2H), 2.00-1.87(m, 2H), 1.74-1.57(m, 2H), 1.50-1.42(m, 5H), 1.22-1.10(m, 3H), 0.85-0.80(m, 6H).

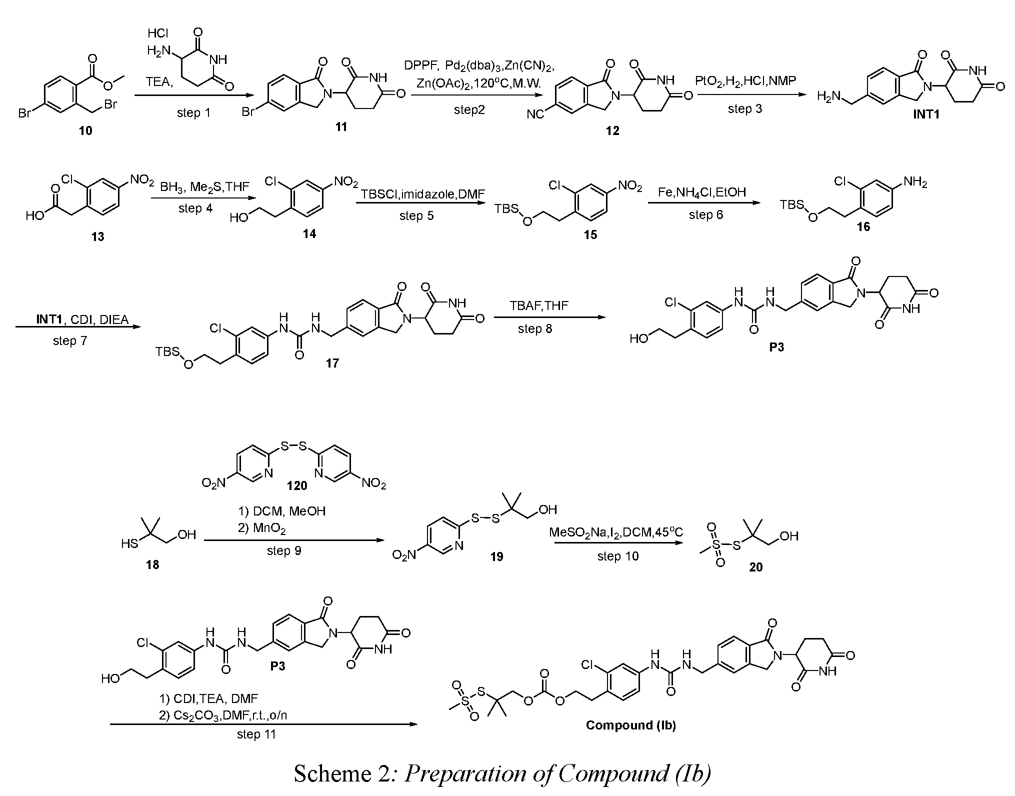 [SMILES: O=C1CCC(N2Cc3cc(Br)ccc3C2=O)C(=O)N1]

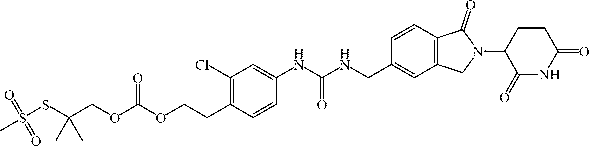

### Example 2: Synthesis of Compound (Ib)

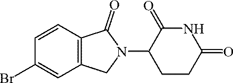 [SMILES: NCc1ccc2c(c1)CN(C1CCC(=O)NC1=O)C2=O]

### Step 1: Synthesis of Compound 11

To a stirred mixture of methyl 4-bromo-2-(bromomethyl)benzoate (Compound 10, 20.0 g, 64.8 mmol, 1.00 equiv) and 3-aminopiperidine-2,6-dione hydrochloride (10.64 g, 83.0 mmol, 1.28 equiv) in DMF (80 ml) was added TEA (22.4 mL, 162.2 mmol, 2.50 equiv) dropwise at 25° C. under nitrogen atmosphere. The mixture was stirred at 25° C. for 16 h. This was followed by addition of H2O (60 mL), AcOH (23 mL) and Et2O (60 mL) in sequence at 25° C. The mixture was stirred at 25° C. for 2 h. The precipitated solids were collected by filtration and washed with Et2O (60 mL). This resulted in 3-(5-bromo-1-oxo-3H-isoindol-2-yl)piperidine-2,6-dione (9.0 g, 42%) as a light blue solid. LCMS (ESI): 323.32 (M+H)⁺

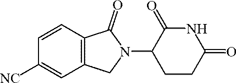 [SMILES: O=[N+]([O-])c1ccc(CCO)c(Cl)c1]

### Step 2: Synthesis of Compound 12

To a stirred mixture of 3-(5-bromo-1-oxo-3H-isoindol-2-yl)piperidine-2,6-dione (Compound 11, 1.00 g, 3.09 mmol, 1.00 equiv) and dppf (51 mg, 0.093 mmol, 0.03 equiv) in DMF (8 mL) were added Zn(OAc)₂ (170 mg, 0.928 mmol, 0.30 equiv), Zn(CN)₂ (545 mg, 4.64 mmol, 1.50 equiv) and Pd₂(dba)₃ (28 mg, 0.031 mmol, 0.01 equiv) at 25° C. under nitrogen atmosphere. The final reaction mixture was irradiated with microwave radiation for 2 h at 120° C. The mixture was cooled down to room temperature and filtered. The filter cake was washed with MeOH (3×30 mL). The filtrate was concentrated under reduced pressure. The residue was subjected to flash chromatography (silica gel, 80 g, DCM: MeOH=10: 1) to give the desired product 2-(2,6-dioxopiperidin-3-yl)-1-oxo-3H-isoindole-5-carbonitrile (400 mg, 47%) as a brown solid. LCMS (ESI): 270 (M+H)⁺

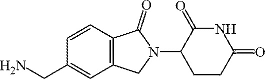

### Step 3: Synthesis of INT1

To a stirred mixture of 2-(2,6-dioxopiperidin-3-yl)-1-oxo-3H-isoindole-5-carbonitrile (Compound 12, 3.0 g, 11.14 mmol, 1.00 equiv) and HCl (12 M) (3.6 mL) in MeOH (25 mL) was added PtO₂ (1.25 g, 5.5 mmol, 0.49 equiv) at 25° C. The mixture was hydrogenated at room temperature for 16 h under hydrogen atmosphere using a hydrogen balloon. The resulting mixture was filtered, and the filter cake was washed with MeOH (2 × 30 mL). The filtrate was concentrated under reduced pressure. The resulting solid was washed with DCM: MeOH (3:1) (3×30 mL) and dried. This resulted in 3-[5-(aminomethyl)-1-oxo-3H-isoindol-2-yl]piperidine-2,6-dione (2.5 g, 80%) as grey solid. LCMS (ESI): 274 (M+H)⁺. ¹H NMR (400 MHz, DMSO-d₆) δ 11.02 (s, 1H), 8.15 (s, 1H), 7.98 (d, J=8.4 Hz, 1H), 7.89(d, J=8.4 Hz, 1H),5.16-5.11 (m, 1H), 4.52 (d, J=17.2 Hz, 1H), 4.40 (d, J=17.2 Hz, 1H), 2.96-2.90 (m, 1H), 2.60-2.54 (m, 1H), 2.43-2.34 (m, 1H), 2.06-1.96 (m, 1H)

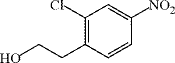

### Step 4: Synthesis of Compound 14

To a stirred solution of (2-chloro-4-nitrophenyl)acetic acid (Compound 13, 5.00 g, 22.50 mmol, 1.00 equiv) in THF (75 mL) was added BH₃—Me₂S (10 M in THF) (5.60 mL, 56 mmol, 2.50 equiv) dropwise at 0° C. under nitrogen atmosphere. The mixture was stirred at 70° C. for 2 h. The resulting mixture was concentrated under vacuum. The residue was applied onto silica gel column and eluted with PE / EtOAc (5:1) to afford 2-(2-chloro-4-nitrophenyl)ethanol (4.44 g, 88%) as a yellow solid. ¹H NMR (400 MHz, CDCl₃) δ 8.26 (d, J = 4.0 Hz, 1H), 8.10-8.05 (m, 1H), 7.50 (d, J = 8.0 Hz, 1H), 3.99-3.91 (m, 2H), 3.16-3.09 (m, 2H)

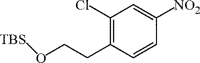

### Step 5: Synthesis of Compound 15

To a stirred mixture of 2-(2-chloro-4-nitrophenyl)ethanol (Compound 14, 4.44 g, 22.02 mmol, 1.00 equiv) and imidazole (4.50 g, 66.06 mmol, 3.00 equiv) in DMF (50.00 mL) was added TBSC1 (6.97 g, 46.25 mmol, 2.10 equiv) at 25° C. The mixture was stirred at 25° C. for 16 h. The resulting mixture was diluted with water (100 mL). The resulting mixture was extracted with EtOAc (3 × 100 mL). The combined organic layers were washed with brine (3×100 mL), dried over anhydrous Na₂SO₄. After filtration, the filtrate was concentrated under reduced pressure. The residue was applied onto silica gel column and eluted with PE/ EtOAc (10:1) to afford tert-butyl[2-(2-chloro-4-nitrophenyl)ethoxy]dimethylsilane (6.6 g, 90%) as a colorless oil. ¹H NMR (400 MHz, CDCl₃) δ 8.24(s, 1H), 8.06-8.04 (m, 1H), 7.46 (d, J = 8.4 Hz, 1H), 3.89-3.86 (m, 2H), 3.06-0.04 (m, 2H), 0.85(s, 9H), 0.04(s, 6H).

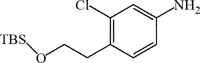 [SMILES: O=C1CCC(N2Cc3cc(CNC(=O)Nc4ccc(CCO)c(Cl)c4)ccc3C2=O)C(=O)N1]

### Step 6: Synthesis of Compound 16

To a mixture of tert-butyl[2-(2-chloro-4-nitrophenyl)ethoxy]dimethylsilane (Compound 15, 5.70 g, 18.05 mmol, 1.00 equiv) and Fe (10.08 g, 180.45 mmol, 10.00 equiv) in EtOH (110 mL) /water (55 mL) was added NH4Cl (9.65 g, 180.45 mmol, 10 equiv). The mixture was stirred at 80° C. for 2 h. The mixture was cooled down to room temperature. The resulting mixture was filtered, and the filter cake was washed with EtOH (3×50 mL). The filtrate was concentrated under reduced pressure. The residue was diluted with water (100 mL) and extracted with EtOAc (50 mLx3).The combined organic layer was dried over anhydrous sodium sulfate and evaporated to dryness in vacuo to give 4-[2-[(tert-butyldimethylsilyl)oxy]ethyl]-3-chloroaniline(5.2 g, crude) as a pale brown oil. LCMS (ESI): 286.29 (M+H)⁺

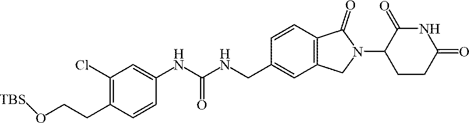 [SMILES: CC(C)(CO)SSc1ccc([N+](=O)[O-])cn1]

### Step 7: Synthesis of Compound 17

To a solution of 4-[2-[(tert-butyldimethylsilyl)oxy]ethyl]-3-chloroaniline (Compound 16, 200.00 mg, 0.70 mmol, 1.00 equiv) and TEA ( 141 mg, 1.40 mmol, 2.00 equiv) in DMF (3 mL) was added CDI (113 mg, 0.70 mmol, 1.00 equiv) in DMF (1 mL) dropwise under nitrogen dropwise at 0° C. The resulting mixture was stirred at 25° C. for 1 hour. Then the above solution and TEA (141 mg, 1.40 mmol) were added dropwise into solution of 3-[5-(aminomethyl)-1-oxo-3H-isoindol-2-yl]piperidine-2,6-dione (INT1, 192 mg, 0.70 mmol, 1.00 equiv) in DMF (2 mL). The same reaction was repeated for twice. The resulting mixture was stirred at 25° C. for 1 hour. The reaction was diluted with water (20 mL), extracted with EtOAc (20 mL x3). The combined organic layer was washed with water, brine, dried over anhydrous sodium sulfate and evaporated to dryness in vacuum. The residue was purified with silica gel column (DCM:MeOH=10:1) to give 1-(4-[2-[(tert-butyldimethylsilyl)oxy]ethyl]-3-chlorophenyl)-3-[[2-(2,6-dioxopiperidin-3-yl)-1-oxo-3H-isoindol-5-yl]methyl]urea (170 mg, 21%) as a white solid. LCMS (ESI): 585.59 (M+H)⁺

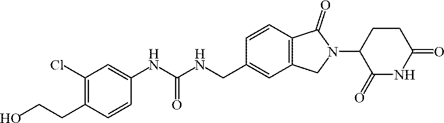

### Step 8: Synthesis of neoDegrader P3

To a solution of 1-(4-[2-[(tert-butyldimethylsilyl)oxy]ethyl]-3-chlorophenyl)-3-[[2-(2,6-dioxopiperidin-3-yl)-1-oxo-3H-isoindol-5-yl]methyl]urea (Compound 17, 170.00 mg, 0.29 mmol, 1.00 equiv) in THF (2.00 mL) was added TBAF (1 N in THF, 0.58 mL, 0.58 mmol, 2.00 equiv) at 0° C. The resulting mixture was stirred at 25° C. for 8 hours. The reaction was purified with Prep-TLC (DCM:MeOH=10:1) to give 147 mg of the crude 1-(3-chloro-4-(2-hydroxyethyl)phenyl)-3-((2-(2,6-dioxopiperidin-3-yl)-1-oxoisoindolin-5-yl)methyl)urea as a white solid. LCMS (ESI): 471.47 (M+H)⁺

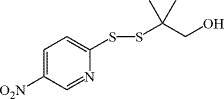 [SMILES: CC(C)(COC(=O)OCCc1ccc(NC(=O)NCc2ccc3c(c2)CN(C2CCC(=O)NC2=O)C3=O)cc1Cl)SS(C)(=O)=O]

### Step 9: Synthesis of Compound 19

2-Methyl-2-sulfanylpropan-1-ol (Compound 18, 1.4 g, 13.2 mmol, 1.00 equiv) and 5-nitro-2-[(5-nitropyridin-2-yl)disulfanyl]pyridine (Compound 120, 2.05 g, 6.67 mmol, 0.50 equiv) were added into a mixture of solvent of dichloromethane (3.50 mL) and MeOH (3.50 mL). The resulting mixture was stirred at 15° C. Then manganese dioxide (2.29 g, 26.2 mmol, 2 equiv) was added in portions. The resulting mixture was stirred at 15° C. for 15 min. LCMS traces showed the reaction was completed. The reaction was evaporated to dryness and the residue was purified by reverse flash chromatography with the following conditions: column, C18 silica gel; mobile phase, ACN in water (0.1% NH₄HCO₃), 10% to 100% gradient in 30 min; detector, UV 254 nm. The collected fraction was concentrated to dryness under vacuum to afford 2-methyl-2-[(5-nitropyridin-2-yl)disulfanyl]propan-1-ol (2.2 g, 58%) as a yellow solid. LCMS (ESI): 261 (M+H)⁺.

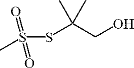 [SMILES: CC(C)[C@H](NC(=O)CCCCCN1C(=O)C=CC1=O)C(=O)N[C@@H](CCCNC(N)=O)C(=O)Nc1ccc(COC(=O)N(C)c2ccccc2NC(=O)NCc2ccc3c(c2)CN(C2CCC(=O)NC2=O)C3=O)cc1]

### Step 10: Synthesis of Compound 20

To a solution of 2-methyl-2-[(5-nitropyridin-2-yl)disulfanyl]propan-1-ol (Compound 20, 1.0 g, 3.84 mmol, 1.00 equiv) in anhydrous DCM (30 mL) was added MeSO₂Na (1.57 g, 15.4 mmol, 4.00 equiv) and iodine (1.95 g, 7.68 mmol, 2.00 equiv) in portions. The reaction mixture was stirred at 45° C. for 24 h. The mixture was concentrated, and the residue was purified by column chromatography on silica gel (TLC: PE:EA=3:1, Rf = 0.60; 0-35% EtOAc in petroleum ether) to afford 2-(methanesulfonylsulfanyl)-2-methylpropan-1-ol (80 mg, 10%) as a yellow oil. ¹H NMR (400 MHz, CD₃Cl)— δ 3.50(s, 2H), 3.33(s, 3H), 2.16(br s, 1H), 1.47(s, 6H).

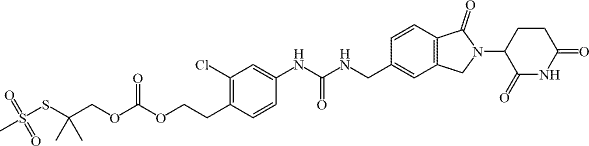 [SMILES: CN(C(=O)OC(C)(C)C)c1ccccc1NC(=O)NCc1ccc2c(c1)CN(C1CCC(=O)NC1=O)C2=O]

### Step 11: Synthesis of Compound (Ib)

To a solution of 1-[3-chloro-4-(2-hydroxyethyl)phenyl]-3-2-(2,6-dioxopiperidin-3-yl)-1-oxo-3H-isoindol-5-yl]methyl]urea (neoDegrader P3, 200.00 mg, 0.42 mmol, 1.00 equiv) and TEA (129 mg, 1.26 mmol, 3.00 equiv) in DMF (4 mL) was added a solution of CDI (138 mg, 0.84 mmol, 2.00 equiv) in DMF (1 mL). The reaction mixture was stirred at room temperature for 2 hours. The reaction was diluted with water (50 mL) and extracted with EtOAc (20 mLx3). The combined organic layer was washed with water (20 mLx3), brine (20 mL), dried over sodium sulfate and evaporated to dryness in vacuum to give the crude product (2-[2-chloro-4-[([[2-(2,6-dioxopiperidin-3-yl)-1-oxo-3H-isoindol-5-yl]methyl]carbamoyl)amino]phenyl]ethyl imidazole-1-carboxylate, 200 mg) as pale yellow solid. To a solution of the crude product (100.00 mg, 0.18 mmol, 1.00 equiv) and Cs₂CO₃ (115 mg, 0.35 mmol, 2.00 equiv) in DMF (8 mL) was added 2-(methanesulfonylsulfanyl)-2-methylpropan-1-ol (Compound 20, 59 mg, 0.32 mmol, 1.80 equiv) in DMF (2 mL) dropwise at room temperature. The reaction was stirred at 15° C. for 22 hours. The reaction was diluted with EtOAc (50 ml) and ice-cooled water (100 mL). The organic layer was separated out. The water phase was extracted with EtOAc (30 mLx3). The combined organic layer was washed with brine (30 mLx3), dried over anhydrous sodium sulfate and evaporated to dryness in vacuum to give the crude product (150 mg) as a yellow solid. The crude product was purified with Prep-HPLC (Column: Xselect CSH OBD Column 30×150 mm 5 um; Mobile Phase A:Water(0.1%FA), Mobile Phase B:ACN; Flow rate:60 mL/min; Gradient:38 B to 58 B in 7 min; 220 nm; RT1:5.12 min). The collected fraction was lyophilized to give 1-[3-chloro-4-[2-([[2-(methanesulfonylsulfanyl)-2-methylpropoxy]carbonyl]-oxy)ethyl]phenyl]-3-[[2-(2,6-dioxopiperidin-3-yl)-1-oxo-3H-isoindol-5-yl]methyl]urea (15.7 mg, 11%) as a white solid. LCMS (ESI): 681.68 (M+H)⁺. ¹H NMR (400 MHz, DMSO-d₆) δ 10.99 (s, 1H), 8.86 (s, 1H), 7.70 (d, J = 2.4 Hz, 1H), 7.51 (s, 1H), 7.44 (d, J = 8.0 Hz, 1H), 7.24-7.17 (m, 1H), 6.87-6.84 (m, 1H), 5.76 (s, 2H), 5.13-5.11 (m, 1H), 4.42-4.40 (m, 2H), 4.32-4.28 (m, 4H), 3.54 (s, 3H), 3.00-2.87 (m, 3H), 2.62-2.58 (m, 1H), 2.44-2.34 (m, 1H), 2.01-1.95 (m, 1H), 1.45 (s, 6H).

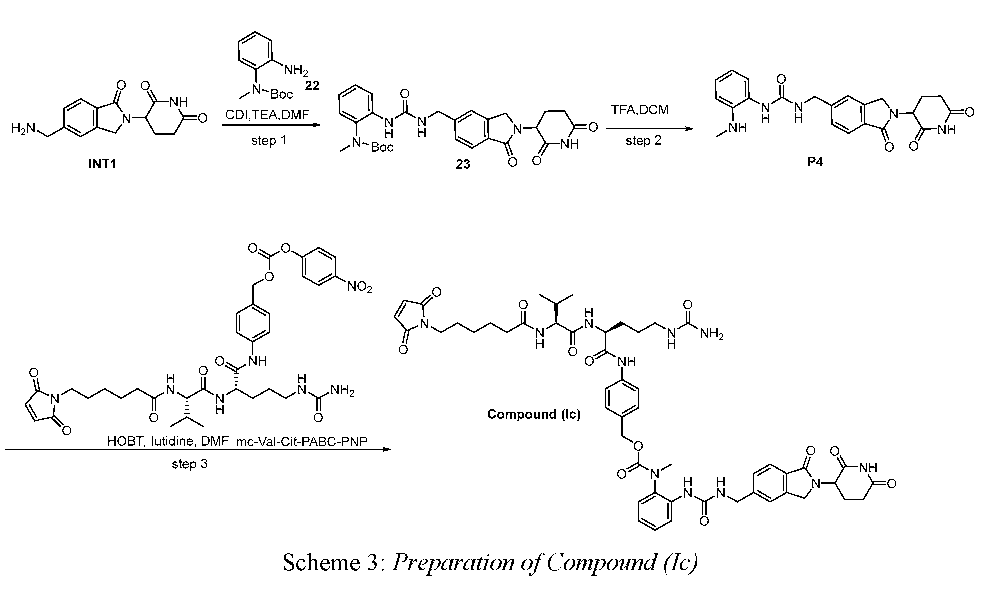 [SMILES: CNc1ccccc1NC(=O)NCc1ccc2c(c1)CN(C1CCC(=O)NC1=O)C2=O]

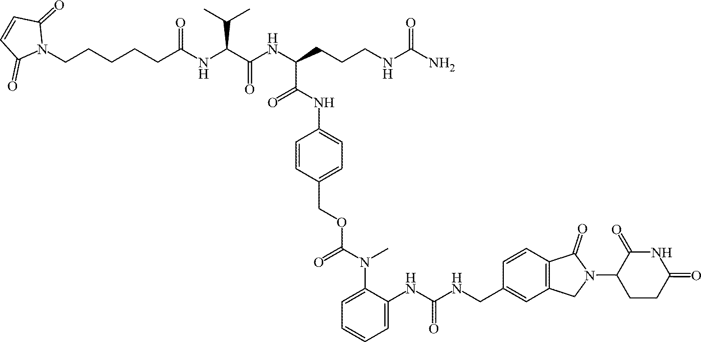 [SMILES: CC(C)[C@H](NC(=O)CCCCCN1C(=O)C=CC1=O)C(=O)N[C@@H](CCCNC(N)=O)C(=O)Nc1ccc(COC(=O)N(C)c2ccccc2NC(=O)NCc2ccc3c(c2)CN(C2CCC(=O)NC2=O)C3=O)cc1]

### Example 3: Synthesis of Compound (Ic)

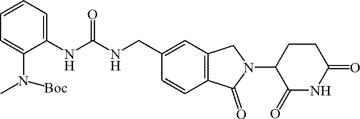

### Step 1: Synthesis of Compound 23

To a stirred solution of tert-butyl (2-aminophenyl)(methyl)carbamate (Compound 22, 300 mg, 1.35 mmol, 1.00 equiv) in DMF (20 mL) was added CDI (218 mg, 1.35 mmol, 1.00 equiv) and TEA (68 mg, 1.35 mmol, 1.00 equiv) dropwise at 0° C. under nitrogen atmosphere. The mixture was stirred at 0° C. for 2 h. To the above mixture was added 3-[5-(aminomethyl)-1-oxo-3H-isoindol-2-yl]piperidine-2,6-dione (INT1, 368 mg, 1.35 mmol, 1.00 equiv) in portions. The resulting mixture was stirred for overnight at 75° C. Then the reaction mixture was cooled down to room temperature. The resulting mixture was quenched with water (30 mL) and extracted with DCM (3 ×30 mL). The combined organic layers were washed with brine (30 mL), dried over anhydrous Na₂SO₄, filtered and concentrated under reduced pressure. The residue was purified by silica gel column chromatography ( DCM/MeOH =10:1) to afford tert-butyl N-[2-[([[2-(2,6-dioxopiperidin-3-yl)-1-oxo-3H-isoindol-5-yl]methyl]carbamoyl)amino]phenyl]-N-methylcarbamate (300 mg, 42%) as white solid. LCMS (ESI): 522 (M+H)⁺

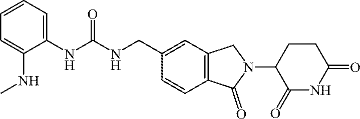

### Step 2. Synthesis of neoDegrader P4

To a stirred solution tert-butyl N-[2-[([[2-(2,6-dioxopiperidin-3-yl)-1-oxo-3H-isoindol-5-yl]methyl]carbamoyl)amino]phenyl]-N-methylcarbamate (Compound 23, 300 mg, 1.00 equiv) in DCM (20 mL) was added TFA (5 mL) at 0° C. The mixture was stirred at 0° C. for 2 h. The resulting mixture was concentrated under vacuum. The crude product was purified by reverse phase with the following conditions (C18, Mobile Phase A: Water (0.1% FA), Mobile Phase B: ACN; Flow rate:60 mL/min). The collected fraction was concentrated under vacuum to afford 3-[[2-(2,6-dioxopiperidin-3-yl)-1-oxo-3H-isoindol-5-yl]methyl]-1-[2-(methylamino)phenyl]urea (210 mg, 87%) as a white solid. LCMS (ESI): 422 (M+H)⁺. ¹H NMR (300 MHz, DMSO-d₆) δ 10.99(s, 1H), 7.69 (d, J = 7.8 Hz, 1H), 7.60(s, 1H), 7.53(s, 1H), 7.45 (d, J = 8.4 Hz, 1H), 7.26-7.24(m, 1H), 6.99-6.93(m, 1H), 6.76-6.72(m, 1H), 6.60-6.55(m, 2H), 5.14-5.08(m, 1H), 5.00-4.85(br s, 1H), 4.48-4.28(m, 4H), 2.92-2.82(m, 1H), 2.70(s, 3H), 2.62-2.57(m, 1H), 2.49-2.41(m, 1H), 2.02-1.95(m, 1H).

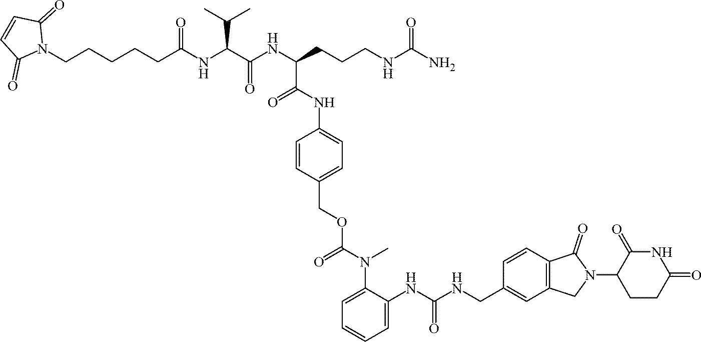

### Step 3. Synthesis of Compound (Ic)

To a stirred mixture of 3-[[2-(2,6-dioxopiperidin-3-yl)-1-oxo-3H-isoindol-5-yl]methyl]-1-[2-(methylamino)phenyl]urea (P4, 150.00 mg, 0.36 mmol, 1.00 equiv), 2,6-lutidine (76 mg, 0.71 mmol, 2.00 equiv) and HOBT (96 mg, 0.71 mmol, 2.00 equiv) in DMF (3.00 mL) was added [4-[(2S)-5-(carbamoylamino)-2-[(2S)-2-[6-(2,5-dioxopyrrol-1-yl)hexanamido]-3-methylbutanamido]pentanamido]phenyl]methyl 4-nitrophenyl carbonate (394 mg, 0.53 mmol, 1.50 equiv) at room temperature under nitrogen atmosphere. The reaction mixture was purified by reverse flash chromatography with the following conditions: column, C18 silica gel; mobile phase, Mobile Phase A: water (0.1%FA), Mobile Phase B: ACN;) to afford crude product (60 mg) as a white solid. The crude product (60 mg) was purified by Prep-HPLC with the following conditions (Column: Xselect CSH OBD Column 30×150 mm 5 um, n; Mobile Phase A:Water (0.1% FA), Mobile Phase B:ACN; Flow rate:60 mL/min; Gradient:24 B to 44 B in 7 min; 220 nm; RT1:6.33; RT2:).The collected fraction was lyophilized to afford [4-[(2S)-5-(carbamoylamino)-2-[(2S)-2-[6-(2,5-dioxopyrrol-1-yl)hexanamido]-3-methylbutanamido]pentanamido]phenyl]methyl N-[2-[([[2-(2,6-dioxopiperidin-3-yl)-1-oxo-3H-isoindol-5-yl]methyl]carbamoyl)amino]phenyl]-N-methylcarbamate (18.1 mg, 5%) as a white solid. LCMS (ESI): 1020 (M+H)⁺. ¹H NMR (400 MHz, DMSO-d₆) δ 10.99 (s, 1H), 9.96(s, 1H), 8.19-8.06 (m, 3H), 7.79 (d, J = 8.8 Hz, 1H), 7.70 (d, J = 8.0 Hz, 1H), 7.53-7.41 (m, 5H), 7.20-7.05 (m, 4H), 7.00(s, 2H), 6.95-6.90(m, 1H), 5.95(br s, 1H), 5.41(s, 2H), 5.18-4.89(m, 3H), 4.44-4.20(m, 5H), 4.19-4.17(m, 1H), 3.09(s, 3H), 3.07-2.85(m, 3H), 2.22-2.02(m, 2H), 2.00-1.85(m, 2H), 1.71-1.25(m, 10H), 1.20-1.12(m, 3H), 0.84-0.80(m, 6H)

Scheme 4 shows how Compound (Id) was prepared from neoDegrader P1.

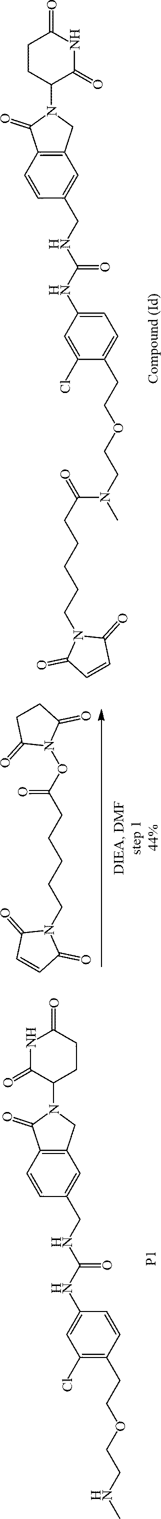

Scheme 4: Preparation of Compound (Id)

### Synthesis of Compound (Id)

To a stirred mixture of 1-(3-chloro-4-[2-[2-(methylamino)ethoxy]ethyl]phenyl)-3-[[2-(2,6-dioxopiperidin-3-yl)-1-oxo-3H-isoindol-5-yl]methyl]urea (P1, 40.00 mg, 0.076 mmol, 1.00 equiv) and 2,5-dioxopyrrolidin-1-yl 6-(2,5-dioxopyrrol-1-yl)hexanoate(25.00 mg, 0.081 mmol, 1.07 equiv) in DMF (2.00 mL) was added DIEA (20.00 mg, 0.16 mmol, 2.04 equiv) dropwise at room temperature. The resulting mixture was stirred for 3h at room temperature under nitrogen atmosphere. The resulting mixture was quenched with water (30 mL), and extracted with DCM (3 × 30 mL). The combined organic layers were washed with water (30 mL), brine (30 mL), dried over Na₂SO₄. After filtration, the filtrate was concentrated to dryness under vacuum. The residue was purified by the following condition: Column: SunFire C18 OBD Prep Column, 100 um, 19 mm x 250 mm; Mobile Phase A: water (0.05% TFA), Mobile Phase B: ACN; Flow rate: 25 mL/min; Gradient:25 B to 55 B in 8.5 min; 220 nm; RT1:8min; The collected fraction was lyophilized to afford N-[2-(2-[2-chloro-4-[([[2-(2,6-dioxopiperidin-3-yl)-1-oxo-3H-isoindol-5-yl]methyl]-carbamoyl)amino]phenyl]ethoxy)ethyl]-6-(2,5-dioxopyrrol-1-yl)-N-methylhexanamide ( Compound (Id), 24 mg, 43%) as a white solid. LCMS: (ES, m/s): 721,723 (M+H)⁺; ¹H NMR (400 MHz, DMSO-d₆) δ 10.99 (s, 1H), 8.78 (s, 1H), 7.70-7.66 (m, 2H), 7.51 (s, 1H), 7.41 (d, J = 9.6 Hz, 1H), 7.18-7.16 (m, 2H), 7.00(d, J = 5.6 Hz, 2H), 6.85-6.80 (m, 1H), 5.12-5.05 (m, 1H), 4.42-4.33 (m, 5H), 3.39-3.36 (m, 3H), 2.91-2.76 (m, 7H), 2.68-2.52 (m, 1H), 2.48-2.35 (m, 1H), 2.33-2.20 (m, 3H), 2.05-1.95 (m, 1H), 1.48-1.44 (m, 5H), 1.28-1.12 (m, 3H).

Schemes 5A and 5B show how to prepare a complex of neoDegrader P1 with an alternative tripeptide linker.

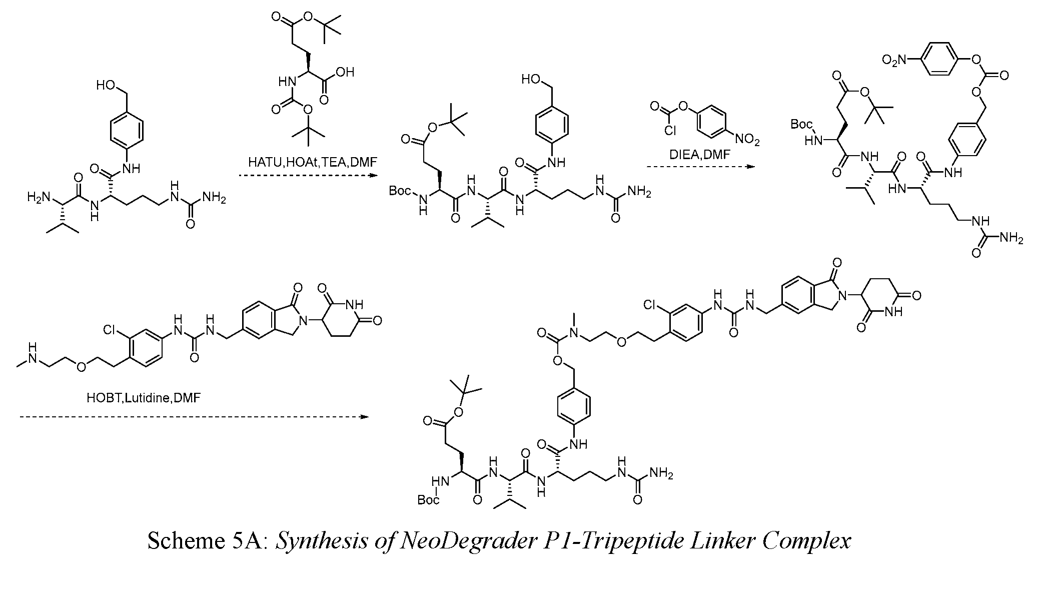

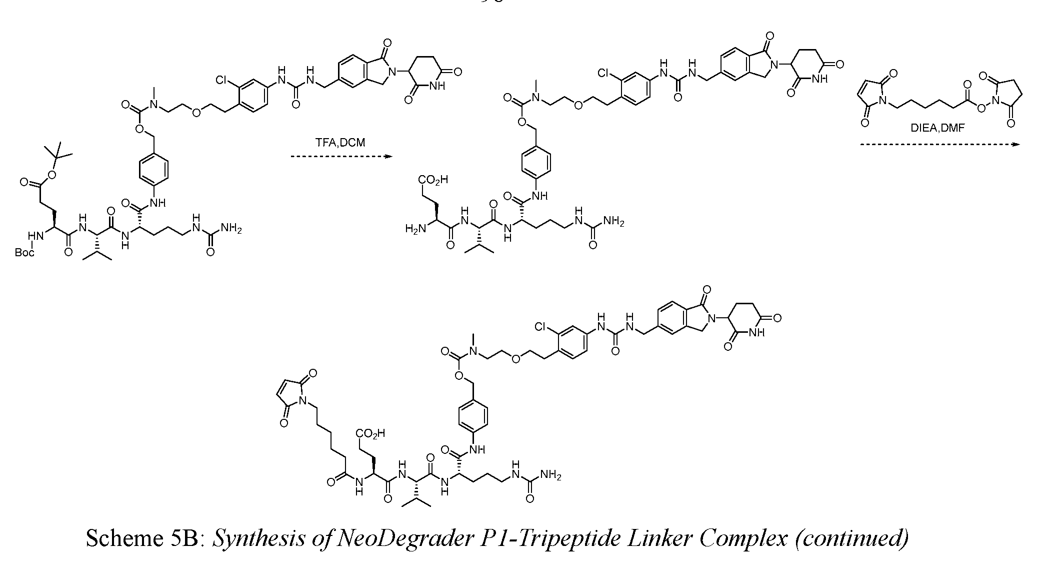

Schemes 6A and 6B show how to prepare a complex of neoDegrader P1 with a β-glucuronide linker.

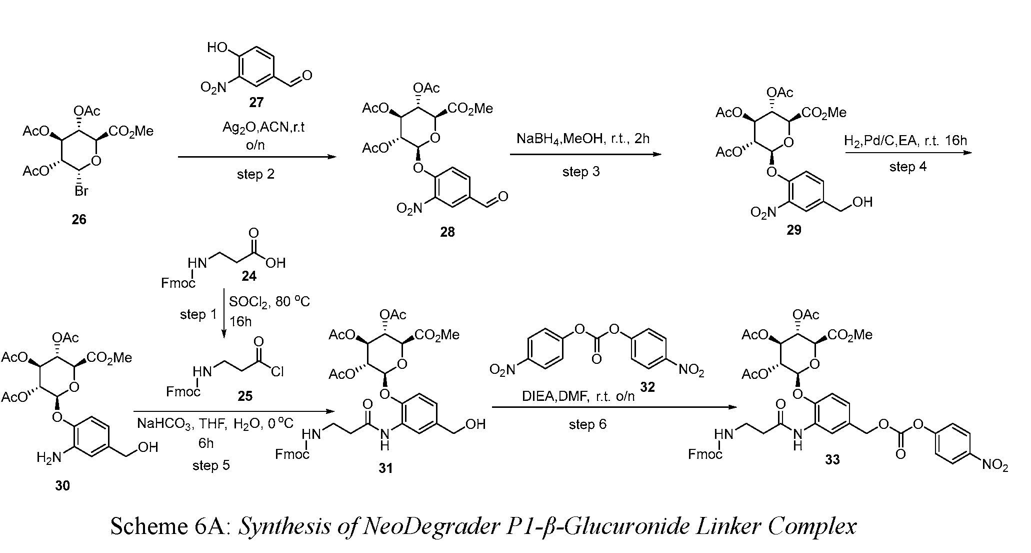

### Step 1. Synthesis of Compound 25

To a stirred mixture of 3-[[(9H-fluoren-9-ylmethoxy)carbonyl]amino]-propanoic acid (Compound 24, 5.00 g, 16.06 mmol, 1.00 equiv) in SOCl₂ (25 mL) at room temperature. The resulting mixture was stirred 16 h at 80° C. The desired product could be detected by LCMS (derivative with MeOH MS=326). LCMS indicated the reaction was completed. The resulting mixture was concentrated under vacuum to afford 9H-fluoren-9-ylmethyl N-(3-chloro-3-oxopropyl)carbamate (Compound 25, 7.5 g, crude) as a yellow oil. The crude product was used in the next step directly without further purification. ¹H-NMR analysis indicated it was the desired product (derivative with MeOH). ¹H-NMR (300 MHz, CDCl₃) δ 7.81-7.77 (m, 2H), 7.63-7.59 (m, 2H), 7.46-7.40 (m, 2H), 7.40-7.31 (m, 2H), 5.33 (s, 1H), 4.42 (d, J=3.0 Hz, 2H), 4.24 (t, J=6.0 Hz, 1H), 3.74-3.67 (m, 3H), 3.50 (d, J=3.0 Hz, 2H), 2.59 (t, J=6.0 Hz, 2H).

### Step 2. Synthesis of Compound 28

To a stirred solution of 4-formyl-2-nitrophenol (Compound 27, 4.21 g, 25.19 mmol, 1.00 equiv) and Ag₂O (7.00 g, 30.20 mmol, 1.20 equiv) in ACN (100 mL, 190.24 mmol, 75.00 equiv) were added Compound 26 (10.00 g, 25.17 mmol, 1.00 equiv) in portions at room temperature under N₂ atmosphere. The resulting mixture was stirred for overnight at room temperature under N₂ atmosphere. LCMS indicated the reaction was completed. The resulting mixture was filtered, the filter cake was washed with DCM (50 mlx3). The filtrate was concentrated under reduced pressure. The residue was purified by silica gel column chromatography, eluted with PE/EA (PE:EA=1:2) to afford methyl (2S,3S,4S,5R,6S)-3,4,5-tris(acetyloxy)-6-(4-formyl-2-nitrophenoxy)oxane-2-carboxylate (Compound 28, 10.5 g, 86%) as a white solid. ¹H-NMR analysis indicated it was the desired product. LCMS (ES, m/z):484 [M+1]⁺. ¹H-NMR (300 MHz, CDCl₃) δ 10.00 (s, 1H), 8.34 (s, 1H), 8.13-8.09 (m, 1H), 7.52 (d, J=3.0 Hz, 1H), 5.47-5.29 (m, 4H), 4.37-4.35 (m, 1H), 3.75-3.73 (m, 3H), 2.17-2.06 (m, 9H).

### Step 3. Synthesis of Compound 29

To a stirred solution of methyl (2S,3S,4S,5R,6S)-3,4,5-tris(acetyloxy)-6-(4-formyl-2-nitrophenoxy)oxane-2-carboxylate (Compound 28, 6.00 g, 12.41 mmol, 1.00 equiv) in MeOH (50 mL) were added NaBH₄ (0.47 g, 12.42 mmol, 1.00 equiv) in portions at RT under N₂ atmosphere. The resulting mixture was stirred for 2h at room temperature under N₂ atmosphere. LCMS indicated the reaction was completed. The reaction was quenched with water at room temperature. The resulting was dried by Na₂SO₄. The resulting mixture was filtered, the filter cake was washed with DCM. The resulting mixture was concentrated under vacuum to afford methyl (2S,3S,4S,5R,6S)-3,4,5-tris(acetyloxy)-6-[4-(hydroxymethyl)-2-nitrophenoxy]oxane-2-carboxylate (Compound 29, 5.5 g, 91%) as a solid. LCMS (ES, m/z):486 [M+H]+.

### Step 4. Synthesis of Compound 30

To a stirred mixture of methyl (2S,3S,4S,5R,6S)-3,4,5-tris(acetyloxy)-6-[4-(hydroxymethyl)-2-nitrophenoxy]oxane-2-carboxylate (Compound 29, 5.50 g, 11.33 mmol, 1.00 equiv) in EA (60 mL) were added Pd/C (1.10 g, 10%) in portions at room temperature. The resulting mixture was stirred for 16 h at room temperature under H₂ atmosphere. LCMS indicated the reaction was completed. The resulting mixture was filtered, the filter cake was washed with DCM and MeOH, The filtrate was concentrated under vacuum to afford methyl (2S,3 S,4S,5R,6S)-3,4,5-tris(acetyloxy)-6-[2-amino-4-(hydroxymethyl)phenoxy]oxane-2-carboxylate (Compound 30, 4.0 g, 77%) as a solid. The crude product was used in the next step directly without further purification. LCMS (ES, m/z):456[M+H]⁺.

### Step 5. Synthesis of Compound 31

To a stirred solution of methyl (2S,3S,4S,5R,6S)-3,4,5-tris(acetyloxy)-6-[2-amino-4-(hydroxymethyl)phenoxy]oxane-2-carboxylate (Compound 30, 1.00 g, 2.19 mmol, 1.00 equiv) and NaHCO₃ (0.20 g, 2.40 mmol, 1.1 equiv) in THF (10 mL) was added Compound 25 (0.87 g, 2.62 mmol, 1.20 equiv) in portions at 0° C. under N₂ atmosphere. The resulting mixture was stirred for 6 h at 0° C. under N₂ atmosphere. LCMS indicated the reaction was completed. The reaction was quenched with water at room temperature. The resulting mixture was extracted with DCM. The combined organic layers were concentrated under reduced pressure. The residue was purified by silica gel column chromatography, eluted with PE/EA (EA=100 %) to afford methyl (2S,3S,4S,5R,6S)-3,4,5-tris(acetyloxy)-6-[2-(3-[[(9H-fluoren-9-ylmethoxy)carbonyl]amino]-propanamido)-4-(hydroxymethyl)phenoxy]oxane-2-carboxylate (Compound 31, 1.1 g, 66%) as a light yellow solid. LCMS (ES, m/z):749 [M+H]⁺.

### Step 6. Synthesis Compound 33

To a stirred mixture of methyl (2S,3S,4S,5R,6S)-3,4,5-tris(acetyloxy)-6-[2-(3-[[(9H-fluoren-9-ylmethoxy)carbonyl]amino]propanamido)-4-(hydroxymethyl)phenoxy]oxane-2-carboxylate (Compound 31, 1.50 g, 2.00 mmol, 1.00 equiv) and bis(4-nitrophenyl) carbonate (Compound 32, 0.68 g, 2.24 mmol, 1.12 equiv) in DMF (15 mL) was added DIEA (0.52 g, 4.01 mmol, 2.00 equiv) in portions at 0° C. under N₂ atmosphere. The resulting mixture was stirred overnight at room temperature under nitrogen atmosphere. LCMS indicated the reaction was completed. The reaction mixture was purified by reverse flash chromatography with the following conditions: column, C18 silica gel; mobile phase, ACN in water (0.1% FA), 10% to 90% gradient in 40 min; detector, UV 254 nm. The collected fraction was concentrated to dryness in vacuum to afford methyl (2S,3S,4S,5R,6S)-3,4,5-tris(acetyloxy)-6-[2-(3-[[(9H-fluoren-9-ylmethoxy)carbonyl]amino]propanamido)-4-[[(4-nitrophenoxycarbonyl)oxy]methyl]phenoxy]oxane-2-carboxylate (Compound 33, 1.4 g, 48%) as a yellow solid. LCMS (ES, m/z):914 [M+H]⁺.

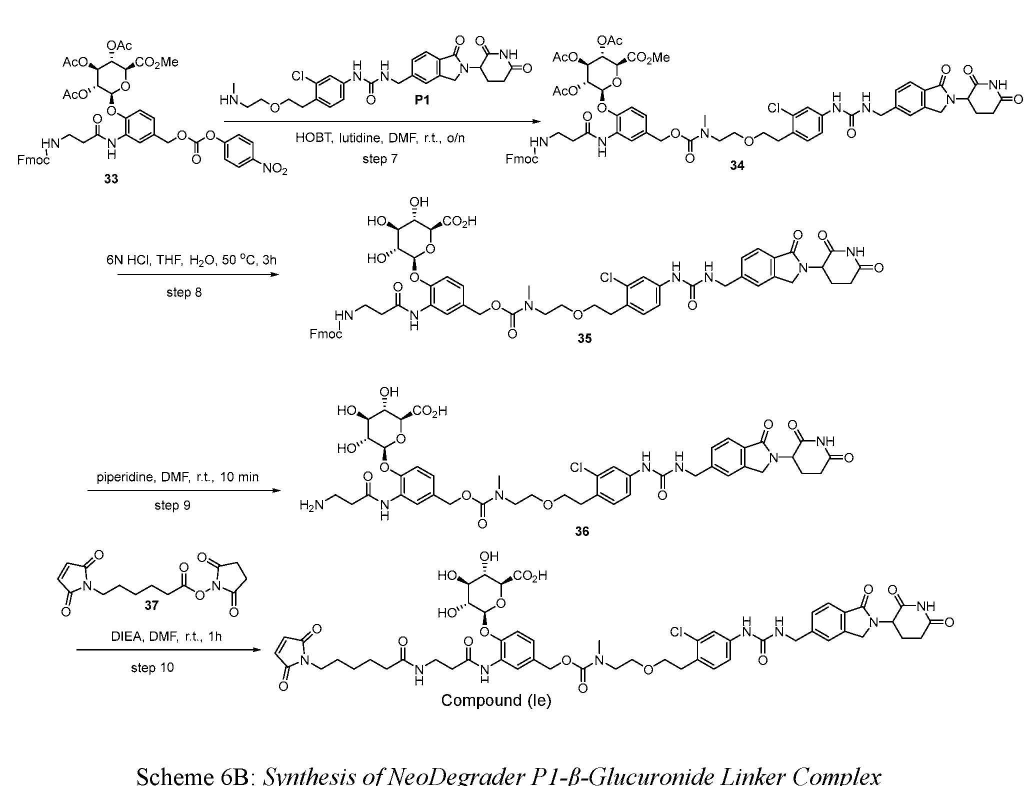

### Step 7. Synthesis Compound 34

To a stirred mixture of methyl (2S,3S,4S,5R,6S)-3,4,5-tris(acetyloxy)-6-[2-(3-[[(9H-fluoren-9-ylmethoxy)carbonyl]amino]propanamido)-4-[[(4-nitrophenoxycarbonyl)oxy]methyl]phenoxy]oxane-2-carboxylate (Compound 33, 1.00 g, 1.09 mmol, 1.00 equiv) and 1-(3-chloro-4-[2-[2-(methylamino)ethoxy]ethyl]phenyl)-3-[[2-(2,6-dioxopiperidin-3-yl)-1-oxo-3H-isoindol-5-yl]methyl]urea (neodegrader P1, 0.58 g, 1.09 mmol, 1.00 equiv) in DMF (10 mL) were added HOBT (1.18 g, 8.72 mmol, 8.00 equiv) and 2,4-dimethylpyridine (1.07 g, 8.72 mmol, 8.00 equiv) in portions at room temperature under N₂ atmosphere. The resulting mixture was stirred for 16 h at room temperature under N₂ atmosphere. LCMS indicated the reaction was completed. The resulting mixture was used further purification. The residue was purified by reverse flash chromatography with the following conditions: column, C18 silica gel; mobile phase, ACN in water (0.1% FA), 10% to 80% gradient in 40 min; detector, UV 254 nm. The collected fraction was concentrated under vacuum to afford methyl (2S,3S,4S,5R,6S)-3,4,5-tris(acetyloxy)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]oxane-2-carboxylate (Compound 34, 800 mg, 56%) as a solid. LCMS (ES, m/z):1302[M+H]⁺.

### Step 8. Synthesis Compound 35

To a stirred mixture of methyl (2S,3S,4S,5R,6S)-3,4,5-tris(acetyloxy)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]oxane-2-carboxylate (Compound 34, 800.00 mg, 0.61 mmol, 1.00 equiv) in THF (80 mL) was added HCl (6 N, 80 mL) in portions at room temperature under N₂ atmosphere. The resulting mixture was stirred for 3 h at degrees 50° C. under nitrogen atmosphere. LCMS indicated the reaction was completed. The resulting mixture was concentrated under vacuum. The residue was purified by reverse flash chromatography with the following conditions: column, C18 silica gel; mobile phase, ACN in water (0.1% FA), 0% to 80% gradient in 40 min; detector, UV 254 nm. The collected fraction was lyophilized to afford (2S,3S,4S,5R,6S)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]-3,4,5-trihydroxyoxane-2-carboxylic acid (Compound 35, 230 mg, 32%) as a white solid. LCMS (ES, m/z):1162[M+H]⁺.

### Step 9. Synthesis of Compound 36

To a stirred solution of (2S,3S,4S,5R,6S)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]-3,4,5-trihydroxyoxane-2-carboxylic acid (Compound 35, 230 mg, 0.2 mmol,1.00 equiv) in DMF (2 mL) was added piperidine (0.4 mL) in portions at room temperature under nitrogen atmosphere. The resulting mixture was stirred for 10 min at room temperature under nitrogen atmosphere. LCMS indicated the reaction was completed. The resulting mixture was used directly further purification by Prep HPLC with the following conditions (Column: XSelect CSH Prep C18 OBD Column, 19×250 mm, 5um; Mobile Phase A: water (0.05% TFA), Mobile Phase B: ACN; Flow rate: 25 mL/min; Gradient:20 B to 40 B in 7 min; 220 nm; RT 1:5.78 min) to afford (2S,3S,4S,5R,6S)-6-[2-(3-aminopropanamido)-4-[([[2-(2-[2-chloro-4-[([[2-(2,6-dioxopiperidin-3-yl)-1-oxo-3H-isoindol-5-yl]methyl]carbamoyl)amino]phenyl]-ethoxy)ethyl](methyl)carbamoyl]oxy)methyl]phenoxy]-3,4,5-trihydroxyoxane-2-carboxylic acid (Compound 36, 35 mg,18%) as a white solid. LCMS (ES, m/z): 940[M+H]+.

### Step 10. Synthesis of Compound (Ie)

To a stirred solution of (2S,3S,4S,5R,6S)-6-[2-(3-aminopropanamido)-4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]phenoxy]-3,4,5-trihydroxyoxane-2-carboxylic acid (Compound 36, 30 mg, 0.03 mmol, 1.00 equiv) in DMF (3 mL) were added DIEA (13 mg, 0.10 mmol, 3.00 equiv) and Compound 37 (30 mg, 0.10 mmol, 3.00 equiv) in portions at room temperature under nitrogen atmosphere. The resulting mixture was stirred for 1 h at room temperature under nitrogen atmosphere. LCMS indicated the reaction was completed. The resulting mixture was purified by Prep-HPLC with the following conditions (Column: Xselect CSH OBD Column 30 × 150 mm 5 um, Mobile Phase A:water (0.1% FA), Mobile Phase B:ACN; Flow rate:60 mL/min; Gradient:21 B to 36 B in 10 min; 220 nm; RT 1:11.15 min). The collected fraction was lyophilzed to afford (2S,3S,4S,5R,6S)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl]-(methyl)carbamoyl]oxy)methyl]-2-[3-[6-(2,5-dioxopyrrol-1-yl)hexanamido]propanamido]phenoxy]-3,4,5-trihydroxyoxane-2-carboxylic acid (Compound (Ie), 10.5 mg, 28%) as a white a solid. LCMS (ES, m/z):1133[M+H]⁺. ¹H-NMR (300 MHz, DMSO-d₆) δ 10.9 (s, 1H), 9.13 (s, 1H), 8.16 (s, 1H), 7.92-7.68 (m, 4H), 7.52 (s, 1H), 7.44 (d, J=3.0 Hz, 1H), 7.18-6.99 (m, 7H), 5.76 (s, 1H), 5.20-5.10 (m, 2H), 4.98 (br s, 2H), 4.76-4.74 (m, 1H), 4.42-4.33 (m, 4H), 3.65 (br s, 1H), 3.58-3.54 (m, 5H), 3.35 (d, J=6 Hz, 2H), 2.90-2.83 (m, 7H), 2.57-2.55 (m, 3H), 2.45-2.30 (m, 1H), 2.02-1.98 (m, 4H), 1.48-1.42 (m, 5H), 1.40-1.20 (m, 3H).

Scheme 7 shows how to prepare a complex of neoDegrader P6 with a hydrazine linker.

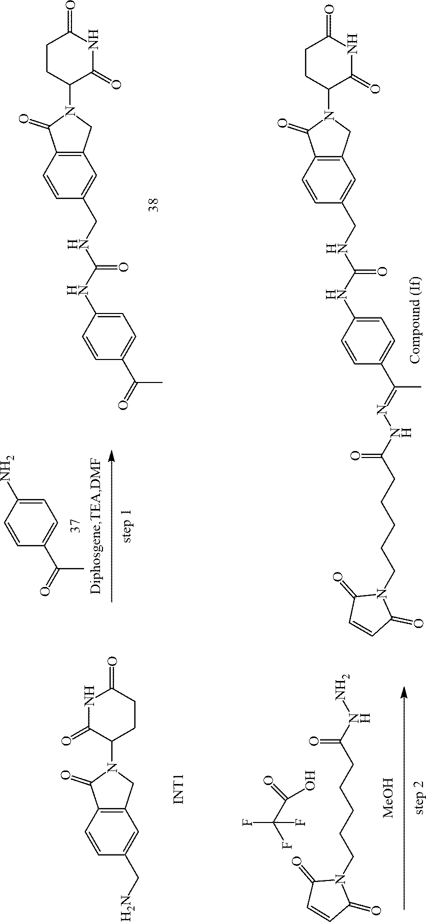

Scheme 7: Synthesis of NeoDegrader P6-Hydrazone Linker Complex

### Step 1. Synthesis of Compound 38

To a stirred solution of 4-aminoacetophenone (Compound 37, 100 mg, 0.73 mmol, 1.00 equiv) in THF (2.00 mL) were added diphosgene (0.40 mL) dropwise at room temperature. The resulting mixture was stirred for 30 min at 0° C. The resulting mixture was concentrated under vacuum. The resulting solid was re-dissolved in DMF (1.50 mL). To the stirred solution was 3-[5-(aminomethyl)-1-oxo-3H-isoindol-2-yl]piperidine-2,6-dione (INT1, 200 mg, 0.73 mmol, 1.00 equiv) in DMF (3.00 mL) and TEA (0.50 mL) dropwise at room temperature. The resulting mixture was stirred for 1h at 0° C. LCMS indicated the reaction was completed. The mixture was added water (5 mL) and extracted with CH₂Cl₂ (3×10 mL). The organic layer was concentrated under vacuum. The residue was purified by reverse flash chromatography with the following conditions: column, C18 silica gel; mobile phase, ACN in water (0.05% TFA), 10% to 50% gradient in 35 min; detector, UV 254 nm. The collection fraction was concentrated to dryness to afford 1-(4-acetylphenyl)-3-[[2-(2,6-dioxopiperidin-3-yl)-1-oxo-3H-isoindol-5-yl]methyl]urea (Compound 38, 80 mg, 25%) as a light yellow solid. LCMS:(ES.m/z):435[M+1]⁺.

### Step 2. Synthesis of Compound (If)

The mixture of 1-(4-acetylphenyl)-3-[[2-(2,6-dioxopiperidin-3-yl)-1-oxo-3H-isoindol-5-yl]methyl]urea (Compound 38, 80.00 mg, 0.18 mmol, 1.00 equiv) and 6-(2,5-dioxopyrrol-1-yl)hexanehydrazide; trifluoroacetic acid (75 mg, 1.20 equiv) in methanol (5.00 mL) was stirred for overnight at 50° C. The mixture was cooled down to room temperature. LCMS indicated the reaction was completed. The precipitated solids were collected by filtration and washed with MeOH (2×5 mL). The crude solid was purified by reverse flash chromatography with the following conditions: C18 column; mobile phase, ACN in water (0.1%FA), 10% to 50% gradient in 30 min; detector, UV 254 nm. The collected fraction was extracted with DCM (3×5 mL) and concentrated under vacuum. This resulted in 3-[[2-(2,6-dioxopiperidin-3-yl)-1-oxo-3H-isoindol-5-yl]methyl]-1-[4-[(1E)-1-[[6-(2,5-dioxopyrrol-1-yl)hexanamido]imino]ethyl]phenyl]urea (Compound (If), 4.4 mg, 3.7%) as an off-white solid. LCMS:(ES.m/z): 642[M+1]⁺.¹H NMR (400 MHz, DMSO-d₆) δ 10.99 (s, 1H), 10.26-10.15 (m, 1H), 8.82 (s, 1H),7.69-7.62(m, 3H), 7.52-7.43 (m, 4H), 7.01-6.99 (m, 2H), 5.13-5.09 (m, 1H), 4.42-4.33 (m, 4H), 2.98-2.82 (m, 1H), 2.62-2.58 (m, 2H), 2.20-2.12 (m, 2H), 1.58-1.51 (m, 6H),1.26-1.09 (m,6H)

Scheme 8 shows how to prepare a complex of neoDegrader P2 with a quaternary amine linker.

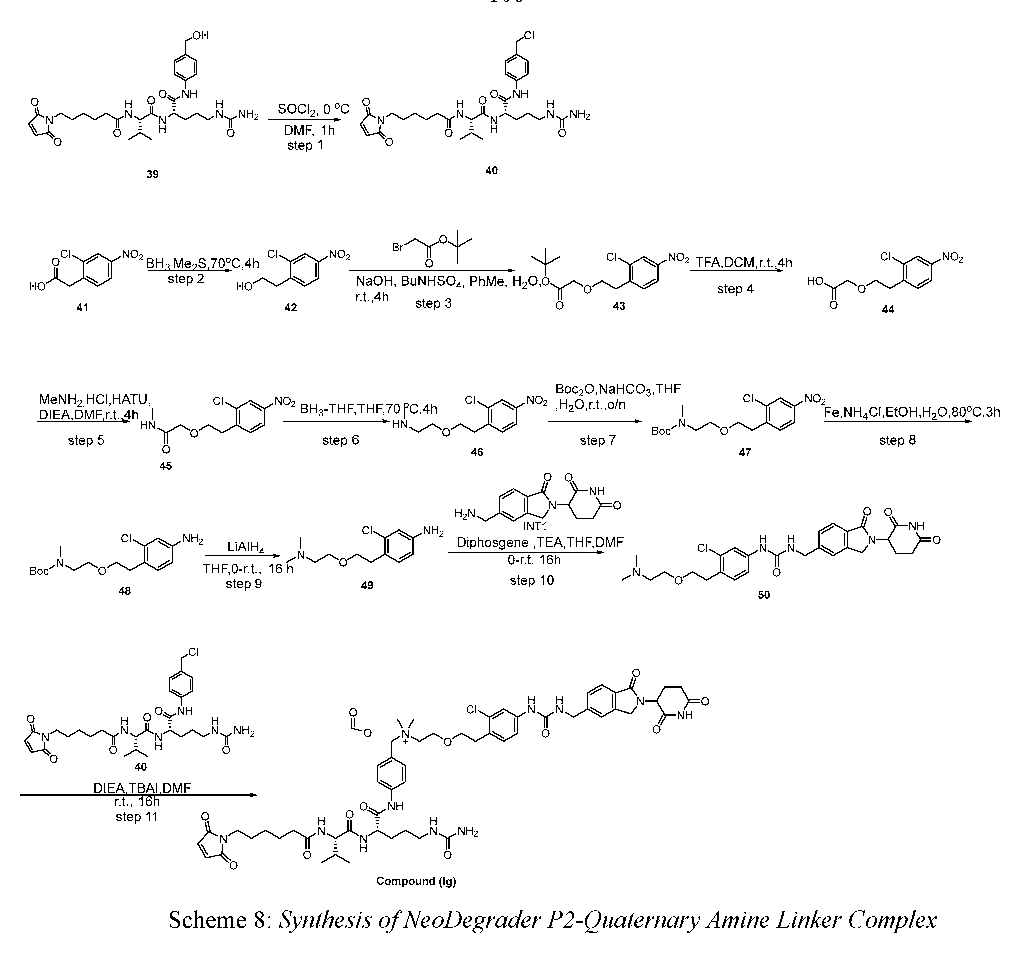

### Step 1. Synthesis of Compound 40

To a stirred solution of N-[(1S)-1-[[(1S)-4-(carbamoylamino)-1-[[4-(hydroxymethyl)phenyl]carbamoyl]butyl]carbamoyl]-2-methylpropyl]-6-(2,5-dioxopyrrol-1-yl)hexanamide (Compound 39, 100 mg, 0.18 mmol, 1.00 equiv) in DMF (2 mL) was added SOCl₂ (20 mg, 0.18 mmol, 1 equiv) in DCM (2 mL) dropwise under N₂ at 0° C. The resulting mixture was stirred at 0° C. for 1h. LCMS indicated the reaction was completed. The reaction mixture was diluted with ice-cooled water (20 mL), extracted with DCM (10 mL*3), the combined organic layer was washed with water (10 mL), brine (10 mL), dried over anhydrous sodium sulfate and concentrated to dryness under vacuum to give N-[(1S)-1-[[(1S)-4-(carbamoylamino)-1-[[4-(chloromethyl)phenyl]-carbamoyl]butyl]carbamoyl]-2-methylpropyl]-6-(2,5-dioxopyrrol-1-yl)hexanamide (Compound 40, 80 mg, 53%) of the product as a white solid. LCMS (ES, m/z): 591,593 [M+H]⁺

### Step 2. Synthesis of Compound 42

To a stirred mixture of (2-chloro-4-nitrophenyl)acetic acid (Compound 41, 8.60 g, 39.9 mmol, 1.00 equiv) in THF (130 mL) was added BH₃—Me₂S (10.00 mL, 105.4 mmol, 2.64 equiv) dropwise at 0° C. The resulting mixture was stirred for 4h at 70° C. under nitrogen atmosphere. TLC (PE:EA=1:2) indicated the reaction was completed. The mixture was allowed to cool down to room temperature. The resulting mixture was concentrated under vacuum. The residue was purified by silica gel column chromatography, eluted with PE/EtOAc (1:1) to afford 2-(2-chloro-4-nitrophenyl)ethanol (Compound 42, 7.7 g, 96%) as a yellow solid. ¹H NMR (400 MHz, CDCl₃) δ 8.27 (d, J = 4.0 Hz, 1H), 8.11-8.07 (m, 1H), 7.53 (d, J = 8.0 Hz, 1H), 3.99 (t, J = 8.0 Hz, 2H), 3.15 (t, J= 8.0 Hz, 2H).

### Step 3. Synthesis of Compound 43

To a stirred mixture of 2-(2-chloro-4-nitrophenyl)ethanol, (Compound 42, 7.70 g, 38.2 mmol, 1.00 equiv) and tert-butyl 2-bromoacetate (57.74 g, 296.0 mmol, 7.75 equiv) in toluene (70 mL) was added Bu₄NHSO₄ (10.37 g, 30.6 mmol, 0.80 equiv) in portions at 0° C. To the above mixture was added NaOH (15.00 g, 375.0 mmol, 9.82 equiv) in H₂O (90 mL) dropwise over 30 h at 0° C. The resulting mixture was stirred for additional 4h at room temperature. TLC (PE:EA=3:1) indicated the reaction was completed The resulting mixture was extracted with EtOAc (3 × 200 mL). The combined organic layers were washed with brine (200 mL), dried over anhydrous Na₂SO₄. After filtration, the filtrate was concentrated under reduced pressure. The residue was purified by silica gel column chromatography, eluted with PE/EtOAc (5:1) to afford tert-butyl 2-[2-(2-chloro-4-nitrophenyl)ethoxy]acetate (Compound 43, 12.2 g, 91%) as a yellow oil. ¹H NMR (300 MHz, CDCl₃) δ 8.20 (d, J = 4.0 Hz, 1H), 8.07-8.03 (m, 1H), 7.61 (d, J= 8.1 Hz, 1H), 4.11 (s, 2H), 3.83 (t, J= 8.1 Hz, 2H), 3.16(t, J= 8.1 Hz, 2H), 1.45(s, 9H).

### Step 4. Synthesis of Compound 44

To a stirred mixture of tert-butyl 2-[2-(2-chloro-4-nitrophenyl)ethoxy]acetate (Compound 43, 12.20 g, 38.6 mmol, 1.00 equiv) in DCM (120 mL) was added TFA (20 mL) dropwise at 0° C. The resulting mixture was stirred for 4h at room temperature. LCMS indicated the reaction was completed. The resulting mixture was concentrated under reduced pressure. This resulted in [2-(2-chloro-4-nitrophenyl)ethoxy]acetic acid (Compound 44, 8.4 g, 83%) as a yellow solid. LCMS: (ES, m/s): 517 (2 M-H)⁻ ¹H NMR (400 MHz, DMSO-d₆) δ 12.64(s, 1H), 8.20 (d, J= 4.0 Hz, 1H), 8.11-8.08 (m, 1H), 7.72 (d, J = 8.0 Hz, 1H), 4.06 (s, 2H), 3.74 (t, J = 8.0 Hz, 2H), 3.06(t, J= 8.0 Hz, 2H).

### Step 5. Synthesis of Compound 45

To a stirred mixture of [2-(2-chloro-4-nitrophenyl)ethoxy]acetic acid, (Compound 44, 8.40 g, 32.35 mmol, 1.00 equiv) and HATU (19.19 g, 50.47 mmol, 1.56 equiv) in DMF (80 mL) were added CH₃NH₂.HCl (2.69 g, 39.79 mmol, 1.23 equiv) and DIEA (17.31 g, 133.93 mmol, 4.14 equiv) at 0° C. under nitrogen atmosphere. The resulting mixture was stirred for 4 h at room temperature under nitrogen atmosphere. LCMS indicated the reaction was completed. The reaction was quenched with water/ice. The resulting mixture was extracted with DCM (3 × 50 mL). The combined organic layers were washed with brine (50 mL), dried over anhydrous Na₂SO₄. After filtration, the filtrate was concentrated under reduced pressure. The residue was purified by silica gel column chromatography, eluted with (DCM: MeOH = 10:1) to afford 2-[2-(2-chloro-4-nitrophenyl)ethoxy]-N-methylacetamide (Compound 45, 7.2 g, 81%) as a yellow oil. LCMS: (ES, m/s): 273,275 (M+H)⁺

### Step 6. Synthesis of Compound 46

To a stirred mixture of 2-[2-(2-chloro-4-nitrophenyl)ethoxy]-N-methylacetamide (Compound 45, 7.20 g, 26.40 mmol, 1.00 equiv) in THF (70 mL) was added BH₃-THF (10 M in THF, 52.0 mL, 520.0 mmol, 20 equiv) dropwise at room temperature. The resulting mixture was stirred for 4 h at 70° C. LCMS indicated the reaction was completed. The mixture was allowed to cool down to room temperature. The reaction was quenched with MeOH. The residue was acidified to pH 6 with 1 N HCl. The resulting mixture was extracted with EtOAc (20 mL). The aqueous phase was basified to pH 8 with saturated NaHCO₃ (sat., aq.). The resulting mixture was extracted with EtOAc (3 × 100 mL), washed with brine (50 mL) and dried over anhydrous Na₂SO₄. After filtration, the filtrate was concentrated under reduced pressure. The residue was purified by silica gel column chromatography, eluted with (DCM: MeOH = 8:1) to afford [2-[2-(2-chloro-4-nitrophenyl)ethoxy]ethyl](methyl)amine (Compound 46, 5.4 g, 79%) as a yellow solid. LCMS: (ES, m/s): 259,261 (M+H)⁺; ¹H NMR (400 MHz, DMSO-d₆) δ 8.26 (d, J= 4.0 Hz, 1H), 8.15-8.12 (m, 1H), 7.73 (d, J= 8.0 Hz, 1H), 3.72 (t, J= 8.0 Hz, 2H), 3.61(t, J= 8.0 Hz, 2H), 3.10 (t, J= 8.0 Hz, 2H), 2.87 (t, J = 8.0 Hz, 2H), 2.40 (s, 3H).

### Step 7. Synthesis of Compound 47

To a stirred mixture of [2-[2-(2-chloro-4-nitrophenyl)ethoxy]ethyl](methyl)amine (Compound 46, 4.00 g, 15.46 mmol, 1.00 equiv) and Boc₂O (3.80 g, 17.41 mmol, 1.13 equiv) in THF (20.00 mL) was added NaHCO₃ (4.00 g, 47.61 mmol, 3.08 equiv) in H₂O (20.00 mL) dropwise at room temperature. The resulting mixture was stirred for overnight at room temperature. LCMS indicated the reaction was completed. The resulting mixture was extracted with EtOAc (3 × 20 mL). The combined organic layers were washed with brine (20 mL), dried over anhydrous Na₂SO₄. After filtration, the filtrate was concentrated under reduced pressure. The residue was purified by silica gel column chromatography, eluted with (DCM: MeOH = 12:1) to afford tert-butyl N-[2-[2-(2-chloro-4-nitrophenyl)ethoxy]ethyl]-N-methylcarbamate (Compound 47, 4.8 g, 77%) as a yellow solid. LCMS: (ES, m/s): 359,361(M+H)⁺; ¹H NMR (400 MHz, DMSO-d₆) δ 8.24 (d, J = 4.0 Hz, 1H), 8.13-8.10 (m, 1H), 7.67 (d, J = 8.0 Hz, 1H), 4.05-4.00(m, 1H), 3.69 (t, J= 8.0 Hz, 2H), 3.50(t, J = 8.0 Hz, 2H), 3.28 (t, J = 8.0 Hz, 2H), 3.07(t, J = 8.0 Hz, 2H), 2.75(s, 3H), 1.36(s, 9H).

### Step 8. Synthesis of Compound 48

To a stirred mixture of tert-butyl N-[2-[2-(2-chloro-4-nitrophenyl)ethoxy]ethyl]-N-methylcarbamate, (Compound 47, 5.60 g, 15.6 mmol, 1.00 equiv) in EtOH (112.00 mL) were added NH₄Cl (2.50 g, 46.74 mmol, 2.99 equiv) in H₂O (12.00 mL) and Fe (4.40 g, 78.79 mmol, 5.05 equiv) at room temperature. The resulting mixture was stirred for 3h at 80° C. LCMS indicated the reaction was completed. The mixture was allowed to cool down to room temperature. The resulting mixture was concentrated under reduced pressure. The resulting mixture was extracted with DCM (3 × 30 mL). The combined organic layers were washed with brine (30 mL), dried over anhydrous Na₂SO₄. After filtration, the filtrate was concentrated under reduced pressure. The residue was purified by silica gel column chromatography, eluted with (DCM: MeOH = 10:1) to afford tert-butyl N-[2-[2-(4-amino-2-chlorophenyl)ethoxy]ethyl]-N-methylcarbamate (Compound 48, 4.2 g, 81%) as a yellow oil. LCMS: (ES, m/s): 329,331 (M+H)⁺; ¹H NMR (400 MHz, DMSO-d₆) δ 6.96 (d, J = 8.0 Hz, 1H), 6.59(d, J = 4.0 Hz, 1H), 6.46-6.43 (m, 1H), 5.18(br s, 2H), 3.50-3.45(m, 4H), 3.29-3.26(m, 2H), 2.75-2.71(m, 5H), 1.38(s, 9H).

### Step 9. Synthesis of Compound 49

To a solution of tert-butyl N-[2-[2-(4-amino-2-chlorophenyl)ethoxy]ethyl]-N-methylcarbamate (Compound 48, 100 mg, 0.30 mmol, 1.00 equiv) in THF (3 mL) was added LiAlH₄ (92 mg, 2.43 mmol, 8.00 equiv) in THF (2 mL) at 0° C. under nitrogen atmosphere. The resulting mixture was stirred at room temperature for 16 hours. The five reactions were run in parallel. LCMS indicated the reaction was completed. Then the reaction was quenched with 1N NaOH (10 mL), filtered, concentrated to dryness under vacuum and then the residue was purified by reverse flash chromatography with the following conditions: column, C18 silica gel; mobile phase, ACN in water (0.1%FA), 0% to 60% gradient in 30 min; detector, UV 254 nm. The collected fraction was concentrated to dryness to give 3-chloro-4-[2-[2-(dimethylamino)ethoxy]ethyl]aniline, 49 (180 mg, 44%) as a yellow oil. LCMS (ES, m/z): 243,245 [M+H]⁺

### Step 10. Synthesis of Compound 50

To a solution of 3-chloro-4-[2-[2-(dimethylamino)ethoxy]ethyl]aniline (Compound 49, 140 mg, 0.58 mmol, 1.00 equiv) in THF (9 mL) was added diphosgene(137 mg, 0.69 mmol, 1.20 equiv) at 0 oC under nitrogen atmosphere. The resulting mixture was stirred at 0° C. for 1 hour. Then the reaction solution was concentrated to dryness under vacuum. The residue was re-dissolved in DMF (2 mL) and then added into a solution of 3-[5-(aminomethyl)-1-oxo-3H-isoindol-2-yl]piperidine-2,6-dione(158 mg, 0.58 mmol, 1.00 equiv) and TEA (117 mg, 1.15 mmol, 2.00 equiv) in DMF (4 mL) dropwise under nitrogen atmosphere. The resulting mixture was stirred at room temperature for 16 h. LCMS indicated the reaction was completed. The reaction mixture was diluted with methanol and the resulting solution was purified by reverse flash chromatography with the following conditions: column, C18 silica gel; mobile phase, ACN in water (0.1%FA), 0% to 50% gradient in 30 min; detector, UV 254 nm to give 100 mg of the product as a colorless solid. The crude product was purified by Prep-HPLC with the following conditions: column: XBridge Shield RP18 OBD Column, 19×250 mm, 10 um; Mobile Phase A: Water (0.1%FA), Mobile Phase B: ACN; Flow rate: 25 mL/min; Gradient: 14% to 32 % in 7 min; 220 nm; RT1: 5.25 min. The collected fraction was lyophilized to give 1-(3-chloro-4-[2-[2-(dimethylamino)ethoxy]ethyl]phenyl)-3-[[2-(2,6-dioxopiperidin-3-yl)-1-oxo-3H-isoindol-5-yl]methyl]urea (Compound 50, 60 mg, 18%) as a colorless solid. LCMS (ES, m/z): 542,544 [M+H]+

### Step 11. Synthesis of Compound (Ig)

To a solution of N-[(1S)-1-[[(1S)-4-(carbamoylamino)-1-[[4-(chloromethyl)phenyl]-carbamoyl]butyl]carbamoyl]-2-methylpropyl]-6-(2,5-dioxopyrrol-1-yl)hexanamide, (Compound 40, 66 mg, 0.11 mmol, 1.00 equiv), 1-(3-chloro-4-[2-[2-(dimethylamino)ethoxy]ethyl]phenyl)-3-[[2-(2,6-dioxopiperidin-3-yl)-1-oxo-3H-isoindol-5-yl]methyl]urea (Compound 50, 60 mg, 0.11 mmol, 1.00 equiv) and DIEA (29 mg, 0.22 mmol, 2.00 equiv) in DMF (1 mL) was added TBAI (4 mg, 0.01 mmol, 0.10 equiv) at room temperature in air. The resulting mixture was stirred at room temperature for 16 hours. LCMS traces showed the reaction was completed. The resulting mixture was purified by reverse phase column chromatography with the following conditions: column, C18 silica gel; mobile phase, ACN in water(0.05%TFA), 5% to 45% gradient in 40 min; detector, UV 254 nm to give 90 mg of the crude product as a yellow oil. Then the crude product was re-purified by the following condition : Column: Xselect CSH OBD Column 30*150 mm 5 um, n; Mobile Phase A: Water(0.1%FA), Mobile Phase B: ACN; Flow rate:60 mL/min; Gradient:15 B to 35 B in 7 min; 220 nm; RT1:6.00 min to afford ([4-[(2S)-5-(carbamoylamino)-2-[(2S)-2-[6-(2,5-dioxopyrrol-1-yl)hexanamido]-3-methylbutanamido]pentanamido]phenyl]methyl)[2-(2-[2-chloro-4-[([[2-(2,6-dioxopiperidin-3-yl)-1-oxo-3H-isoindol-5-yl]methyl]carbamoyl)amino]phenyl]ethoxy)ethyl]dimethylazanium, Compound (Ig) (19 mg, 14.8%) as a white solid. LCMS (ES, m/z): 1096 [M-FA]⁺, 549 [½(M-FA)]⁺; ¹H NMR (400 MHz, CD₃OD) δ 8.48 (s, 1H), 7.77-7.72 (m, 3H), 7.55 - 7.47 (m, 3H), 7.37-7.35 (d, J = 8.4 Hz, 2H), 7.18 - 7.14 (m, 2H), 6.77 (s, 2H), 5.17-5.13 (q, J = 8, 4 Hz, 1H), 4.51 -4.46 (m, 5H), 4.35 (s, 2H), 4.12 (d, J = 8.0 Hz, 1H), 3.90 (s, 2H), 3.79 (t, J = 5.6 Hz, 2H), 3.45 (t, J = 7.2 Hz, 4H), 3.22-3.15 (m, 1H), 3.11-3.05 (m, 1H), 3.00 (t, J = 6.0 Hz, 2H), 2.92 (s, 6H), 2.89 - 2.84 (m, 1H), 2.81 - 2.73 (m, 1H), 2.54-2.43 (m, 1H), 2.27 (t, J = 7.2 Hz, 2H), 2.21 - 2.12 (m, 1H), 2.10 - 2.02 (m, 1H), 1.95 - 1.82 (m, 1H), 1.78-1.69 (m, 1H), 1.64-1.59 (m, 7H), 1.32-1.25 (m, 2H), 0.98-0.96 (m, 6H).

Schemes 9A and 9B show how to prepare a complex of neoDegrader P13 with a peptide-containing linker.

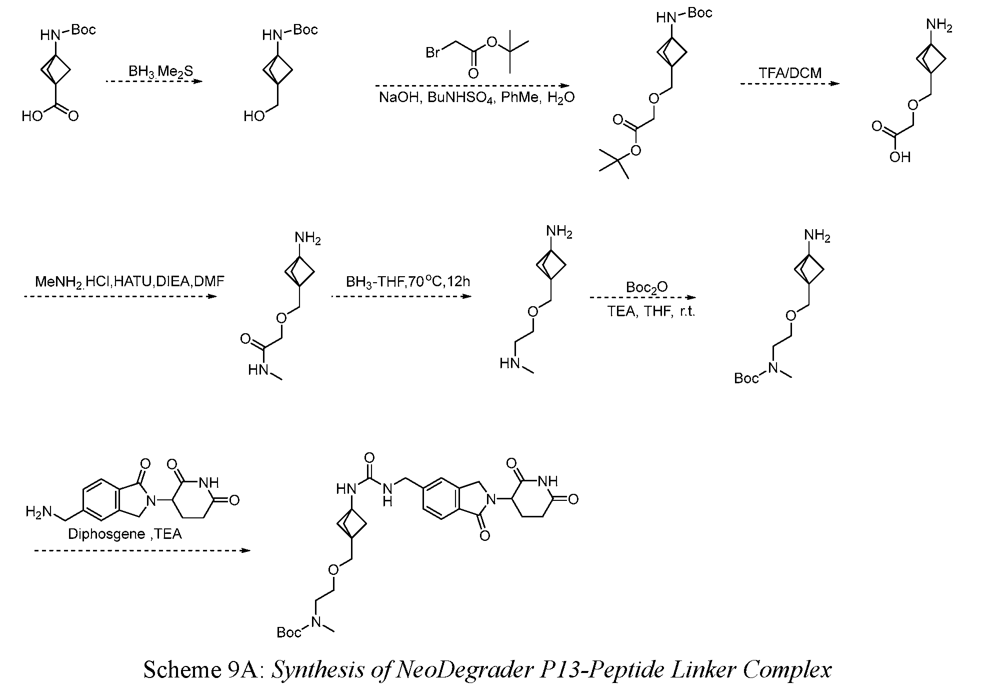

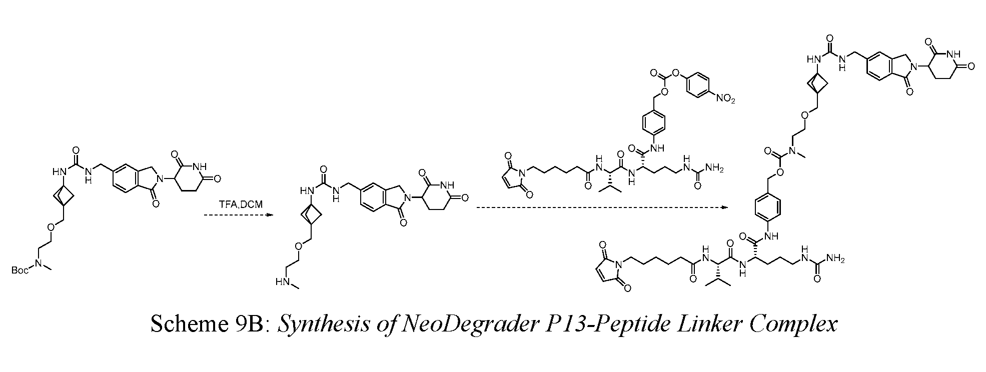

Scheme 10 shows the synthesis of compounds of formula (Ih).

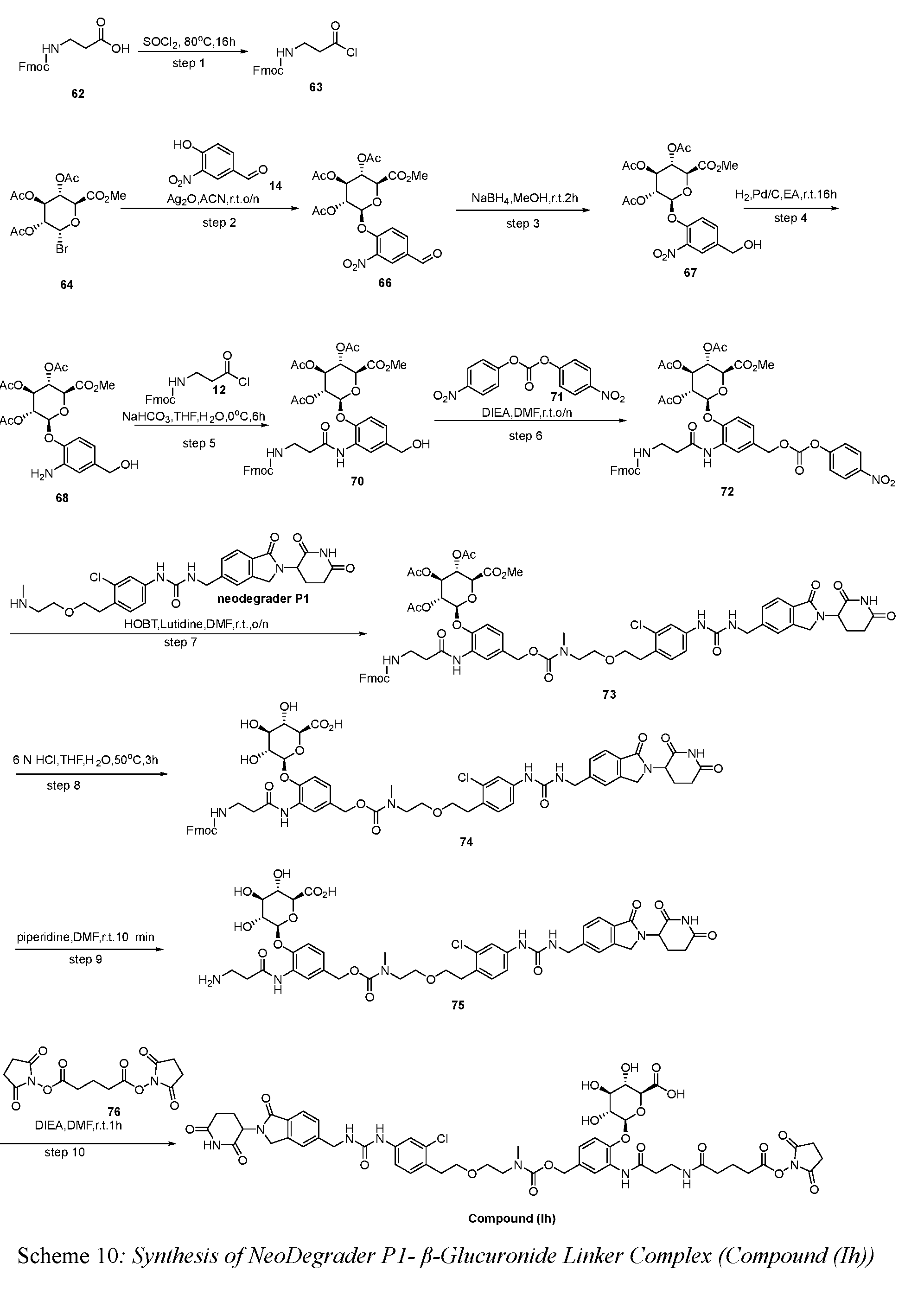

### Step 1. Synthesis of Compound 63

To a stirred mixture of 3-[[(9H-fluoren-9-ylmethoxy)carbonyl]amino]propanoic acid, (Compound 62, 5.00 g, 16.06 mmol, 1.00 equiv), SOCl₂ (25 mL) was added at room temperature. The resulting mixture was stirred 16h at 80° C. Desired product could be detected by LCMS (derivative with MeOH MS=326). LCMS indicated the reaction was completed. The resulting mixture was concentrated under vacuum to afford 9H-fluoren-9-ylmethyl N-(3-chloro-3-oxopropyl)carbamate (Compound 63, 7.5 g, crude) as a yellow oil. The crude product was used directly in the next step without further purification. ¹H NMR analysis indicated it was the desired product (derivative with MeOH). ¹H-NMR (300 MHz, CDCl₃) δ 7.81-7.77 (m, 2H), 7.63-7.59 (m, 2H), 7.46-7.40 (m, 2H), 7.40-7.31 (m, 2H), 5.33 (s, 1H), 4.42 (d, J=3.0 Hz, 2H), 4.24 (t, J=6.0 Hz, 1H), 3.74-3.67 (m, 3H), 3.50 (d, J=3.0 Hz, 2H), 2.59 (t, J=6.0 Hz, 2H).

### Step 2. Synthesis of Compound 66

To a stirred solution of 4-formyl-2-nitrophenol (Compound 65, 4.21 g, 25.19 mmol, 1.00 equiv) and Ag₂O (7.00 g, 30.20 mmol, 1.20 equiv) in ACN (100 mL, 190.24 mmol, 75.00 equiv) were added methyl (2S,3S,4S,5R,6R)-3,4,5-tris(acetyloxy)-6-bromooxane-2-carboxylate (Compound 64, 10.00 g, 25.17 mmol, 1.00 equiv) in portions at room temperature under N₂ atmosphere. The resulting mixture was stirred for overnight at room temperature under N₂ atmosphere. LCMS indicated the reaction was completed. The resulting mixture was filtered, the filter cake was washed with DCM (50 mL x 3). The filtrate was concentrated under reduced pressure. The residue was purified by silica gel column chromatography, eluted with PE/EA (PE:EA=1:2) to afford methyl (2S,3S,4S,5R,6S)-3,4,5-tris(acetyloxy)-6-(4-formyl-2-nitrophenoxy)oxane-2-carboxylate (Compound 66, 10.5 g, 86%) as a white solid. ¹H-NMR analysis indicated it was the desired product. LCMS (ES, m/z):484 [M+1]⁺. ¹H-NMR (300 MHz, CDCl₃) δ 10.00 (s, 1H), 8.34 (s, 1H), 8.13-8.09 (m, 1H), 7.52 (d, J=3.0 Hz, 1H), 5.47-5.29 (m, 4H), 4.37-4.35 (m, 1H), 3.75-3.73 (m, 3H), 2.17-2.06 (m, 9H).

### Step 3. Synthesis of Compound 67

To a stirred solution of methyl (2S,3S,4S,5R,6S)-3,4,5-tris(acetyloxy)-6-(4-formyl-2-nitrophenoxy)oxane-2-carboxylate (Compound 66, 6.00 g, 12.41 mmol, 1.00 equiv) in MeOH (50 mL) was added NaBH₄ (0.47 g, 12.42 mmol, 1.00 equiv) in portions at room temperature under N₂ atmosphere. The resulting mixture was stirred for 2 h at room temperature under N₂ atmosphere. LCMS indicated the reaction was completed. The reaction was quenched with water at room temperature. The resulting was dried by Na₂SO₄. The resulting mixture was filtered, the filter cake was washed with DCM. The resulting mixture was concentrated under vacuum to afford methyl (2S,3S,4S,5R,6S)-3,4,5-tris(acetyloxy)-6-[4-(hydroxymethyl)-2-nitrophenoxy]oxane-2-carboxylate, (Compound 67, 5.5 g, 91%) as a solid. LCMS (ES, m/z):486 [M+H]⁺.

### Step 4. Synthesis of Compound 68

To a stirred mixture of methyl (2S,3S,4S,5R,6S)-3,4,5-tris(acetyloxy)-6-[4-(hydroxymethyl)-2-nitrophenoxy]oxane-2-carboxylate (Compound 67, 5.50 g, 11.33 mmol, 1.00 equiv) in EA (60 mL) were added Pd/C (1.10 g, 10%) in portions at room temperature. The resulting mixture was stirred for 16h at room temperature under H₂ atmosphere. LCMS indicated the reaction was completed. The resulting mixture was filtered, the filter cake was washed with DCM and MeOH, The filtrate was concentrated under vacuum to afford methyl (2S,3S,4S,5R,6S)-3,4,5-tris(acetyloxy)-6-[2-amino-4-(hydroxymethyl)phenoxy]oxane-2-carboxylate (Compound 68, 4.0 g, 77%) as a solid. The crude product was used in the next step directly without further purification. LCMS (ES, m/z):456[M+H]⁺.

### Step 5. Synthesis of Compound 70

To a stirred solution of methyl (2S,3S,4S,5R,6S)-3,4,5-tris(acetyloxy)-6-[2-amino-4-(hydroxymethyl)phenoxy]oxane-2-carboxylate (Compound 68, 1.00 g, 2.19 mmol, 1.00 equiv) and NaHCO₃ (0.20 g, 2.40 mmol, 1.1 equiv) in THF (10 mL) were added 9H-fluoren-9-ylmethyl N-(3-chloro-3-oxopropyl)carbamate (Compound 69, 0.87 g, 2.62 mmol, 1.20 equiv) in portions at 0° C. under N₂ atmosphere. The resulting mixture was stirred for 6h at 0° C. under N₂ atmosphere. LCMS indicated the reaction was completed. The reaction was quenched with water at room temperature. The resulting mixture was extracted with DCM. The combined organic layers were concentrated under reduced pressure. The residue was purified by silica gel column chromatography, eluted with PE/EA (EA=100 %) to afford methyl (2S,3S,4S,5R,6S)-3,4,5-tris(acetyloxy)-6-[2-(3-[[(9H-fluoren-9-ylmethoxy)carbonyl]amino]propanamido)-4-(hydroxymethyl)phenoxy]oxane-2-carboxylate (Compound 70, 1.1 g, 66%) as a light yellow solid. LCMS (ES, m/z):749 [M+H]⁺.

### Step 6. Synthesis of Compound 72

To a stirred mixture of methyl (2S,3S,4S,5R,6S)-3,4,5-tris(acetyloxy)-6-[2-(3-[[(9H-fluoren-9-ylmethoxy)carbonyl]amino]propanamido)-4-(hydroxymethyl)phenoxy]oxane-2-carboxylate (Compound 70, 1.50 g, 2.00 mmol, 1.00 equiv) and bis(4-nitrophenyl) carbonate (Compound 71, 0.68 g, 2.24 mmol, 1.12 equiv) in DMF (15 mL) were added DIEA (0.52 g, 4.01 mmol, 2.00 equiv) in portions at 0° C. under N₂ atmosphere. The resulting mixture was stirred for overnight at room temperature under nitrogen atmosphere. LCMS indicated the reaction was completed. The reaction mixture was purified by reverse flash chromatography with the following conditions: column, C18 silica gel; mobile phase, ACN in water(0.1% FA), 10% to 90% gradient in 40 min; detector, UV 254 nm. The collected fraction was concentrated to dryness in vacuum to afford methyl (2S,3S,4S,5R,6S)-3,4,5-tris(acetyloxy)-6-[2-(3-[[(9H-fluoren-9-ylmethoxy)carbonyl]-amino]propanamido)-4-[[(4-nitrophenoxycarbonyl)oxy]methyl]phenoxy]oxane-2-carboxylate (Compound 72, 1.4 g, 48%) as a yellow solid. LCMS (ES, m/z):914 [M+H]⁺.

### Step 7. Synthesis of Compound 73

To a stirred mixture of methyl (2S,3S,4S,5R,6S)-3,4,5-tris(acetyloxy)-6-[2-(3-[[(9H-fluoren-9-ylmethoxy)carbonyl]amino]propanamido)-4-[[(4-nitrophenoxycarbonyl)oxy]methyl]phenoxy]oxane-2-carboxylate (Copmound 72, 1.00 g, 1.09 mmol, 1.00 equiv) and 1-(3-chloro-4-[2-[2-(methylamino)ethoxy]ethyl]phenyl)-3-[[2-(2,6-dioxopiperidin-3-yl)-1-oxo-3H-isoindol-5-yl]methyl]urea (neoDegrader P1, 0.58 g, 1.09 mmol, 1.00 equiv) in DMF (10 mL) were added HOBT (1.18 g, 8.72 mmol, 8.00 equiv) and 2,4-dimethylpyridine (1.07 g, 8.72 mmol, 8.00 equiv) in portions at room temperature under N₂ atmosphere. The resulting mixture was stirred for 16 h at room temperature under N₂ atmosphere. LCMS indicated the reaction was completed. The resulting mixture was used further purification. The residue was purified by reverse flash chromatography with the following conditions: column, C18 silica gel; mobile phase, ACN in water (0.1% FA), 10% to 80% gradient in 40 min; detector, UV 254 nm. The collected fraction was concentrated under vacuum to afford methyl (2S,3S,4S,5R,6S)-3,4,5-tris(acetyloxy)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]oxane-2-carboxylate (Compound 73 (800 mg, 56%) as a solid. LCMS (ES, m/z):1302[M+H]⁺.

### Step 8. Synthesis of Compound 74

To a stirred mixture of methyl (2S,3S,4S,5R,6S)-3,4,5-tris(acetyloxy)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]oxane-2-carboxylate (Compound 73 (800.00 mg, 0.61 mmol, 1.00 equiv) in THF (80 mL) were added HCl (6N, 80 mL) in portions at room temperature under N₂ atmosphere. The resulting mixture was stirred for 3h at degrees 50° C. under nitrogen atmosphere. LCMS indicated the reaction was completed. The resulting mixture was concentrated under vacuum. The residue was purified by reverse flash chromatography with the following conditions: column, C18 silica gel; mobile phase, ACN in water (0.1% FA), 0% to 80% gradient in 40 min; detector, UV 254 nm. The collected fraction was lyophilized to afford (2S,3S,4S,5R,6S)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]-3,4,5-trihydroxyoxane-2-carboxylic acid (Compound 74, 230 mg, 32%) as a white solid. LCMS (ES, m/z):1162[M+H]⁺.

### Step 9. Synthesis of Compound 75

To a stirred solution of (2S,3S,4S,5R,6S)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]-3,4,5-trihydroxyoxane-2-carboxylic acid, 74 (230 mg, 0.2 mmol, 1.00 equiv) in DMF (2 mL) were added piperidine (0.4 mL) in portions at room temperature under nitrogen atmosphere. The resulting mixture was stirred for 10 min at room temperature under nitrogen atmosphere. LCMS indicated the reaction was completed. The resulting mixture was used directly further purification by Prep-HPLC with the following conditions (Column: XSelect CSH Prep C18 OBD Column,, 19×250 mm,5 um; Mobile Phase A:water (0.05%TFA), Mobile Phase B:ACN; Flow rate: 25 mL/min; Gradient:20 B to 40 B in 7 min; 220 nm; RT1:5.78 min) to afford (2S,3S,4S,5R,6S)-6-[2-(3-aminopropanamido)-4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]phenoxy]-3,4,5-trihydroxyoxane-2-carboxylic acid (Compound 75, 35 mg, 18%) as a white solid. LCMS (ES, m/z): 940[M+H]⁺.

### Step 10. Synthesis of Compound (Ih)

To a stirred solution of (2S,3S,4S,5R,6S)-6-[2-(3-aminopropanamido)-4-[({[2-(2-{2-chloro-4-[({[2-(2,6-dioxopiperidin-3-yl)-1-oxo-3H-isoindol-5-yl]methyl}carbamoyl)amino]phenyl}ethoxy)ethyl](methyl)carbamoyl}oxy)methyl]phenoxy]-3,4,5-trihydroxyoxane-2-carboxylic acid (Compound 75, 110 mg, 0.12 mmol, 1.00 equiv) and bis(2,5-dioxopyrrolidin-1-yl) pentanedioate (Compound 76, 46 mg, 0.14 mmol, 1.2 equiv) in DMF (2.0 mL) was added DIEA (30 mg, 0.23 mmol, 2.0 equiv) in portions at room temperature under nitrogen atmosphere. The resulting mixture was stirred for 1h at room temperature under nitrogen atmosphere. LCMS indicated the reaction was completed. The reaction mixture was purified by Prep-HPLC with the following conditions (Column: Kinetex EVO prep C18, 30*150, 5 um; Mobile Phase A: Water(0.05%TFA), Mobile Phase B: ACN; Flow rate: 60 mL/min; Gradient: 21% B to 41% B in 7 min, 41% B; Wave Length: 254 nm; RT1(min): 5.8. The collected fraction was lyophilized to afford (2S,3S,4S,5R,6S)-6-{4-[({[2-(2-{2-chloro-4-[({[2-(2,6-dioxopiperidin-3-yl)-1-oxo-3H-isoindol-5-yl]methyl}carbamoyl)amino]phenyl}ethoxy)ethyl](methyl)carbamoyl}oxy)methyl]-2-(3-{5-[(2,5-dioxopyrrolidin-1-yl)oxy]-5-oxopentanamido}propanamido)phenoxy}-3,4,5-trihydroxyoxane-2-carboxylic acid (Compound (Ih), 48 mg, 34% as a white solid. LCMS (ES, m/z): 1151 [M+H]⁺, 1173 [M+Na]⁺.¹H-NMR(300 MHz, DMSO-d₆): 12.80 (br s, 1H), 10.98 (s, 1H), 9.08 (s, 1H), 8.79 (s, 1H), 8.18 (s, 1H), 7.96 (s, 1H), 7.68-7.66 (m, 2H), 7.51 (s, 1H), 7.44 (d, J=8.1 Hz,1H), 7.25-7.00 (m, 4H), 6.82-6.80 (m, 1H), 5.86 (s, 1H), 5.39-5.30 (m, 2H), 5.14-5.07 (m, 1H), 4.97 (s, 2H), 4.84 (d, J=7.2 Hz,1H), 4.47-4.27 (m, 4H), 3.90 (d, J=9.6 Hz, 1H), 3.56-3.48 (m, 4H), 3.45-3.36 (m, 6H), 2.95-2.80 (m, 8H), 2.75-2.65 (m, 3H), 2.62-2.55 (m, 2H), 2.49-2.35 (m, 1H), 2.21-2.16 (m, 2H), 2.01-1.95 (m, 1H), 1.85-1..80 (m, 2H).

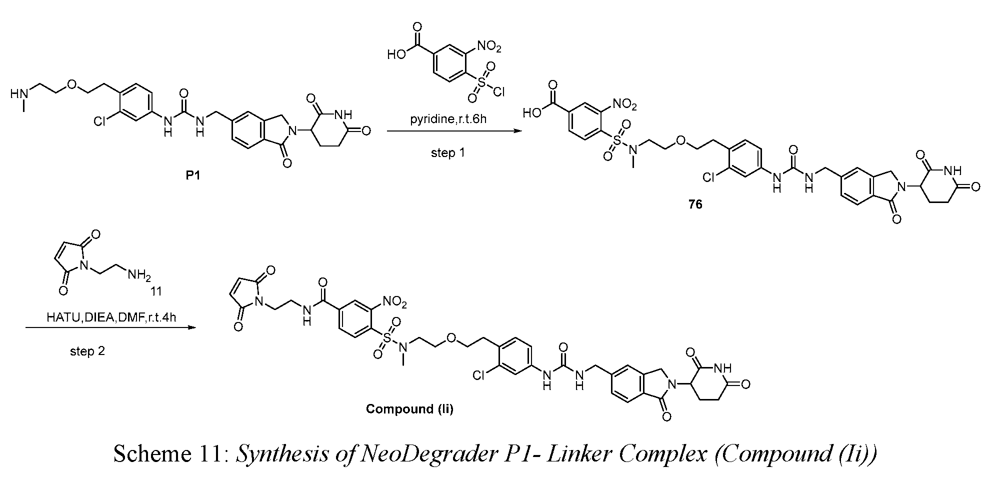

### Step 1. Synthesis of Compound 76

To a stirred solution of 1-(3-chloro-4-[2-[2-(methylamino)ethoxy]ethyl]phenyl)-3-[[2-(2,6-dioxopiperidin-3-yl)-1-oxo-3H-isoindol-5-yl]methyl]urea (Compound P1, 180 mg, 0.34 mmol, 1.00 equiv) in DMF (8 mL) were added TEA (104 mg, 1.02 mmol, 3.0 equiv) and 4-(chlorosulfonyl)-3-nitrobenzoic acid (181 mg, 0.68 mmol, 2.00 equiv) in portions at 0° C. under nitrogen atmosphere. The resulting mixture was stirred for 4h at 0° C. under nitrogen atmosphere. LCMS indicated the reaction was completed. The resulting mixture was used further purification. The residue was purified by reverse flash chromatography with the following conditions: column, C18 silica gel; mobile phase, ACN in water(0.1% FA), 10% to 60% gradient in 10 min; detector, UV 254 nm. The mixture was lyophilized to afford 4-[[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)sulfamoyl]-3-nitrobenzoic acid, (Compound 76, 70 mg, 27%) as a light yellow solid. LCMS (ES, m/z): 757 [M+1]⁺.

### Step 2 Synthesis of Compound (Ii)

To a stirred mixture of 4-[[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)sulfamoyl]-3-nitrobenzoic acid, (Compound 76, 60 mg, 0.08 mmol, 1.00 equiv) in DMF (6 mL) were added HATU (45 mg, 0.12 mmol, 1.5 equiv), 1-(2-aminoethyl)pyrrole-2,5-dione hydrochloride (Compound 77, 17 mg, 0.10 mmol, 1.20 equiv) and DIEA (31 mg, 0.24 mmol, 3.0 equiv) in portions at room temperature under nitrogen atmosphere. The resulting mixture was stirred for 4h at room temperature under nitrogen atmosphere. LCMS indicated the reaction was completed. The residue was purified by Prep-HPLC (Column: XBridge Prep Phenyl OBD Column, 19×150 mm 5um 13 nm; Mobile Phase A:water(0.05%TFA ), Mobile Phase B:ACN; Flow rate:25 mL/min; Gradient:25 B to 43 B in 10 min; 220 nm; RT1:11.97 min). The collected fraction was lyophilized to afford 4-[[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)sulfamoyl]-N-[2-(2,5-dioxopyrrol-1-yl)ethyl]-3-nitrobenzamide (Compound (Ii), 27 mg, 36%) as a white solid. LCMS (ES, m/z): 879,881 [M+H]. ¹H NMR (300 MHz, DMSO-d₆) δ 11.00 (s, 1H), 9.01 (t, J=6.0 Hz, 1H), 8.82 (s, 1H), 8.20 (s, 1H), 8.11 (s, 2H), 7.71-7.67 (m, 2H), 7.52 (s, 1H), 7.44 (d, J=3.0 Hz, 1H), 7.21-7.12 (m, 2H), 7.02 (s, 2H), 6.84 (t, J=6.0 Hz, 1H), 5.14-5.08 (m, 1H), 4.48-4.28 (m, 4H), 3.62-3.50(m, 6H), 3.40-3.28 (m, 2H), 2.95-2.85(m, 4H), 2.80-2.73 (m, 2H), 2.65-2.60 (s, 1H), 2.41-2.27 (m, 1H), 2.05-1.95 (m, 1H).

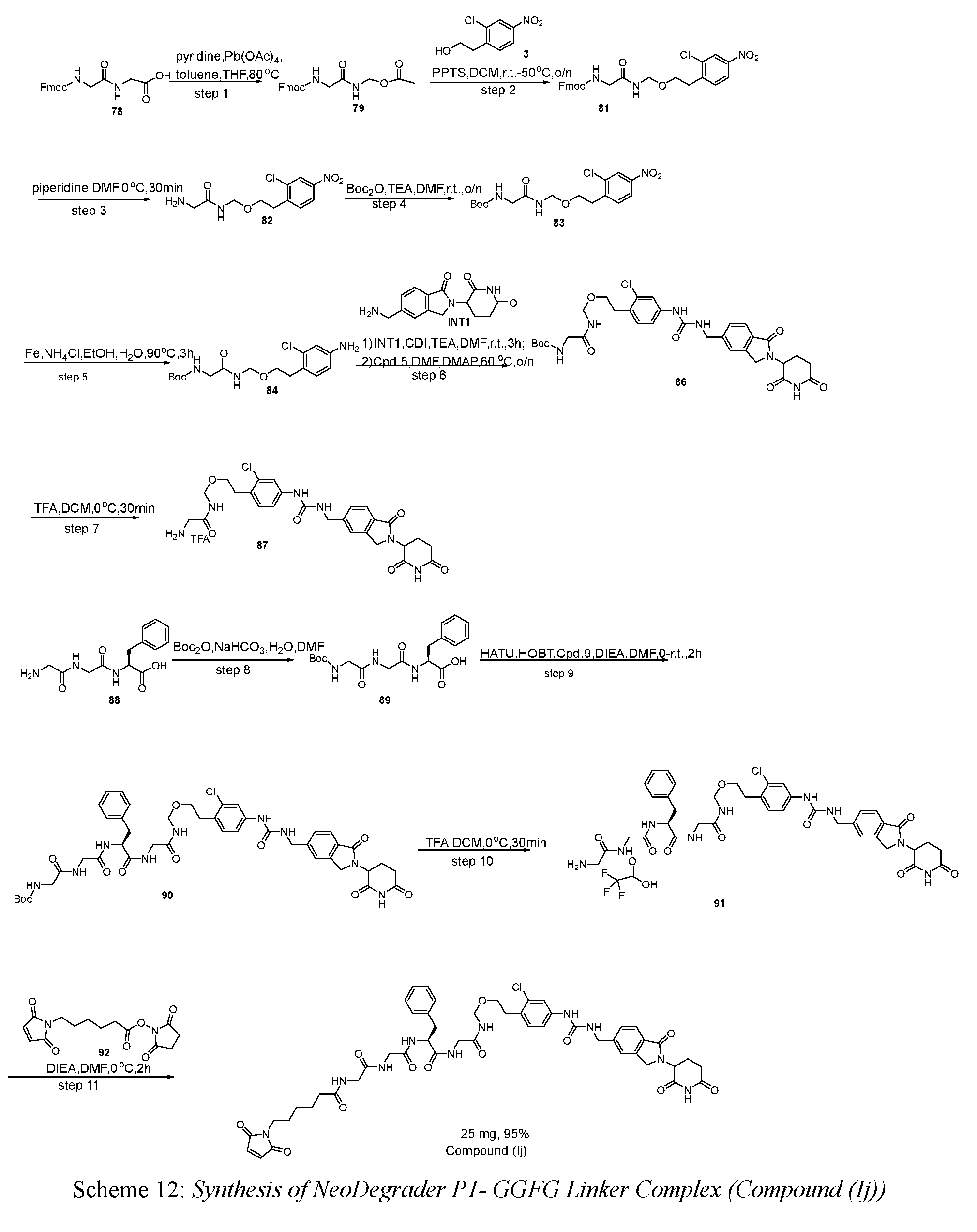

### Step 1. Synthesis of Compound 79

To a stirred mixture of (2-[[(9H-fluoren-9-ylmethoxy)carbonyl]amino]-acetamido)acetic acid (Compound 78, 10.00 g, 28.22 mmol, 1.00 equiv) and Pb(OAc)₄ (15.02 g, 33.86 mmol, 1.20 equiv) in THF(300 mL) and toluene(100 mL) were added pyridine (2.59 g, 32.74 mmol, 1.16 equiv) dropwise at room temperature under nitrogen atmosphere. The resulting mixture was stirred for overnight at 80° C. under nitrogen atmosphere. LCMS indicated the reaction was completed. The mixture was allowed to cool down to room temperature. The resulting mixture was filtered, and the filter cake was washed with ethyl acetate (20 mL). The filtrate was concentrated under reduced pressure. The residue was dissolved in ethyl acetate (20 mL), washed with water, brine, dried over anhydrous Na₂SO₄. After filtration, the filtrate was concentrated under reduced pressure. The residue was purified by silica gel column chromatography, eluted with PE/EtOAc (1:4) to afford (2-[[(9H-fluoren-9-ylmethoxy)carbonyl]amino]acetamido)methyl acetate (Compound 79, 6.5 g, 56%) as a white solid. LCMS (ESI, ms): 391[M+Na]⁺. ¹HNMR (300 MHz, CDCl₃) δ 7.80(d, J=7.5 Hz, 2H), 7.62(d, J=7.5 Hz, 2H), 7.45(t, J=7.5 Hz, 2H), 7.36(d, J=7.5 Hz, 2H), 7.18(br s, 1H),5.48(br s, 1H), 5.28(d, J=7.2 Hz, 2H), 4.48(d, J=6.6 Hz, 2H), 4.26(t, J=6.6 Hz, 1H), 3.93(d, 5.4 Hz, 2H), 2.08(s, 3H).

### Step 2. Synthesis of Compound 81

To a stirred mixture of (2-[[(9H-fluoren-9-ylmethoxy)carbonyl]-amino]acetamido)methyl acetate, 79 (2.00 g, 5.43 mmol, 1.00 equiv) and 2-(2-chloro-4-nitrophenyl)ethanol (Compound 3, 3.20 g, 15.85 mmol, 2.92 equiv) in DCM (40 mL) was added PPTS (400 mg, 1.59 mmol, 0.29 equiv) dropwise at 0° C. under nitrogen atmosphere. The resulting mixture was stirred for overnight at 45° C. under nitrogen atmosphere. 40% desired product could be detected by LCMS. The mixture was allowed to cool down to room temperature. The reaction was quenched with water/ice. The resulting mixture was extracted with EtOEt (3 × 20 mL). The combined organic layers were washed with brine (30 mL), dried over anhydrous Na₂SO₄. After filtration, the filtrate was concentrated under reduced pressure. The residue was purified by silica gel column chromatography, eluted with PE/EtOAc (1:9) to afford 9H-fluoren-9-ylmethyl N-[([[2-(2-chloro-4-nitrophenyl)ethoxy]methyl]carbamoyl)methyl]carbamate (Compound 81, 1.7 g, 55%) as a white solid. LCMS (ESI, ms): 510,512[M+H]⁺. ¹HNMR (300 MHz, DMSO-d₆): δ 8.58 (t, J=5.1 Hz, 1H), 8.22 (dd, J=12, 2.4 Hz, 1H), 7.89 (d, J=7.5 Hz, 1H), 7.71-7.54 (m, 4H), 7.43-7.29 (m, 4H), 4.56 (d, J=6.9 Hz, 2H), 4.30-4.16 (m, 3H), 3.70-3.61(m, 4H), 3.04 (t, J=6.3 Hz, 2H).

### Step 3. Synthesis of Compound 82

To a stirred mixture of 9H-fluoren-9-ylmethyl N-[([[2-(2-chloro-4-nitrophenyl)ethoxy]methyl]carbamoyl)methyl]carbamate (Compound 81, 1.60 g, 3.14 mmol, 1.00 equiv) in DMF(5.0 mL) was added piperidine(1.0 mL) in portions at 0° C. under nitrogen atmosphere. The resulting mixture was stirred for 1h at room temperature under nitrogen atmosphere. LCMS indicated the reaction was completed. The reaction mixture was purified by reverse flash chromatography with the following conditions: column, C18 silica gel; mobile phase, ACN in water(0.05%TFA), 0% to 50% gradient in 40 min; detector, UV 254 nm. This resulted in 2-amino-N-[[2-(2-chloro-4-nitrophenyl)ethoxy]methyl]acetamide (Compound 82, 750 mg, 76%) as a yellow oil. LCMS (ESI, ms) 288[M+H]⁺, 329[M+H+ACN]⁺

### Step 4. Synthesis of Compound 83

To a stirred mixture of 2-amino-N-[[2-(2-chloro-4-nitrophenyl)ethoxy]-methyl]acetamide (Compound 82, 750 mg, 2.61 mmol, 1.00 equiv) and Boc₂O (580 mg, 2.66 mmol, 1.02 equiv) in DMF(10.00 mL) was added NaHCO₃(477 mg, 5.68 mmol, 2.18 equiv) in H₂O (10.00 mL) dropwise at 0° C. The resulting mixture was stirred for 3h at room temperature. LCMS indicated the reaction was completed. The reaction was quenched by the addition of water (20 mL). The resulting mixture was extracted with EtOEt (3 × 20 mL). The combined organic layers were washed with brine (20 mL), dried over anhydrous Na₂SO₄. After filtration, the filtrate was concentrated under reduced pressure. The residue was purified by silica gel column chromatography, eluted with PE/EtOAc (1:2) to afford tert-butyl N-[([[2-(2-chloro-4-nitrophenyl)ethoxy]methyl]carbamoyl)methyl]carbamate (Compound 83, 650 mg, 58%) as a yellow oil. LCMS(ESI, ms), 388[M+H]⁺, 332[M+H-56]⁺. ¹HNMR (400 MHz, CDCl₃) δ 8.21(d, J=2.4 Hz, 1H), 8.04(d, J=8.4 Hz, 2H), 7.46(d, J=8.4 Hz, 1H), 7.05(br s, 1H), 5.25(br s, 1H), 4.73(d, J=7.2 Hz, 2H), 3.81-3.73(m, 4H), 3.34-3.32(m, 2H), 3.08(t, J=6.8 Hz, 2H), 1.42(s, 9H).

### Step 5. Synthesis of Compound 84

To a stirred mixture of tert-butyl N-[([[2-(2-chloro-4-nitrophenyl)ethoxy]methyl]-carbamoyl)methyl]carbamate (Compound 83, 650 mg, 1.68 mmol, 1.00 equiv) and Fe(260 mg, 4.66 mmol, 2.78 equiv) in EtOH (9.00 mL) was added NH₄Cl (910 mg, 17.01 mmol, 10.1 equiv) in H₂O (3.00 mL) dropwise at room temperature. The resulting mixture was stirred for 4h at 90° C. LCMS indicated the reaction was completed. The mixture was allowed to cool down to room temperature. The resulting mixture was concentrated under vacuum. The resulting mixture was extracted with EtOAc (3 × 20 mL). The combined organic layers were washed with brine (20 mL), dried over anhydrous Na₂SO₄. After filtration, the filtrate was concentrated under reduced pressure. The residue was purified by silica gel column chromatography, eluted with PE/EtOAc (1:1) to afford tert-butyl N-[([[2-(4-amino-2-chlorophenyl)ethoxy]methyl]-carbamoyl)methyl]carbamate (Compound 84, 500 mg, 83%) as a yellow solid. LCMS(ESI, ms): 358[M+H]⁺, 380[M+Na]⁺. ¹HNMR (300 MHz, CDCl₃) δ 7.02-6.96(m, 2H), 6.68(d, J=2.4 Hz, 1H), 6.52-6.49(m, 1H), 5.29(br s, 1H), 4.74(d, J=6.9 Hz, 2H), 3.80-3.78(m, 2H), 3.69-3.63(m, 2H), 2.88(t, J=7.2 Hz, 2H), 1.45(s, 9H).

### Step 6. Synthesis of Compound 86

To a stirred mixture of 3-[5-(aminomethyl)-1-oxo-3H-isoindol-2-yl]piperidine-2,6-dione hydrochloride (Compound 85, 398 mg, 1.28 mmol, 0.92 equiv) and CDI(450 mg, 2.78 mmol, 1.99 equiv) in DMF(5.00 mL) was added TEA(300 mg, 2.96 mmol, 2.12 equiv) at 0° C. The resulting mixture was stirred for 2h at room temperature. To the above mixture was added tert-butyl N-[([[2-(4-amino-2-chlorophenyl)ethoxy]methyl]carbamoyl)methyl]carbamate (Compound 84, 500 mg, 1.40 mmol, 1.00 equiv) and DMAP (550 mg, 4.50 mmol, 3.22 equiv) in portions. The resulting mixture was stirred for additional overnight at 60° C. LCMS indicated the reaction was completed. The mixture was allowed to cool down to room temperature. The reaction mixture was purified by reverse flash chromatography with the following conditions: column, C18 silica gel; mobile phase, ACN in water (0.1% FA), 0% to 50% gradient in 30 min; detector, UV 254 nm. This resulted in tert-butyl N-([[(2-[2-chloro-4-[([[2-(2,6-dioxopiperidin-3-yl)-1-oxo-3H-isoindol-5-yl]methyl]-carbamoyl)amino]phenyl]ethoxy)methyl]carbamoyl]methyl)carbamate (Compound 86, 550 mg, 60%) as a light brown solid. LCMS (ESI, ms): 657[M+H]⁺, 601[M+H-56]⁺, 557[M+H-100]⁺.

### Step 7. Synthesis of Compound 87

To a stirred mixture of tert-butyl N-([[(2-[2-chloro-4-[([[2-(2,6-dioxopiperidin-3-yl)-1-oxo-3H-isoindol-5-yl]methyl]carbamoyl)amino]phenyl]ethoxy)methyl]carbamoyl]methyl)-carbamate (Compound 86, 530 mg, 0.80 mmol, 1.00 equiv) in DCM (5.00 mL) was added TFA (1.00 mL) at 0° C. The resulting mixture was stirred for 30 min at 0° C. LCMS indicated the reaction was completed. The resulting mixture was concentrated under reduced pressure. This resulted in 2-amino-N-[(2-[2-chloro-4-[([[2-(2,6-dioxopiperidin-3-yl)-1-oxo-3H-isoindol-5-yl]methyl]carbamoyl)amino]phenyl]ethoxy)methyl]acetamide; trifluoroacetic acid (Compound 87, (510 mg, purity:64%, yield: 60%) as an off-white solid. LCMS (ESI, ms):557[M+H-TFA]⁺

### Step 8. Synthesis of Compound 89

To a stirred mixture of (2S)-2-[2-(2-aminoacetamido)acetamido]-3-phenylpropanoic acid (Compound 88, 2.00 g, 7.16 mmol, 1.00 equiv) and NaHCO₃ (1.80 g, 21.41 mmol, 3.00 equiv) in H₂O (40.00 mL) were added Boc₂O (1.86 g, 8.52 mmol, 1.20 equiv) in DMF (40.00 mL) dropwise at 0° C. The resulting mixture was stirred for overnight at room temperature. LCMS indicated the reaction was completed. The reaction was quenched with water at room temperature. The resulting mixture was extracted with EtOEt (3 × 50 mL). The combined organic layers were washed with brine (50 mL), dried over anhydrous Na₂SO₄. After filtration, the filtrate was concentrated under reduced pressure. The residue was purified by reverse flash chromatography with the following conditions: column, C18 silica gel; mobile phase, ACN water(0.05% TFA), 5% to 60% gradient in 30 min; detector, UV 220 nm. This resulted in (2S)-2-(2-[2-[(tert-butoxycarbonyl)amino]acetamido]acetamido)-3-phenylpropanoic acid (Compound 89, 1.8 g, 60%) as a white semi-solid. LCMS (ESI,ms):380[M+H]⁺, 324[M+H-56]⁺. ¹HNMR:(300 MHz, DMSO-d₆) δ 8.17(d, J=8.1 Hz, 1H), 7.93(t, J=5.7 Hz, 1H), 7.31-7.20(m, 5H), 7.00(t, J =6.0 Hz, 1H), 4.46-4.39(m, 1H),3.78-3.67(m, 2H), 3.56(d, J =5.7 Hz, 2H), 3.09-3.02(m, 1H), 2.92-2.73(m, 1H), 1.39(s, 9H).

### Step 9. Compound 90

To a stirred mixture of (2S)-2-(2-[2-[(tert-butoxycarbonyl)amino]acetamido]-acetamido)-3-phenylpropanoic acid (Compound 89, 340 mg, 0.90 mmol, 1.00 equiv) and HATU (340 mg, 0.90 mmol, 1.00 equiv) in DMF (5.00 mL) was added HOBT (102 mg, 0.75 mmol, 0.84 equiv) in portions at 0° C. The resulting mixture was stirred for 30 min at 0° C. To the above mixture was added 2-amino-N-[(2-[2-chloro-4-[([[2-(2,6-dioxopiperidin-3-yl)-1-oxo-3H-isoindol-5-yl]methyl]carbamoyl)amino]phenyl]ethoxy)methyl]acetamide; trifluoroacetic acid (Compound 87, 511 mg, purity: 64%, 0.48 mmol, 0.54 equiv) and DIEA (340 mg, 2.63 mmol, 2.94 equiv) at 0° C. The resulting mixture was stirred for additional 2h at room temperature. LCMS indicated the reaction was completed. The reaction mixture was purified by reverse flash chromatography with the following conditions: column, C18 silica gel; mobile phase, ACN in water (0.1% FA), 0% to 50% gradient in 30 min; detector, UV 220 nm. The collected fraction was concentrated under vacuum. This resulted in tert-butyl N-[[([[(1S)-1-[([[(2-[2-chloro-4-[([[2-(2,6-dioxopiperidin-3-yl)-1-oxo-3H-isoindol-5-yl]methyl]carbamoyl)amino]phenyl]ethoxy)methyl]carbamoyl]methyl)carbamoyl]-2-phenylethyl]carbamoyl]methyl)carbamoyl]methyl]carbamate (Compound 90, 210 mg, 48%) as an off-white solid. LCMS (ESI, ms):918[M+H]⁺, 818[M+H-100]⁺. ¹HNMR:(400 MHz, DMSO-d₆): δ 10.97(s, 1H), 8.79(s, 1H), 8.50(t, J=6.4 Hz, 1H), 8.31(t, J=4.4 Hz, 1H), 8.15(d, J = 9.6 Hz, 1H), 7.910(t, J=8.0 Hz, 1H), 7.68-7.64(m, 2H), 7.49(s, 1H), 7.43(d, J=9.6 Hz, 1H), 7.24-7.12(m, 7H), 7.00-6.95(m, 1H), 6.84(t, J=6.4 Hz, 1H), 5.13-5.06(m, 1H), 4.55-4.27(m, 7H), 3.72-3.60(m, 6H), 3.75-3.67(m, 3H), 3.59-3.49(m, 5H), 3.07-3.01(m, 1H), 2.94-2.73(m, 4H), 2.62-2.54(m, 1H), 2.40-2.31(m, 1H), 2.01-1.94(m, 1H), 2.00-1.91(m, 1H), 1.35(s, 9H)

### Step 10. Synthesis of Compound 91

To a stirred mixture of tert-butyl N-[[([[(1S)-1-[([[(2-[2-chloro-4-[([[2-(2,6-dioxopiperidin-3-yl)-1-oxo-3H-isoindol-5-yl]methyl]carbamoyl)amino]phenyl]ethoxy)-methyl]carbamoyl]methyl)carbamoyl]-2-phenylethyl]carbamoyl]methyl)carbamoyl]-methyl]carbamate (Compound 90, 140 mg, 0.15 mmol, 1.00 equiv) in DCM (5.00 mL) was added TFA (1.00 mL) dropwise at 0° C. The resulting mixture was stirred for 30 min at 0° C. LCMS indicated the reaction was completed. The resulting mixture was concentrated under reduced pressure. This resulted in (2S)-2-[2-(2-aminoacetamido)acetamido]-N-([[(2-[2-chloro-4-[([[2-(2,6-dioxopiperidin-3-yl)-1-oxo-3H-isoindol-5-yl]methyl]carbamoyl)amino]phenyl]ethoxy)methyl]-carbamoyl]methyl)-3-phenylpropanamide; trifluoroacetic acid (Compound 91, 140 mg, 79%) as an off-white solid. LCMS(ESI, ms):818[M+H-TFA]⁺.

### Step 11. Synthesis of Compound (Ij)

To a stirred mixture of (2S)-2-[2-(2-aminoacetamido)acetamido]-N-([[(2-[2-chloro-4-[([[2-(2,6-dioxopiperidin-3-yl)-1-oxo-3H-isoindol-5-yl]methyl]carbamoyl)amino]phenyl]-ethoxy)methyl]carbamoyl]methyl)-3-phenylpropanamide; trifluoroacetic acid (Compound 91, 140 mg, 0.15 mmol, 1.00 equiv) and DIEA(70 mg, 0.54 mmol, 3.61 equiv) in DMF(2.00 mL) was added 2,5-dioxopyrrolidin-1-yl 6-(2,5-dioxopyrrol-1-yl)hexanoate (Compound 92, 70 mg, 0.23 mmol, 1.50 equiv) in portions at 0° C. The resulting mixture was stirred for 2 h at room temperature. LCMS indicated the reaction was completed. The reaction mixture was directly purified by the following condition: Column: XSelect CSH Prep C18 OBD Column, 19×250 mm,5 um; Mobile Phase A:Water(0.1%FA), Mobile Phase B:ACN; Flow rate:25 mL/min; Gradient:25 B to 50 B in 7 min; 254 nm; RT1:6.35 min; The collected fraction was lyophilized to give the crude product. The crude product was re-purified by the following condition:Column: Kinetex EVO C18 Column, 30×150.5 um; Mobile Phase A:Water(0.05%TFA ), Mobile Phase B:ACN; Flow rate:60 mL/min; Gradient:20 B to 40 B in 7 min, 220 nm; RT1:6.77 min; The collected fraction was lyophilized to give the N-[[([[(1S)-1-[([[(2-[2-chloro-4-[([[2-(2,6-dioxopiperidin-3-yl)-1-oxo-3H-isoindol-5-yl]methyl]carbamoyl)amino]phenyl]ethoxy)methyl]carbamoyl]methyl)carbamoyl]-2-phenylethyl]carbamoyl]methyl)carbamoyl]methyl]-6-(2,5-dioxopyrrol-1-yl)hexanamide (Compound (Ik), 22.8 mg, 14%) as off-white solid. LCMS (ESI, ms):1011 [M+H]⁺. ¹HNMR:(400 MHz, DMSO-d₆): δ 10.95(s, 1H), 8.79(s, 1H), 8.51(t, J=8.4Hz, 1H), 8.29(t, J=8.0Hz, 1H), 8.12-8.01(m, 3H), 7.70-7.66(m, 2H), 7.44(s, 1H), 7.42(d, J=8.0Hz, 1H), 7.23-7.16(m, 7H), 6.99(s, 2H), 6.82(t, J=8.0Hz, 1H), 5.13-5.09(m, 1H), 4.55-4.28(m, 7H), 3.72-3.60(m, 6H), 3.55-3.51(m, 2H), 3.36-3.34(m, 2H), 3.05-3.00(m, 1H), 2.94-2.72(m, 4H), 2.62-2.54(m, 1H), 2.40-2.32(m, 1H), 2.12-2.05(m, 2H), 2.00-1.91(m, 1H), 1.50-1.38(m, 4H), 1.19-1.10(m, 2H)

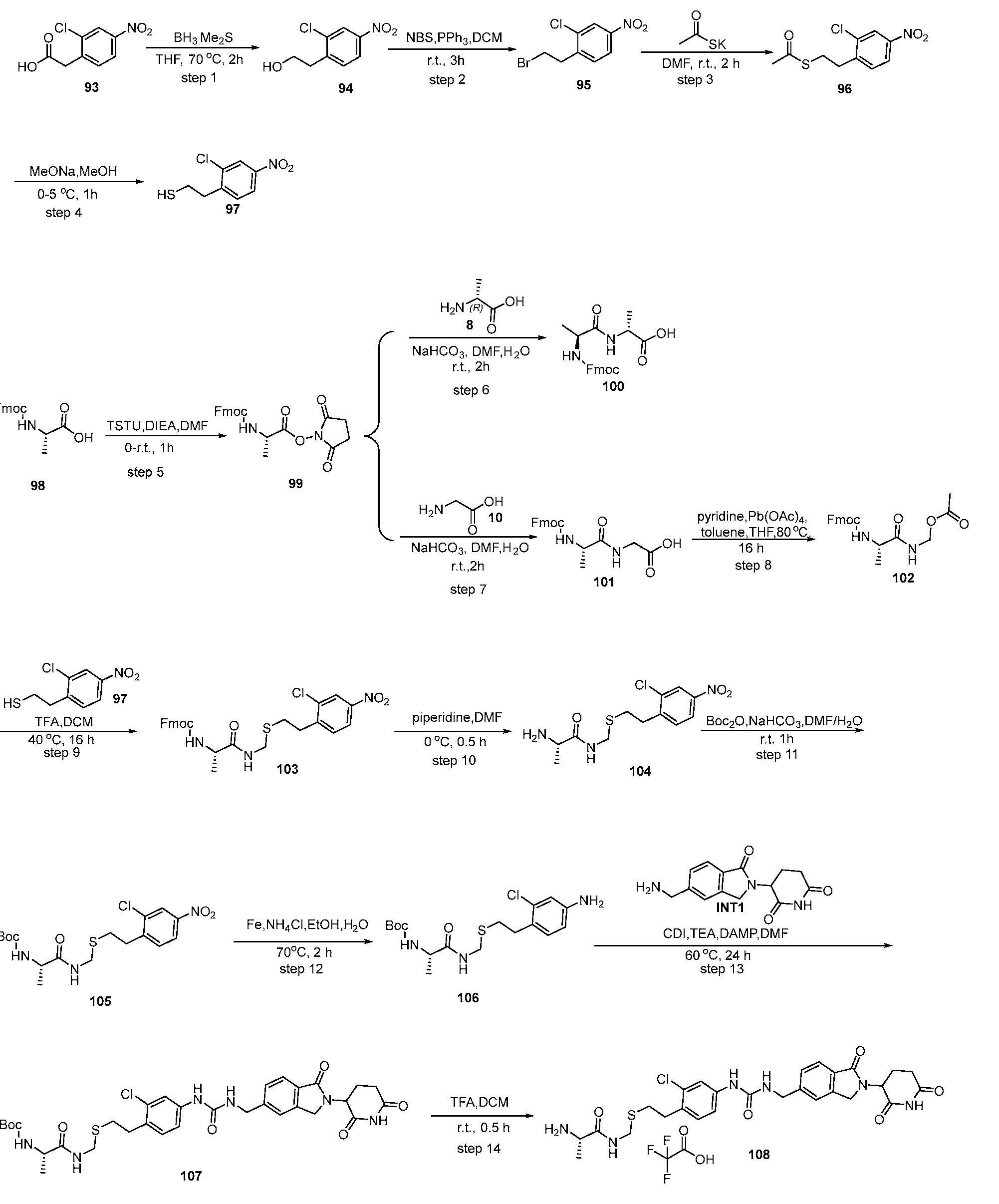 [SMILES: CNCCOCCc1ccc(NC(=O)NCc2ccc3c(c2)CN(C2CCC(=O)NC2=O)C3=O)cc1Cl]

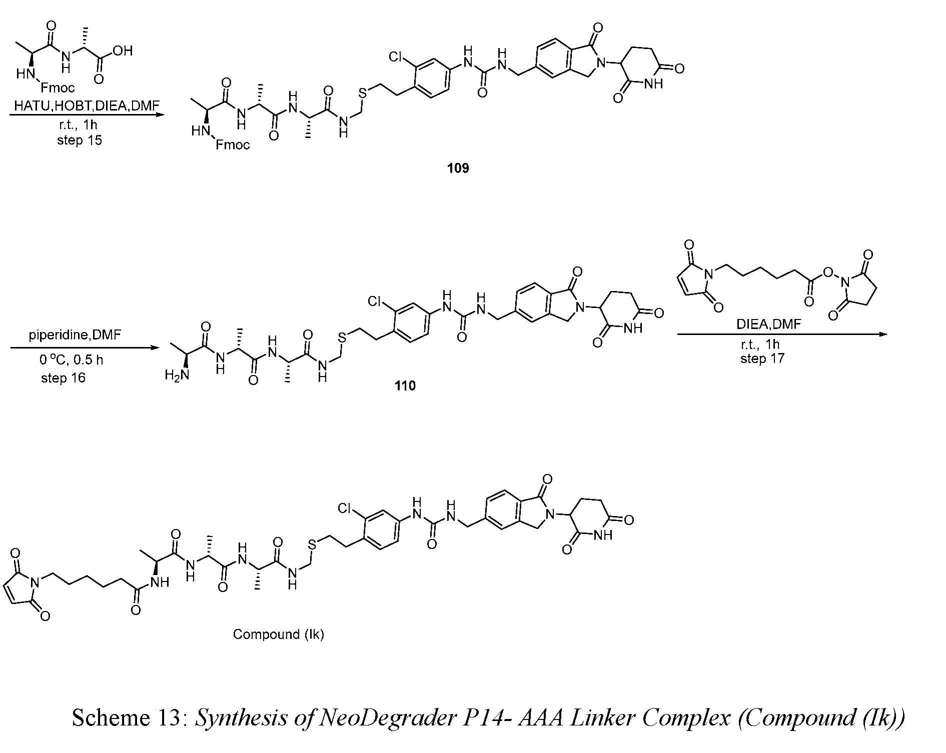 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CCOCCc2ccc(NC(=O)NCc3ccc4c(c3)CN(C3CCC(=O)NC3=O)C4=O)cc2Cl)cc1]

### Step 1. Synthesis of Compound 94

To a stirred solution of (2-chloro-4-nitrophenyl)acetic acid (Compound 93, 24.00 g, 111.32 mmol, 1.00 equiv) in THF (240.00 mL) were added BH₃—Me₂S (28.00 mL, 295.23 mmol, 2.65 equiv) dropwise under nitrogen atmosphere. The resulting mixture was stirred for 2 hours at 70° C. under nitrogen atmosphere. TLC (PE: EtOAc = 3:1) indicated the reaction was completed. After cooled to room temperature, the resulting mixture was concentrated under vacuum. The residue was purified by silica gel column chromatography, eluted with PE/ EtOAc (3:1) to afford 2-(2-chloro-4-nitrophenyl)ethanol (Compound 94, 18.00 g, 80%) as a light yellow solid. ¹H NMR (300 MHz, CDCl₃) δ 8.27 (s, 1H), 8.10-8.07 (m, 1 H), 7.52 (d, J= 3 Hz, 1H), 3.96 (t, J= 6 Hz, 2H), 3.13 (t, J= 6 Hz, 2H).

### Step 2. Synthesis of Compound 95

To a stirred solution of 2-(2-chloro-4-nitrophenyl)ethanol (Compound 94, 5.00 g, 24.80 mmol, 1.00 equiv) in DCM (100.00 mL) were added NBS (6.62 g, 1.50 equiv) and PPh₃ (9.76 g, 37.21 mmol, 1.50 equiv) in portions at room temperature under under N₂. The resulting mixture was stirred overnight at room temperature under N₂. TLC (PE: EtOAc = 10:1) indicated the reaction was completed. The reaction was concentrated to dryness under vacuum. The residue was purified by silica gel column chromatography, eluted with PE/ EtOAc (4:1) to afford 1-(2-bromoethyl)-2-chloro-4-nitrobenzene (Compound 95, 5.10 g, 72%) as a red oil. ¹H NMR (400 MHz, DMSO-d₆) δ 8.28 (d, J= 2.4 Hz, 1H), 8.18 (dd, J= 8.4, 2.4 Hz, 1H), 7.73 (d, J= 8.4 Hz, 1H), 3.79 4(t, J = 6.8 Hz, 2H), 3.38 (t, J = 6.8 Hz, 2H).

### Step 3. Synthesis of Compound 96

To a solution of 1-(2-bromoethyl)-2-chloro-4-nitrobenzene (Compound 95, 5.00 g, 18.90 mmol, 1.00 equiv) in DMF (50.00 mL) was added potassium thioacetate (2.16 g, 18.90 mmol, 1.00 equiv) at room temperature under nitrogen atmosphere. The resulting mixture was stirred at room temperature for 2 hours. TLC (PE: EtOAc= 10:1) indicated the reaction was completed. The reaction was diluted with water (600.00 mL), and extracted with EtOAc (2000 mLx3). The combined organic layer was washed with water (200.00 mL), brine (200.00 mL), dried over anhydrous Na₂SO₄ and concentrated to dryness under vacuum to afford 1-[[2-(2-chloro-4-nitrophenyl)ethyl]sulfanyl]ethenone (Compound 96, 4.50 g, 85%) as a red oil. ¹H NMR (400 MHz, CDCl₃) δ 8.24 (d, J = 2.4 Hz, 1H), 8.07 (dd, J = 8.4, 2.4 Hz, 1H), 7.45 (d, J = 8.4 Hz, 1H), 3.20 -3.05 (m, 4H), 2.34 (s, 3H).

### Step 4. Synthesis of Compound 97

To a stirred solution of 1-[[2-(2-chloro-4-nitrophenyl)ethyl]sulfanyl]ethenone (Compound 96, 2.00 g, 7.70 mmol, 1.00 equiv) in MeOH (300.00 mL) was added MeONa (6.93 mL, 37.33 mmol, 5.00 equiv, 30% in MeOH) at 0° C. under N₂. The resulting mixture was stirred at 0° C. under N₂ for 1 h. TLC (PE: EtOAc =10:1) indicated the reaction was completed. The reaction was quenched with AcOH to pH value to 3-4. The resulting mixture was concentrated to dryness under vacuum. The residue was diluted with DCM (50.00 mL) and filtered. The filtrate was purified with Prep-TLC (PE: EtOAc = 10:1) to give 2-(2-chloro-4-nitrophenyl)ethanethiol (Compound 97, 1.35 g, 72%) as a light yellow oil. ¹H NMR (400 MHz, CDCl₃) δ 8.26 (d, J = 2.4 Hz, 1H), 8.09 (dd, J= 8.4, 2.4 Hz, 1H), 7.45 (d, J= 8.4 Hz, 1H), 3.14 (t, J= 8.0 Hz, 2H), 2.85 (dt, J = 8.0, 7.2 Hz, 2H), 1.43 (t, J= 7.2 Hz, 1H).

### Step 5. Synthesis Compound 99

To a stirred solution of (2S)-2-[[(9H-fluoren-9-ylmethoxy)carbonyl]amino]propanoic acid (Compound 98, 20.00 g, 64.24 mmol, 1.00 equiv) in DMF (200.00 mL) were added TSTU (25.18 g, 83.52 mmol, 1.30 equiv) and DIEA (16.60 g, 128.48 mmol, 2.00 equiv) at room temperature under air atmosphere. The resulting mixture was stirred for 1 h at room temperature. LCMS indicated the reaction was completed. The reaction was diluted with water (200.00 mL), was extracted with EtOAc (100.00 mLx3). The combined organic layer was washed with water (100.00 mL), brine (100.00 mL), dried over anhydrous Na₂SO₄ and concentrated to dryness in vacuum. The residue was purified by silica gel column chromatography, eluted with (PE: EtOAc =1:2) to give 2,5-dioxopyrrolidin-1-yl (2S)-2-[[(9H-fluoren-9-ylmethoxy)carbonyl]amino]propanoate (Compound 99, 25.00 g,83%) t as a white solid. LCMS (ES, m/z):431[M+Na]⁺.

### Step 6. Synthesis of Compound 100

To a solution of D-alanine (1.09 g, 0.012 mmol, 1.00 equiv) and NaHCO₃ (3.09 g, 0.04 mmol, 3.00 equiv) in water (50.00 mL) was added a solution of 2,5-dioxopyrrolidin-1-yl (2S)-2-[[(9H-fluoren-9-ylmethoxy)carbonyl]amino]propanoate (Compound 99, 5.00 g, 12.24 mmol, 1.00 equiv) in DMF (50.00 mL,). The resulting mixture was stirred at room temperature for 2 h. LCMS indicated the reaction was completed. The reaction was adjusted to pH value to 2-3 with 2 NHCl. The resulting mixture was extracted with EtOAc (100.00 mLx3), and the combined organic layer was washed with brine (100.00 mLx3), dried over anhydrous Na₂SO₄ and concentrated to dryness under vacuum to give (2R)-2-[(2S)-2-[[(9H-fluoren-9-ylmethoxy)carbonyl]-amino]propanamido]propanoic acid (Compound 100, 4.00 g, 71%) as a white solid. LCMS (ES, m/z): 383 [M+H]⁺

### Step 7. Synthesis of Compound 101

To a solution of glycine (3.68 g, 48.97 mmol, 1.00 equiv) and NaHCO₃ (12.34 g, 146.89 mmol, 3.00 equiv) in water (200.00 mL) was added solution of 2,5-dioxopyrrolidin-1-yl (2S)-2-[[(9H-fluoren-9-ylmethoxy)carbonyl]amino]propanoate (Compound 99, 20.00 g, 48.97 mmol, 1.00 equiv) in DMF (200.00 mL). The reaction was stirred at room temperature for 2 h. LCMS indicated the reaction was completed. The reaction was adjusted to pH value to 2-3 with 2 N HCl. The resulting mixture was extracted with EtOAc (500.00 mLx3), and the combined organic layer was washed with brine (500.00 mL), dried over anhydrous Na₂SO₄ and concentrated to dryness in vacuum to give [(2S)-2-[[(9H-fluoren-9-ylmethoxy)carbonyl]amino]propanamido]acetic acid (Compound 101, 15.00 g,71%) as a white solid. LCMS (ES, m/z): 369 [M+H]⁺

### Step 8. Synthesis of Compound 102

A solution of [(2S)-2-[[(9H-fluoren-9-ylmethoxy)carbonyl]amino]propanamido]-acetic acid (Compound 101, 5.00 g, 13.57 mmol, 1.00 equiv), Pb(OAc)₄ (7.22 g, 16.28 mmol, 1.20 equiv) and pyridine (1.29 g, 16.31 mmol, 1.20 equiv) in THF (300.00 mL)/Toluene (100.00 mL) under N₂ was stirred at 80° C. for 16 h. LCMS indicated the reaction was completed. After cooled to room temperature, the reaction was filtered. The filter cake was washed with THF (100.00 mL).The combined organic layer was concentrated to dryness under vacuum. The residue was purified by silica gel column chromatography, eluted with (PE: EtOAc=1:2) to give [(2S)-2-[[(9H-fluoren-9-ylmethoxy)carbonyl]amino]propanamido]methyl acetate (Compound 102, 2.50 g, 45%) as a white solid. LCMS (ES, m/z): 405 [M+Na]⁺. ¹H NMR (400 MHz, Chloroform-d) δ 7.77 (t, J = 7.6 Hz, 2H), 7.58 (d, J= 7.6 Hz, 2H), 7.43 - 7.37 (m, 2H), 7.36 - 7.29 (m, 2H), 7.10 (s, 1H), 5.24 (d, J= 7.6 Hz, 2H), 4.51 - 4.35 (m, 2H), 4.23-4.09 (m, 2H), 2.04 (s, 3H), 1.39 (d, J= 6.8 Hz, 3H).

### Step 9. Synthesis of Compound 103

To a stirred solution of [(2S)-2-[[(9H-fluoren-9-ylmethoxy)carbonyl]amino]-propanamido]methyl acetate (Compound 102, 2.25 g, 5.88 mmol, 1.00 equiv) and 2-(2-chloro-4-nitrophenyl)ethanethiol (Compound 97, 1.28 g, 5.88 mmol, 1.00 equiv) in DCM(120 mL) was added TFA (0.27 mL, 2.37 mmol, 0.62 equiv) under N₂ at room temperature. The resulting mixture was stirred at room temperature for 16 hours. LCMS indicated the reaction was completed. The reaction was concentrated to dryness in vacuum. The residue was purified by silica gel column chromatography, eluted with (PE: EtOAc=1:4) to give to give 9H-fluoren-9-ylmethyl N-[(1S)-1-[([[2-(2-chloro-4-nitrophenyl)ethyl]sulfanyl]methyl)carbamoyl]ethyl]carbamate (Compound 103, 3.10 g, 90%) as a yellow solid. LCMS (ES, m/z): 540 [M+H]⁺

### Step 10. Synthesis of Compound 104

To a solution of 9H-fluoren-9-ylmethyl N-[(1S)-1-[([[2-(2-chloro-4-nitrophenyl)ethyl]sulfanyl]methyl)carbamoyl]ethyl]carbamate (Compound 103, 3.10 g, 5.74 mmol, 1.00 equiv) in DMF(155.00 mL) was added piperidine (31.00 mL) at 0° C. under N₂. The resulting mixture was stirred at 0 oC for 0.5 h under N₂. LCMS indicated the reaction was completed. The reaction was diluted with water (600.00 ml). The resulting mixture was extracted with EtOAc (200.00 mLx3). The combined organic layer was washed with brine (200.00 ml), dried over anhydrous Na2SO4 and concentrated to dryness under vacuum to give 3.00 g of the crude product. The crude product was re-purified by silica gel column chromatography, eluted with (DCM: MeOH =3: 1) to give (2S)-2-amino-N-([[2-(2-chloro-4-nitrophenyl)ethyl]sulfanyl]methyl)propenamide, 104 (1.50 g, 78%) as a yellow oil. LCMS (ES, m/z): 318 [M+H]+.

### Step 11. Synthesis of Compound 105

To a solution of (2S)-2-amino-N-([[2-(2-chloro-4-nitrophenyl)ethyl]sulfanyl]methyl)-propenamide (Compound 104, 1.50 g, 4.72 mmol, 1.00 equiv) in DMF (75.00 mL) was added a solution of NaHCO₃ (0.59 g, 7.08 mmol, 1.50 equiv) in H₂O (10.00 mL) and Boc₂O (1.03 g, 4.72 mmol, 1.00 equiv) at room temperature. The reaction was stirred at room temperature for 1 h. LCMS indicated the reaction was completed. The reaction was diluted with water (500.00 mL), extracted with EtOAc (200.00 mLx3). The combined organic layer was washed with brine (200.00 mLx3), dried over anhydrous Na₂SO₄ and concentrated to dryness under vacuum to give tert-butyl N-[(1S)-1-[([[2-(2-chloro-4-nitrophenyl)ethyl]sulfanyl]methyl)carbamoyl]ethyl]carbamate (Compound 105, (1.82 g, 83) as a red oil. LCMS (ES, m/z): 418 [M+H]⁺, 318 [M+H-100]⁺

### Step 12. Synthesis of Compound 106

A slurry of tert-butyl N-[(1S)-1-[([[2-(2-chloro-4-nitrophenyl)ethyl]sulfanyl]-methyl)carbamoyl]ethyl]carbamate (Compound 105, 1.82 g, 4.36 mmol, 1.00 equiv), iron powder (2.43 g, 0.04 mmol, 10.00 equiv) and NH₄Cl (2.33 g, 0.04 mmol, 10.00 equiv) in EtOH (100.00 mL)/H₂O (50.00 mL) was stirred at 70° C. for 2 h. LCMS indicated the reaction was completed. The reaction was filtered. The filtrate was concentrated to dryness under vacuum. The residue was dissolved with DCM (50.00 mL) and filtered. The filtrate was concentrated to dryness and the residue was purified by silica gel column chromatography, eluted with (DCM: MeOH = 13: 1) to give tert-butyl N-[(1S)-1-[([[2-(4-amino-2-chlorophenyl)ethyl]sulfanyl]methyl)carbamoyl]ethyl]-carbamate (Compound 106, 1.20 g, 68%) as a yellow oil. LCMS (ES, m/z): 388 [M+H]⁺

### Step 13. Compound 107

To a stirred solution of 3-[5-(aminomethyl)-1-oxo-3H-isoindol-2-yl]piperidine-2,6-dione (INT 1, 352 mg, 1.29 mmol, 1.00 equiv) in DMF (5.00 mL) at 0° C. was added CDI (209.00 mg, 1.29 mmol, 1 equiv) and TEA (260 mg, 2.58 mmol, 2 equiv). The resulting mixture was stirred at 0° C. for 2 h. Then tert-butyl N-[(1S)-1-[([[2-(4-amino-2-chlorophenyl)ethyl]sulfanyl]-methyl)-carbamoyl]ethyl]carbamate (Compound 106, 500.00 mg, 1.29 mmol, 1.00 equiv) and DMAP (472 mg, 3.87 mmol, 3.00 equiv) were added. The resulting mixture was stirred at 60° C. for 24 h. LCMS indicated the reaction was completed. After cooled to room temperature, The reaction mixture was purified by reverse flash chromatography with the following conditions: column, C18 silica gel; mobile phase, ACN in water(0.1%FA), 0% to 60% gradient in 30 min; detector, UV 254 nm to give tert-butyl N-[(1S)-1-([[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]carbamoyl)ethyl]carbamate (Compound 107, 450.00 mg,48%) t as a yellow solid. LCMS (ES, m/z): 687 [M+H]⁺

### Step 14. Compound 108

To a stirred solution of tert-butyl N-[(1S)-1-([[(2-[2-chloro-4-[([[2-(2,6-dioxopiperidin-3-yl)-1-oxo-3H-isoindol-5-yl]methyl]carbamoyl)amino]phenyl]ethyl)sulfanyl]-methyl]carbamoyl)ethyl]carbamate (Compound 107, 440.00 mg, 0.64 mmol, 1.00 equiv) in DCM (22.00 mL) was added TFA(2.20 mL) at room temperature. The resulting mixture was stirred at room temperature for 0.5 h. LCMS indicated the reaction was completed. The reaction was concentrated to dryness under vacuum to give (2S)-2-amino-N-[[(2-[2-chloro-4-[([[2-(2,6-dioxopiperidin-3-yl)-1-oxo-3H-isoindol-5-yl]methyl]carbamoyl)amino]phenyl]ethyl)sulfanyl]-methyl]propanamide; trifluoroacetic acid (Compound 108, 400.00 mg, crude) as a red oil. The residue was used to next step without further purification. LCMS (ES, m/z): 587 [M+H-TFA]⁺

### Step 15. Synthesis of Compound 109

A solution of (2R)-2-[(2S)-2-[[(9H-fluoren-9-ylmethoxy)carbonyl]-amino]propanamido]propanoic acid (218 mg, 0.57 mmol, 1.00 equiv), HOBT (77 mg, 0.57 mmol, 1.00 equiv) and HATU (216 mg, 0.01 mmol, 1.00 equiv) was stirred at room temperature in air for 1 hour, then (2S)-2-amino-N-[[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]propanamide; trifluoroacetic acid (Compound 108, 400 mg, 0.57 mmol, 1.00 equiv) and DIEA (663 mg, 5.14 mmol, 9.00 equiv) was added at room temperature. The reaction was stirred at room temperature for 2 h. LCMS indicated the reaction was completed. The reaction mixture was purified by reverse flash chromatography with the following conditions: column, C18 silica gel; mobile phase, ACN in water (0.05%TFA), 0% to 50% gradient in 30 min; detector, UV 254 nm to give 9H-fluoren-9-ylmethyl N-[(1S)-1-[[(1R)-1-[[(1S)-1-([[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]carbamoyl)ethyl]carbamoyl]ethyl]car bamoyl]ethyl]carbamate (Compound 109, 480.00 mg, 75%) as a green solid. LCMS (ES, m/z): 951 [M+H]⁺

### Step 16. Compound 110

To a solution of 9H-fluoren-9-ylmethyl N-[(1S)-1-[[(1R)-1-[[(1S)-1-([[(2-[2-chloro-4-[([[2-(2,6-dioxopiperidin-3-yl)-1-oxo-3H-isoindol-5-yl]methyl]carbamoyl)amino]phenyl]-ethyl)sulfanyl]methyl]carbamoyl)ethyl]carbamoyl]ethyl]carbamoyl]ethyl]carbamate (Compound 109, 110.00 mg) in DMF(5.00 mL) was added piperidine (1.00 mL) at 0° C. .The resulting mixture was stirred at 0° C. for 0.5 h. LCMS indicated the reaction was completed. The reaction mixture was purified by reverse flash chromatography with the following conditions: column, C18 silica gel; mobile phase, ACN in water (0.05%TFA), 0% to 60% gradient in 40 min; detector, UV 254 nm to give (2S)-2-[(2R)-2-[(2S)-2-aminopropanamido]propanamido]-N-[[(2-[2-chloro-4-[([[2-(2,6-dioxopiperidin-3-yl)-1-oxo-3H-isoindol-5-yl]methyl]carbamoyl)amino]-phenyl]ethyl)sulfanyl]methyl]propenamide (Compound 110, 80.00 mg, 60%) as a red solid. LCMS (ES, m/z): 729 [M+H]⁺. ¹H NMR (400 MHz, DMSO-d₆) δ 9.00 (br s, 1H), 8.53 br (s, 1H), 8.24 (d, J = 7.6 Hz, 1H), 8.10 (br s, 1H), 7.69 - 7.62 (m, 2H), 7.49 (s, 1H), 7.42 (d, J = 8.0 Hz, 1H), 7.26-7.13 (m, 3H), 7.00 (br s, 1H), 5.11-5.06 (m, 1H), 4.45 - 4.36 (m, 3H), 4.35 - 4.13 (m, 6H), 2.90-2.83 (m, 3H), 2.73-2.71 (m, 2H), 2.05-1.90 (m, 1H), 1.70-1.53 (m, 4H), 1.22-1.17(m, 6H), 1.14 -1.05 (m, 3H).

### Step 17. Synthesis of Compound (Ik)

To a solution of (2S)-2-[(2R)-2-[(2S)-2-aminopropanamido]propanamido]-N-[[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]propenamide (Compound 110, 63.00 mg, 0.09 mmol, 1.00 equiv) and 2,5-dioxopyrrolidin-1-yl 6-(2,5-dioxopyrrol-1-yl)hexanoate (26 mg, 0.09 mmol, 1.00 equiv) in DMF (1.50 mL, 19.38 mmol, 224.36 equiv) was added DIEA (22.33 mg, 0.17 mmol, 2.00 equiv) at room temperature in air. The reaction was stirred at room temperature for 1 h. The reaction mixture was purified by reverse flash chromatography with the following conditions: Column: Kinetex EVO C18 Column, 30×150.5 um; Mobile Phase A:xater (0.05%TFA), Mobile Phase B:ACN; Flow rate:60 mL/min; Gradient:23 B to 43 B in 7 min, 254 nm; RT1:6.58).The collected fraction was lyophilized to give N-[(1S)-1-[[(1R)-1-[[(1S)-1-([[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]carbamoyl)ethyl]carbamoyl]ethyl]car bamoyl]ethyl]-6-(2,5-dioxopyrrol-1-yl)hexanamide (Compound (Ik), 16.10 mg, 20%) as a white solid. LCMS (ES, m/z): 922,924 [M+H]⁺. ¹HNMR (400 MHz, DMSO- d₆) δ 11.00 (s, 1H), 8.80 (s, 1H), 8.44-8.41 (m, 1H), 8.15 (d, J=7.2 Hz, 1H), 8.03-8.00 (m, 2H), 7.7-7.65 (m, 2H), 7.51 (s, 1H), 7.44 (d, J =8.0 Hz, 1H), 7.22-7.14(m, 2H), 6.98 (s, 2H), 6.83-6.81 (m, 1H), 5.13-5.08 (m, 1H), 4.48-4.40 (m, 3H), 4.29-4.17 (m, 6H), 2.96-2.85 (m, 3H), 2.75-2.70 (m, 2H), 2.67-2.57 (m, 1H), 2.40-2.33 (m, 1H), 2.09-1.98 (m, 3H), 1.52-1.45 (m, 5H), 1.26-1.16 (m, 12H).

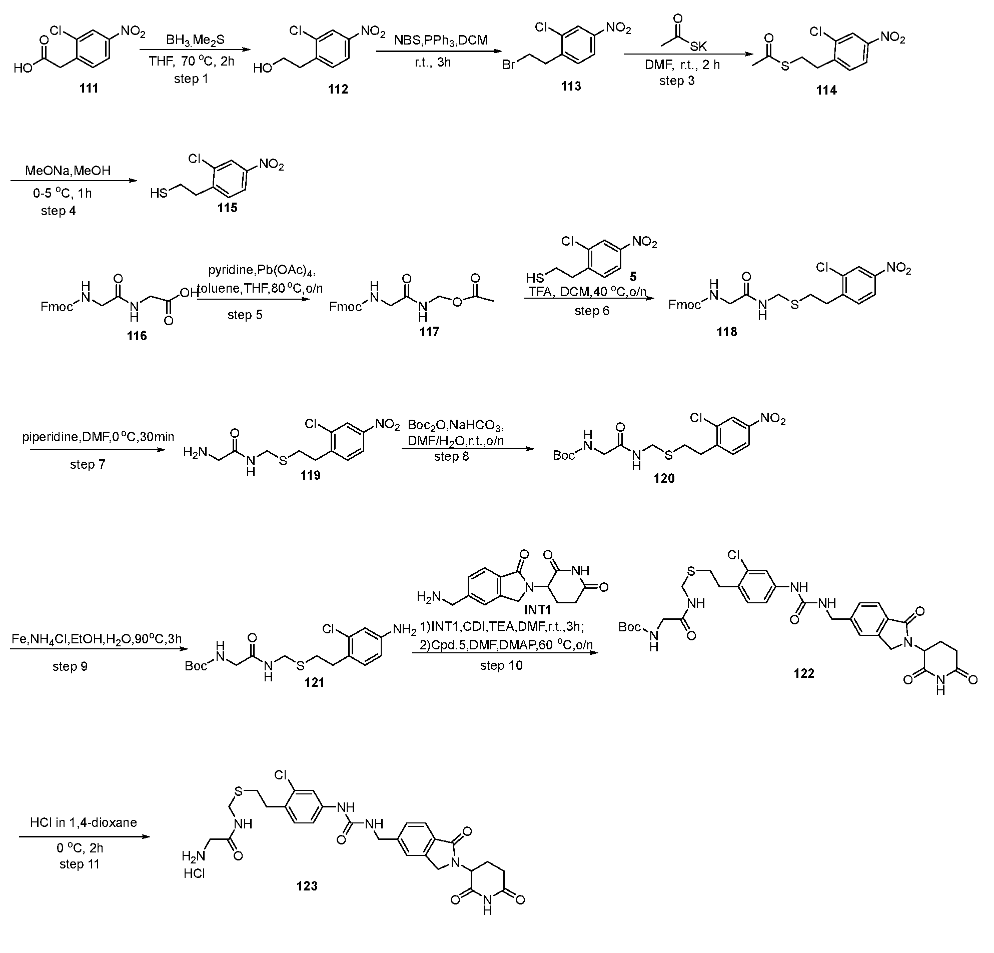

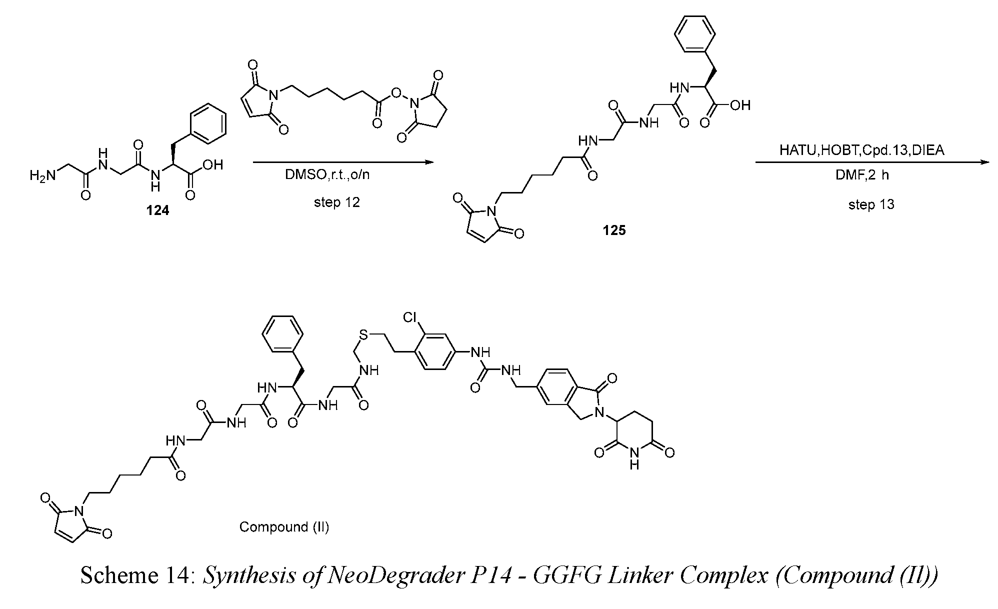

### Step 1. Synthesis of Compound 112

To a stirred solution of (2-chloro-4-nitrophenyl)acetic acid (Compound 111, 5.00 g, 23.19 mmol, 1.00 equiv) in THF (50 mL) were added BH₃—Me₂S (5.50 mL, 57.99 mmol, 2.50 equiv) in portions at room temperature under nitrogen atmosphere. The resulting mixture was stirred for 2 h at 70° C. under nitrogen atmosphere. TLC (PE: EtOAc = 3:1) indicated the reaction was completed. The residue was purified by silica gel column chromatography, eluted with PE/EtOAc (2:1) to afford 2-(2-chloro-4-nitrophenyl)ethanol (Compound 112, 4.8 g, 92%) as a light yellow solid. ¹H NMR (400 MHz, Chloroform-d) δ 8.27 (d, J = 2.4 Hz, 1H), 8.10 (dd, J = 8.4, 2.4 Hz, 1H), 7.46 (s, 1H), 3.20 -3.09 (m, 4H).

### Step 2. Synthesis of Compound 113

To a stirred solution of 2-(2-chloro-4-nitrophenyl)ethanol (Compound 112, 4.80 g, 23.81 mmol, 1.00 equiv) in DCM (100 mL) were added NBS (6.36 g, 35.71 mmol, 1.50 equiv) and PPh₃(9.37 g, 35.72 mmol, 1.50 equiv) in portions at room temperature under air atmosphere. The resulting mixture was stirred for overnight at room temperature under air atmosphere. TLC (PE: EtOAc = 10:1) indicated the reaction was completed. The reaction was concentrated to dryness under vacuum. The residue was purified by silica gel column chromatography, eluted with PE/EtOAc (4:1) to afford 1-(2-bromoethyl)-2-chloro-4-nitrobenzene (Compound 113, 3.9 g, 57%) as a red oil. ¹H NMR (400 MHz, Chloroform-d) δ8.29 (d, J = 2.4 Hz, 1H), 8.13 (dd, J = 8.4, 2.4 Hz, 1H), 7.50 (d, J = 8.4 Hz, 1H), 3.67 (t, J = 7.2 Hz, 2H), 3.42 (t, J = 7.2 Hz, 2H).

### Step 3. Synthesis of Compound 114

To a solution of 1-(2-bromoethyl)-2-chloro-4-nitrobenzene (Compound 113, 3.90 g, 14.75 mmol, 1.00 equiv) in DMF (39 mL) was added potassium thioacetate (1.68 g, 14.75 mmol, 1.00 equiv) at room temperature. The resulting mixture was stirred at room temperature for 2 hours. TLC ((PE: EtOAc = 10:1) indicated the reaction was completed. The reaction was diluted with water (600 mL). The resulting mixture was extracted with EA (200 mL*3). The combined organic layer was washed with water (200 mL), brine (200 mL), dried over anhydrous sodium sulfate and concentrated to dryness under vacuum to give 1-[[2-(2-chloro-4-nitrophenyl)ethyl]sulfanyl]ethenone (Compound 114, 3.7 g, 85%) as a red oil. ¹H NMR (400 MHz, Chloroform-d) δ 8.27 (d, J = 2.4 Hz, 1H), 8.10 (dd, J = 8.4, 2.4 Hz, 1H), 7.46 (s, 1H), 3.21 -3.02 (m, 4H), 2.37 (s, 3H).

### Step 4. Synthesis of Compound 115

To a stirred solution of 1-[[2-(2-chloro-4-nitrophenyl)ethyl]sulfanyl]ethenone (Compound 114, 4.00 g, 15.40 mmol, 1.00 equiv) in MeOH (600 mL) was added MeONa (14.31 mL, 77.00 mmol, 5.00 equiv, 30%) at 0° C. N₂ for 1 h. The reaction mixture was stirred at 0° C. for 1 h. TLC indicate (PE:EA=10:1) the reaction was completed. The reaction was quenched with AcOH. The resulting mixture was concentrated to dryness under vacuum. The residue was diluted with DCM (100 mL) and filtered. The filtrate was purified by silica gel column chromatography, eluted with (PE: EtOAc =10:1) to give 2-(2-chloro-4-nitrophenyl)ethanethiol (Compound 115, 3 g, 80%) as a yellow oil. ¹H NMR (400 MHz, Chloroform-d) δ 8.28 (d, J = 2.4 Hz, 1H), 8.11 (dd, J = 8.4, 2.4 Hz, 1H), 7.48 (d, J = 8.4 Hz, 1H), 3.17 (t, J = 7.2 Hz, 2H), 2.87 (dt, J = 8.0, 7.2 Hz, 2H), 1.46 (t, J = 8.0 Hz, 1H).

### Step 5. Synthesis of Compound 117

To a stirred mixture of (2-[[(9H-fluoren-9-ylmethoxy)carbonyl]amino]-acetamido)acetic acid (Compound 116, 10 g, 28.22 mmol, 1.00 equiv) and Pb(OAc)₄(15 g, 33.86 mmol, 1.20 equiv) in THF(300 mL) and toluene(100 mL) were added pyridine(2.59 g, 32.74 mmol, 1.16 equiv) dropwise at room temperature under nitrogen atmosphere. The resulting mixture was stirred for overnight at 80° C. under nitrogen atmosphere. LCMS indicated the reaction was completed. The mixture was allowed to cool down to room temperature. The resulting mixture was filtered, the filter cake was washed with EA (20 mL). The filtrate was concentrated under vacuum. The residue was dissolved in EA (20 mL). The resulting mixture was washed with water, brine, dried over anhydrous Na₂SO₄. After filtration, the filtrate was concentrated under vacuum. The residue was purified by silica gel column chromatography, eluted with PE/EtOAc (1:4) to give (2-[[(9H-fluoren-9-ylmethoxy)carbonyl]amino]acetamido)methyl acetate (Compound 117, 6.5 g, 56%) as a white solid. ¹HNMR(300 MHz, CDCl₃) δ7.80(d, J=7.5 Hz, 2H), 7.62(d, J=7.5 Hz, 2H), 7.45(t, d=7.5 Hz, 2H), 7.36(d, d=7.5 Hz, 2H), 7.18(br s, 1H), 5.48(br s, 1H), 5.28(d, J=7.2 Hz, 2H), 4.48(d, J=6.6 Hz, 2H), 4.26(t, J=6.6 Hz, 1H), 3.93(d, 5.4 Hz, 2H), 2.08(s, 3H). LCMS (ESI, ms): 391[M+Na]⁺

### Step 6. Synthesis of Compound 118

To a solution of (2-[[(9H-fluoren-9-ylmethoxy)carbonyl]amino]acetamido)methyl acetate (Compound 117, 3.00 g, 8.14 mmol, 1.00 equiv) and 2-(2-chloro-4-nitrophenyl)ethanethiol (Compound 115, 1.77 g, 8.13 mmol, 1.00 equiv)) in DCM (300 mL) was added TFA(0.56 g, 4.91 mmol, 0.60 equiv) at room temperature. The resulting mixture was stirred at 60° C. for 16 h. LCMS indicated the reaction was completed. The reaction was concentrated to dryness under vacuum. The residue was purified by silica gel column chromatography, eluted with (PE: EtOAc =2:3) to give 9H-fluoren-9-ylmethyl N-[[([[2-(2-chloro-4-nitrophenyl)ethyl]sulfanyl]methyl)carbamoyl]-methyl]carbamate (Compound 118, 3.7 g, 67%) as an off-white solid. LCMS (ES, m/z): 526,528 [M+H]⁺

### Step 7. Synthesis of Compound 119

To a solution of 9H-fluoren-9-ylmethyl N-[[([[2-(2-chloro-4-nitrophenyl)ethyl]sulfanyl]methyl)carbamoyl]methyl]carbamate (Compound 118, 3.70 g, 7.03 mmol, 1.00 equiv) in DMF (40 mL) was added piperidine (8 mL) at 0° C. The resulting mixture was stirred at 0° C. for 0.5 h. LCMS indicated the reaction was completed. The resulting mixture was diluted with water (400 mL), extracted with EA (200 mLx 3). The combined organic layer was washed with water (200 mL), brine (200 mL),dried over anhydrous sodium sulfate and concentrated to dryness under vacuum The residue was purified by silica gel column chromatography, eluted with (DCM: MeOH =10:1) to give 2-amino-N-([[2-(2-chloro-4-nitrophenyl)ethyl]sulfanyl]-methyl)acetamide (Compound 119, 1.01 g, 40%) as a yellow oil. LCMS (ES, m/z): 304,306 [M+H]⁺

### Step 8. Synthesis of Compound 120

To a solution of 2-amino-N-([[2-(2-chloro-4-nitrophenyl)ethyl]sulfanyl]methyl)-acetamide (Compound 119, 1.00 g, 3.29 mmol, 1.00 equiv) in DMF (50 mL) was added solution of NaHCO₃(0.33 g, 3.92 mmol, 1.20 equiv) in water(10 mL), Boc₂O (0.72 g, 3.30 mmol, 1.00 equiv) at room temperature. The resulting mixture was stirred at room temperature for 1 h. LCMS indicated the reaction was completed. The reaction was diluted with water (500 mL), extracted with EtOAc (200 mL x3). The combined organic layer was washed with brine (200 mL), dried over anhydrous Na₂SO₄ and concentrated to dryness under vacuum. The residue was purified by silica gel column chromatography, eluted with (PE: EtOAc =1:3) to give tert-butyl N-[[([[2-(2-chloro-4-nitrophenyl)ethyl]sulfanyl]methyl)carbamoyl]methyl]carbamate (Compound 120, 810 mg, 54%) as a white solid. LCMS (ES, m/z): 404,406 [M+H]⁺, 304,306 [M+H-100]⁺

### Step 9. Synthesis of Compound 121

To a solution of tert-butyl N-[[([[2-(2-chloro-4-nitrophenyl)ethyl]sulfanyl]-methyl)carbamoyl]methyl]carbamate (Compound 120, 800.00 mg, 1.98 mmol, 1.00 equiv) in EtOH(40) was added iron powder(1106 mg, 19.81 mmol, 10.00 equiv) and solution of NH₄Cl (1059 mg, 19.81 mmol, 10.00 equiv) in water (10 mL) at room temperature. The resulting mixture was stirred at 70° C. for 2 h. LCMS indicated the reaction was completed. The reaction was filtered. The filtrate was concentrated to dryness under vacuum. The residue was dissolved with DCM (50.00 mL) and filtered. The filtrate was purified by silica gel column chromatography, eluted with (DCM: MeOH = 13: 1) to give tert-butyl N-[[([[2-(4-amino-2-chlorophenyl)ethyl]sulfanyl]methyl)-carbamoyl]methyl]carbamate (Compound 121, 610 mg, 74%) as a yellow oil. LCMS (ES, m/z): 374,376 [M+H]⁺, 374,376 [M+H-100]⁺

### Step 10. Synthesis of Compound 122

To a solution of 3-[5-(aminomethyl)-1-oxo-3H-isoindol-2-yl]piperidine-2,6-dione (INT 1, 219 mg, 0.80 mmol, 1.00 equiv) in DMF(10 mL) was added CDI (130 mg, 0.80 mmol, 1.00 equiv) and TEA (81 mg, 0.80 mmol, 1.00 equiv) at 0° C. in air. The resulting mixture was stirred at room temperature for 2 h. Then tert-butyl N-[[([[2-(4-amino-2-chlorophenyl)ethyl]sulfanyl]-methyl)carbamoyl]methyl]carbamate (Compound 121, 300 mg, 0.80 mmol, 1.00 equiv) and DMAP (294 mg, 2.41 mmol, 3.00 equiv) was added at room temperature in air. The resulting mixture was stirred at 60° C. for 48 h. LCMS indicated the reaction was completed. The resulting mixture purified by reverse flash chromatography with the following conditions: column, C18 silica gel; mobile phase, ACN in water(0.05%TFA), 0% to 60% gradient in 30 min; detector, UV 254 nm to give tert-butyl N-[([[(2-[2-chloro-4-[([[2-(2,6-dioxopiperidin-3-yl)-1-oxo-3H-isoindol-5-yl]methyl]-carbamoyl)amino]phenyl]ethyl)sulfanyl]methyl]carbamoyl)methyl]carbamate (Compound 122, 270 mg, 49%) as a yellow solid. LCMS (ES, m/z): 673,675 [M+H]⁺, 573,575 [M+H-100]⁺

### Step 11. Synthesis of Compound-123

To a solution of tert-butyl N-[([[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]carbamoyl)methyl]-carbamate (Compound 122, 250 mg, 0.37 mmol) in 1,4-dioxane(12 mL) was added HCl (4N in 1,4-dioxane, 6 mL) at 0° C. under N₂. The reaction was stirred at room temperature for 2 h. LCMS indicated the reaction was completed. The reaction mixture was concentrated to dryness under vacuum to give 2-amino-N-[[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]acetamide (Compound 123, 260 mg, crude ) as a brown solid. LCMS (ES, m/z): 573,575 [M+H-HCl]⁺

### Step 12. Synthesis of Compound-125

A solution of (2S)-2-[2-(2-aminoacetamido)acetamido]-3-phenylpropanoic acid (Compound 124, 500 mg, 1.79 mmol, 1.00 equiv) and 2,5-dioxopyrrolidin-1-yl 6-(2,5-dioxopyrrol-1-yl)hexanoate (552 mg, 1.79 mmol, 1.00 equiv) in DMSO (5.00 mL) was stirred at room temperature in air for 16 h. LCMS indicated the reaction was completed. The reaction mixture was purified by reverse flash chromatography with the following conditions: column, C18 silica gel; mobile phase, ACN in water (0.1%FA), 0% to 60% gradient in 30 min; detector, UV 220 nm to give (2S)-2-(2-[2-[6-(2,5-dioxopyrrol-1-yl)hexanamido]acetamido]acetamido)-3-phenylpropanoic acid (Compound 125, 760 mg, 83%) as a white solid. LCMS (ES, m/z): 473 [M+H]⁺

### Step 13. Synthesis of Compound (Il)

To a solution of (2S)-2-(2-[2-[6-(2,5-dioxopyrrol-1-yl)hexanamido]acetamido]-acetamido)-3-phenylpropanoic acid (Compound 125, 175 mg, 0.37 mmol, 1.00 equiv) in DMF(5.00 mL) were added HATU (141 mg, 0.37 mmol, 1.00 equiv) and HOBT (50 mg, 0.37 mmol, 1.00 equiv) at room temperature in air. The resulting mixture was stirred at room temperature for 1 h. Then 2-amino-N-[[(2-[2-chloro-4-[([[2-(2,6-dioxopiperidin-3-yl)-1-oxo-3H-isoindol-5-yl]methyl]-carbamoyl)amino]phenyl]ethyl)sulfanyl]methyl]acetamide (Compound 123, 250 mg, 0.37 mmol, 1.00 equiv, 85%) and DIEA(240 mg, 1.85 mmol, 5.00 equiv) were added. The resulting mixture was stirred at room temperature for 1 h. LCMS indicated the reaction was completed. The reaction mixture was purified by the following condition: Column: XSelect CSH Prep C18 OBD Column, 19*250 mm,5 um; Mobile Phase A:water(0.05%FA), Mobile Phase B:ACN; Flow rate:25 mL/min; Gradient:30 B to 60 B in 7 min, 254 nm; RT1:6.67 min to give 75 mg of the crude product. The crude product was re-purified by reverse flash chromatography with the following conditions: Column: XBridge Shield RP18 OBD Column, 19^{∗}250 mm,10 um; Mobile Phase A:water(0.1%FA), Mobile Phase B:ACN; Flow rate:25 mL/min; Gradient:25 B to 44 B in 10 min; 254 nm; RT1:10.52 min. The collected fraction was lyophilized to give Compound (Il) (41.6 mg, 10%) as a white solid. ¹HNMR(400 MHz, DMSO-d₆) δ10.99 (s, 1H), 8.79 (s, 1H), 8.38 (t, J=6.0 Hz, 1H), 8.31 (t, J=6.0 Hz, 1H), 8.12 (d, J=8.4 Hz, 1H), 8.06 (t, J=5.6 Hz, 1H), 8.01 (t, J=6.0 Hz, 1H), 7.70-7.66 (m, 2H), 7.51 (s, 1H), 7.44 (d, J=8.0 Hz, 1H), 7.25-7.21 (m, 5H), 7.19-7.14 (m, 2H), 6.99 (s, 2H), 6.82 (t, J=6.0 Hz,1H), 5.13-5.08 (m, 1H), 4.47-4.40 (m, 4H), 4.33-4.29 (m, 3H), 3.76-3.70 (m, 3H), 3.67-3.55 (m, 3H), 3.38-3.36 (m, 2H), 3.06-3.02 (m, 1H), 2.91-2.86 (m,3H), 2.82-2.70 (m, 3H), 2.62-2.57 (m, 1H), 2.50-2.45 (m, 1H), 2.10 (m, 2H), 2.05-1.95 (m,1H), 1.50-1.44 (m,4H), 1.20-1.16 (m, 2H). LCMS (ES, m/z):1027,1029 [M+H]⁺

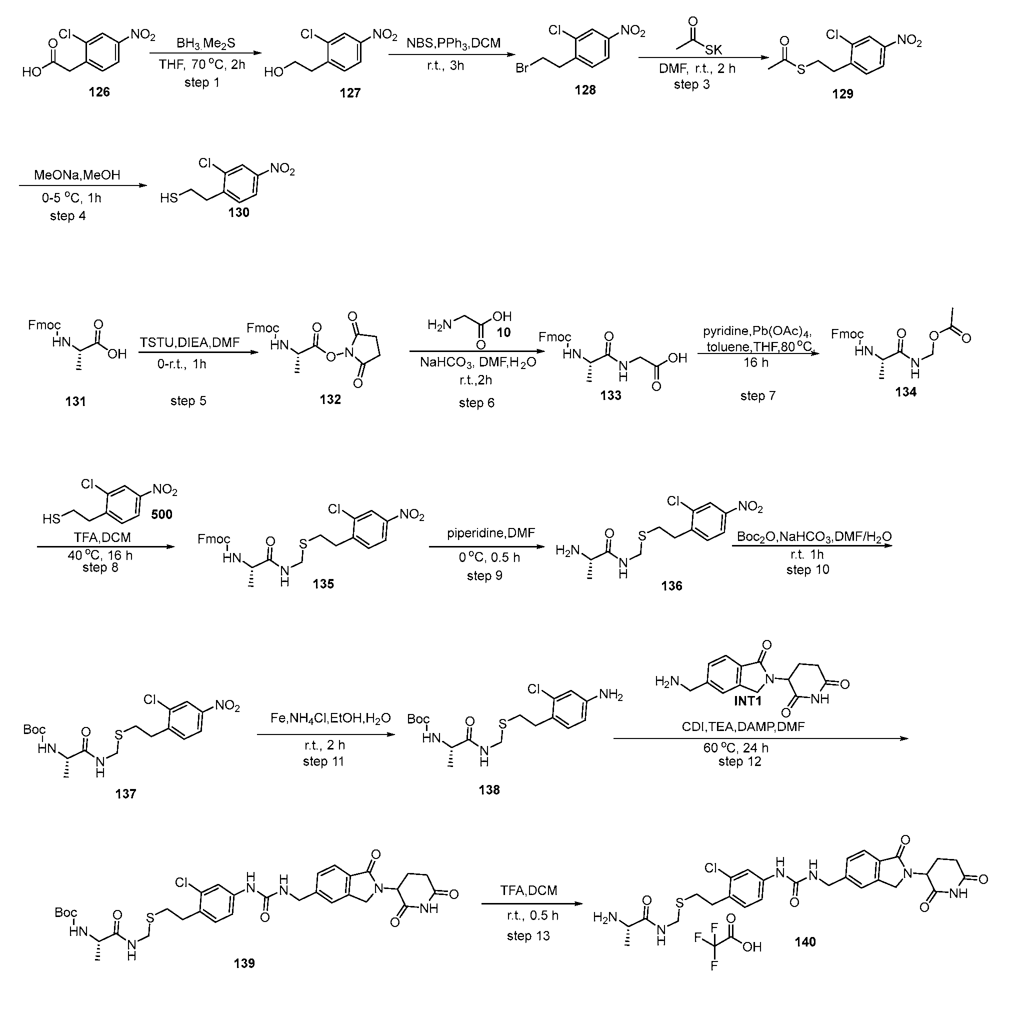

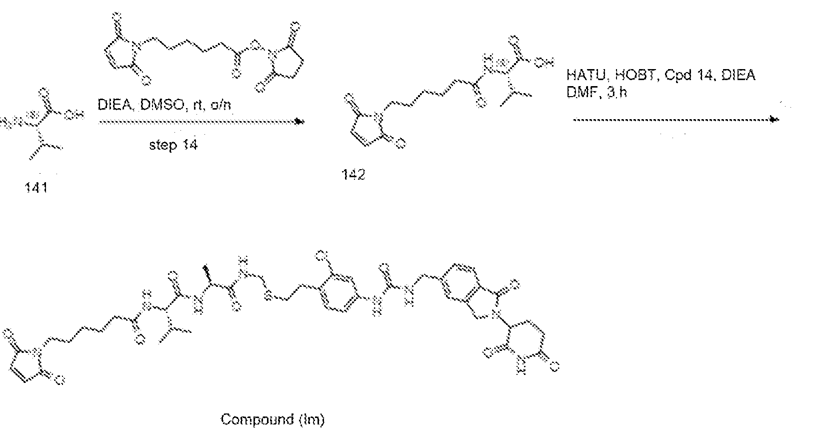

### Step 1. Synthesis of Compound 127

To a stirred solution of (2-chloro-4-nitrophenyl)acetic acid (Compound 126, 24.00 g, 111.32 mmol, 1.00 equiv) in THF (240.00 mL) were added BH₃—Me₂S (28.00 mL, 295.23 mmol, 2.65 equiv) dropwise under nitrogen atmosphere. The resulting mixture was stirred for 2 hours at 70° C. under nitrogen atmosphere. TLC (PE: EtOAc = 3:1) indicated the reaction was completed. After cooled to room temperature, the resulting mixture was concentrated under vacuum. The residue was purified by silica gel column chromatography, eluted with PE/ EtOAc (3:1) to afford 2-(2-chloro-4-nitrophenyl)ethanol (Compound 127, 18.00 g, 80%) as a light yellow solid. ¹H NMR (300 MHz, CD₃Cl) δ8.27 (s, 1H), 8.10-8.07 (m, 1 H), 7.52 (d, J = 3 Hz, 1H), 3.96 (t, J = 6 Hz, 2H), 3.13 (t, J = 6 Hz, 2H).

### Step 2. Synthesis of Compound 128

To a stirred solution of 2-(2-chloro-4-nitrophenyl)ethanol (Compound 127, 5.00 g, 24.80 mmol, 1.00 equiv) in DCM (100.00 mL) were added NBS (6.62 g, 1.50 equiv) and PPh₃ (9.76 g, 37.21 mmol, 1.50 equiv) in portions at room temperature under N₂. The resulting mixture was stirred overnight at room temperature under N₂. TLC (PE: EtOAc = 10:1) indicated the reaction was completed. The reaction was concentrated to dryness under vacuum. The residue was purified by silica gel column chromatography, eluted with PE/ EtOAc (4:1) to afford 1-(2-bromoethyl)-2-chloro-4-nitrobenzene (Compoud 128, 5.10 g, 72.31%) as a red oil. ¹H NMR (400 MHz, DMSO-d₆) δ 8.28 (d, J = 2.4 Hz, 1H), 8.18 (dd, J = 8.4, 2.4 Hz, 1H), 7.73 (d, J = 8.4 Hz, 1H), 3.79 (t, J = 7.2 Hz, 2H), 3.38 (t, J = 7.2 Hz, 2H).

### Step 3. Synthesis of Compound 129

To a solution of 1-(2-bromoethyl)-2-chloro-4-nitrobenzene (Compound 128, 5.00 g, 18.90 mmol, 1.00 equiv) in DMF (50.00 mL) was added potassium thioacetate (2.16 g, 18.91 mmol, 1.00 equiv) at room temperature under nitrogen atmosphere. The resulting mixture was stirred at room temperature for 2 hours. TLC (PE: EtOAc= 10:1) indicated the reaction was completed. The reaction was diluted with water (600.00 mL). The resulting mixture was extracted with EtOAc (200 .00 mL*3). The combined organic layer was washed with water (200.00 mL), brine (200.00 mL*3), dried over anhydrous Na₂SO₄ and concentrated to dryness under vacuum to afford 1-[[2-(2-chloro-4-nitrophenyl)ethyl]sulfanyl]ethenone (Compound 129, 4.50 g, 85%) as a red oil. ¹H NMR (400 MHz, CDCl₃) δ 8.24 (d, J= 2.4 Hz, 1H), 8.07 (dd, J= 8.4, 2.4 Hz, 1H), 7.45 (d, J= 8.4 Hz, 1H), 3.20 - 3.05 (m, 4H), 2.34 (s, 3H).

### Step 4. Synthesis of Compound 130

To a stirred solution of 1-[[2-(2-chloro-4-nitrophenyl)ethyl]sulfanyl]ethenone (Compound 129, 2.00 g, 7.70 mmol, 1.00 equiv) in MeOH (300.00 mL) was added MeONa (6.93 mL, 37.33 mmol, 5.00 equiv, 30%) at 0° C. under N₂. The resulting mixture was stirred at 0° C. under N₂ for 1 h. TLC (PE: EtOAc =10:1) indicated the reaction was completed. The reaction was quenched with AcOH to pH value to 3-4. The resulting mixture was concentrated to dryness under vacuum. The residue was diluted with DCM (50.00 mL) and filtered. The filtrate was purified with Prep-TLC (PE: EtOAc = 10:1) to give 2-(2-chloro-4-nitrophenyl)ethanethiol (Compound 130, 1.35 g, 72%) as a light yellow oil. ¹H NMR (400 MHz, CDCl₃) δ 8.26 (d, J = 2.4 Hz, 1H), 8.09 (dd, J = 8.4, 2.4 Hz, 1H), 7.45 (d, J = 8.4 Hz, 1H), 3.14 (dd, J = 8.0, 6.8 Hz, 2H), 2.85 (dt, J = 8.0, 7.2 Hz, 2H), 1.43 (t, J= 8.0 Hz, 1H).

### Step 5. Synthesis of Compound 132

To a stirred solution of (2S)-2-[[(9H-fluoren-9-ylmethoxy)carbonyl]amino]propanoic acid (Compound 131, 20.00 g, 64.24 mmol, 1.00 equiv) in DMF (200.00 mL) were added TSTU (25.18 g, 83.52 mmol, 1.30 equiv) and DIEA(16.60 g, 128.48 mmol, 2.00 equiv) at room temperature under air atmosphere. The resulting mixture was stirred for 1 h at room temperature. LCMS indicated the reaction was completed. The reaction was diluted with water (200.00 mL), the resulting mixture was extracted with ETOAC (100.00 mL*3). The combined organic layer was washed with water (100.00 mL), brine (100.00 mL), dried over anhydrous Na₂SO₄ and concentrated to dryness in vacuum. The residue was purified by silica gel column chromatography, eluted with (PE: EtOAc =1:2) to give 2,5-dioxopyrrolidin-1-yl (2S)-2-[[(9H-fluoren-9-ylmethoxy)carbonyl]amino]propanoate (Compound 132, 25.00 g, 83%) t as a white solid. LCMS (ES, m/z):431[M+Na]⁺

### Step 6. Synthesis of Compound 133

To a solution of glycine (3.68 g, 48.97 mmol, 1.00 equiv) and NaHCO₃ (12.34 g, 146.89 mmol, 3.00 equiv) in water (200.00 mL) was added solution of 2,5-dioxopyrrolidin-1-yl (2S)-2-[[(9H-fluoren-9-ylmethoxy)carbonyl]amino]propanoate (Compound 132, 20.00 g, 48.97 mmol, 1.00 equiv) in DMF (200.00 mL).The reaction was stirred at room temperature for 2 h. LCMS indicated the reaction was completed . The reaction was adjusted to pH value to 2-3 with 2 N HCl. The resulting mixture was extracted with EtOAc (500.00 mL*3), the combined organic layer was washed with brine (500.00 mL), dried over anhydrous Na₂SO₄ and concentrated to dryness in vacuum to give [(2S)-2-[[(9H-fluoren-9-ylmethoxy)carbonyl]amino]propanamido]acetic acid (Compound 133, 15.00 g, 71%) as a white solid. LCMS (ES, m/z): 369 [M+H]⁺

### Step 7. Synthesis of Compound 134

A solution of [(2S)-2-[[(9H-fluoren-9-ylmethoxy)carbonyl]amino]-propanamido]acetic acid (Compound 133, 5.00 g, 13.57 mmol, 1.00 equiv), Pb(OAc)₄ (7.22 g, 16.28 mmol, 1.20 equiv) and pyridine (1.29 g, 16.31 mmol, 1.20 equiv) in THF (300.00 mL)/Toluene (100.00 mL) under N₂ was stirred at 80° C. for 16 h. LCMS indicated the reaction was completed. After cooled to room temperature, the reaction was filtered. The filter cake was washed with THF (100.00 mL). The combined organic layer was concentrated to dryness under vacuum. The residue was purified by silica gel column chromatography, eluted with (PE: ETOAC=1:2) to give [(2S)-2-[[(9H-fluoren-9-ylmethoxy)carbonyl]amino]propanamido]methyl acetate (Compound 134, 2.50 g,45%) as a white solid. LCMS (ES, m/z): 405 [M+Na]⁺. ¹H NMR (400 MHz, Chloroform-d) δ 7.77-7.73 (m, 2H), 7.58 (d, J= 7.6 Hz, 2H), 7.43 - 7.37 (m, 2H), 7.36 - 7.29 (m, 2H), 7.10 (s, 1H), 5.24 (d, J = 7.6 Hz, 2H), 4.51 - 4.35 (m, 2H), 4.22 (t, J= 6.8 Hz, 2H), 2.04 (s, 3H), 1.39 (d, J= 6.8 Hz, 3H).

### Step 8. Synthesis of Compound 135

To a stirred solution of [(2S)-2-[[(9H-fluoren-9-ylmethoxy)carbonyl]amino]-propanamido]methyl acetate (Compound 134, 2.25 g, 5.88 mmol, 1.00 equiv) and 2-(2-chloro-4-nitrophenyl)ethanethiol (Compound 500, 1.28 g, 5.88 mmol, 1.00 equiv) in DCM(120 mL) was added TFA (0.27 mL, 2.376 mmol, 0.62 equiv) under N₂ at room temperature. The resulting mixture was stirred at 40° C. for 16 hours. LCMS indicated the reaction was completed. The reaction was concentrated to dryness in vacuum to give 9H-fluoren-9-ylmethyl N-[(1S)-1-[([[2-(2-chloro-4-nitrophenyl)ethyl]sulfanyl]methyl)carbamoyl]ethyl]carbamate (Compound 135, 3.10 g, 90%) as a yellow solid. LCMS (ES, m/z): 540,542 [M+H]⁺

### Step 9. Synthesis of Compound 136

To a solution of 9H-fluoren-9-ylmethyl N-[(1S)-1-[([[2-(2-chloro-4-nitrophenyl)ethyl]sulfanyl]methyl)carbamoyl]ethyl]carbamate (Compound 135, 3.10 g, 5.74 mmol, 1.00 equiv) in DMF(155.00 mL) was added piperidine (31.00 mL) at 0° C. under N₂. The resulting mixture was stirred at 0° C. for 0.5 h under N₂. LCMS indicated the reaction was completed. The reaction was diluted with water (600.00 ml). The resulting mixture was extracted with EA (200.00 mLx3). The combined organic layer was washed with brine (200.00 ml), dried over anhydrous Na₂SO₄ and concentrated to dryness under vacuum to give 3.00 g of the crude product. The crude product was re-purified by silica gel column chromatography, eluted with (DCM: MeOH =3: 1) to give (2S)-2-amino-N-([[2-(2-chloro-4-nitrophenyl)ethyl]sulfanyl]methyl)propenamide (Compound 136, 1.50 g,78%) as a yellow oil. LCMS (ES, m/z): 318,320 [M+H]⁺

### Step 10. Synthesis of Compound 137

To a solution of (2S)-2-amino-N-([[2-(2-chloro-4-nitrophenyl)ethyl]sulfanyl]-methyl)propenamide (Compound 136, 1.50 g, 4.72 mmol, 1.00 equiv) in DMF (75.00 mL) was added a solution of NaHCO₃ (0.59 g, 7.08 mmol, 1.50 equiv) in H₂O (10.00 mL) and Boc₂O( 1.03 g, 4.72 mmol, 1.00 equiv) at room temperature in air. The reaction was stirred at room temperature for 1 h. LCMS indicated the reaction was completed The reaction was diluted with water (500.00 mL), extracted with EtOAc (200.00 mLx3).The combined organic layer was washed with brine (200.00 mL*3),dried over anhydrous Na₂SO₄ and concentrated to dryness under vacuum to give tert-butyl N-[(1S)-1-[([[2-(2-chloro-4-nitrophenyl)ethyl]sulfanyl]methyl)-carbamoyl]ethyl]carbamate (Compound 137, 1.82 g, 83%) as a red oil. LCMS (ES, m/z): 418,420 [M+H]⁺, 318,320 [M+H-100]⁺

### Step 11. Synthesis of Compound 138

A slurry of tert-butyl N-[(1S)-1-[([[2-(2-chloro-4-nitrophenyl)ethyl]-sulfanyl]methyl)carbamoyl]ethyl]carbamate (Compound 137, 1.82 g, 4.36 mmol, 1.00 equiv), iron powder (2.43 g, 0.04 mmol, 10.00 equiv) and NH₄Cl (2.33 g, 0.04 mmol, 10.00 equiv) in EtOH (100.00 mL)/H₂O (50.00 mL) was stirred at 70° C. for 2 h. LCMS indicated the reaction was completed. The reaction was filtered. The filtrate was concentrated to dryness under vacuum. The residue was dissolved with DCM (50.00 mL) and filtered. The filtrate was purified by silica gel column chromatography, eluted with (DCM: MeOH = 13: 1) to give tert-butyl N-[(1S)-1-[([[2-(4-amino-2-chlorophenyl)ethyl]sulfanyl]methyl)carbamoyl]ethyl]carbamate (Compound 138, 1.20 g, 68%) as a yellow oil. LCMS (ES, m/z): 388,390 [M+H]⁺, 288,290 [M+H-100]⁺

### Step 12. Synthesis of Compound 139

To a stirred solution of 3-[5-(aminomethyl)-1-oxo-3H-isoindol-2-yl]piperidine-2,6-dione (INT 1, 352 mg, 1.29 mmol, 1.00 equiv) in DMF (5.00 mL) at 0° C. was added CDI (209.00 mg, 1.29 mmol, 1 equiv) and TEA(260 mg, 2.58 mmol, 2 equiv). The resulting mixture was stirred at 0° C. for 2 h. Then tert-butyl N-[(1S)-1-[([[2-(4-amino-2-chlorophenyl)ethyl]sulfanyl]-methyl)carbamoyl]-ethyl]carbamate (Compound 138, 500.00 mg, 1.29 mmol, 1.00 equiv) and DMAP (472 mg, 3.87 mmol, 3.00 equiv) were added. The resulting mixture was stirred at 60° C. for 24 h. LCMS indicated the reaction was completed. After cooled to room temperature, The reaction mixture was purified by reverse flash chromatography with the following conditions: column, C18 silica gel; mobile phase, ACN in water(0.1%FA), 0% to 60% gradient in 30 min; detector, UV 254 nm to give tert-butyl N-[(1S)-1-([[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]carbamoyl)ethyl]carbamate, (Compound 139, 450.00 mg, 48%) t as a yellow solid. LCMS (ES, m/z): 687,689 [M+H]⁺, 587,589 [M+H-100]⁺

### Step 13. Synthesis of Compound 140

To a stirred solution of tert-butyl N-[(1S)-1-([[(2-[2-chloro-4-[([[2-(2,6-dioxopiperidin-3-yl)-1-oxo-3H-isoindol-5-yl]methyl]carbamoyl)amino]phenyl]ethyl)sulfanyl]-methyl]carbamoyl)ethyl]carbamate (Compound 139, 440.00 mg, 0.64 mmol, 1.00 equiv) in DCM (22.00 mL) was added TFA(2.20 mL) at room temperature. The resulting mixture was stirred at room temperature for 0.5 h. LCMS indicated the reaction was completed. The reaction was concentrated to dryness under vacuum to give (2S)-2-amino-N-[[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]propanamide; trifluoroacetic acid (Compound 140, 400.00 mg ) as a red oil. LCMS (ES, m/z): 578,589 [M+H-TFA]⁺

### Step 14. Synthesis of Compound 142

To a slurry of L-valine (Compound 141, 0.50 g, 4.27 mmol, 1.00 equiv) in DMSO (10 mL) was added 2,5-dioxopyrrolidin-1-yl 6-(2,5-dioxopyrrol-1-yl)hexanoate (1.32 g, 4.28 mmol, 1.00 equiv) and DIEA (1103 mg, 8.54 mmol, 2.00 equiv) .The resulting mixture was stirred at room temperature for 4 h. LCMS indicated the reaction was completed. The reaction mixture was purified by reverse flash chromatography with the following conditions: column, C18 silica gel; mobile phase, ACN in water (0.1%FA), 0% to 60% gradient in 30 min; detector, UV 220 nm to give (2S)-2-[6-(2,5-dioxopyrrol-1-yl)hexanamido]-3-methylbutanoic acid (Compound 142, 1.2 g, 72%) as a brown solid. LCMS (ES, m/z): 311 [M+H]⁺

### Step 15. Synthesis of Compound (Im)

A solution of (2S)-2-[6-(2,5-dioxopyrrol-1-yl)hexanamido]-3-methylbutanoic acid, (Compound 142, 59 mg, 0.19 mmol, 1.00 equiv), HOBT (26 mg, 0.19 mmol, 1.00 equiv) and HATU (72 mg, 0.19 mmol, 1.00 equiv) in DMF (2 mL) was stirred at room temperature in air for 1 hour. Then (2S)-2-amino-N-[[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]propanamide trifluoroacetic acid (Compound 140, 200 mg, 0.19 mmol, 1.00 equiv, 66.70%) and DIEA (197 mg, 1.52 mmol, 8.00 equiv) was added at room temperature. The reaction mixture was stirred at room temperature for 2 h. LCMS indicated the reaction was completed. The resulting mixture was purified by reverse flash chromatography with the following conditions: Column: YMC-Actus Triart C18, 30 mm X 150 mm, 5um; Mobile Phase A:Water(0.1%FA), Mobile Phase B:ACN; Flow rate:60 mL/min; Gradient:28 B to 45 B in 10 min, 254 nm; RT1:9.67 min. The collected fraction was lyophilized to give N-[(1S)-1-[[(1S)-1-([[(2-[2-chloro-4-[([[2-(2,6-dioxopiperidin-3-yl)-1-oxo-3H-isoindol-5-yl]methyl]carbamoyl)amino]-phenyl]ethyl)sulfanyl]methyl]carbamoyl)ethyl]carbamoyl]-2-methylpropyl]-6-(2,5-dioxopyrrol-1-yl)hexanamide (Compound (Im), 27.8 mg, 16%) as a white solid. LCMS (ES, m/z): 879,881 [M+H]⁺. ¹H NMR (400 MHz, DMSO-d₆) δ10.99 (s, 1H), 8.80 (s, 1H), 8.47 (t, J=6.0 Hz, 1H), 8.03 (d, J=7.2 Hz, 1H), 7.78 (d, J=8.8 Hz,1H), 7.70-7.66 (m, 2H), 7.51 (s, 1H), 7.44 (d, J=8.0 Hz, 1H), 7.21-7.14 (m, 2 H), 6.99 (s, 2H), 6.82 (t, J=6.0 Hz, 1H), 5.13-5.10 (m,1 H), 4.47-4.40 (m, 3H), 4.33-4.29 (m, 3H), 4.24 (t, J=7.2 Hz, 1H), 4.14 (t, J=6.8 Hz, 1H), 3.38-3.36 (m, 1H), 2.97-2.90 (m, 1H), 2.86 (t, J=7.6 Hz, 2H), 2.73-2.67 (m, 2H), 2.62-2.57 (m, 1H), 2.40-2.35 (m, 1H), 2.20-2.05 (m, 2H), 2.02-1.96 (m, 1H), 1.95-1.88 (m, 1H), 1.48-1.46 (m, 4H), 1.23-1.16 (m, 6H), 0.83-0.78 (m, 6H).

### Example 4: General Procedure for Preparation and Characterization of NeoDegrader Conjugates

A solution of antibody was treated with 30 equivalents of tris-(2-carboxyethyl)phosphine (TCEP) and incubated at 37° C. for 1 hour to reduce the interchain disulfides. The reduced antibody was purified into 50 mM EPPS, 5 mM EDTA pH 7.0 buffer using illustra NAP columns (GE Healthcare).

Conjugation was effected by treatment of a solution of reduced antibody at 2-5 mg/mL in 50 mM EPPS, 5 mM EDTA pH 7.0 with 12 equivalents of linker-neoDegrader added as a stock solution in N,N-dimethylacetamide (DMA) such that the final concentration of DMA was 15% (v/v). The resulting reaction mixture was left overnight at 4° C. The resulting newDegrader conjugate was purified into 20 mM succinate, 8% sucrose, 0.01% Tween-20 pH 5.5 using illustra NAP columns (GE Healthcare) and concentrated using Amicon Ultra centrifugal concentrators with 50 kD molecular weight cutoff (Millipore).

Concentration and monomer were determined by size exclusion chromatography using a 7.8 × 300 mM TSKGel 3000SWXL column with 5 µm particles (Tosoh Bioscience), eluting isocratically with 400 mM sodium perchlorate, 50 mM sodium phosphate, 5% (v/v) isopropanol mobile phase running at 0.5 mg/mL for 30 min. NeoDegrader conjugates were quantitated from antibody standard curves, detecting at 214 nm.

Drug to antibody ratio (DAR) was determined by hydrophobic interaction chromatography using a 4.6 × 35 mm TSKgel Butyl-NPR column with 2.5 µm particles. Mobile phase A was 1.5 M ammonium sulfate, 25 mM sodium phosphate pH 7.0. Mobile phase B was 25 mM sodium phosphate pH 7.0, 25% (v/v) isopropanol. Analytes were eluted with a linear gradient of 0-100% B in 12 min. at a flow rate of 0.6 mL/min. Detection was at 214 nm.

Free linker-payload was determined by mixed-mode chromatography using a 4.6 × 250 mm HISEP column with 2.5 µm particles (Supelco). Mobile phase A was 100 mM ammonium acetate. Mobile phase B was 100% acetonitrile. Analytes were eluted with a gradient of 25-40% B in 25 min., then 40-100% B in 2 min at a flow rate of 0.7 mL/min. Column temperature was 35° C. Free linker-payload was quantitated using an external standard curve, detecting at 254 nm.

### Example 5: General Procedure 1 for In Vitro Antiproliferation Assay for NeoDegraders and NeoDegrader Conjugates

The ability of neoDegrader conjugates to inhibit cell growth was measured using in vitro anti-proliferation assay. Target cells were plated at 1,500 - 5,000 cells per well in 100 µL complete cell growth medium (RPMI 1640, 10% fetal bovine serum and 1% Penicillin-streptomycin for most cell lines; Hybri-care medium, 1.5 g/L sodium bicarbonate, 10% fetal bovine serum and 1% Penicillin-streptomycin for BT-474; RPMI 1640, 20% fetal bovine serum and 1% Penicillin-streptomycin for HL-60). Conjugates were diluted in complete cell growth medium using 4-fold serial dilutions and 100 µL was added per well. The final concentration typically ranged from 1 × 10⁻⁸ M to 1.53 × 10⁻¹³ M or 1 × 10⁻⁷ M to 1.53 × 10⁻¹² M. Cells were incubated at 37° C. in a humidified 5% CO₂ incubator for 5 days. Viability of remaining cells was determined by colorimetric WST-8 assay (Dojindo Molecular Technologies, Inc., Rockville, MD, US). WST-8 was added to 10% of the final volume and plates were incubated at 37° C. in a humidified 5% CO₂ incubator for 2-4 hours. Plates were analyzed by measuring the absorbance at 450 nm (A450) in a multi-well plate reader. Background A450 absorbance of wells with media and WST-8 only was subtracted from all values. The percent viability was calculated by dividing each treated sample value by the average value of wells with untreated cells. The percent viability value was plotted against the test sample concentration in a semi-log plot for each treatment. IC50 values were calculated automatically.

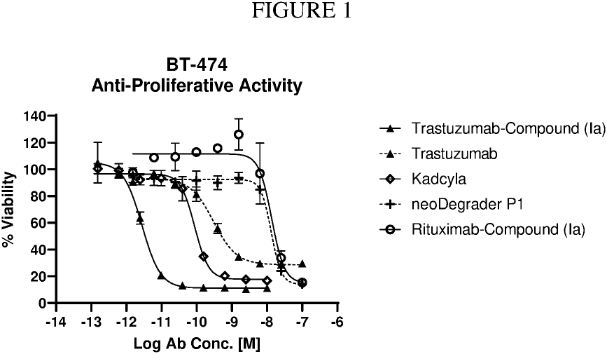

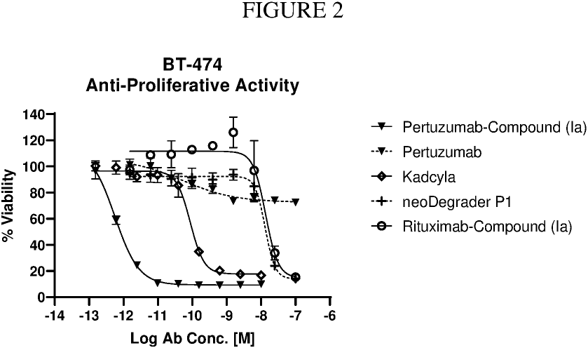

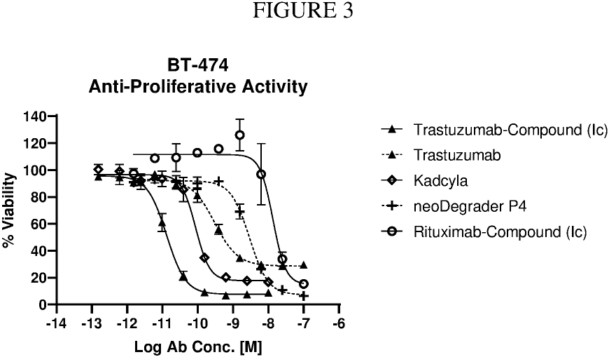

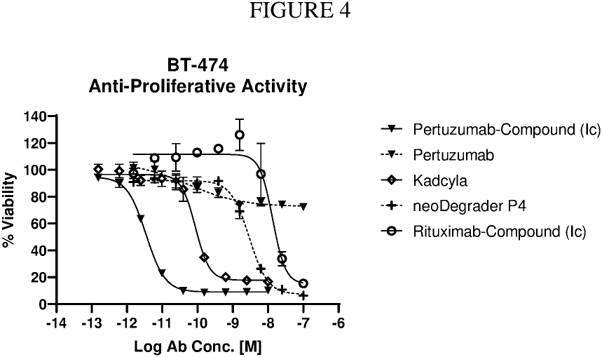

The anti-proliferative activity of trastuzumab and pertuzumab conjugates of Compound (Ia) and (Ic) against the BT-474 breast cancer cell line is shown in FIGS. 1-4 (drug:antibody ratio= 8 in each neoDegrader conjugate). Antibody drug conjugate Kadcyla and unconguaged antibodies trastuzumab and pertuzumab were found to be >100-fold less active than the antibody neoDegrader conjugates while non cell-binding control neoDegrader conjugate Rituximab-Compound (Ia) and released neoDegraders P1 and P4 a were found to be >1000-fold less active against BT-474 cells.

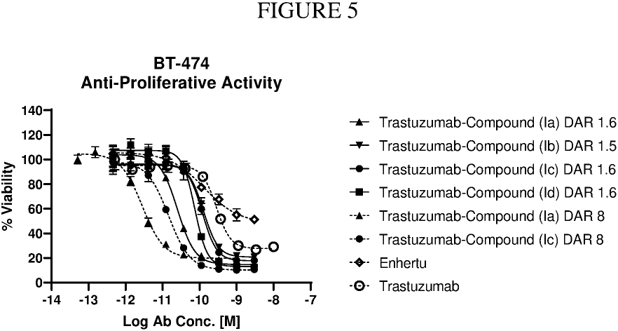

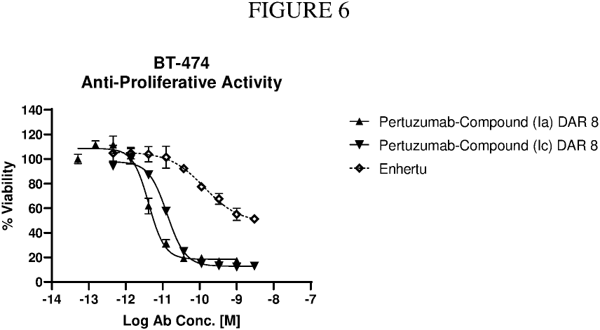

The anti-proliferative activity of trastuzumab and pertuzumab conjugates of Compound (Ia) and Compound (Ic) against the BT-474 breast cancer cell line is shown in FIGS. 5-6 (drug:antibody ratios are specified). The antibody drug conjugate ENHERTU^{®} and unconguaged antibody trastuzumab were found to be less active than the antibody neoDegrader conjugates against BT-474 cells.

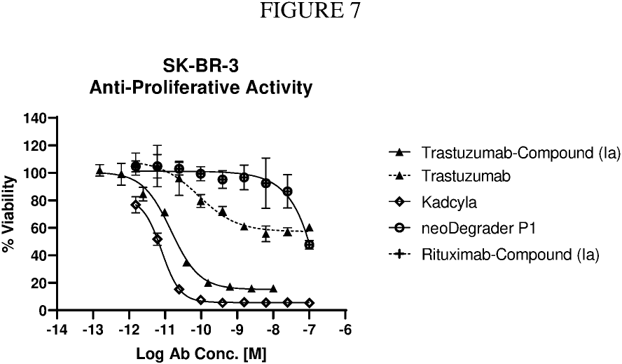

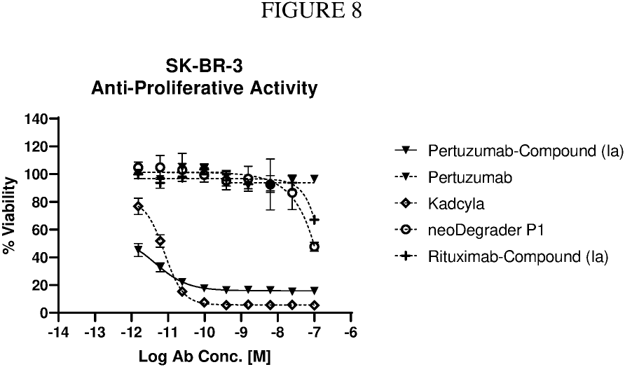

The anti-proliferative activity of trastuzumab and pertuzumab conjugates of Compound (Ia) against the SK-BR-3 breast cancer cell line is shown in FIGS. 7 and 8 (drug:antibody ratio= 8 in each neoDegrader conjugate). The conjugated neoDegraders had similar activity to antibody drug conjugate Kadcyla while unconguaged antibodies trastuzumab and pertuzumab were found to be significantly less active than the neoDegrader conjugates. Non cell-binding control neoDegrader conjugate Rituximab-Compound (Ia) and released neoDegraders P1 and P4 a were found to be significantly less active against SK-BR-3 cells.

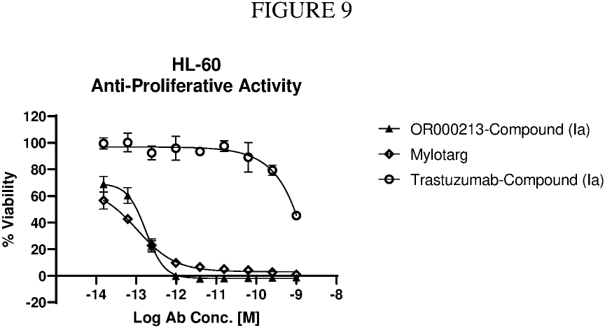

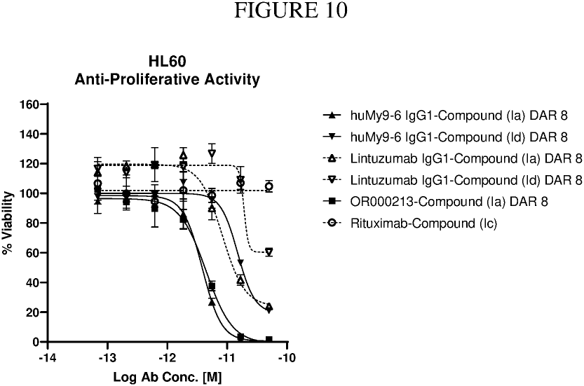

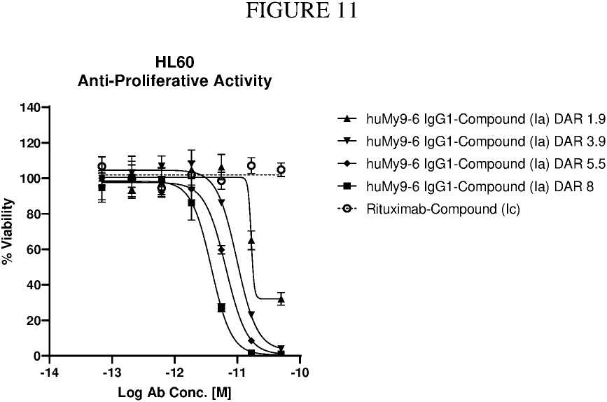

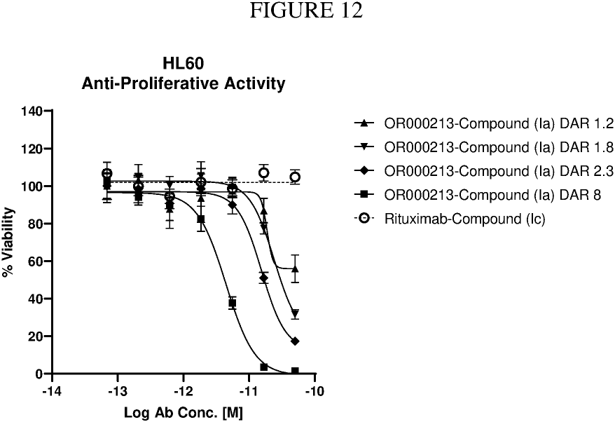

The antiproliferative activity of OR000213, huMy9-6, and Linztuzumab IgG1 conjugates of Compounds (Ia), (Id) against the HL-60 (acute myeloid leukemia) cell line is shown in FIGS. 9-12 (drug:antibody ratio =8 where unspecified). The neoDegrader conjugate was shown to have a similar activity as approved agent MYLOTARG^{®} against the cell line, while the non cell-binding control neoDegrader conjugates trastuzumab-Compound (Ia and Rituximab-Compound (Id) were significantly less active.

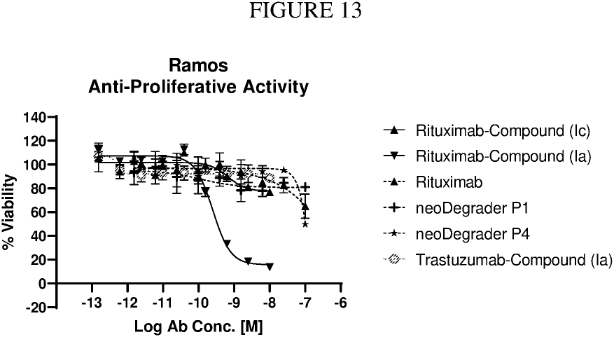

The antiproliferative activity of the rituximab conjugate of Compounds (Ia) and (Ic) against the Ramos (non-Hodgkins lymphoma) cell line is shown in FIG. 13 (drug:antibody ratio =8 where unspecified). Unconjugated rituximab, the non-cell binding control neoDegrader conjugate trastuzumab-Compound (Ia) and released neoDegraders P1 and P4 were shown to be less active than the neoDegrader conjugates against this cell line.

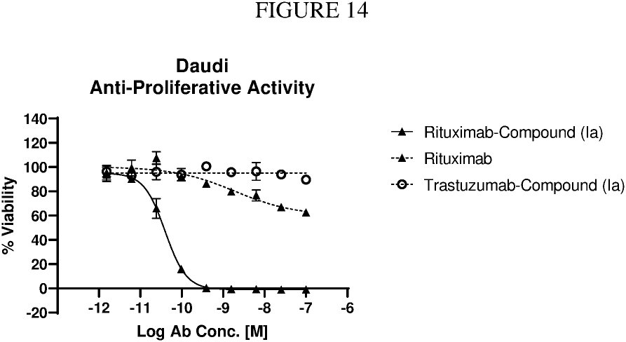

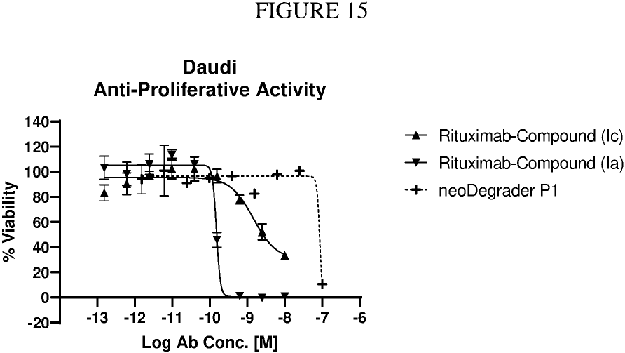

The antiproliferative activity of the rituximab conjugate of Compounds (Ia) and (Ic), against the Daudi lymphoma cell line is shown in FIGS. 14 and 15 (drug:antibody ratio =8 where unspecified). Unconjugated rituximab, the non-cell binding control neoDegrader conjugate trastuzumab-Compound (Ia) and released neoDegrader P1 were shown to be less active than the neoDegrader conjugates against this cell line.

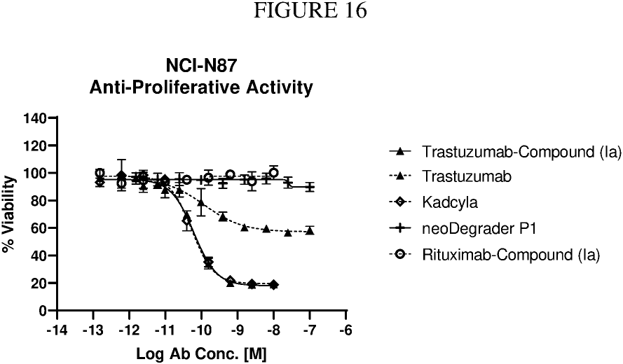

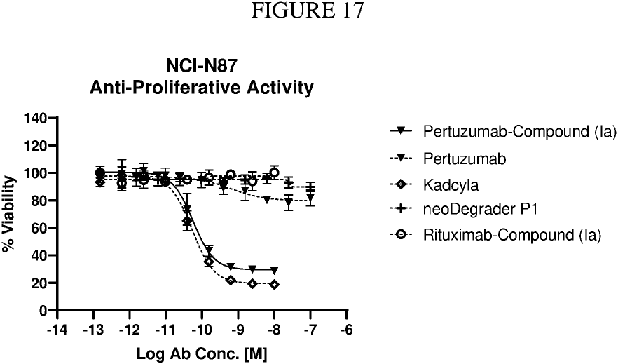

The anti-proliferative activity of trastuzumab and pertuzumab conjugates of Compound (Ia) against the NCI-N87 gastric cancer cell line is shown in FIGS. 16 and 17 (drug:antibody ratio= 8 in each neoDegrader conjugate). The conjugated neoDegraders had similar activity to antibody drug conjugate Kadcyla while unconguaged antibodies trastuzumab and pertuzumab were found to be significantly less active than the neoDegrader conjugates. Non cell-binding control neoDegrader conjugate Rituximab-Compound (Ia) and released neoDegrader P1 were found to be significantly less active against NCI-N87 cells.

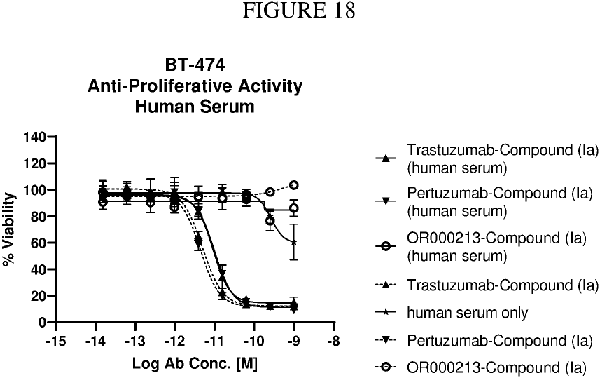

FIG. 18 shows the anti-proliferative activity of trastuzumab and pertuzumab conjugates of Compound (Ia) after a three day incubation with human serum versus the activity of the conjuages in the absence of serum in BT-464 breast cancer cells. As shown in the graph, the activity of neoDegrader conjugates were similar in the presence and absence of serum, showing that human serum does not affect activity. The non cell-binding control neoDegrader conjugate OR000213-Compound (Ia) is >1000-fold less active against this cell line.

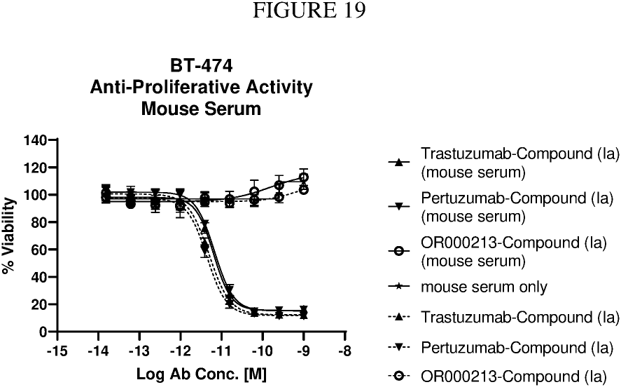

FIG. 19 shows the anti-proliferative activity of trastuzumab and pertuzumab conjugates of Compound (Ia) after a three day incubation with mouse serum versus the activity of the conjuages in the absence of serum in BT-464 breast cancer cells. As shown in the graph, the activity of the neoDegrader conjugates were similar in the presence and absence of serum, showing that mouse serum does not affect activity. The non cell-binding control neoDegrader conjugate OR000213-Compound (Ia) is >1000-fold less active against this cell line.

Tables 1 and 2 show the IC50 values of trastuzumab conjugates of Compounds (Ia), (Ib), (Ic), and (Id) and pertuzumab conjugates of Compounds (Ia) and (Ic) against various Her2 cell lines. As shown in Table 1, neoDegrader conjugates showed improved activity in the BT-474 cell line compared to the unconjugated antibodies and also showed improved activity against released payloads and the antiobody drug conjuages Kadcyla and ENHERTU^{®}. The neoDegrader conjugates also showed better activity against the SK-BR-3 breast cancer cell line and the NCI-N87 gastric cell line as compared to the unconjugated antibodies. As shown in Table 2, the antibody neoDegrader conjugates also had improved activity against the SNU-182 liver cell line compared to unconjugated antibody or Kadcyla.

TABLE 1

IC50 of Anti-Her2 NeoDegrader Conjugates

| Sample | | Breast | | | Gastric | Ovarian | |
|---|---|---|---|---|---|---|---|
| | DAR | BT-474 | SK-BR-3 | HCC1954 | NCI-N87 | SK-OV-3 | FU-OV-1 |
| Trastuzmab-Compound (Ia) | 8 | 2.8E-12 | 1.5E-11 | >1.0E-07 | 6.1E-11 | >1.0E-07 | >1.0E-07 |
| Trastuzmab-Compound (Ia) | 1.6 | 2.5E-11 | | | | | |
| Trastuzmab-Compound (Ib) | 1.5 | 1.3E-10 | | | | | |
| Trastuzmab-Compound (Ic) | 1.6 | 1.2E-10 | | | | | |
| Trastuzmab-Compound (Ic) | 8 | 1.3E-11 | | | | | |
| Trastuzmab-Compound (Id) | 1.6 | 7.4E-11 | | | | | |
| Pertuzumab-Compound (Ia) | 8 | 5.6E-13 | <1.5E-12 | >1.0E-07 | 5.7E-11 | | |
| Pertuzumab-(Compound Ic) | 8 | 3.5E-12 | | | | | |
| Enhertu | | 3.0E-09 | | | | | |
| Rituximab-Compound (Ia) | 8 | 1.4E-08 | 1.0E-07 | >1.0E-07 | >1.0E-07 | >1.0E-07 | >1.0E-07 |
| Trastuzumab | | 3.0E-10 | 8.6E-11 | >1.0E-07 | >1.0E-07 | >1.0E-07 | |
| Pertuzumab | | >1.0E-07 | >1.0E-07 | >1.0E-07 | >1.0E-07 | | |
| Kadcyla | | 8.7E-11 | 8.0E-12 | 7.9E-11 | 5.4E-11 | 4.0E-11 | >1.0E-07 |
| P1 | | 1.3E-08 | 1.0E-07 | >1.0E-07 | >1.0E-07 | | |
| P4 | | 2.9E-09 | | | | | |

TABLE 2

IC50 of Anti-Her2 NeoDegrader Conjugates

| Sample | Uterus | Lung | Pancreatic | Liver |
|---|---|---|---|---|
| | JHUEM-3 | NCI-H2030 | CFPAC-1 | SNU-182* |
| Trastuzmab-Compound (Ia) | >1.0E-07 | >1.0E-07 | >1.0E-07 | 5.0E-09 |
| Rituximab-Compound (Ia) | | | | 1.1E-08 |
| Trastuzumab | >1.0E-07 | >1.0E-07 | >1.0E-07 | >1.0E-07 |
| Kadcyla | | | | 1.7E-08 |
| NeoDegrader P1 | >1.0E-07 | >1.0E-07 | >1.0E-07 | |

*HER2 expression unknown

Table 3 shows the activity of antibody neoDegrader conjugates in anti-CD20 cell lines. Antibody neoDegrader conjugates had superior activity against Daudi and Ramos lymphocyte cell lines compared to the unconjugated antibody, the non cell-binding control neoDegrader conjugate trastuzumab-Compound I(a), and released payload.

TABLE 3

IC50 of Anti-CD20 NeoDegrader Conjugates

| Sample | | | |
|---|---|---|---|
| | Drug: Antibody Ratio | Daudi | Ramos |
| Rituximab-Compound (Ia) | ~8 | 1.0E-10 | 8.0E-10 |
| Rituximab-Compound (Ia) | ~4 | 4.4E-10 | |
| Trastuzumab-Compound (Ia) | | >1.0E-07 | >1.0E-07 |
| Rituximab | | >1.0E-07 | >1.0E-07 |
| NeoDegrader P1 | | >1.0E-07 | >1.0E-07 |
| CD20 (antibodies bound per cell) | | 300 K | 200 K |

Table 4 shows the IC50 values of huMy9-6 and OR000213 conjugates of Compounds (Ia) and (Id) and lintuzumab IgG1 conjugates of Compounds (Ia) and (Id) against AML HL-60 lines. As shown in Table 4, neoDegrader conjugates showed comparable activity in the HL-60 cell line compared to MYLOTARG^{®} and improved activity over the non-binding conjugate Rituximab-Compound (Ic).

TABLE 4

IC50 of Anti-HL-60 NeoDegrader Conjugates

| Sample | | AML |
|---|---|---|
| | DAR | HL-60 |
| huMy9-6 IgG1-Compound (la) | 1.9 | 1.7×10⁻¹¹ |
| huMy9-6 IgG1-Compound (la) | 3.9 | 9.9×10⁻¹² |
| huMy9-6 IgG1-Compound (la) | 5.5 | 6.6×10⁻¹² |
| huMy9-6 IgG1-Compound (la) | 8 | 3.8×10⁻¹² |
| huMy9-6 IgG1-Compound (Id) | 8 | 1.5×10⁻¹¹ |
| OR000213-Compound (Ia) | 1.2 | 1.9×10⁻¹¹ |
| OR000213-Compound (la) | 1.8 | 2.4×10⁻¹¹ |
| OR000213-Compound (Ia) | 2.3 | 1.6×10⁻¹¹ |
| OR000213-Compound (la) | 8 | 4.4×10⁻¹² |
| Lintuzumab IgG1-Compound (Ia) | 8 | 1.2×10⁻¹¹ |
| Lintuzumab IgG1-Compound (Id) | 8 | >5.0×10⁻¹¹ |
| Mylotarg | | 1.1×10⁻¹² |
| Rituximab-Compound (Ic) | 8 | >5.0×10⁻¹¹ |

Table 5 shows the antiproliferative activity of the trastuzumab and pertuzumab Compound (Ia) conjugates against the BT-474 breast cancer cell line after incubation with human serum or mouse serum compared to non cell-binding control neoDegrader conjugate OR000213-Compound (Ia). As shown in the table, activity of neoDegraders incubated in human or mouse serum was consistent with activity where no serum was introduced.

TABLE 5

IC50 of Serum Stability Test

| Sample | BT-474 | | |
|---|---|---|---|
| | Human serum | Mouse serum | No serum |
| Trastuzumab-Compound (Ia) | 9.1E-12 | 6.4E-12 | 4.9E-12 |
| Pertuzumab-Compound (Ia) | 9.8E-12 | 6.7E-12 | 4.4E-12 |
| OR000213-Compound (Ia) | >1.0E-09 | >1.0E-09 | >1.0E-09 |

### Example 6: General Procedure 2 for in Vitro Antiproliferation Assay for NeoDegraders and NeoDegrader Conjugates

Cell Culture: Cell lines were obtained from the American Type Culture Collection (ATCC, Manassas, VA, USA) or Deutsche Sammlung von Mikroorganismen und Zellkulturen (DSMZ, Braunschweig, Germany) and maintained according to the culturing conditions specified by ATCC or DSMZ. Cells were thawed out and maintained in culture for at least two passages before proceeding with experimental conditions.

Cytotoxicity assays: For adherent cell lines, the cells were dissociated with enzyme-free PBS-based cell dissociation buffer (Gibco, USA) and plated on tissue culture treated 96-well flat bottom polystyrene plates (Costar, Corning, USA) at appropriate cell densities depending on doubling time of the cells. 18 hours after plating, cells were treated with test articles at appropriate concentrations starting at 100 nM and diluted in 4-fold serial dilutions. For suspension cell lines, the cells were seeded on the same day of treatment and cells were treated as described above. Adherent cells were treated for 5 days and suspension cells were treated for 3 days. Cell proliferation was assessed using the Cell Counting Kit-8 (CCK-8, Dojindo Laboratories, Japan) and measurements were obtained using the Promega GloMax Discover plate reader (Promega, USA). Data were analyzed using GraphPad Prism software (GraphPad Software, San Diego, CA). All data points were obtained from technical triplicates and the experiments were validated using three biological replicates.

Table 6 shows the activities of neoDegraders P1, P3, and P4 against various cancer cell lines. As shown in the table, the neoDegraders have activity against each of the cell lines.

TABLE 6

IC50 of Representative NeoDegraders in Various Cancer Cell Lines

| | | | | |
|---|---|---|---|---|
| Cell line | Tissue | P3 | P1 | P4 |
| AGS | Gastric | 6.50E-07 | >1.00E-06 | 6.50E-07 |
| BT474 | Breast | 6.37E-09 | 1.3E-08 | 2.9E-09 |
| Daudi | NHL | 9.17E-09 | 4.74E-08 | 1.43E-08 |
| JHUEM3 | Endometrial | 3.74E-08 | >1.00E-06 | 5.86E-08 |
| KE39 | Gastric | 6.50E-07 | >1.00E-06 | 1.19E-07 |
| LOVO | Colon | 7.50E-07 | >1.00E-06 | 1.19E-07 |
| MOLM13 | AML | 8.50E-09 | 2.48E-08 | 9.50E-09 |
| Molp8 | MM | 6.63E-09 | 2.37E-08 | 7.42E-09 |
| RPMI7951 | Skin | 2.60E-08 | 9.71E-08 | 3.02E-08 |
| SKBR3 | Breast | 3.50E-08 | 1.0E-07 | 6.46E-08 |
| SKOV3 | Ovarian | 9.97E-08 | 1.14E-07 | 1.38E-07 |
| SNU182 | Liver | 8.86E-09 | 1.27E-08 | 1.30E-08 |
| SU-DHL-8 | NHL | 1.84E-08 | 1.02E-07 | 1.98E-08 |
| THLE2 | Human liver normal | 5.99E-08 | >1.00E-06 | 6.27E-09 |
| THP-1 | AML | 6.50E-08 | 3.50E-07 | 8.50E-08 |
| U251MG | Brain | 3.58E-08 | >1.00E-06 | 4.60E-08 |
| HL-60 | AML | 3.02E-09 | 1.23E-08 | 5.45E-09 |
| CMK | Acute megakaryocytic leukemia | 1.23E-08 | 4.08E-07 | 1.00E-06 |
| EOL-1 | Acute myeloid leukemia | 7.33E-10 | 3.10E-09 | 1.88E-09 |
| MV4-11 | Biphenotypic B myelomonocytic leukemia | 7.83E-10 | 2.72E-09 | 1.91E-09 |
| ML-2 | Acute myelomonocytic leukemia | 5.87E-10 | 1.86E-09 | 1.27E-09 |
| NB-4 | Acute promyelocytic leukemia | 2.20E-09 | 8.63E-09 | 2.63E-09 |
| OCI-M1 | Acute myeloid leukemia | 1.04E-07 | >1.00E-06 | 1.24E-07 |
| OCI-M2 | Acute myelocytic leukemia | 2.11E-08 | 6.50E-07 | 2.09E-08 |
| OCI-AML2 | Acute myeloid leukemia | 2.91E-08 | 9.59E-08 | >1.00E-06 |
| OCI-AML4 | Acute myeloid leukemia | 7.00E-09 | 2.24E-08 | 1.69E-08 |
| SKM-1 | Acute myeloid leukemia | 1.89E-09 | 3.21E-09 | 3.77E-09 |
| PL-21 | Acute myeloid leukemia | 2.09E-09 | 7.55E-09 | 2.28E-09 |

Table 7 shows the activities of the pertuzumab-Compound (Ia) conjugate and the known antibody drug conjugate ENHERTU^{®} against various breast cancer cell lines. As shown in the table, the pertuzumab-Compound(Ia) conjugate was more active in all of the reported cell lines.

TABLE 7

IC50 of Pertuzumab-Compound (Ia) Conjugate in Various Breast Cancer Cell Lines

| | | | |
|---|---|---|---|
| Cell Line | Description | Pertuzumab-Compound (Ia) IC50 (M) | ENHERTU (M) |
| ZR-75-30 | breast, ductal carcinoma | 1.10E-10 | 9.42E-09 |
| BT-474 | breast, ductal carcinoma | 5.6E-13 | 3.0E-09 |
| SK-BR-3 | breast, adenocarcinoma (pleural effusion) | <1.5E-12 | 7.53E-10 |
| MDA-MB-453 | breast, metastatic carcinoma | 3.38E-10 | >2.00E-8 |
| HCC202 | breast, primary ductal carcinoma | 5.75E-10 | >2.00E-8 |
| HCC1569 | breast, primary metaplastic carcinoma | 6.24E-10 | 8.37E-10 |
| UACC-812 | breast, ductal carcinoma | 8.15E-10 | >2.00E-8 |
| MDA-MB-361 | breast, adenocarcinoma | 8.64E-10 | 4.74E-09 |
| T-47D | breast, ductal carcinoma | 9.96E-10 | >2.00E-8 |
| HCC1419 | breast, primary ductal carcinoma | 1.11E-09 | >2.00E-8 |
| HCC2218 | breast, primary ductal carcinoma | 1.20E-09 | 4.36E-09 |
| MDA-MB-175-VII | breast, ductal carcinoma | 1.20E-09 | >2.00E-8 |
| AU565 | breast, adenocarcinoma | 1.69E-09 | 5.59E-10 |
| HCC2157 | breast, primary ductal carcinoma | 1.44E-08 | >2.00E-8 |

Table 8 shows the activities of the pertuzumab-Compound (Ia) conjugate and the known antibody drug conjugate ENHERTU against three gastric cancer cell lines. As shown in the table, the pertuzumab-Compound(Ia) conjugate was more active in all of the reported cell lines.

TABLE 8

IC50 of Pertuzumab-Compound I(a) Conjugate in Various Gastric Cancer Cell Lines

| | | | |
|---|---|---|---|
| Cell Line | Description | Pertuzumab Compound(Ia) IC50 (M) | ENHERTU (M) |
| NCC-StC-K140 | stomach, stomach cancer | 3.42E-10 | >2.00E-8 |
| NCI-N87 | stomach, gastric carcinoma | 5.7E-11 | 8.82E-10 |
| NUGC-4 | stomach, gastric carcinoma | 9.59E-09 | >2.00E-8 |

Table 9 shows the activities of the OR000213-Compound (Ia) conjugate and the known antibody drug conjugate MYLOTARG against various acute myeloid leukemia cell lines. As shown in the table, the OR000213-Compound (Ia) conjugate had better activity in several of the cell lines.

TABLE 9

IC50 of OR000213-Compound I(a) Conjugate in Various Acute Myeloid Leukemia Cell Lines

| | | | |
|---|---|---|---|
| Cell Line | Description | OR000213-Compound(Ia) IC50 (M) | MYLOTARG (M) |
| HL-60 | leukemia, acute promyelocytic | 2.41E-11 | 3.26E-10 |
| ML-2 | leukemia, acute myelomonocytic leukemia | 2.63E-11 | 5.02E-11 |
| MV-4-11 | leukemia, biphenotypic B myelomonocytic | 2.64E-11 | 5.32E-11 |
| SKNO-1 | leukemia, acute myeloid leukemia | 2.89E-11 | 1.34E-08 |
| EoL-1 | Leukemia, Human eosinophilic leukaemia | 5.18E-11 | 6.87E-11 |
| HNT-34 | leukemia, acute myeloid leukemia | 5.36E-11 | 3.77E-09 |
| OCI-AML-4 | leukemia, acute myeloid leukemia | 5.45E-11 | 2.37E-10 |
| Kasumi-6 | leukemia, acute myeloid, myeloblast | 9.64E-11 | >2.00E-8 |
| PLB-985 | leukemia, acute myeloid leukemia (derivative of HL-60) | 1.11E-10 | 7.14E-09 |
| AML-193 | leukemia, acute monocytic, monocyte | 1.23E-10 | >2.00E-8 |
| Molm-13 | leukemia, acute,myeloid,leukemia suspension | 1.40E-10 | 2.70E-11 |
| OCI-AML-5 | leukemia, acute myeloid leukemia | 1.50E-10 | 7.83E-11 |
| SKM-1 | leukemia, myelodysplastic syndrome | 1.85E-10 | 2.24E-10 |
| OCI-AML-2 | leukemia, acute myeloid leukemia | 5.65E-09 | 3.94E-11 |
| OCI-AML-6 | leukemia, acute myeloid leukemia | 7.12E-09 | >2.00E-8 |
| F-36P | leukemia, myelodysplastic syndrome | 7.24E-09 | >2.00E-8 |
| Kopn-8 | leukemia, B cell precursor leukemia | 1.51E-08 | 6.87E-10 |
| OCI-AML-1 | leukemia, acute myeloid leukemia | 1.80E-08 | 2.24E-10 |

Table 10 shows the activities of three anti-CD38 neoDegrader conjugates against various multiple myeloma cell lines. As shown in the table, the conjugates had good activity in all of the cell lines.

TABLE 10

IC50 of Anti-CD38 NeoDegrader Conjugates in Multiple Myeloma Cell Lines

| Sample | Multiple Myeloma | | | | | | |
|---|---|---|---|---|---|---|---|
| | MOLP-2 | NCI-H929 | KMS-12-BM | MM.1S | OPM-2 | LP-1 | MM.1R |
| HuAT13/5-Compound (Ia) | 3.40E-12 | 5.99E-12 | 8.23E-12 | 3.82E-11 | 6.66E-11 | 3.38E-12 | 2.38E-10 |
| Isatuximab-Compound (Ia) | 8.22E-11 | 6.76E-12 | 3.04E-11 | 4.04E-11 | 1.95E-10 | 1.82E-10 | 7.42E-11 |
| Daratumumab-Compound (Ia) | 9.24E-12 | 1.66E-12 | 1.66E-12 | 6.19E-09 | 4.07E-11 | 4.07E-11 | 2.80E-11 |

Table 11 shows the activity of an anti-CD 138 neoDegrader conjugate against various multiple myeloma cell lines. As shown in the table, the conjugate had good activity in all of the cell lines.

TABLE 11

IC50 of Anti-CD38 NeoDegrader Conjugates in Multiple Myeloma Cell Lines

| Sample | Multiple Myeloma | | | | |
|---|---|---|---|---|---|
| | MOLP-2 | KMS-12-BM | MM.1S | OPM-2 | MM.1R |
| Indatuximab-Compound (Ia) | 9.06E-11 | 3.59E-10 | 5.26E-11 | 3.57E-11 | 1.21E-10 |

Table 12 shows the activities of an anti-BCMA neoDegrader conjugate against various multiple myeloma cell lines. As shown in the table, the conjugate had good activity in all of the cell lines.

TABLE 12

IC50 of Anti-BCMA NeoDegrader Conjugate in Multiple Myeloma Cell Lines

| Sample | Multiple Myeloma | | | | | | |
|---|---|---|---|---|---|---|---|
| | MOLP-2 | NCI-H929 | KMS-12-BM | MM.1S | OPM-2 | LP-1 | MM.1R |
| Belantamab-Compound (Ia) | 1.97E-10 | 1.19E-08 | 8.70E-11 | 2.09E-10 | 1.19E-10 | 3.17E-09 | 6.84E-10 |

Table 13 shows the activities of an anti-Trop-2 neoDegrader conjugate against various cancer cell lines. As shown in the table, the conjugate had good activity in all of the cell lines.

TABLE 13

IC50 of Anti-Trop-2 NeoDegrader Conjugate in Various Cancer Cell Lines

| Sample | urothelial | Urinary bladder | TNBC |
|---|---|---|---|
| | SW780 | KMBC-2 | HCC2157 |
| Sacituzumab-Compound (Ia) | 3.85E-11 | 2.30E-11 | 5.62E-11 |

Table 14 shows the activities of an anti-FGFR4 neoDegrader conjugate against two cancer cell lines. As shown in the table, the conjugate had good activity in both cell lines and had better activity than the US-1784 antibody and the unconjugated neoDegrader alone.

TABLE 14

IC50 of Anti-FGFR4 NeoDegrader Conjugate in Various Cancer Cell Lines

| Sample | Rhabdomyosarcoma | |
|---|---|---|
| | RH30 | RH41 |
| NeoDegrader P1 | 1.741E-07 | 1.210E-06 |
| U3-1784 | >2.0E-08 | >2.0E-08 |
| U3-1784-Compound (Ia) | 2.052E-11 | 1.703E-11 |

Table 15 shows the activities of an anti-EGFR neoDegrader conjugate against two synovial sarcoma cell lines. As shown in the table, the conjugate had good activity in both cell lines and had better activity cetuximab and the unconjugated neoDegrader alone.

TABLE 15

IC50 of Anti-EGFR NeoDegrader Conjugate in Synovial Sarcoma Cell Lines

| Sample | Synovial Sarcoma | |
|---|---|---|
| | ASKA | HS-SY-II |
| NeoDegrader P1 | 7.120E-08 | 1.171E-08 |
| Cetuximab | >3.0E-09 | >3.0E-09 |
| Cetuximab-Compound (Ia) | 6.571E-013 | 1.057E-011 |

Table 16 shows the activities of an anti-PDGF-R α neoDegrader conjugate against two cancer cell lines. As shown in the table, the conjugate had good activity in both cell lines.

TABLE 16

IC50 of Anti-PDGF-R α NeoDegrader Conjugate in Cancer Cell Lines

| Sample | rhabdomyosarcoma | chondrosarcoma |
|---|---|---|
| | A-204 | H-EMC-SS |
| Olaratumab-Compound (Ia) | 2.36E-09 | 3.70E-10 |

Table 17 shows the activities of an anti-TEM1/CD248 neoDegrader conjugate against the rhabdomyosarcoma cell line. As shown in the table, the conjugate had good activity agains the cell line.

TABLE 17

IC50 of Anti-TEM1/CD248 NeoDegrader Conjugate in Rhabdomyosarcoma Cell Line

| Sample | Ewing’s sarcoma |
|---|---|
| | RD-ES |
| Ontuxizumab-Compound (Ia) | 2.73E-11 |

### Example 7: Treatment of Breast Cancer With Anti-Her2 Antibody-neoDegrader Conjugate

Trastuzumab-compound (Ia) conjugate and pertuzumab-compound (Ia) conjugate were tested in immunodeficient mice (Fox Chase SCID^{®}, CB17/Icr-Prkdc^{scid}/IcrIcoCrl, Charles River). 1 mm³ BT474 human breast carcinomas fragments were implanted subcutaneously in the right flank of the mice. The mice were dosed with anti-Her2 antibody-neoDegrader conjugates, non-targeting neoDegrader conjugates, and vehicle control once tumors reached an average size of 100 - 150 mm³.

The stock solutions of trastuzumab-Compound (Ia) conjugate, rituximab-Compound (Ia) conjugate, and pertuzumab-Compound (Ia) conjugate were diluted with vehicle to obtain 0.5 mg/mL dosing solutions, which provided 5 mg/kg in a dosing volume of 10 mL/kg (0.2 mL per 20 g mouse), adjusted to the body weight of each animal.

Mice were divided into 4 treatment groups (N=8/group), as follows: 1) vehicle; 2) trastuzumab-Compound (Ia) conjugate (5 mg/kg, iv, qd x 1); 3) rituximab-Compound (Ia) conjugate (5 mg/kg, iv, qd x 1); 4) pertuzumab-Compound (Ia) conjugate (5 mg/kg, iv, qd x 1). All test articles were administered intravenously (i.v.) as single doeses (qd x 1) in volumes adjusted for body weight (0.200 mL/20 g mouse).

Tumors were measured using calipers twice per week, and each animal was euthanized when its tumor reached the endpoint volume (1,000 mm³) or on the last day (Day 60) of the study, whichever came first. The MTV(n) was defined as the median tumor volume on the last day of the study in the number of animals remaining (n) whose tumors had not attained the endpoint volume.

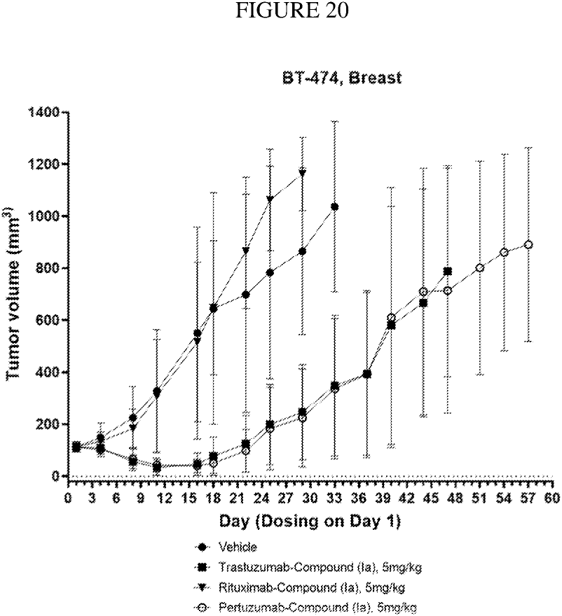

As shown in FIG. 20, the pertuzumab and rituximab conjugates provided slower tumor growth over time compared to vehicle and non cell-binding control neoDegrader conjugate rituximab-Compound (Ia).

### Example 8: Treatment of Non-Hodgkin’s Lymphoma (NHL) With Anti-CD20 Antibody-neoDegrader Conjugates

Rituximab-compound (Ia) conjugate was tested in immunodeficient mice (Fox Chase SCID^{®}, CB17/Icr-Prkdc^{scid}/IcrIcoCrl, Charles River). 1×10⁷ Daudi Burkitt’s B cell lymphoma cells (ATCC^{®} CCL-213™) were injected subcutaneously in the right flank (0.1 mL cell suspension) of the mice. The mice were dosed with the anti-CD20 antibody-neoDegrader conjugates, non-targeting neodegrader conjugates and vehicle control were initiated when the tumors reached an average size of 100 - 150 mm³.

The stock solutions of trastuzumab-Compound (Ia) and rituximab-Compound (Ia) were diluted with vehicle to obtain 0.5 mg/mL and 0.1 mg/mL dosing solutions, which provided 5 and 1 mg/kg in a dosing volume of 10 mL/kg (0.2 mL per 20 g mouse), adjusted to the body weight of each animal.

Mice were divided into 4 treatment groups (N=8/group), as follows: 1) vehicle; 2) trastuzumab-Compound (Ia) (5 mg/kg, iv, qd x 1); 3) rituximab-Compound (Ia) (1 mg/kg, iv, qd x 1); 4) rituximab-Compound (Ia) (5 mg/kg, iv, qd x 1). All test articles were administered intravenously (i.v.) as a single dose (qd x 1) in volumes adjusted for body weight (0.200 mL/20 g mouse).

Tumors were measured using calipers twice per week, and each animal was euthanized when its tumor reached the endpoint volume (1,500 mm³) or on the last day (Day 45) of the study, whichever came first. The MTV(n) was defined as the median tumor volume on the last day of the study in the number of animals remaining (n) whose tumors had not attained the endpoint volume.

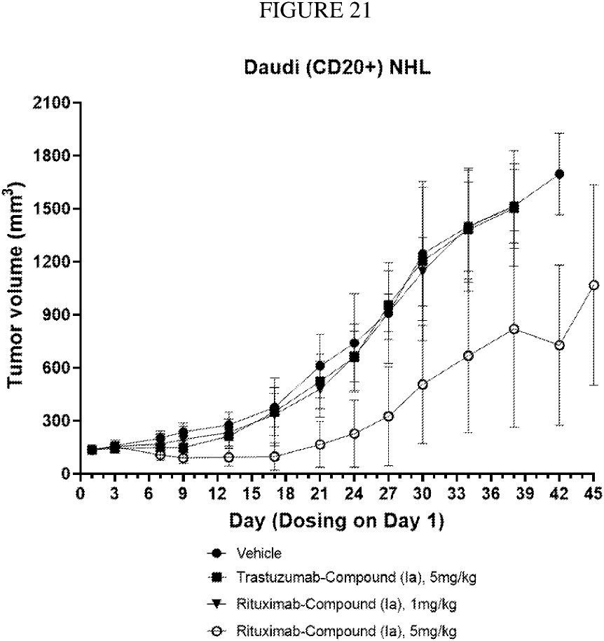

As shown in FIG. 21, the 5 mg/kg dose of the rituximab conjugate provided slower tumor growth over time compared to the 1 mg/kg dose, vehicle, and non cell-binding control neoDegrader conjugate trastuzumab-Compound (Ia).

### Example 9. Treatment of Acute Myeloid Leukemia (AML) With Anti-CD33 Antibody-neoDegrader Conjugate

OR000213-compound (Ia) was tested in athymic nude mice (Crl:NU(NCr)-Foxn1nu, Charles River). 1 × 107 HL-60 acute promyelocytic leukemia cells (ATCC^{®} CCL-240 ™) were injected subcutaneously in the right flank of the mice (0.1 mL cell suspension). The mice were dosed with anti-CD33 antibody-neoDegrader conjugates, non-targeting neodegrader conjugates, and vehicle control once tumors reached an average size of 100 - 150 mm³.

The stock solutions of trastuzumab-Compound (Ia) and OR000213-Compound (Ia) were diluted with vehicle to obtain 0.5 mg/mL and 0.1 mg/mL dosing solutions, which provided 5 and 1 mg/kg in a dosing volume of 10 mL/kg (0.2 mL per 20 g mouse), adjusted to the body weight of each animal.

Mice were divided into 4 treatment groups (N=8/group), as follows: 1) vehicle; 2) trastuzumab-Compound (Ia) (5 mg/kg, iv, qd x 1); 3) rituximab-Compound (Ia) (1 mg/kg, iv, qd x 1); 4) rituximab-Compound (Ia) (5 mg/kg, iv, qd x 1). All test articles were administered intravenously (i.v.) as a single dose (qd x 1) in volumes adjusted for body weight (0.200 mL/20 g mouse).

Tumors were measured using calipers twice per week, and each animal was euthanized when its tumor reached the endpoint volume (2,000 mm3) or on the last day (Day 45) of the study, whichever came first. The MTV(n) was defined as the median tumor volume on the last day of the study in the number of animals remaining (n) whose tumors had not attained the endpoint volume.

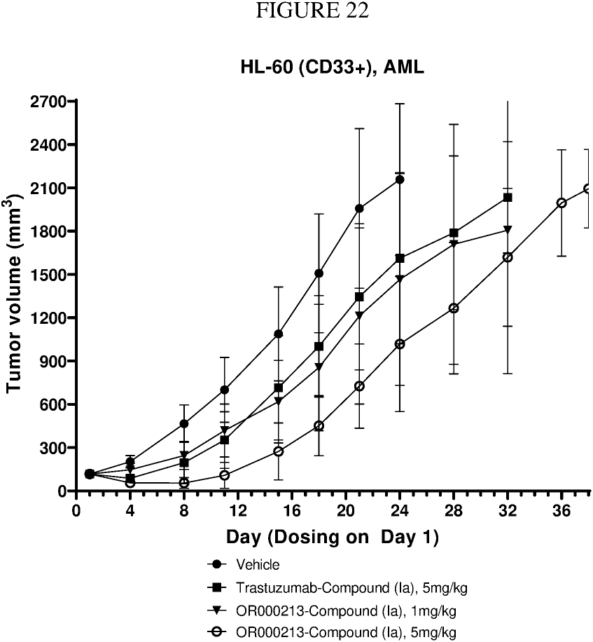

As shown in FIG. 22, all of the neoDegrader conjugates provided slower tumor growth over time compared to the vehicle.

### Example 10. Treatment of Multiple Myeloma With Anti-CD38 Antibody-neoDegrader Conjugate

HuAT13/5-compound (Ia) was tested in CB.17 SCID mice (CB17/Icr-Prkdcscid/IcrIcoCrl, Charles River). 1 × 107 NCI-H929 myeloma cells (ATCC® CRL-9068™) in 50% Matrigel were injected subcutaneously in the axillary site of the mice (0.1 mL cell suspension). The mice were dosed with anti-CD38 antibody-neoDegrader conjugates and vehicle control once tumors reached an average size of 100 - 150 mm³.

The stock solution of HuAT13/5-Compound (Ia) was diluted with vehicle to obtain 0.5 mg/mL dosing solution, which provided 5 mg/kg in a dosing volume of 10 mL/kg (0.2 mL per 20 g mouse), adjusted to the body weight of each animal.

Mice were divided into 2 treatment groups (N=10/group), as follows: 1) vehicle; 2) HuAT13/5-compound (Ia) (5 mg/kg, iv, qd x 1). All test articles were administered intravenously (i.v.) as a single dose (qd x 1) in volumes adjusted for body weight (0.200 mL/20 g mouse).

Tumors were measured using calipers twice per week, and each animal was euthanized when its tumor reached the endpoint volume (2,000 mm3) or on the last day (Day 45) of the study, whichever came first. The MTV(n) was defined as the median tumor volume on the last day of the study in the number of animals remaining (n) whose tumors had not attained the endpoint volume.

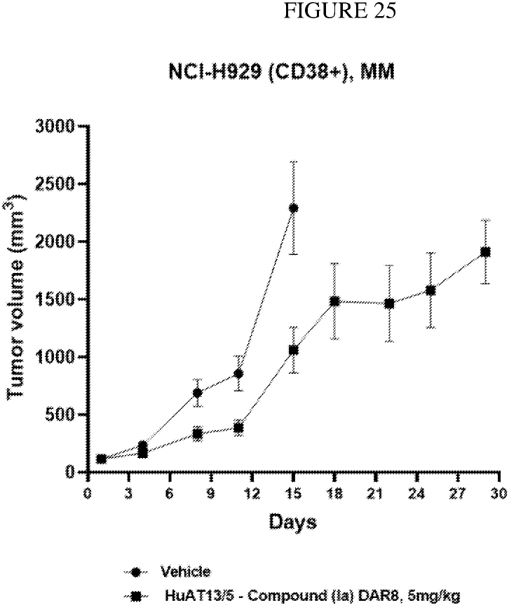

As shown in FIG. 25, the 5 mg/kg dose of the HuAT13/5 neoDegrader conjugate provided slower tumor growth over time compared to the vehicle.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A conjugate of formula (I): 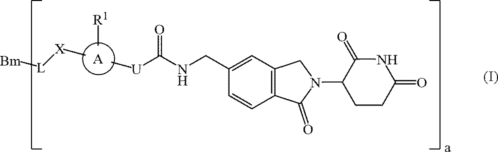
or a pharmaceutically acceptable salt thereof, wherein:
a is an integer from 1 to 10;
A is phenyl or a C₄-C₁₀cycloalkyl ring;
U is selected from NH and CF₂;
R¹ is independently selected from hydrogen and halo;
X is selected from —NR²—,═C(CH₃)—, —Q—(CH₂)ₙ—, and —Q(CH₂)ₘQ′(CH₂)ₙ—; wherein
Q and Q′ are each independently O, S, or N(R²)v;
v is 1 or 2;
each R² is independently hydrogen or C₁-C₆alkyl;
n is an integer from 1 to 6; and
m is an integer from 2 to 6;
wherein the left side of each group is attached to L and the right side is attached to A;
provided that when X is NH or —Q—(CH₂)ₙ—, R¹ is halo;
L is a cleavable linker or non-cleavable linker; and
Bm is a binding moiety that is capable of specifically binding to a protein.

2-8. (canceled)

9. The conjugate of claim 1, or a pharmaceutically acceptable salt thereof, wherein the cleavable linker is cleavable by a protease.

10. The conjugate of claim 9, or a pharmaceutically acceptable salt thereof, wherein L is selected from the group consisting of 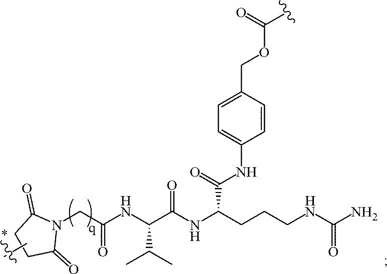 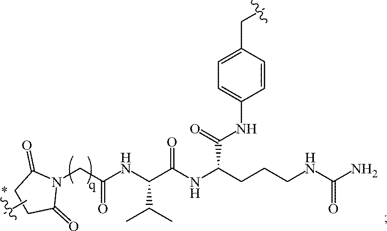 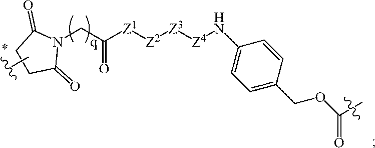 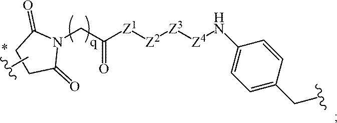 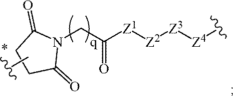 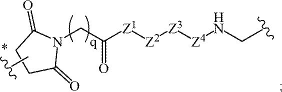 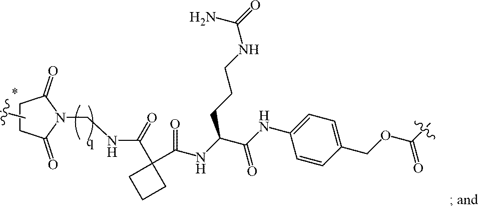 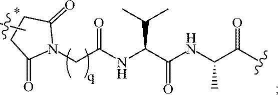
wherein:
q is an integer from 2 to 10;
Z¹, Z², Z³, and Z⁴ are each independently absent or a naturally-occurring amino acid residue in the L- or D-configuration, provided that at least two of Z¹, Z², Z³, and Z⁴ are amino acid residues; 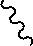
is the point of attachment to X; and 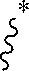
is the point of attachment to the binding moiety.

11. The conjugate of claim 10, or a pharmaceutically acceptable salt thereof, wherein Z¹, Z², Z³, and Z⁴ are independently absent or selected from the group consisting of L-valine, D-valine, L-citrulline, D-citrulline, L-alanine, D-alanine, L-glutamine, D-glutaimine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, and Z⁴ are amino acid residues.

12. The conjugate of claim 11, or a pharmaceutically acceptable salt thereof, wherein:
Z¹ is absent or glycine;
Z² is absent or selected from the group consisting of L-glutamine, D-glutamine, L-glutamic acid, D-glutamic acid, L-aspartic acid, D-aspartic acid, L-alanine, D-alanine, and glycine;
Z³ is selected from the group consisting of L-valine, D-valine, L-alanine, D-alanine, L-phenylalanine, D-phenylalanine, and glycine; and
Z⁴ is selected from the group consisting of L-alanine, D-alanine, L-citrulline, D-citrulline, L-asparagine, D-asparagine, L-lysine, D-lysine, L-phenylalamine, D-phenylalanine, and glycine.

13. The conjugate of claim 10, or a pharmaceutically acceptable salt thereof, wherein L is 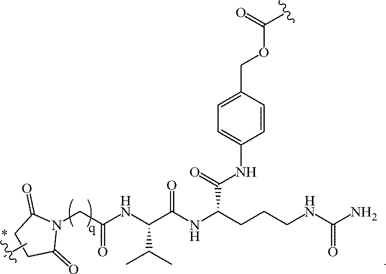
.

14. The conjugate of claim 13, or a pharmaceutically acceptable salt thereof, wherein q is 5.

15-22. (canceled)

23. The conjugate of claim 1, or a pharmaceutically acceptable salt thereof, wherein L is a beta-glucuronidase cleavable linker.

24. (canceled)

25. The conjugate of claim 1, or a pharmaceutically acceptable salt thereof, wherein Bm is an antibody or antigen binding portion thereof.

26. The conjugate of claim 25, wherein the protein that the binding moiety binds to is a surface antigen.

27. The conjugate of claim 26, wherein the surface antigen comprises 5T4, ACE, ADRB3, AKAP-4, ALK, Androgen receptor, AOC3, APP, Axin1, AXL, B7H3, B7-H4, BCL2, BCMA, bcr-abl, BORIS, BST2, C242, C4.4a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudin 3, Claudin 18.2, CLDN6, CLEC12A, CLL-1, cll3, c-MET, Crypto 1 growth factor, CS1, CTLA-4, CXCR2, CXORF61, Cyclin Bl, CYP1B1, Cadherin-3, Cadherin-6, DLL3, E7, EDNRB, EFNA4, EGFR, EGFRvIII, ELF2M, EMR2, ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GloboH, GM3, GPC1, GPC2, GPC3, gplOO, GPNMB, GPR20, GPRC5D, GUCY2C, HAVCR1, HER2, HER3, HGF, HMI.24, HMWMAA, HPV E6, hTERT, human telomerase reverse transcriptase, ICAM, ICOS-L, IFN- α, IFN-y, IGF-I receptor, IGLL1, IL-2 receptor, IL-4 receptor, IL-13Ra2, IL-1 IRa, IL-1, IL-12, IL-23, IL-13, IL-22, IL-4, IL-5, IL-6, interferon receptor, integrins (including α₄, αᵥβ₃, αᵥβ₅, αᵥβ₆, α₁β₄, α₄β₁, α₄β₇, α₅β₁, α₆β₄, α_{IIb}β₃ interginsintegrins), Integrin alphaV, intestinal carboxyl esterase, KIT, LAGE-la, LAIR1, LAMP-1, LCK, Legumain, LewisY, LFA-1(CD11a), L-selectin(CD62L), LILRA2, LIV-1, LMP2, LRRC15, LY6E, LY6K, LY75, MAD-CT-1, MAD-CT-2, MAGE Al, MelanA/MARTl, Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2, OY-TES1, p53, p53 mutant, PANX3, PAP, PAX3, PAX5, p-CAD, PCTA- ⅟Galectin 8, PD-L1, PD-L2, PDGFR, PDGFR-beta, phosphatidylserine, PIK3CA, PLAC1, Polysialic acid, Prostase, prostatic carcinoma cell, prostein, Pseudomonas aeruginosa, rabies, survivin and telomerase, PRSS21, PSCA, PSMA, PTK7, RAGE-1, RANKL, Ras mutant, respiratory syncytial virus, Rhesus factor, RhoC, RON, ROR1, ROR2, RU1, RU2, sarcoma translocation breakpoints, SART3, SLAMF7, SLC44A4, sLe, SLITRK6, sperm protein 17, sphingosine-1-phosphate, SSEA-4, SSX2, STEAP1, TAG72, TARP, TCRβ, TEM1/CD248, TEM7R, tenascin C, TF, TGF-1, TGF- β2, TNF-α, TGS5, Tie 2, TIM-1, Tn Ag, TRAC, TRAIL-R1, TRAIL-R2, TROP-2, TRP-2, TRPV1, TSHR, tumor antigen CTAA16.88, tyrosinase, UPK2, VEGF, VEGFR1, VEGFR2, vimentin, WTl, XAGE1, or combinations thereof.

28. (canceled)

29. The conjugate of claim 25, or a pharmaceutically acceptable salt thereof, wherein the antibody is selected from the group consisting of rituximab, trastuzumab, gemtuzumab, pertuzumab, obinutuzumab, ofatumumab, olaratumab, ontuximab, isatuximab, Sacituzumab, U3-1784, daratumumab, STI-6129, lintuzumab, huMy9-6, OR000213, balantamab, indatuximab, cetuximab, dinutuximab, anti-CD38 A2 antibody, HuAT13/5 antibody, alemtuzumab, ibritumomab, tositumomab, bevacizumab, panitumumab, tremelimumab, ticilimumab, catumaxomab, oregovomab, and veltuzumab.

30. (canceled)

31. The conjugate of claim 1, or a pharmaceutically acceptable salt thereof, wherein:
A is phenyl;
U is NH;
R¹ is halo; and
X is —N(R²)v(CH₂)mO(CH₂)n—; wherein:
v is 1;
m and n are 2; and
R² is methyl.

32. The conjugate of claim 1, wherein:
A is phenyl;
U is NH;
R¹ is halo; and
X is —N(R²)v(CH₂)mO(CH₂)n—; wherein:
v is 2;
m and n are 2; and
each R² is methyl.

33. The conjugate of claim 1, wherein:
A is phenyl;
U is NH;
R¹ is halo; and
X is —O(CH₂)ₙ—; wherein:
n is 2.

34. The conjugate of claim 1, wherein:
A is phenyl;
U is NH;
R¹ is halo; and
X is —S(CH₂)ₙ—; wherein:
n is 2.

35. The conjugate of claim 1, wherein:
A is phenyl;
U is NH;
R¹ is hydrogen; and
X is —NR²—; wherein:
R² is methyl.

36. The conjugate of claim 1, wherein:
A is phenyl;
U is NH;
R¹ is halo; and
X is —NR²—; wherein:
R² is hydrogen.

37. The conjugate of claim 1, wherein:
A is phenyl;
U is NH;
R¹ is hydrogen; and
X is —C(CH₃)═.

38. The conjugate of claim 1, wherein:
A is a C₄-C₁₀cycloalkyl ring;
U is NH;
R¹ is hydrogen; and
X is —N(R²)(CH₂)ₘO(CH₂)ₙ—; wherein:
n is 1;
m is 2; and
R² is methyl.

39. A compound of formula (II): 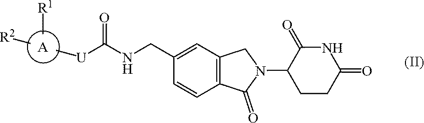
or a pharmaceutically acceptable salt thereof, wherein:
A is phenyl or a C₄-C₁₀cycloalkyl ring;
R¹ is independently selected from hydrogen and halo;
U is selected from NH and CF₂; and
R² is selected from —C(O)R³, —N(R⁴)₂, —(CH₂)ₙOH, —(CH₂)ₙSH, —(CH₂)ₙN(R⁴)₂, —(CH₂)ₙQ′(CH₂)ₘOH, —(CH₂)ₙQ′(CH₂)ₘSH, and —(CH₂)ₙQ′(CH₂)ₘN(R⁴)₂; wherein
R³ is hydrogen or C₁-C₆alkyl;
each R⁴ is independently hydrogen or C₁-C₆alkyl;
Q′ is O, S, or NR⁴;
n is 1-6; and
m is 2-5;
provided that when R² is NH₂, —(CH₂)ₙNH₂, or —(CH₂)ₙOH then R¹ is halo.

40. A compound of formula (III): 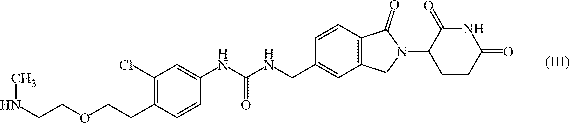
or a pharmaceutically acceptable salt thereof.

41. A compound of formula (IV): 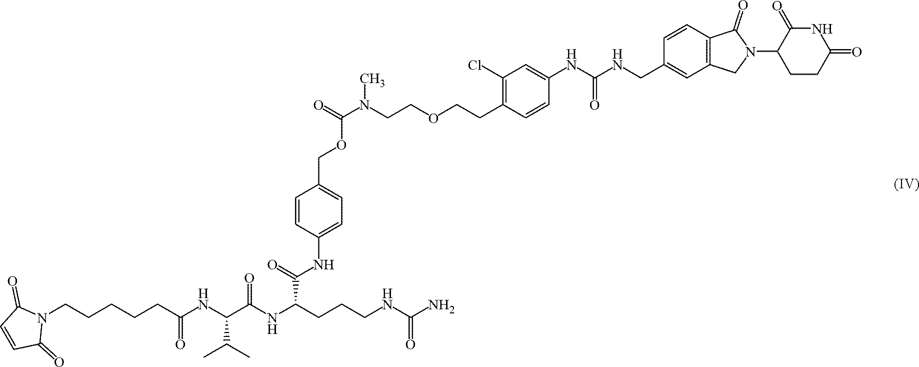
or a pharmaceutically acceptable salt thereof.

42. A conjugate of formula (V): 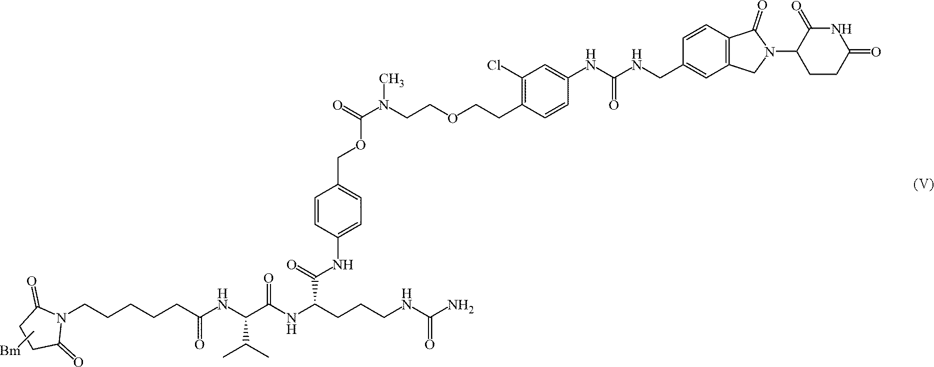
or a pharmaceutically acceptable salt thereof, wherein Bm is a binding moiety that specifically binds to a protein.

43-48. (canceled)

49. A pharmaceutical composition comprising a conjugate or compound of claim 1, or a pharmaceutically acceptable salt thereof, and one or more pharmaceutically acceptable carriers.

50. A method of treating cancer in a subject in need thereof, the method comprising administering to the subject a pharmaceutically acceptable amount of a conjugate; of claim 1, or a pharmaceutically acceptable salt thereof.

51-55. (canceled)

56. A method of preparing the conjugate of claim 1, or a pharmaceutically acceptable salt thereof, the process comprising reacting a binding moiety with a compound of formula (I-1): 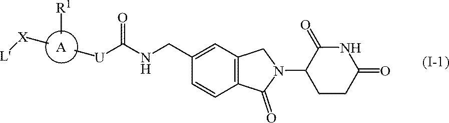
or a pharmaceutically acceptable salt thereof, wherein:
a is an integer from 1 to 10;
A is phenyl or a C₄-C₁₀cycloalkyl ring;
R¹ is independently selected from hydrogen and halo;
U is selected from NH and CF₂;
X is selected from —N(R²)ᵥ—,═C(CH₃)—, —Q—(CH₂)ₙ—, and —Q(CH₂)ₘQ′(CH₂)ₙ—; wherein
v is 1 or 2;
Q and Q′ are each independently O, S, or NR²;
each R² is independently hydrogen or C₁-C₆alkyl;
n is an integer from 1 to 6; and
m is an integer from 2 to 6;
wherein the left side of each group is attached to L′ and the right side is attached to A;
provided that when X is NH or —Q—(CH₂)ₙ—, R¹ is halo; and
L′ is a cleavable or non-cleavable linker precursor that conjugates to the binding moiety.

57. The method of claim 56, further comprising reducing the binding moiety prior to reacting with the compound of formula (I-1).

58-93. (canceled)
